SEQUENCE LISTING (122 sequences)

SEQ ID NO: 1 - protein, 3776 aa, synthetic construct
MTQLKPPNGTTPIGFSATTSLNASGSSSVKNGTIKPSNGIFKPSTRDTMDPCSGNAADGSIRVRFRGGIERWKECVNQVPERCDLSGLTTDSTRYQLASTGFGDASAAYQERLMTVPVDVHAALQELCLERRVSVGSVINFSVHQMLKGFGNGTHTITASLHREQNLQNSSPSWVVSPTIVTHENRDGWSVAQAVESIEAGRGSEKESVTAIDSGSSLVKMGLFDLLVSFVDADDARIPCFDFPLAVIVRECDANLSLTLRFSDCLFNEETICNFTDALNILLAEAVIGRVTPVADIELLSAEQKQQLEEWNNTDGEYPSSKRLHHLIEEVVERHEDKIAVVCDERELTYGELNAQGNSLARYLRSIGILPEQLVALFLDKSEKLIVTILGVWKSGAAYVPIDPTYPDERVRFVLDDTKARAIIASNQHVERLQREVIGDRNLCIIRLEPLLASLAQDSSKFPAHNLDDLPLTSQQLAYVTYTSGTTGFPKGIFKQHTNVVNSITDLSARYGVAGQHHEAILLFSACVFEPFVRQTLMALVNGHLLAVINDVEKYDADTLLPFIRRHSITYLNGTASVLQEYDFSDCPSLNRIILVGENLTEARYLALRQRFKNRILNEYGFTESAFVTALKIFDPESTRKDTSLGRPVRNVKCYILNPSLKRVPIGATGELHIGGLGISKGYLNRPELTPHRFIPNPFQTDCEKQLGINSLMYKTGDLARWLPNGEVEYLGRADFQIKLRGIRIEPGEIETMLAMYPRVRTSLVVSKKLRNGPEETTNEHLVGYYVCDSASVSEADLLSFLEKKLPRYMIPTRLVQLSQIPVNVNGKADLRALPAVDISNSTEVRSDLRGDTEIALGEIWADVLGARQRSVSRNDNFFRLGGHSITCIQLIARIRQRLSVSISVEDVFATRTLERMADLLQNKQQEKCDKPHEAPTELLEENAATDNIYLANSLQQGFVYHYLKSMEQSDAYVMQSVLRYNTTLSPDLFQRAWKHAQQSFPALRLRFSWEKEVFQLLDQDPPLDWRFLYFTDVAAGAVEDRKLEDLRRQDLTERFKLDVGRLFRVYLIKHSENRFTCLFSCHHAILDGWSLPLLFEKVHETYLQLLHGDNLTSSMDDPYTRTQRYLHAHREDHLDFWAGVVQKINERCDMNALLNERSRYKVQLADYDQVQEQRQLTIALSGDAWLADLRQTCSAQGITLHSILQFVWHAVLHAYGGGTHTITGTTISGRNLPILGIERAVGPYINTLPLVLDHSTFKDKTIMEAIEDVQAKVNVMNSRGNVELGRLHKTDLKHGLFDSLFVLENYPNLDKSRTLEHQTELGYSIEGGTEKLNYPLAVIAREVETTGGFTVSICYASELFEEVMISELLHMVQDTLMQVARGLNEPVGSLEYLSSIQLEQLAAWNATEAEFPDTTLHEMFENEASQKPDKIAVVYEETSLTYRELNERANRMAHQLRSDVSPNPNEVIALVMDKSEHMIVNILAVWKSGGAYVPIDPGYPNDRIQYILEDTQALAVIADSCYLPRIKGMAASGTLLYPSVLPANPDSKWSVSNPSPLSRSTDLAYIIYTSGTTGRPKGVTVEHHGVVNLQVSLSKVFGLRDTDDEVILSFSNYVFDHFVEQMTDAILNGQTLLVLNDGMRGDKERLYRYIEKNRVTYLSGTPSVVSMYEFSRFKDHLRRVDCVGEAFSEPVFDKIRETFHGLVINGYGPTEVSITTHKRLYPFPERRMDKSIGQQVHNSTSYVLNEDMKRTPIGAVGELYLGGEGVVRGYHNRADVTAERFIPNPFQSEEDKREGRNSRLYKTGDLVRWIPGSSGEVEYLGRNDFQVKIRGLRIELGEIEAILSSYHGIKQSVVIAKDCREGAQKFLVGYYVADAALPSAAIRRFMQSRLPGYMVPSRLILVSKFPVTPSGKLDTKALPPAEEESEIDVVPPRSEIERSLCDIWAELLEMHPEEIGIYSDFFSLGGDSLKSTKLSFMIHESFNRAVSVSALFCHRTVEAQTHLILNDAADVHEITPIDCNDTQMIPVSRAQERLLFIHEFENGSNAYNIDAAFELPGSVDASLLEQALRGNLARHEALRTLLVKDHATGIYLQKVLSPDEAQGMFSVNVDTAKQVERLDQEIASLSQHVFRLDDELPWEARILKLESGGLYLILAFHHTCFDAWSLKVFEQELRALYAALQKTKSAANLPALKAQYKEYALYHRRQLSGDRMRNLSDFWLRKLIGLEPLQLITDRPRPVQFKYDGDDLSIELSKKETENLRGVAKRCKSSLYVVLVSVYCVMLASYANQSDVSVGIPVSHRTHPQFQSVIGFFVNLVVLRVDISQSAICGLIRRVMKELVDAQLHQDMPFQEVTKLLQVDNDPSRHPLVQNVFNFESRANGEHDARSEDEGSLAFNQYRPVQPVDSVAKFDLNATVTELESGLRVNFNYATSLFNKSTIQGFLHTYEYLLRQLSELSAEGINEDTQLSLVRPTENGDLHLPLAQSPLATTAEEQKVASLNQAFEREAFLAAEKIAVVQGDRALSYADLNGQANQLARYIQSVSCIGADDGIALMLEKSIDTIICILAIWKAGAAYVPLDPTYPPGRVQLILEEIKAKAVLVHSSHASKCERHGAKVIAVDSPAIETAVSQQSAADLPTIASLGNLAYIIFTSGTSGKPKGVLVEQKAVLLLRDALRERYFGRDCTKHHGVLFLSNYVFDFSVEQLVLSVLSGHKLIVPPAEFVADDEFYRMASTHGLSYLSGTPSLLQKIDLARLDHLQVVTAAGEELHATQYEKMRRRFNGPIYNAYGVTETTVYNIIAEFTTNSIFENALREVLPGTRAYVLNAALQPVPFDAVGELYLAGDSVTRGYLNQPLLTDQRFIPNPFCKEEDIAMGRFARLYKTGDLVRSRFNRQQQPQLEYLGRGDLQIKMRGYRIEISEVQNVLTSSPGVREGAVVAKYENNDTYSRTAHSLVGYYTTDNETVSEADILTFMKARLPTYMVPSHLCCLEGALPVTINGKLDVRRLPEIINDSAQSSYSPPRNIIEAKMCRLWESALGMERCGIDDDLFKLGGDSITSLHLVAQIHNQVGCKITVRDIFEHRTARALHDHVFMKDSDRSNVTQFRTEQGPVIGEAPLLPIQDWFLSKALQHPMYWNHTFYVRTPELDVDSLSAAVRDLQQYHDVFRMRLKREEVGFVQSFAEDFSPAQLRVLNVKDVDGSAAVNEILDGWQSGFNLENGPIGSIGYLHGYEDRSARVWFSVHHMAIDTVSWQILVRDLQTLYRNGSLGSKGSSFRQWAEAIQNYKASDSERNHWNKLVMETASSISALPTSTGSRVRLSRSLSPEKTASLIQGGIDRQDVSVYDSLLTSVGLALQHIAPTGPSMVTIEGHGREEVDQTLDVSRTMGWFTTMYPFEIPRLSTENIVQGVVAVSERFRQVPARGVGYGTLYGYTQHPLPQVTVNYLGQLARKQSKPKEWVLAVGDNEFEYGLMTSPEDKDRSSSAVDVTAVCIDGTMIIDVDSAWSLEESEQFISSIEEGLNKILDGRASQQTSRFPDVPQPAETYTPYFEYLEPPRQGPTLFLLPPGEGGAESYFNNIVKRLRQTNMVVFNNYYLHSKRLRTFEELAEMYLDQVRGIQPHGPYHFIGWSFGGILAMEMSRRLVASDEKIGFLGIIDTYFNVRGATRTIGLGDTEILDPIHHIYNPDPANFQRLPSATDRIVLFKAMRPNNKYESENQRRLYEYYDGTRLNGLDSLLPSDSDVQLVPLTDDTHFSWVGNPQQVEQMCATIKEHLARY

SEQ ID NO: 2 - protein, 331 aa, synthetic construct
MASTPKANVPKIDVSPLFGDNMEEKMKVARAIDAASRDTGFFYAVNHGVDVKRLSNKTREFHFSITDEEKWDLAIRAYNKEHQDQIRAGYYLSIPEKKAVESFCYLNPNFKPDHPLIQSKTPTHEVNVWPDEKKHPGFREFAEQYYWDVFGLSSALLRGYALALGKEEDFFSRHFKKEDALSSVVLIRYPYLNPYPPAAIKTAEDGTKLSFEWHEDVSLITVLYQSDVANLQVEMPQGYLDIEADDNAYLVNCGSYMAHITNNYYPAPIHRVKWVNEERQSLPFFVNLGFNDTVQPWDPSKEDGKTDQRPISYGDYLQNGLVSLINKNGQT

SEQ ID NO: 3 - protein, 357 aa, synthetic construct
MLHILCQGTPFEIGYEHGSAAKAVIARSIDFAVDLIRGKTKKTDEELKQVLSQLGRVIEERWPKYYEEIRGIAKGAERDVSEIVMLNTRTEFAYGLKAARDGCTTAYCQLPNGALQGQNWDFFSATKENLIRLTIRQAGLPTIKFITEAGIIGKVGFNSAGVAVNYNALHLQGLRPTGVPSHIALRIALESTSPSQAYDRIVEQGGMAASAFIMVGNGHEAFGLEFSPTSIRKQVLDANGRMVHTNHCLLQHGKNEKELDPLPDSWNRHQRMEFLLDGFDGTKQAFAQLWADEDNYPFSICRAYEEGKSRGATLFNIIYDHARREATVRLGRPTNPDEMFVMRFDEEDERSALNARL

SEQ ID NO: 4 - protein, 548 aa, synthetic construct
MEAPRSDQAHTDATTPMEAIRTTSLGTNNYGPVPDDYLDLPVREVNDGADLREYITETRTGEIIKPIKSNVTGKTEDWKMVTFTIDDPENPKNWSKAFKWYCTMVVAFTCFVVAFCSSVITADVEGPIEEFGIGREASLVVITVFVIGFGLGPMVFAPMSEIVGRRPVYALTLALAVIFVIPCAVSKNIGTLIVCRLIDGIAFSAPMTLVGGTLADLWKSEERGVPMAAFSAAPFIGPAIGPLVGGYLADNCGWRWLYWIQLILAFVAWVMITFTVPETFAPILLKKRAQKLRKAEDDPKYTTETELDARPMGEKLRIFLFRPFQLLFLEPIVLFISLYMSVIYGLLYMFFVAYPIVYMGGKGWSASNTGLMFIPLAIGVIFSACCAPFVNNHYLKVSVAYGGKPPAEKRLIPMMWACWCIPSGLFVFAWTSYPDLHWMGPAMGGFLIGVGVILLYNSANNYLVDTYQHQAASALAAKTFIRSIWGACTVLFTEQMYERLGDQWASTLLAFIGLACCAIPYVFYFKGESIRRFSKFAFSDDEEKAIKA

SEQ ID NO: 5 - protein, 832 aa, synthetic construct
MSLTASAASAALTLLADGPDDGSKRPWRPNDPVKEQRDLYTQFVISTALGLSAFLAFCILRPKWTELYAARRRQRNAASRLPELPDTLFGWIPVLHQITEEEVLQSAGLDAYVFLSFFKFAIRFLLAVFIFAVAIILPMHYKYTGQYGVPGWDNPPGNKTTSPIDGSEKEKPVTDPAYLWIYVLFAYVFSGLAIYMLLDETKVIIRTRQTYLGNQTSTTDRTIRLSGIPHDLGTEDKIKEFVEGLRVGKVESITVCRKWRELDELIDERMKVIRELERAWTKHIGYKRPKNDGNALPLTEQQPRDADDERSGLLSGHDNEHVSGYSNERPKVRIWYGLFKLRFRMIDAIDYYEEKLRKIDEYIQNAREKEYRTTEIAFVTMESIAASQMLVQAILDPHPMQMFARLAPAPADVIWKNTYLSRTRRMVQSWSITFVIGFLTVFWSVLLVPIASLLELKTLETIVPRLAEFLQEHPIIKSLVQTGLPTLAFSLLTVAVPYLYEWLSNNQGMVSRGDVELSVISKNFFFSFFNLFLLFTVFGTASGFYGFWESLRDAFKDSTTIALALANSLEGLAPFYINLLILQGLGLFPFRLLEFGSVALYPFQFLSARTPREYAELSTPPKFSYGFSIPQTILILVICVVYSVFPSSWLICLFGLIYFTVGKFIYKYQLLYAMDHQQHSTGRAWPMICNRVFVGLLVHQLAMIGVLALRRAITRSLLLVPLLGFTVWFSYWFGRTYEPLMKFIALKSINRDQPGGGDISPSPSSTLSPPSGLDRDSLPIRIGGQDLELRLKKYVNPSLIVPLHAAWLPGRNPARGNGNGASAFEVHQTQNV

SEQ ID NO: 6 - protein, 508 aa, synthetic construct
MKDGEETPSVDGSTSASNREKLGTDLEIGPVDLSDGGKEEKVKDPNLVDWDGPDDPENPLNWTSKRKITATCSIALITFLTPLGSSMFAPGVGQLVKDFNVTSTELSSFVVSVYLLGYCFGPLIIAPLSELYGRQYVYHVCNILYVIWTIACAFAPEIGSLVVFRFFAGLAGSCPLTIGAGSIADMFVQEQRGGAMAAWALGPLIGPVVGPVAGAYLAQAKGWRWSFYVLAMAAGAITISSLFSIRESYAPTLLARKTKKLQKETGNMNLRSALDTGRTPKELFLYSIVRPTKMLFRSPIVFLLSLYVGVIYGYLYLLFTTITSVFQQQYNFSQGAVGLTYLGLGVGSLIGLFLIGATSDRLLNYLAAKNGEKKPEYRLPPMVPGAIFVPISLFMYGWTAYYQTHWIVPIIGTSFLGTGMMITFMCVSTYLVDAFTNYAASVMAANTVFRSLAGALLPLAGPKMYAVLGLGWGNSLLGFIALAFCALPVIFWIYGERIRTSPKFQVTF

SEQ ID NO: 7 - protein, 344 aa, synthetic construct
MVQDTSSASTSPILTRWYIDTRPLTASTAALPLLETLQPADQISVQKYYHLKDKHMSLASNLLKYLFVHRNCRIPWSSIVISRTPDPHRRPCYIPPSGSQEDSFKDGYTGINVEFNVSHQASMVAIAGTAFTPNSGGDSKLKPEVGIDITCVNERQGRNGEERSLESLRQYIDIFSEVFSTAEMANIRRLDGVSSSSLSADRLVDYGYRLFYTYWALKEAYIKMTGEALLAPWLRELEFSNVVAPAAVAESGDSAGDFGEPYTGVRTTLYKNLVEDVRIEVAALGGDYLFATAARGGGIGASSRPGGGPDGSGIRSQDPWRPFKKLDIERDIQPCATGVCNCLS

SEQ ID NO: 8 - protein, 2190 aa, synthetic construct
MNFHKGQPKEDLRVLFGPQCPDITDSITHIRDAISKDPTGLGFLTNILDELPSLWPTIAGAWPALKNVEGENQLLALGRLFEHESEVRVEASNLMMTPITVMRHVVDFWNLQDVATHPAFPSSSLSETEMPRIVDTQGFCVGLLAAIAVACSRNTQEFQYVASNAIRLSLCIGALVDLDEILCGSTTSLAVRWESVEDFNHLEKILNNNTEIPKGYTSCYTDVKSVTITIPNDSAERVKQEIHDHGLRTKQLSLRGRFHHEAHREGIQHIMKLCMNDSRFKLPRSDALLTPLRSSQGGEIFQQEALLHTVALDSILCAKANWYDVVSALINSTEMTVDQSRLLSIGPEEFVPRSARSRSVARRELESYGMQGFANESPQPSTASLSNSVQTFDSRPQAAEASPIAITGMACRYPNADTLAQLWELLELGRCTVKSPPESRFHMSDLQREPKGPFWGHFLERPDVFDHRFFNISAREAESMDPQQRVALQVAYEAMESAGYLGWQPNRLSQDIGCYVGVGSEDYTENVASRNANAFSITGTLQSFIAGRISHHFGWSGPSISLDTACSSAAVAIHLACKALQTNDCKIALAGGVNVLTNPRVYQNLSAASFLSPSGACKPFDASADGYCRGEGAGLFVLRPLQDAIDNGDPILGVIAGSAVNQGSNNSPITVPDAEAQRFLYNKAMSLAGVSPDEVTYVEAHGTGTQVGDPIELDSLRRTFGGPHRRNNLHIGSIKGNIGHTETSSGAAGLLKTILMLQQQRIPRQANFNQLNPKVKSLTPDRLVIASESTEWVSTKRVAMVSNYGASGSNAALIVKEHAPIGSEQNGTAPEYIQNKLAYNLAMKQNRDLPLNLTFSTSSDTTSLGARLEAISTGASADLIQKRPSNEPPVVLCFGGQNGLTATISKEVFDASALLRTHLEDCEEVGRTLGLLSLFPTIFSSAPITNIIHLHFILFSIQYASAKAWLDSGLRVSRIVGHSFGQLTALSVAGSLSVRDGIHLVTERARLIESSWGPESGIMLAVEGTEIEVQQLLDQTVESIEAIENAAARTPSASNLRLTRLQNSHAFHSRLVDSIVPGIMEVAGSLVYQTPIIPIEACSASGDWSTITAAEIVEHSRMPVYFRRAVERVAEKLQAPAVWLEAGSASPIIPMVRRVLESSSVANTYHKIDLGGSSGAQNLANVTSALWAQGVHVQFWPFDRAQHASFKWMNLPPYQFAQNSHWVDFDPAAFSSAGPSSGKQSAGQEAGLLCQLSESPDERLYHVNIQDALYRACTQGHAVLNQTLCPASMYMEMVLRAAASIFPTGNASEPAMSHIEDLTISSPLVLDPQGKVFLRLTRDGAGPTRPWLFSIFSSESNDHTLVHAEGTVCLHQERSRALARFQSMDRLLDSARSKTIEADPASNGLKGSTVYAALESVTNYGDYFRGVKQVFANGREASGLVSMMPSTTETNCNPILLDNFLQVAGIHVNCLSDRQSSEVFVCNAIGETFVINSLLKQENGASPSTWKVYTSYVRPSKTEIACDIYVMDCQTDTLSAAMMGVRFTSVSIRSLTRALAKLNNNVLETAEAQSVVEAAIPAEQSVVTATPSAPAADGHAANDLATVQEMLCELFGVSVAEVSPSVSLVDIGVDSLMSTEVLSEIKKRFQVDMSYTTLVDIPNIQGLVEHIFPGHSHAAPSRPVVEKAPVQSVAPQAVSHVPTPASNGPPLVSVARQCFDTTHAAVSHTSDAHWAGFFHTTYPKQMSLLTAYILEAFRALGSSLEASEPNEVLTPIAVLPRHEQLRKHLYKILDSVGLVRQMSTGELTLRQSGFEWVDWTNNETVESNALRVIVASPTGNSSAATMSPSKPIKMETVVWGERDNLQLRADIYYPETVDTTRKQRPIALMIHGGGHVMLSRKDIRPAQTQTLLDAGFLPVSIDYRLCPEVSLAEGPMADARDALSWVRRVLPNIPLLRADIRPDGNQVVAIGWSTGGHLAMTLPFTAPAAGIPAPDAVLAFYCPTNYEDPFWSNPNFPFGQTVASNEMEYDVWEGLQSMPIAGYNPALKERPLGGWMSTRDPRSRIALHMNWTGQTLPVLLKACTIKGNTEKCSPDDLSRPTEEDIQAVSPNYQIRVGRYNTPTFLIHGTSDDLVPCAQTESTHGALTASGVEAELRVVQEAAHLFDLYPASHAGQEAKAAVAEGYEFLRTHVQQ

SEQ ID NO: 9 - protein, 853 aa, synthetic construct
MESLSLTWITAIAVVLYLVQRYVRSYWRLKDIPGPVLAKLTDLQRVWWVKTGRAHEFHRDMHAMYGPIVRFGPNMVSVSDPRVIPTIYPSRPGFPKGDFYRTQKPYTRNKGAMPAVFNTQDEDLHKQLRSPIASLYSMTNVVRLEPLVDETLTVLSKQLDERFVGTNDKPFDLGDWLQYFAFDSMGTLTFSRRYGFLEQGRDMHGILQEIWNFMTRVAVMGQIPWFDEIWNKNSFITLFKRPTGFGVLKVVDNFISQRVSSRENDEKADEKDMLSQFLNIQASNPHSIMPWAPRAWTFSNVMAGSDSTANVMRTMMYNLLVDRDTLKSLRAELLEAESSNGLSRSLPSWDGVRSLPYLDACVLEALRLHPPFCLPFERVVPEGGITVCETYLPAGTVVGISPYLANRDKQTFGDDADKWRPSRWLDLSREDRVKLENSILTFGAGRRTCLGKNIAILEIKKLFPMLLLNYEIEIVNPENYQTTNAWFFRQWGLHAVIRKLPAPERDDTIEQKASIPPALNIPPSSSTVDVRIIDSGTLLDLRPDLFWTPDLPGLLKVTAPTYCFLISNGSRHVLFDLAVRQDWENLPPSIVAMIKSQTVIQEPRNISDVLDSDESSLGIRSKDIEAIIWSHAHFDHIGDPSTFPPSTELVVGPGIRDTHWPGFPTNPDAINLNTDIQGRNVREISFEKTQKGATKIGSFDAMDYFGDGSFYLLDAAGHSVGHIGALARVTTSPDSFVFMGGDSCHHAGVLRPTKYLPCPLDSGDTSLPCKSDSVFTLSPALPTDYTAALRTVENIKELDACEDVFVVLAHDATLKGKVDFYPSKINDWKAKEYGKKTKWLFYKDIENAIEGQK

SEQ ID NO: 10 - protein, 331 aa, synthetic construct
MTNAVEDSGPRDLLFLLISTSRFNRYMPYYTMMAAVWATFIAGALKLQQDPESLSIEFILYKAGLCFVHCLLLCGAGNTWNDLVDRDIDARVARTKMRPLASGKVTLTEALLWMTGQYFLSVKMLDLILDGRNIWTLMLPLTASIMLYPYLKRPIFSKVFVYPQYILGLAIGYPAITGWASITGSEEPLGDIIKHCIPICLLVFFWCVYFNTAYSHQDSVDDRKMNINSAYVIAGQRIRLFLAFLSVLPLLTIPYIISTINSPWLWVSWMATWTVSIVMQIAQFDSQKLESGGRIHWDNFLLGLWTIVACMVEVGLQKVEFWKNVEGYIKL

SEQ ID NO: 11 - protein, 423 aa, synthetic construct
MSLSLPPALSELARALPYSRTQWLPILVGFLIGYPLLIKALRYKRLGEMKKKFYFPTRESMAEMTDEEAFLIQKEMAQLEFPFMFLTSGQFALFRTYGIPTISHLLTKTGQFSKPETSFKRYTDTAALIGEMVENSPTSQRAFISVARTRFLHSGYQASGKILDADLLYTLALFAVQPVRFIENFEWRTLSDLELCAIGTFWKSLGDALGISSEILPSGKTGFKDGIQWLEEVDVWSQDYEAKYMVPDPKNRESADQATAVLLYNLPKILHPIGLQFTSYMMDDRLRKAMLYEAPSPGWSAVFSSLLATRKFVLRYLSPPRPAALAVSNIAQKPDKDDRYHRMSWDALPFYIRPTFWNRWGPMAWISWLMAHPVPGDHGQKYYPQGYHIQDIGPKYFEGKGHKEIQEMMKELKISRTGKCPFH

SEQ ID NO: 12 - protein, 398 aa, synthetic construct
MSAASPASIIQELASAAKQYENNESGAREALIAQSRALIASLEVPSEFIQHTFWSQPALSAIVRLATDVNLFQYLKDAQEEGLNAEALASKTGMDVSLFARLARHLVAMNVITSRNGVFYGTALSNGLAAENYQQSIRFCHDVSRPSFGAFPSFFKGNGYKTPALGTTDGPFQSAHKVDISFPQWLVGNPPYLQYFNSYMSAYRAGKPNWCDNGFYPVADRLLNGFDASVSDVLLVDVGGGRGHDIATFGSQFSPLPGRLVLQDREQVINSIPADESRQFEATTHDIFTTQPVKHARAYYMHSVPHGFGDEDAVKIMANLVPALAKGYSRVLLNEIVVDEERPVMSATNMDLIMLAHMGAKERTEADWRSILTRAGLKVVNIYSYPGVAESLIEAELA

SEQ ID NO: 13 - protein, 707 aa, synthetic construct
MFFTQPPHLVKAEELKQEPPKGTAYSVAIPGTEQPGRSRVYRAWNAQKELLTTLDPQVTTAHDMFESTANRQPKNHCLGWRPYNSTTKTWDPYQWLTYETVQKRRAAFGAGLVELHQKHNCHRPGQYGVGLWAQNRPEWQITDLACISQSLYSVSIYDVLAPDATEYIINHAELNCVVTSLPHIPTLLKLKSSLPNLKMIVSLDPLDGPEQNGHSKRALLESMAAGLDLAIYTIDQVEELGLASKRGYNAPSASDIVTINYTSGTTGPPKGVVLTHGNAVAATSCGLTTIGQARGDTMCSYLPLAHIYARLAEHTAFWGGARIGYFHGNIVELVDDLKLLKPTGFMSVPRLYSRFGTAIRAATVEQPGFKGALSRHIVAAKMANMKNPDPSKATIKHALYDRIWAKKVAAALGLERAKYMISGSAPLDPTLHNFLRVATGTDVVQGYGLTESYASATAQSTQDLSSGNCGRLAPCTEACLVSLPDMEYSVEDKPFPRGELLLRGNNMFREYYKNDDETSKAVTEDGWFRTGDVCTVDAQGRFIIIDRRKNVLKLAQGEYISPERLEGVILSELGYVAQAYVHGDSSETFLVGIFGVAPDLFAPYASKVLGKTIAPTDVEGLKEHLNDDKLRRAVLRDLERVAKKHKFAGYERVRNVSLKVDPFTVENNLLTPTLKLKRPPVVKMYRTLLDQLYGQANEEQSAPRAKL

SEQ ID NO: 14 - protein, 398 aa, synthetic construct
MSTEKFTITEHLVPGSHIREYPGSTVNQEDVLKIHVKQYTPKREGPVPDDAITFIATHGVGLPKELYEPLWDELLDQASGFHIRAIWMADVASMNQSGIHNEDKLSMDCITNLAYLHPRLFTTLLLLDPLIQLSPPSLGFGTDAPSAINYTLWRDDVWPSREVAIRANRAIMQGMDPRCLDRMTKHFFRDLPTPLYPDVEAIKALFGTTADSTTTPVTLTTPKYHELVAQIRQNFNARDPKTGRIEVPRDTHADMDPLVAYIPLYRPEPRSTFRRLETLRPSCLWVIAGATFLNIDEIREGVKICGSGIGGSGGVPDGRVREVVLPGFGHLMPFQEVKTVAETCIVWLQQEMDRFRQTERQWKEDRDGKSHLAVEENWYKVLKPIPSGRKKRNDKGKL

SEQ ID NO: 15 - protein, 548 aa, synthetic construct
MVEILDYTKALEVLKEYPSGDGLHVDTLLDSDNHGALTYNDFLILPGSITFSAADVSLDTKVTRRFTIKAPLLSSPMDTVTEHNMAIHMALLGGLGVIHNNCPPDDQAEMVRKVKRYENGFILDPVVLSPSTTVAEAKELKTKWNFGGFPVTGKTHYLSSFGKLASSDSFSLLEKGTLHSKLLGIVTSRDIQFHKTPEDPVTAVMSTELVTAPAGTTLAEANEVLRSSKKGKLPIVDKDGLLVSLLSRSDLMKNIHYPLASKLPSKQLLCAAAISTHDADKVRLQKLVDAGLDIVVVDSSQGNSMYQIAMIKWIKSTFPDIDIIAGNIVTREQAAALIAAGADGLRIGMGSGSACITQEVMAVGRPQAASVRSVSAFAARFGVPTIADGGVQNLGHIVKGLALGASAVMMGSLLAGTTESPGEYYVSNEGQLVKAFRGMGSIAVMEDKGKSGGGKNAGASRYFSENDKVKVAQGVAGSVVDRGSITQYVPYLVAGIQHSLQDIGVQDLEALHNGVNNGQVRFEMRSASAQTEGNVHGLHSHEKKLYSS

SEQ ID NO: 16 - protein, 15281 aa, unidentified
  source 1..15281: note = Tolypocladium inflatum
MGAIGQDMAYDRLANPSRASSISSNRYSEPVEQSFAQGRLWFLHQLKLGASWDITPAAIRLRGHLDIDALNAASRALTQRHETLRTTFKEQDGVGVQVVHASGLERGLRIVDASSRDLAQLLAEEQTMKFDLESEPAWRVALLKVAEDHHILSIVVHHIISDSRSLDIIQQELGELYTAASQGKSISACPLGPIPIQYRDLTTWQNQDEQVAEQERQLGYWIEQLDNNTPAELLTELPRPAIPSGETGKISFQIDGSVHKELLAFCRSQQVTAYAVLLAAFRVAHFRLTGAEDATIGAPVANRDRPELENMVAPLATLQCMRVVLDEDDTFESVLRQIMSVMTEAHANRDVPFERIVSALLPGSTDTSRHPLVQLMFALHPAQDTGRARWGFLEAETLQSAAPTRFDMEMHLFEGDDRFDANVLFSTGLFDAEAIRSVVSIFREVLRRGISEPAVHVKTMPLTDGLAAIRDMGLLDIGTTDYPREASVVDMFQEQVALNPSATAVADASSRLSYSELDHKSDQLAAWLRRRQLKPETLIGVLSPPSCETMVSFLGILKAHLAYLPLDINVPLARIESILSAVDGHKLVLLGSNVPQPKVDVPDVELLRISDALNGSQVNGLAGKQATAKPSATDLAYVIFTSGSTGKPKGVMIEHRGIVRLVKGTNIISPAQAAVPTAHLANIAFDLSTWEIYTPILNGGTLVCIEHSVTLDSKALEAVFTKEGIRVAFLAPALIKQCLADRPAIFAGLDSLYAIGDRFDRRDALHAKSLVKHGVYNAYGPTENSVVSTIYSVSEASPFVTGVPVGRAISNSGAYVMDQDQQLVSPGVMGELVVSGDGLARGYTDSALDKNRFVVVQIDGESIRGYRTGDRARYSLKGGQIEFFGRMDQQVKIRGHRIEPAEVEHALLNSDQVRDAAVVIRRQEEEEPAMIAFVTTQGTLPDHLVNINGNGHVPDGNGSKNDQFAVHVESELRRRLQMLLPSYMMPARIVVLDHLPLNPNGKVDRKALGQSAKTVQKSKLVSQRVAPRNEIEAVLCEEYRSVLGVEVGITDNFFDLGGHSLTAMKLAARISQRLDIQASVATVFEQPMLADLAATIQRGSTLYSVIPTTEYTGPVEQSFAQGRLWFLEQLNTGASWYNVMLTVRLRGHLDVDALGTALLALEKRHETLRTTFEERDGVGMQVVHSSLMGELRLIDISEKSGTAAHEALMKEQSTRFDLTREPGWRVALLKLADHHIFSIVMHHIVSDGWSLDLLRHELGQLYSAALRGQDPLSRLEPLPIQYRDFAVWQKQDSQQKAAHQRQLEYWTKQLADSTPAELLTDFPRPSILSGKAGKVPVAIEGSLYDTLQVFSRTHQVTSFAVLLAAFRAAHFRLTGSDNATIGVPSANRNRPELENVIGFFVNTQCIRITIDENDNFESLVRQVRSTTTAAQDNQDVPFEQVVSSLMPSSSRDASRNPLVQLMFALHGQQDLFKIQLEGTEEEVIPTEEVTRFDIEFHLYQGASKLSGDIIFAADLFEAETIRGVVSVFQEVLRRGLQQPQTPIMTMPLTDGIPELERMGLLHMVKTDYPRNMSVVDVFQQQVRLSAEATAVIDSSSRMSYAELDQRSDQVAAWLRQRQLPAETFVAVLAPRSCEAVIALFGILKAGHAYLPLDVNVPAARLRAILAEVKGEKLVLLGAGEPSPEGQSPEVSIVRIADATSPAGHASLRDGKSKPTAGSLAYVIFTSGSTGKPKGVMIEHRGVLRLVKQTNILSSLPPAQTFRMAHMSNLAFDASIWEVFTALLNGGSLVCIDRFTILDAQALEALFLREHINIALFPPALLKQCLTDAAATIKSLDLLYVGGDRLDTADAALAKALVKSEVYNAYGPTENTVMSTLYSIADTERFVNGVPIGRAVSNSGVYVMDQNQQLVPLGVMGELVVTGDGLARGYTNPALDSDRFVDVIARGQLLRAYRTGDRARYRPKDGQVEFFGRMDHQVKVRGHRIELAEVEHALLSSAGVHDAVVVSNSQEDNQGVEMVAFITAQDNETLQEAQSSNQVQEWESHFETTAYADITAIDQNTLGRDFTSWTSMYDGTLIDKREMQEWLDDTMRTFLDGQAAGHVLEIGTGTGMVLFNLGQAGLKSYIGLEPSQSAVQFVNKAAQTFPGLEGKAQVHVGTAMDTGRLSALSPDLIVINSVAQYFPSREYLAEVVEALVRIPGVRRIFFGDMRTYATHKDFLVARAVHTNGSKVTRSKVQQEVARLEELEEELLVDPAFFTSLKESLSEEIEHVEILPKNMKVNNELSSYRYGAVLHIRNHNQNQSRSIHKINAESWIDFASSQMDRQGLARLLKENKDAESIAVFNIPYSKTIVERHIAKSLADDHDGDDTHSSIDGVAWISAAREKASQCPSLDVHDLVQLAEDAGFRVEVSWARQRSQNGALDVFFHHFQPTENESRALVDFPTDYKGQQARSLTNRPLQRVESRRIEAQVREQLQVLLPAYMIPARIVVLQNMPLNTSGKVDRKELTLRAKVTPQATPSSELVAPRDSIEAIICKEFKDVLGVEVGITDNFFNVGGHSLLATKLAARLSRQLNAQIAVKDIFDRPVIADLAATIQQDTTEHNPILPTSYTGPVEQSFAQGRLWFLDQLNVGATWYLMPFAVRLRGPLVVSALAAALLALEERHETLRTTFIEQEGIGMQVIHPFAPKELRVIDVSGEEESTIQKILEKEQTTPFNLASEPGFRLALLKTGEDEHILSTVMHHAISDGWSVDIFQQEIGQFYSAILRGHDPLAQIAPLSIQYRDFATWQRQIFQVAEHRRQLAYWTKQLADNKPAELLTDFKRPPMLSGRAGEIPVVVDGLIYEKLQDFCRIRQVTAFTVLLAAFRAAHYRMTGTEDATIGTPIANRNRPELEGLIGFFVNTQCMRITVDVEDSFETLVHQVRETTLAAHANQDVPFEQIVSNILPGSSDTSRNPLVQLMFALHSQQNLGKVRLEGIEEEIISIAETTRFDIEFHLYQEAERLNGSIVYAADLFVPETIQSVITIFQGILQKGLGEPDMPVASMALDGGLESLRSTGLLHPQQTDYPCDASVVQIFKQQVAVNPDVIAVRDESTRLSYADLDRKSDQVACWLSRRGIAPETFVAILAPRSCETIVAILGVLKANLAYLPLDVNVPASRLEAILSEVSGSMLVLVGAETPIPEGMAEAETIRITEILADAKTDDINGLAASQPTAASLAYVIFTSGSTGRPKGVMVEHRGIVRLTKQTNITSKLPESFHMAHISNLAFDASVWEVFTTLLNGGTLVCIDYFTLLESTALEKVFFDQRVNVALLPPALLKQCLDNSPALVKTLSVLYIGGDRLDASDAAKARGLVQTQAFNAYGPTENTVMSTIYPIAEDPFINGVPIGHAVSNSGAFVMDQNQQITPPGAMGELIVTGDGLARGYTTSSLNTGRFINVDIDGEQVRAYRTGDRVRYRPKDLQIEFFGRIDHQVKIRGHRIEPAEVEYALLSHDLVTDAAVVTHSQENQDLEMVGFVAARVADVREDESSNQVQEWQTHFDSIAYADITTIDQQSLGRDFMSWTSMYDGSLIKKSQMQEWLDDTMRSLLDSQPPGHVLEVGTGTGMVLFNLGREGGLQSYVGLEPSPSATAFVNKAAKSFPGLEDRIRVEVGTATDIDRLGDDLHAGLVVVNSVAQYFPSQDYLAQLVRDLTKVPGVERIFFGDMRSHAINRDFLVARAVHALGDKATKAEIQREVVRMEESEDELLVDPAFFTSLTTQVENIKHVEILPKRMRATNELSSYRYAAVLHVNDLAKPAHKVSPGAWVDFAATKMDRDALIRLLRGTKISDHIAIANIPNSKTIVERTICESVYDLGGDAKDSNDRVSWLSAARSNAVKVASLSAIDLVDIAQEAGFRVEISCARQWSQNGALDAVFHHLGPSPQSSHVLIDFLTDHQGRPEEALTNHPLHRAQSRRVERQIRERLQTLLPAYMIPAQIMVLDKLPLNANGKVDRKQLTQRAQTVPKAKQVSAPVAPRTEIERVLCQEFSDVLGVDIGIMENFFDLGGHSLMATKLAARISRRLETHVSVKEIFDHPRVCDLVLIVQQGSAPHDPIVSTKYTGPVPQSFAQGRLWFLDQLNFGATWYLMPLAVRLRGAMNVHALTAALLALERRHELLRTTFYEQNGVGMQKVNPVVTETLRIIDLSNGDGDYLPTLKKEQTAPFHLETEPGWRVALLRLGPGDYILSVVMHHIISDGWSVDVLFQELGQFYSTAVKGHDPLSQTTPLPIHYRDFALWQKKPTQESEHERQLQYWVEQLVDSAPAELLTDLPRPSILSGQAGEMSVTIEGALYKNLEEFCRVHRVTSFVVLLAALRAAHYRLTGSEDATIGTPIANRNRPELEQIIGFFVNTQCIRITVNEDETFESLVQQVRSTATAAFAHQDVPFEKIVSTLLPGSRDASRNPLVQLMFAVHSQKNLGELKLENAHSEVVPTEITTRFDLEFHLFQQDDKLEGSILYSTDLFEAVSVQSLLSVFQEILRRGLNGPDVPISTLPLQDGIVDLQRQGLLDVQKTEYPRDSSVVDVFHEQVSINPDSIALIHGSEKLSYAQLDRESDRVARWLRHRSFSSDTLIAVLAPRSCETIIAFLGILKANLAYLPLDVKAPAARIDAIVSSLPGNKLILLGANVTPPKLQEAAIDFVPIRDTFTTLTDGTLQDGPTIERPSAQSLAYAMFTSGSTGRPKGVMVQHRNIVRLVKNSNVVAKQPAAARIAHISNLAFDASSWEIYAPLLNGGAIVCADYFTTIDPQALQETFQEHEIRGAMLPPSLLKQCLVQAPDMISRLDILFAAGDRFSSVDALQAQRLVGSGVFNAYGPTENTILSTIYNVAENDSFVNGVPIGSAVSNSGAYIMDKNQQLVPAGVMGELVVTGDGLARGYMDPKLDADRFIQLTVNGSEQVRAYRTGDRVRYRPKDFQIEFFGRMDQQIKIRGHRIEPAEVEQAFLNDGFVEDVAIVIRTPENQEPEMVAFVTAKGDNSAREEEATTQIEGWEAHFEGGAYANIEEIESEALGYDFMGWTSMYDGTEIDKDEMREWLNDTMRSLLDGKPAGRVLEVGTGTGMIMFNLGRSQGLERYIGLEPAPSAAEFVNNAAKSFPGLAGRAEVHVGTAADVGTLQGLTSDMAVINSVAQYFPTPEYLAETIKSLVQVPGMKRIYLGDMRSWAMNRDFAAARAAYSLADNASKDRVRQKMMELEEKEEELLVDPAFFTALASQLQDRIQHVEILPKRMKATNELSSYRYAAVLHISDEPLPIYKIDPEAWINFEGSRLTREALAQVLKENENAESVAISNIPYSKTVVERHIVRSLDQEDANAPEESMDGSDWISAVRTRAQQCHTLSASDLFDIAEDAGFRVEVSWARQHSQHGALDAVFHHLKPATEDSRVLIKFPTDHQGRPLKSLTNQPLLPAQSRRAELLIREGLQTLLPPYMIPSQITLIDRMPLNANGKVDRRELARRAKITQKSKPVEDIVPPRNSVEATVCKGFTDVLGVEVGITDNFFNLGGHSLMATKLAARLGRQLNTRISVRDVFDQPVVADLAAVIQRNSAPHEPIKPADYTGPVPQSFAQGRLWFLDQLNVGATWYLMPLGIRLHGSLRVDALATAISALEQRHEPLRTTFHEEDGVGVQVVQDHRPKDLRIIDLSTQPKDAYLAVLKHEQTTLFDLATEPGWRVALIRLGEEEHILSIVMHHIISDGWSVEVLFDEMHRFYSSALRQQDPMEQILPLPIQYRDFAAWQKTEEQVAEHQRQLDYWTEHLADSTPAELLTDLPRPSILSGRANELPLTIEGRLHDKLRAFCRVHQATPFVILLAALRAAHYRLTGAEDATLGTPIANRNRPELENMIGFFVNTQCMRIAIEENDNFESLVRRVRSTATSAFANQDVPFESIVSSLLPGSRDASRNPLVQVILAVHSQQDLGKLTLEGLRDEAVDSAISTRFDVEFHLFEHADRLSGSVLYAKELFKLRTIESVVSVFLETLRRALDQPLTPLAVLPLTDGVGEIASKGLLDVPRTDYPRDANIVEVFQQHVRATPDAIAVKDATSILTYAQLDQQSDRLAIWLSRRHMMPETLVGVLAPRSCETIIAMFGIMKANLAYLPLDINSPAARLRSILSAVDGNKLVLLGSGVTAPEQENPEVEAVGIQEILAGTGLDKTQGSNARPSATSLAYVIFTSGSTGKPKGVMVEHRSVTRLAKPSNVISKLPQGARVAHLANIAFDASIWEIATTLLNGATLVCLDYHTVLDCRTLKEVFERESITVVTLMPALLKQCVAEIPETLAHLDLLYTGGDRVGGHDAMRARSLVKIGMFSGYGPTENTVISTIYEVDADEMFVNGVPIGKTVSNSGAYVMDRNQQLVPSGVVGELVVTGDGLARGYTDPSLNKNRFIYITVNGESIRAYRTGDRVRYRPHDLQIEFFGRMDQQVKIRGHRIEPGEVESALLSHNSVQDAAVVICAPADQDSGAEMVAFVAARNTEDEDTQEEEAVDQVQGWETHFETAAYSEVKDIRQSEVGNDFMGWTSMYDGSEIDKTDMHEWLNDTMRMILDAREPGHVLEIGTGTGMVMFNLAKCPGLQGYVGFEPSKSAAQFVNDAAQSFPALKDGRSIVHVGTATDINKAGPIQPRLVVINSVAQYFPTPEYLFRVVEALVQIPSVERIVFGDMRTNAINRDFVASRALHTLGEKANKRLVRQMIYELEANEEELLTDPAFFTSLRTRLGEKIKHVEILPKTMKATNELSKYRYAAVLHVRGSREQSTIHQVSPNAWIDFAADGLDRQTLINLLKEHKDAGTVAIGNIPYSKTIVERFVNKSLSEDDMEEGQNSLDGSAWVAAVRMAAQSCPSLDAMDVKEIAQEAGYQVEVSWARQWSQNGALDAIFHHFEPPKEGARTLIEFPTDYEGRNVNTLTNRPLNSIQSRRLGTQIREKLQTLLPPYMIPSRIMVLDQMPVNNNGKIDRKELVRRAIVAPKPRSAATRVAPRNEIEAILRDEFEDVLGTEVSVLDNFFDLGGHSLMATKLAARVSRRLDAHISIKDVFDQPVLADLAASIQRESAPHEPIPQRPYTGPAEQSFAQGRLWFLDQLNLGATWYLMPLAIRIRGQLRVAALSAALFALERRHETLRTTFEESDGVGVQIVGEARNSDLRVHDVSTGDDGEYLEVLRREQTVPFDLSSEPGWRVCLVKTGEEDHVLSIVMHHIIYDGWSVDILRGELGQFYSAALRGQDPLLHANPLPIQYRDFAAWQREAKQVEEHQRQLGYWSKQLVDSTPAELLTDLPRPSILSGRAGSVDVTIEGSVYGALQSFCRTRSVTTFVVLLTVFRIAHFRLTAVDDATIGTPIANRNRPELETLVGCFVNTQCMRISIADDDNFEGLVRQVRNVATAAYANQDVPFERIVSALVPGSRNTSRNPLVQLMFAVQSVEDYDQVRLEGLESVMMPGEASTRFDMEFHLVPGDQKLTGSVLYSSDLFEQGTIQNFVDIFQECLRSVLDQPLTPISVLPFSNAISNLESLDLLEMPTSDYPRDRTVVDLFREQAAICPDSIAVKDSSSQLTYAQLDEQSDRVAAWLHERHMPAESLVGVLSPRSCETIIAYFGIMKANLAYLPLDVYAPDARLAAILDTVEGERLLLLGAGVPQPGIQIPRLSTAYIAEALSHATTVDVTSIPQPSATSLAYVIFTSGSTGKPKGVMIEHRGIVRLVRDTNVNVFPESGSALPVSHFSNLAWDAATWEIYTAVLNGGTVVCIDRDTMLDIAALNSTFRKENVRAAFFTPAFLKQCLAETPELVANLEILHTAGDRLDPGDANLAGKTAKGGIFNVLGHTENTAYSTFYPVVGEETFVNGVPVGRGISNSHAYIIDRHQKLVPAGVMGELILTGDGVARGYTDSALNKDRFVYIDINGKSTWSYRTGDKARYRPRDGQLEFFGRMDQMVKIRGVRIEPGEVELTLLDHKSVLAATVVVRRPPNGDPEMIAFITIDAEDDVQTHKAIYKHLQGILPAYMIPSHLVILDQMPVTDNGKVDRKDLALRAQTVQKRRSTAARVPPRDEVEAVLCEEYSNLLEVEVGITDGFFDLGGHSLLATKLAARLSRQLNTRVSVKDVFDQPILADLADIIRRGSHRHDPIPATPYTGPVEQSFAQGRLWFLEQLNLGASWYLMPFAIRMRGPLQTKALAVALNALVHRHEALRTTFEDHDGVGVQVIQPKSSQDLRIIDLSDAVDDTAYLAALKREQTTAFDLTSEPGWRVSLLRLGDDDYILSIVMHHIISDGWTVDVLRQELGQFYSAAIRGQEPLSQAKSLPIQYRDFAVWQRQENQIKEQAKQLKYWSQQLADSTPCEFLTDLPRPSILSGEADAVPMVIDGTVYQLLTDFCRTHQVTSFSVLLAAFRTAHYRLTGTLDATVGTPIANRNRPELEGLIGFFVNTQCMRMAISETETFESLVQQVRLTTTEAFANQDVPFEQIVSTLLPGSRDTSRNPLVQVMFALQSQQDLGRIQLEGMTDEALETPLSTRLDLEVHLFQEVGKLSGSLLYSTDLFEVETIRGIVDVFLEILRRGLEQPKQRLMAMPITDGITKLRDQGLLTVAKPAYPRESSVIDLFRQQVAAAPDAIAVWDSSSTLTYADLDGQSNKLAHWLCQRNMAPETLVAVFAPRSCLTIVAFLGVLKANLAYLPLDVNAPAARIEAILSAVPGHKLVLVQAHGPELGLTMADTELVQIDEALASSSSGDHEQIHASGPTATSLAYVMFTSGSTGKPKGVMIDHRSIIRLVKNSDVVATLPTPVRMANVSNLAFDISVQEIYTALLNGGTLVCLDYLTLLDSKILYNVFVEAQVNAAMFTPVLLKQCLGNMPAIISRLSVLFNVGDRLDAHDAVAASGLIQDAVYNAYGPTENGMQSTMYKVDVNEPFVNGVPIGRSITNSGAYVMDGNQQLVSPGVMGEIVVTGDGLARGYTDSALDEDRFVHVTIDGEENIKAYRTGDRVRYRPKDFEIEFFGRMDQQVKIRGHRIEPAEVEHALLGHDLVHDAAVVLRKPANQEPEMIAFITSQEDETIEQHESNKQVQGWGEHFDVSRYADIKDLDTSTFGHDFLGWTSMYDGVDIPVNEMKEWLDETTASLLDNRPPGHILEIGAGTGMILSNLGKVDGLQKYVGLDPAPSAAIFVNEAVKSLPSLAGKARVLVGTALDIGSLDKNEIQPELVVINSVAQYFPTSEYLIKVVKAVVEVPSVKRVFFGDIRSQALNRDFLAARAVRALGDNASKEQIREKIAELEESEEELLVDPAFFVSLRSQLPNIKHVEVLPKLMKATNELSSYRYAAVLHISHNEEEQLLIQDIDPTAWVDFAATQKDSQGLRNLLQQGRDDVMIAVGNIPYSKTIVERHIMNSLDQDHVNSLDGTSWISDARSAAAICTSFDAPALTQLAKEEGFRVELSWARQRSQNGALDAVFHRLATDANCERSRVLVHFPTDHQGRQLRTLTNRPLQRAQSRRIESQVFEALQTALPAYMIPSRIIVLPQMPTNANGKVDRKQLARRAQVVAKRKAVSARVAPRNDTEIVLCEEYADILGTEVGITDNFFDMGGHSLMATKLAARLSRRLDTRVTVKEVFDKPVLADLAASIEQGSTPHLPIASSVYSGPVEQSYAQGRLWFLDQFNLNATWYHMSLAMRLLGPLNMDALDVALRALEQRHETLRTTFEAQKDIGVQVVHEAGMKRLKVLDLSDKNEKEHMAVLENEQMRPFTLASEPGWRVILLRLGPTEYILSLVMHHIISDGWSVDILRQELGQFYSAALRGRDPLSQVKPLPIQYRDFAAWQKEAAQVAEHERQLAYWENQLADSTPGELLTDFPRPQFLSGKAGVIPVTIEGPVYEKLLKFSKERQVTLFSVLLTAFRATHFRLTGAEDATIGTPIANRNRPELEHIIGFFVNTQCMRLLLDTGSTFESLVQHVRSVATDAYSNQDIPFERIVSALLPGSRDASRSPLIQLMFALHSQPDLGNITLEGLEHERLPTSVATRFDMEFHLFQEPNKLSGSILFADELFQPETINSVVTVFQEILRRGLDQPQVSISTMPLTDGLIDLEKLGLLEIESSNFPRDYSVVDVFRQQVAANPNAPAVVDSETSMSYTSLDQKSEQIAAWLHAQGLRPESLICVMAPRSFETIVSLFGILKAGYAYLPLDVNSPAARIQSILSEVEGKRLVLLGSGIDMPQSDRMDVETARIQDILTNTKVERSDPMSRPSATSLAYVIFTSGSTGRPKGVMIEHRNILRLVKQSNVTSQLPQDLRMAHISNLAFDASIWEIFTAILNGGALICIDYFTLLDSQALRTTFEKARVNATLFAPALLKECLNHAPTLFEDLKVLYIGGDRLDATDAAKIQALVKGTVYNAYGPTENTVMSTIYRLTDGESYANGVPIGNAVSSSGAYIMDQKQRLVPPGVMGELVVSGDGLARGYTNSTLNADRFVDIVINDQKARAYRTGDRTRYRPKDGSIEFFGRMDQQVKIRGHRVEPAEVEQAMLGNKAIHDAAVVVQAVDGQETEMIGFVSMASDRFSEGEEEITNQVQEWEDHFESTAYAGIEAIDQATLGRDFTSWTSMYNGNLIDKAEMEEWLDDTMQSLLDKEEPGRVLEIGTGTGMVLFNLPKNDGLESYVGIEPSRSAALFVDKAAQDFPGLQGKTQILVGTAEDIKLVKDFHPDVVVINSVAQYFPSRSYLVQIASELIHMTSVKTIFFGDMRSWATNRDFLVSRALYTLGDKATKDQIRQEVARLEENEDELLVDPAFFTSLTSQWPGKVKHVEILPKRMRTSNELSSYRYAAVLHICRDGEGRNRYGRRVHSVEENAWIDFASSGMDRHALVQMLDERRDAKTVAIGNIPHSNTINERHFTTSLDTEGEGIAQDSLDGSAWQSATKAMAARCPCLSVTELVEIGQAAGFRVEVSWARQRSQHGALDVVFHHLEDDRVGRVLINFPTDFERLPPSTGLTSRPLQRIQNRRFESQIREQLQTLLPPYMVPSRIVVLERMPLNANSKVDRKELARKARTLQTIKPSATRVAPRNDIEAVLCDEFQAVLGVTVGVMDNFFELGGHSLMATKLAARLSRRLDTRVSVKDIFNQPILQDLADVVQTGSAPHEAIPSTPYSGPVEQSFSQGRLWFLDQLNLNASWYHMPLASRLRGPLRIEALQSALATIEARHESLRTTFEEQDGVPVQIVRAARNKQLRIIDVSGTEDAYLAALKQEQDAAFDLTAEPGWRVALLRLGPDDHVLSIVMHHIISDGWSVDILRQELGQLYSNASSQPAPLPIQYRDFAIWQKQDSQIAEHQKQLNYWKRQLVNSKPAELLADFTRPKALSGDADVIPIEIDDQVYQNLRSFCRARHVTSFVALLAAFRAAHYRLTGAEDATIGSPIANRNRPELEGLIGCFVNTQCLRIPVKSEDTFDTLVKQARETATEAQDNQDVPFERIVSSMVASSRDTSRNPLVQVMFAVHSQHDLGNIRLEGVEGKPVSMAASTRFDAEMHLFEDQGMLGGNVVFSKDLFESETIRSVVAVFQETLRRGLANPHANLATLPLTDGLPSLRSLCLQVNQPDYPRDASVIDVFREQVASIPKSIAVIDASSQLTYTELDERSSQLATWLRRQVTVPEELVGVLAPRSCETIIAFLGIIKANLAYLPLDVNAPAGRIETILSSLPGNRLILLGSDTQAVKLHANSVRFTRISDALVESGSPPTEELSTRPTAQSLAYVMFTSGSTGVPKGVMVEHRGITRLVKNSNVVAKQPAAAAIAHLSNIAFDASSWEIYAPLLNGGTVVCIDYYTTIDIKALEAVFKQHHIRGAMLPPALLKQCLVSAPTMISSLEILFAAGDRLSSQDAILARRAVGSGVYNAYGPTENTVLSTIHNIGENEAFSNGVPIGNAVSNSGAFVMDQNQQLVSAGVIGELVVTGDGLARGYTDSKLRVDRFIYITLDGNRVRAYRTGDRVRHRPKDGQIEFFGRMDQQIKIRGHRIEPAEVEQALARDPAISDSAVITQLTDEEEPELVAFFSLKGNANGTNGVNGVSDQEKIDGDEQHALLMENKIRHNLQALLPTYMIPSRIIHVDQLPVNANGKIDRNELAVRAQATPRTSSVSTYVAPRNDIETIICKEFADILSVRVGITDNFFDLGGHSLIATKLAARLSRRLDTRVSVRDVFDTPVVGQLAASIQQGSTPHEAIPALSHSGPVQQSFAQGRLWFLDRFNLNAAWYIMPFGVRLRGPLRVDALQTALRALEERHELLRTTFEEQDGVGMQIVHSPRMRDICVVDISGANEDLAKLKEEQQAPFNLSTEVAWRVALFKAGENHHILSIVMHHIISDGWSVDIFQQELAQFYSVAVRGHDPLSQVKPLPIHYRDFAVWQRQDKQVAVHESQLQYWIEQLADSTPAEILSDFNRPEVLSGEAGTVPIVIEDEVYEKLSLFCRNHQVTSFVVLLAAFRVAHYRLTGAEDATIGTPIANRNRPELEDLIGFFVNTQCMRIALEEHDNFLSVVRRVRSTAASAFENQDVPFERLVSALLPGSRDASRNPLVQLMFVVHSQRNLGKLQLEGLEGEPTPITATTRFDVEFHLFEQDKGLAGNVVFAADLFEAATIRSVVEVFHEILRRGLDQPDIAISTMPLVDGLAALNSRNLPAVEDIEPDFATEASVVDVFQTQVVANPDALAVTDTSTKLTYAELDQQSDHVAAWLSKQKLPAESIVVVLAPRSSETIVACIGILKANLAYLPMDSNVPEARRQAILSEIPGEKFVLLGAGVPIPDNKTADVRMVFISDIVASKTDKSYSPGTRPSASSLAYVIFTSGSTGRPKGVMVEHRGVISLVKQNASRIPQSLRMAHVSNLAFDASVWEIFTTLLNGGTLFCISYFTVLDSKALSAAFSDHRINITLLPPALLKQCLADAPSVLSSLESLYIGGDRLDGADATKVKDLVKGKAYNAYGPTENSVMSTIYTIEHETFANGVPIGTSLGPKSKAYIMDQDQQLVPAGVMGELVVAGDGLARGYTDPSLNTGRFIHITIDGKQVQAYRTGDRVRYRPRDYQIEFFGRLDQQIKIRGHRIEPAEVEQALLSDSSINDAVVVSAQNKEGLEMVGYITTQAAQSVDKEEASNKVQEWEAHFDSTAYANIGGIDRDALGQDFLSWTSMYDGSLIPREEMQEWLNDTMRSLLDNQPPGKVLEIGTGTGMVLFNLGKVEGLQSYAGLEPSRSVTAWVNKAIETFPSLAGSARVHVGTAEDISSIDGLRSDLVVINSVAQYFPSREYLAELTANLIRLPGVKRIFFGDMRTYATNKDFLVARAVHTLGSNASKAMVRQQVAKLEDDEEELLVDPAFFTSLSDQFPDEIKHVEILPKRMAATNELSSYRYAAVIHVGGHQMPNGEDEDKQWAVKDINPKAWVDFAGTRMDRQALLQLLQDRQRGDDVVAVSNIPYSKTIMERHLSQSLDDDEDGTSAVDGTAWISATQSRAKECPALSVADLIEIGKGIGFEVEASWARQHSQRGGLDAVFHRFEPPRHSGHVMFRFPTEHKGRSSSSLTNRPLHLLQSRRLEAKVRERLQSLLPPYMIPSRITLLDQMPLTSNGKVDRKKLARQARVIPRSAASTLDFVAPRTEIEVVLCEEFTDLLGVKVGITDNFFELGGHSLLATKLSARLSRRLDAGITVKQVFDQPVLADLAASILQGSSRHRSIPSLPYEGPVEQSFAQGRLWFLDQFNIDALWYLIPFALRMRGPLQVDALAAALVALEERHESLRTTFEERDGVGIQVVQPLRTTKDIRIIDVSGMRDDDAYLEPLQKEQQTPFDLASEPGWRVALLKLGKDDHILSIVMHHIISDGWSTEVLQRELGQFYLAAKSGKAPLSQVAPLPIQYRDFAVWQRQEEQVAESQRQLDYWKKQLADSSPAELLADYTRPNVLSGEAGSVSFVINDSVYKSLVSFCRSRQVTTFTTLLAAFRAAHYRMTGSDDATIGTPIANRNRPELENLIGCFVNTQCMRITIGDDETFESLVQQVRSTTATAFENQDVPFERIVSTLSAGSRDTSRNPLVQLLFAVHSQQGLGRIQLDGVVDEPVLSTVSTRFDLEFHAFQEADRLNGSVMFATDLFQPETIQGFVAVVEEVLQRGLEQPQSPIATMPLAEGIAQLRDAGALQMPKSDYPRNASLVDVFQQQAMASPSTVAVTDSTSKLTYAELDRLSDQAASYLRRQQLPAETMVAVLAPRSCETIIAFLAILKANLAYMPLDVNTPSARMEAIISSVPGRRLILVGSGVRHADINVPNAKTMLISDTVTGTDAIGTPEPLVVRPSATSLAYVIFTSGSTGKPKGVMVEHRAIMRLVKDSNVVTHMPPATRMAHVTNIAFDVSLFEMCATLLNGGTLVCIDYLTLLDSTMLRETFEREQVRAAIFPPALLRQCLVNMPDAIGMLEAVYVAGDRFHSRDARATQALAGPRVYNAYGPTENAILSTIYNIDKHDPYVNGVPIGSAVSNSGAYVMDRNQQLLPPGVMGELVVTGEGVARGYTDASLDTDRFVTVTIDGQRQRAYRTGDRVRYRPKGFQIEFFGRLDQQAKIRGHRVELGEVEHALLSENSVTDAAVVLRTMEEEDPQLVAFVTTDHEYRSGSSNEEEDPYATQAAGDMRKRLRSLLPYYMVPSRVTILRQMPLNANGKVDRKDLARRAQMTPTASSSGPVHVAPRNETEAAICDEFETILGVKVGITDNFFELGGHSLLATKLAARLSRRMGLRISVKDLFDDPVPVSLAGKLEQQQGFSGEDESSTVGIVPFQLLPAEMSREIIQRDVVPQIENGHSTPLDMYPATQTQIFFLHDKATGHPATPPLFSLDFPETADCRRLASACAALVQHFDIFRTVFVSRGGRFYQVVLAHLDVPVEVIETEQELDEVALALHEADKQQPLRLGRAMLRIAILKRPGAKMRLVLRMSHSLYDGLSLEHIVNALHALYSDKHLAQAPKFGLYMHHMASRRAEGYNFWRSILQGSSMTSLKRSVGALEAMTPSAGTWQTSKSIRIPPAALKNGITQATLFTAAVSLLLAKHTKSTDVVFGRVVSGRQDLSINCQDIVGPCINEVPVRVRIDEGDDMGGLLRAIQDQYTSSFRHETLGLQEVKENCTDWTDATKEFSCCIAFQNLNLHPEAEIEGQQIRLEGLPAKDQARQANGHAPNGTNGTNGTNGTNGANGTNGTNGTNGTHANGINGSNGVNGRDSNVVSAAGDQAPVHDLDIVGIPEPDGSVKIGIGASRQILGEKVVGSMLNELCETMLALSRT

SEQ ID NO: 17 - protein, 383 aa, unidentified
  source 1..383: note = Tolypocladium inflatum
MKICPILNSKAGHWGDPTLTGSAFDFRSDAITTPSHGMLEAIAGATLNDDVYGEDQLTRSFEEHMASICGKEAALFVVSGTMANQIALGALSRRCTGVLADATSHIVHFEAGGLAGLSGASIQPVRPANGHYLTLDDVERHAVTTNMIERLPTSIISLENTAHGSVIPLQELRKMKAWADEQEIAIHIDGARIWHAVAGGGGSLKEIAGCCDAMTLSFGKGLAAPIGSAIVGSRDLIARARRLRQSIGGGVRKAGAIVAAAWQAMIENFGPGDVDTRGIIQRTHVLARAVANMWTSRGGLLLRPVMTNLVWLDLKSAGLDKSTLDTAAQHRDIRIRAPRIVLHHQISTDAMRRLEAVFEEVLEKKASQSPLRSRAERAGLERL

SEQ ID NO: 18 - protein, 172 aa, unidentified
  source 1..172: note = Tolypocladium inflatum
YLELEWEGPVMKDGKPTATIKSQTGRINLTVYDDVTPLTAENFRALCTGEKGFGYKGSYLHRIIPGFLLQGGDFTRGNGTGGKSIYGTKFKDENFTLKHHKPGMLTMANAGPHTNGSQFVITTAETSWLDGRHVVFGEVADEPSMAVVMAIEATGSESGAGQYSKRPMIVAC

SEQ ID NO: 19 - protein, 1240 aa, unidentified
  source 1..1240: note = Tolypocladium inflatum
MGILNRAKASQDPVLDSELDSARLSGDVASPAIEDPPREREAGLKDYIRVFTYAKRQDIIFLVAAALSSIAAGVTMPLMVAVFGRLVENFSSFDAADGKTQHAFDDTMNRGSLYITGLFIARLGLNYINKFCFRIIGIRISAAIRLHYMQCLLGQTIHVLDSMPPGAAATTITATSNILQIGVSEKLGTFLEYTATIISAIIIAFVYSWSVTLVTSSVLIFILVVLGILLPMILKGHARLTAAETKAGAVAAEALGSVRMITSCGAENRIALKYARLVEQARQAGVWTSPFVAIQTGLVFFSFQVAFALAFWFGSRQYSNGRIDSVGTVVIVLMSVMLMVSSISQVSTPLVVISKAIVAASEFFAVIDAPRPVAGTLRYPSVNPGTDIVFNDVQFAYPSRPSKKVLDGLNLRIVANKNTAIVGPSGSGKSTIVGLIEGWYTLQRQHIIAKTVEKKPPAKKPQQGEEKDAEPPSAAVTGPSIEMDDLGPPVELSGSITIDGKDMTDLDRKWWRSQIGLVQQEPFLFNDTIHSNVAAGLTGTQWADEPEAKKRELVQEACKESFADEFIDRLPEGYDTQVGDSGIKLSGGQRQRIAIARSIIANPKILILDEATSAIDIRSERIVQAALERVSKGRTTITIAHRLSTIKNADRIVVLRHGKVAEAGTHESLLADPEGAYYRLVHAQKLSLGDDDGAEGLPGMKEEKVSIEKQRTRTASSSVADLPSGANHDAQWKDKNIINGFGKLLYEQRSRFPLYVIAIIFSMGAATAIPLQAYFFGNVISIFGKTGQELRDDSSFWASVWVGLAIGVGICYFVVIMVATNVEHFIASVYRREYFDSMLMQKTSYFDQDGNSTGQLTARLSSDPTSLKELLGINTCMVLVGIFSLLGAVIISFIYGWKLALVALCIILPLTCLATFYKLRYELQFAAMNEAVFSESSKFGAEAVGAFRTVTALVLEDAICERYQKLLQGQLTSAYRKSPWPTFIFAFSDSVGLACQALILWYGGRLVSSGEYGIIGFFITYMAVIQGGESAGQWLGFGPNAAQATAAANRILNLRNTRHQDMALGSRPVVPETQNGINIQFQDVHFKYPTRDVPIFQGINLTIEEGQFAAIVGASGSGKTSIVSLLERFYDVTKGRILFNGQDIRETSIPEHRRLLSLVSQEPALFSGTMRDNILLGVDPDQTTDEQLEQCCRDADIYDFISSLPDGFQTEVGNKGEPARTAARRGDELARLGERETGAG

SEQ ID NO: 20 - protein, 313 aa, unidentified
  source 1..313: note = Tolypocladium inflatum
MDENPLRLQFRPRAARFPSAAPLVLIHDAGGTVYSYLRLGDLKRDVWAISDPYFEAVKPWDGGFDEMAEHYQQLIESAGIRGPILFGGWSLGAYVSLAIAKRMLHIKPCRFYVVGILMVDSPIQIPTSTLPPCGPDPDFSDLPDLVRRSFDKFDAMLSVCDLPTWDSPACQGKPVRLAAGGKTFTVETDQVLHLPLDGGWTTASLTKTPQEGRMEVTEQFTGPPPAVLVRCLRRTPTDAPSQGPSQVDRYRDDLLLGWEGRHQSFIKAVIDVDKHHFNVFQPSNVSWPDKNKDDGTLLGLVIRRAINRSFVGL

SEQ ID NO: 21 - protein, 490 aa, unidentified
  source 1..490: note = Tolypocladium inflatum
MTRPISREELGQHNKDGDLWIALNGKVYDFSDFPSSHPGGPEVIYRYAGQDASEEYNTFHAPDLVKSTLGAEKMVGHILDVATAGSDAVKPTSVKDQKPLLDELINLYDLEEVAKRTLTAKAWAYIDGASNDNLTYRFNNRVLKHIWFRPAVMRGVRYVNMSTTLFGCKLHVPIYISPTAAVSVAGPDGELAQARGAASSGIIATIPNMASHPLENILDATPERAFFQLYVSNDRQQSEDIVRKVAASGKIKAIFVTVDSPVVSKREADERTKGNLSSVNPYKGSSPAAAAAAAAASRKGPPRGGFARQSGQQVDPSTSWSDLAWLRSITDLPLVLKGVQRHEDAVKAMELGFEGIILSNHGGRAADGALPAILTLLELHRNCPQVFGAMEILVDGGFRRGSDVVKAICLGASAVGLGRPFMYSVMYGQEGTERVVDILKDEIKTCMQLIGICDLFADASPNYLVTGEIDHLVWSGQHPYARKMTRRSRI

SEQ ID NO: 22 - protein, 2531 aa, unidentified
  source 1..2531: note = Tolypocladium inflatum
MAPQQISPPNGTASPEAGTPRYTYRPASPAPESPWADSAEGAELAKPMAIIGMAMRLPGSVRSGDDLWDLLSTRKSGLCDIPKNRFNADGFYDPARGPGTIPVKKGYFLHDVQIEEFDTNVFPIPKMELERLDPAQRQLLQVAYECIENAGGTSWRGSRTGCYIGEFGEDFADSSARESQQRGNLRYTGLADFAIANRISYELDLQGPSMVVKTACSSSLVCLDLACKAIQSGECESALVGGVSMLFSPATYISVTDLGVISPNGQCRSFDAGADGYARGEAVNMVMIKRLDHALRDKDPIRAVIRGTGVNTDGRTNGMLTPSSAVQADLIRHTYEIAGIDDLSQTAVVECHGTGTPVGDPIETEAVGRCFGDDGVTITSPDQVCPELGAPASAAKYQLRDTESRQKLTYVPQVPFEKYKLRVPTEVESWPQGRAERISINAFGIGGVNAHAIIESPAQFGIKKPANGIAGRGVHTNGDSVPNGSQKQHEHDHRQDQSAYVKGDYMNGTAVNGANGNEKHADRGPNLLVFSAYSSESLDRQISAYRDFAATHQGASLKDLAYTLSSRRDHRPYRAYAIADDASGVQDASATVNAVVDGAPPPVGWIFTGQGAQWPEMGARLIDTSAAFRNRIRKLDKYLQTLKLEPPVSIEAELRKTKEDSRVHRPEMGHLVCVAMQVALVDVLRSWNIVPDFVLGHSSGETAAAYACGAITAEAAVYAATRRGIGNASSQRKGSMAAVGLGRDEIQPFLLEGVNIACENSQSNVTLSGDTEQVESIVATLKAERPGVFARLLRVEKAYHSHHMCEYGPVYEEQLKPVVRSADPEIPFYSSVTGDRLTGEGQLGASYWRANMENTVLFNPALRSALRDRPGKLVLIELGPHPALEGPVGQILRDLGRTDDIYLGTCIRGSECDRSLLHLGGKLYQQSIPMDLAAICPPGAVLTNLPRYAWSQDTTHWEESRVSRGWRLREHPPHELLGSRVLETEGEPCWRKVMALEDALWLDGHEVNGQVLFPAAGYISMVGEALRQLTGDATFTVRNVRIMSGLVLSSDKPVELVTLLRSTTMGTSDDSEWYEFTITSFDGTAWVRNCHGEAMASSDKSFHLDSVSPAAGSFPRKLDRRDSYDILRRVGFNYTGEFEGMADISVSPSSLQAHGVTSPARQVTDRPSRCATYSVHPAVIDQCFQLFTVALCRGLRRSNRRLAVPTFIEELVVSPSASPLSVTAKINRLDEVGSWTGDFVALAAEGPVVRLKGMKSVVLATPAQSEPPLSTELEWKPHSDFVGLGVGLHPRVPRKREWALLEELVLLCNLDHLGQIKTDDSTAPYLIKLLDWMRLVTDRYRSGKNLFVSAGAGLEHLDHDERVARIDFLMAQLADTQDVVFARAIHRLFIEAPSIFAGEGHPLHILMPDNVLSDFYDATSFDMADAVRLVANTNPHMRVLEVGAGTGSMTARLLRALTSSHGERLYSQYGYTDVSAGFMAVGKQRFAAVENIDYDVLDISKDPKEQGFRPGSYDLVIASNSIEAQWTLVPGGTHSRCNVRKLRHVFPKGFFYGWWLGAEDNRVEQPWIAPECWAEKLVAAGFQKPEAIVLDSDKPYQISAGIIASRGVRSTLPSKVAVLCHTADEPHAVEMISRLSAMGIDVETCLWGQPLPSCDIISVLELEKPLLHEMSEETFKTIIAYFQSHKARVIWVTEACQIDCANPEPAMMLGLARTVRNEYSLQMYTVELDKGTTMSRATEAIIDIWGRVSTPDLDPESMDHDYEYALVDGRILIPRFHWQTMSGAFSGAARKETDDVEVALKHLNMSTPGLLRTMKWIDGSVPTAPAKGEVLVEVKAVGLNFRDVILALGVVEGNPSGMGHEGSGVIRAVGPDVQDLSVGDRVMFIHDGCFTTQLTLSKDICVRLDDSTSFVQGAALPAVHATALAALVDVSRLQRGQSVLIHAACGGVGLAAIQIAQNIGSEPKRRYLEDTYNIPADHIFNSRDVTFLPEVMRATNGRGVDVVLNSLSGDLLHASWKCVAEFGLMVEIGKRDFQRRSKLAMEVFEANRSFVGLDIRGLSISRPERAADLMRRCVDMIRSRAIQGPVACTTFPAVEIQDAYRYMQSAKHIGKIVIEMPDDPRDLGAGAAPPEGESTELEVSPRPKPSFRPDRSYLLVGGLGGLGRAVATWMVEHGARVLVFLSRSARENDDNRDFLNDLRSEGCTVLLVPGSVSNLEDVQKGVAVATADQPLGGLINLSMVLRDVTLNKMTYSDWTTAVEPKVRGTWNLHHATANCTSLEFFILFSSQNAQIGQWGQANYAAANTFLDAFAQYRHGHSLVASVIDVGLMGDVGFAAENRAILKKLGRIGMYILQETDLLDAISLALLKSQPMHETEDKTSRYVTPGYVGIGLNTTTPMSAASTRVPWKRDPRMSIYHNMDNSSGDVRGEGSSKGSSLKVVLAAEPSEEKKTEIIAKALAGTLGNFLIKDGSSFPLDKPLKMLGMDSLIAMEVRNWIRQNIGAETSTFTVLQSSSFMHLAGEIRAAMNAASEE

SEQ ID NO: 23 - protein, 331 aa, unidentified
  source 1..331: note = Tolypocladium inflatum
MLTPLFLIKFAVAASVPVTIGGLAYVATSPELREGIYHFLTSPGWPLRLCLVVFAALNWKNLPLAWTARVFYSFACHDFFKRGDLPPHALFGHIVTTTRNSPLDTDYNLHKSNSTYFPDLDVSRTRLVSYLLTPGIQVVTDNARTQVVRDRSGRVMGGGFSLVLGSVFCSYRREVGMLESYELWSRVLTWDRKWLYFVTHFVARDKGKGKASPGGVGTDRDWSQRIIATAISKYVFKLGRFTIHPSIILEESGLLPERPGGGWRGGELEVGDVSEKVESLEGEEEPVEGWKRTELMRRKGMELAEHFAALDAGHSLFDGREDGVLGRFVLG

SEQ ID NO: 24 - protein, 299 aa, unidentified
  source 1..299: note = Tolypocladium inflatum
MLQYSFANILNVHRLLEWAKLLGKIVYTLVYYAFFHPLAKYPGPVLAKFTNLYSAYHAWKGDIHTDILRCHEKYGVYGHGAPVTKGHSYKVLAARAPNTLTMQDKLQHSRRRRVLSQAFSESSLRKFEPVMQARIDRFLRAVRGADLQAGQWSEPSNMAHRTHLAFDTMTTLTFGIDYRTIEDDEYRFVMDAIEKSNVRLAALWQMPNLTFGGLDRKLLPEADKAAKRFVVFLRSLMKIRLGEEKADNNDIFSFLQQCKDPDTEQHLTLRELSTETATFVVAGNLSVFPKSAAGWLTFG

SEQ ID NO: 25 - protein, 366 aa, unidentified
  source 1..366: note = Tolypocladium inflatum
MFPPPPASGIDWTNVDLSLKIPVKGHVEARHNASTGTWSAPVLETNPNISISGISPALNYGQQCFEGMKAYRQTSGDIVIFRPEAHAARIAKSAVAVCLPPPPKDLFIECVRAAVAANAELVPPSSSNGALYIRPVLFGSSAQLALAPAEESILAVYVYPLLPYHGSAATDALVLEDFDRTAPFGTGPFKVGGNYAPVWKHAAAAKEKGYGITLHVDSATRTKIDEFSTSGFMGFKSDGSEKTLVVPLAENAVQSVTSASLTRIAELQGWTIQRDEKVPLHTLSSIEEVVAVGTVAVVVPIRSISRLSTSEKFTFPGTSNPEKIESQLLGLARTLSDIARGNREDTEGWCWKVTEHGPDSSSRSRL

SEQ ID NO: 26 - protein, 473 aa, unidentified
  source 1..473: note = Tolypocladium inflatum
MIPPYFLPARPITPNFFFPSRFSCDDGACIYFTLVAKGSAIMPWNKSAQDFFRKGREKGSGQCTERIDGSIVLKQPQEKILREYKRMIGLWNRYAEEHADDDPSPYDLKSLKEFMKEVAFGIDGAEDDPRRQECTIGRALWLLSVPPLASASTLSPCAAQVLHSLYEGLVPMPLVCNAMLPILAILISAKAFSGYDTIEDLLALEPLEVEMMHLQWIASILEEPFFKSMSLRRTRGSIETAVAVGRRLRALESRAGYANPPTIGDFRAEGLYSLAQRIKHAGQKDPSTYGKHYTPNNSGTDGQGSYFGTEVRSVVNDLFRGLTVARNPHLPERDRLAEGLFKTDTLRSPTGLQALRDMIALCEPNTEVNFRPGLEPNLCQCGKSTRRRKLPQSARAANDLQSIYDWKHTLSVLALIPVIQAYAAKGELKGIASQRDSHCVAPTRWRRKLPGALELALLVSLYPSSRKNLITRF

SEQ ID NO: 27 - protein, 351 aa, unidentified
  source 1..351: note = Tolypocladium inflatum
MSFKRPCPDDSDHESPLGHQRGKKTEQGRFPQRAYRERLKAKLEGLEDEVPAPQELFIDAAPTPRSEIINSSLDSKAASTPYPEVATPAAHHDLMTGAVFDLAITWTFRPPQTFTLHCLRTAPLSRWTREMYHGDYRTTAPKPGPLEYVGPQIPSHVATNPRGGRSIDRAAPVQDRIRHIVDSAVEAGFTTFDEAIEAYYTETFDETSPLDRDQRLSRNRRLPRLLGALRDASKDWSHWERRGFQEQITQGAEDILVDELELFRKEYTDTAFLREMTTSELGGGQTAATSRGSSNRRTFQDNVCAFPPVLPPLSSHLFSSLKHGLILNNLAAPKPMGFGYDACSEDKFIKA

SEQ ID NO: 28 - protein, 3712 aa, Hapsidospora sp.
  source 1..3712: note = Hapsidospora chrysogena
MALEQWKTTVQSVSERCDLSGLSQHPTDYQLASTGVKGAGGSSIEERSAIVSDELFSSLRDVCSQRQLDPRSLMLFSVHQMLKRFGNGSHTVVASLVTSSEGCPSTSAWRAIPSVIHHIEGGDNNNTVASAVEQAANLLNSEGSGQDLLIPIGLTELVKSELIDLLVIFDDETNNIRLPQDFPLILRIHQRQDHWQLSVRYPSPLFDTMVIDSFLSALHNLLSAVTKPSQLVRDIELLPEYQVAQLEKWNNTDGDYPTEKRLHHLFEEAAVRRPQHVALICGDKRITYEELNAMANRLAHHLVSSGIQTEQLVGLFLDKTELMIATILGIWKSGAAHVPIDPGYPDERVKFVLNDTKAQVVIASQRHVDRLRAEAVGGQHLRIIGLESLFDNLAQQTQHSPETSGNLTHLPLNSKQLAYVTYTSGTTGFPKGIYKEHTSVVNSITDLSARYGVAGEDDEVILVFSAYVFEPFVRQMLMALTTGNSLAIISDEDKFDPDTLIPFIQKHKVTYIHATSSVLQEYDFGSCPSLKRMILVGENLTEPRYEALRQRFKSRILNEYGFTESAFVTALNIFEPTSQRKDMSLGRPVRNVKCYILDANLKRVPIGVTGELHIGGLGISRGYMNREELTRQKFLPNPYQTDKERQRGVNSTMYKTGDLARWLPSGEVEYLGRADFQIKLRGIRIEPGEIESTLAMYPGIRASIVVSKKLLSQGQETIQDHLVGYYVCDEGHIPEGDLLSFLEKKLPRYMVPTRLVQLAQIPTNINGKADLRALPAVEVAVAPTHKQDGERGNQLESDLAAIWGNILSVPAQDIGSESNFFRLGGHSIACIQLIARVRQQLGQGITLEEVFQTKTLRAMAALLSEKYTKASNGTNGVTNGTAHVNGHAANGHVSDSYVASSLQQGFVYHSLKNELSEAYTMQSMIHYGVPLKRDIYQAAWQRVQGEHPALRLRFTWEAEVMQIVDPKSELDWRVVDWTDVSSREKQLVALEQLQTEDLAKVYHLDKGPLMRLYLILLPDSKYSCLFSCHHAILDGWSLPLLFNNVHQAYLDLVEGTASPVEQDATYLLGQQYLQSHRDDHLDFWAEQIGRIEERCDMNALLNEASRYKVPLADYDQVREQRQQTISLPWNNSMDAGVREELSSRGITLHSILQTVWHLVLHSYGGGTHTITGTTISGRHLPVPGIERSVGLFINTLPMIFDHTVCQDMTALEAIEHVQGQVNAMNSRGNVELGRMSKNDLKHGLFDTLFVLENYPNLDTEQREKHEEKLKFTIKGGTEKLSYPLAVIAQEDGDSGCSFTLCYAGELFTDESIQALLDTVRDTLSDILGNIHAPIRNMEYLSSNQTAQLDKWNATAFEYPNTTLHAMFESEAQQKPDKVAVVYEDIRLTYRELNSRANALAFYLLSQAAIQPNKLVGLIMDKSEHMITSILAVWKTGGAYVPIDPRYPDQRIQYILEDTAALAVITDSPHIDRLRSITNNRLPVIQSDFALQLPPSPVHPVSNCKPSDLAYIMYTSGTTGNPKGVMVEHHGVVNLCVSLCRLFGLRNTDDEVILSFSNYVFDHFVEQMTDALLNGQTLVVLNDEMRGDKERLYRYIETNRVTYLSGTPSVISMYEFDRFRDHLRRVDCVGEAFSEPVFDKIRETFPGLIINGYGPTEVSITTHKRPYPFPERRTDKSIGCQLDNSTSYVLNDDMKRVPIGAVGELYLGGDGVARGYHNRPDLTADRFPANPFQTEQERLEGRNARLYKTGDLVRWIHNANGDGEIEYLGRNDFQVKIRGQRIELGEIEAVLSSYPGIKQSVVLAKDRKNDGQKYLVGYFVSSAGSLSAQAIRRFMLTSLPDYMVPAQLVPIAKFPVTVSGKLDAKALPVPDDTVEDDIVPPRTEVERILAGIWSELLEIPVDRISIYSDFFSLGGDSLKSTKLSFAATRALGVAVSVRNLFSHPTIEALSQWIIRGSNEVKDVAVVKGGASLDIPLSPAQERLMFIHEFGHSGEDTGAYNVPLQLQLHHDVCLESLEKALRDVVSRHEALRTLITRTQKSSVHCQKILDAEEAQKLFSVDVLRLTSETEMQGRMAESTAHAFKLDEELPIHVRLYQVVRDGRTLSFASIVCHHLAFDAWSWDVFQRDLDAFYAVHTKHKAAANLPTLRVQYKEYAIEHRRALRAEQHRVLADYWLRKLSDMEASYLVPDRPRPAQFDYTGNDLQFSTTPETTAQLKELAKREGSSLYTVVAAAYFLLLYVYTNQRDITIGIPVAHRNHPDFESVVGFFVNLLPLRVNVSQSDIHGLIQAVQKELVDAQIHQDLPFQEITKLLHVQHDPSRHPLLQAVFNWENVPANVHEEQLLQEYKPPSPLPSAAKFDLNVTVKESVNSLNVNFNYPTSLFEEETVQGFMETFHLLLRQLAHNKASTSLSKLSVEDGVLNPEPTNLQPSSRDSGNSLHGLFEDIVASTPDRIAIADGTRSLSYSELNERANQLVHLIISSASIVADDRIALLLDKSIDMVIALLAVWKAGAAYVPLDPTYPSQRTELILEESSARTLITTRKHTPRGGTVANVPSVVLDSPETLACLNQQSKENPTTSTQKPSDLAYVIFTSGTTGKPKGVLVEHQSVVQLRNSLIERYFGETNGSHAVLFLSNYVFDFSLEQLCLSVLGGNKLIIPPEEGLTHEAFYDIGRREKLSYLSGTPSVLQQIELSRLPHLHMVTAAGEEFHASQFEKMRSQFAGQINNAYGITETTVYNIITTFKGDAPFTKALCHGIPGSHVYVLNDRLQRVPFNAVGELYLGGDCLARGYLNQDALTNERFIPNPFYEPKQASDSRPQRLYKTGDLVRFRGPHHLEYLGRKDQQVKLRGFRIELSEVRDAVLAISAVKEAAVIPKYDEDGSDSRRVSAIVCYYTLNAGTVCEASSIRDHLHANLPPYMVPSQIHQLEGSLPVTVNGKLDLNRLSTTQVSQPELYTAPRNSTEETLCQLWASLLGVDHCGIDDDLFARGGDSISSLRLVGDIYRALGRKVTVKDIYLHRSVRALSENVLTDQKDKGTLPASPPLQRAEQGQVEGDAPLLPIQDWFLSKPLDNPAYWNHCFTIRTGALSVEGLRGALKLLQERHDVLRLRLQRRDEGRHVQTFARDCAQPRLTVLDRRSFEDAEDVQEALCEIQSHFDLENGPLYTVAYIHGYEDGSARVWFACHHVMVDTVSWNIILQDLQALYHGDSLGPKSSSVQQWSLAVSDYKMPLSERAHWNVLRKTVAQSFETLPICMGGVLQCQEKFSRETTTALLSKACPALDSGMHEILLMAVGSALQKAAGDVPQVVTIEGHGREDTIDATLDVSRTVGWFTSMYPFEIPKVTDPAQGVVDVKEAMRRVPNRGVGYGPAYGYGGSCLPAVSFNYLGRLDQASSGAQRDWTLVMDEDEYPVGLCTSAEDSGRSSSMVDFTFSISGGQLVMDMSSSWGHGARNEFVRTVRNTLDDLIKTTSSRDFSAPLPPSDQESSFTPYFVFEEGERHGAPLFLLPPGEGGAESYFHNIVKGLPNRNLVVFNNHYREEKTLRTIEALAEYYLSHIRSIQPEGPYHILGWSFGGILGLEAAKRLTGEGHKIATLALIDPYFDIPSASKAIGQPDDACVLDPIYHVYHPSPESFRTVSSLTNHIALFKATETNDQHGNATQQALYEWFATCPLNNLDKFLAADTIKVVPLEGTHFTWVHHPEQVRSMCTMLDEWLG

SEQ ID NO: 29 - protein, 338 aa, Hapsidospora sp.
  source 1..338: note = Hapsidospora chrysogena
MGSVPVPVANVPRIDVSPLFGDDKEKKLEVARAIDAASRDTGFFYAVNHGVDLPWLSRETNKFHMSITDEEKWQLAIRAYNKEHESQIRAGYYLPIPGKKAVESFCYLNPSFSPDHPRIKEPTPMHEVNVWPDEAKHPGFRAFAEKYYWDVFGLSSAVLRGYALALGRDEDFFTRHSRRDTTLSSVVLIRYPYLDPYPEPAIKTADDGTKLSFEWHEDVSLITVLYQSDVQNLQVKTPQGWQDIQADDTGFLINCGSYMAHITDDYYPAPIHRVKWVNEERQSLPFFVNLGWEDTIQPWDPATAKDGAKDAAKDKPAISYGEYLQGGLRGLINKNGQT

SEQ ID NO: 30 - protein, 866 aa, Hapsidospora sp.
  source 1..866: note = Hapsidospora chrysogena
MFGSRDGECDPEEYVRPPSKDRKDIGVQLVLSLIIGVSALVTFCILRPRWPALYAARKRRLEPKIKLPELPTTFLGWMPKLYRITEQEVLATAGLDAFVFLNFFKMAIRLFALMAFFAIVVLLPINNKFAGFDLSFGGNDTDTNRSMIENDFAYDPFQLNLDDTFDVMKKGKGGHKDKSRESTFLWAYVVFTYFFVAMTIYSVNLETFRIIKFRQDYLGTQSTVTDRTFRLTGIPSDLRSEARIKRLIEKLEIGTVTSVTLCRDWREIDDLVEERNRVLRQLEASWARFMKSQHAYKAGQRFDEQSTPSGNGSGTHVDEEAGENVRLLEENTLNPHLMEGDRPQMTIRYGILKLRSRKVDAIDYYEEKLRRLDDRIITARKKEYRPTDMALVTMDSVASCQMVTQGQDRSASRPPLDQVTPAPSDLVWRNTYALRGIRRLKSWVITIFITILTLVWIFPTAFLASLLSICTVKKVAPTFSDWLNQHSIIYSLFANGMPTLIVSLLNVAVPYLYDFLSNHQGMISQGDVELSIISKNFFFTFFNTFFVFAISRTGFNFWSTLQEWMQNTSLIARAIAADVEELSIFYISFIILQGIGLMPFRLLEVGSVVLYPIYRMTSATPRHFAELQQPPTFQYGFYLPTALLVFNLCLIYSVLRWGFAILIFGTVYFILGYFTFKYMVMYAMDQPQHATGGAWRIICYRIIVGLLVFEVVMVGQIASAPAWVQSAFILPLIPFTIWYSYYFGQRFDPLTRYIALRAIRSDEDEVDAGAVENAFADEDGPRPSQGILRRDSTLDEFKERGMRFVNPSLVVPLVQPWVYDDPPPPLPEEESGTEESRQASLIVPSTDSSLGIGEANVWRDTDENNI

SEQ ID NO: 31 - protein, 482 aa, Hapsidospora sp.
  source 1..482: note = Hapsidospora chrysogena
MVKAEEDSAALEAAIDNGRRHIVDFDGPTDPDNAVNWPAARKWLNVFVLAAMTFIAALGSSIAAPGVNQAIVDFDSTSSILSSLIVSVYNVGLAIGPLIVAPLSEMYGRLIPYHVTNVLFVLFTIGCALSPNLPALVIFRMLAGMEASAVMTIGGATVADLFIQEERGRAMAIWTFGPLMGPAVGPAVGGYLAEAKGWRWVFWVVAIGGGFITGMFFLIARETYPPVLLQRKVNRLRQETGNPLLTSALADTSSRRARISRSVRRPLVLLFRSPIVFLFSVFIAVVFSYQFLLFVTIPSVFGEIYDFSLGQIGLSYLGIAAGLLLGNAIFGQASDRILSKKSGGMELKPEFRLPLMIPGAFCIPMCFFIYGWATYYKLHWMMPICATSLLGIGLNLSLMTIQVYLVDTYTLYSASALAAATILRSLFGAFLPLAGPPLYDALGLGWGNSTLGFIAVALIPVPFLFIRYGEAIRTNPRFQPAL

SEQ ID NO: 32 - protein, 609 aa, Hapsidospora sp.
  source 1..609: note = Hapsidospora chrysogena
MAPGGLLTLAGAAAASTAAAAYLDAKLHLTKDLNQLARAERGAQNFARAVEQRKASGFFLFEAAAARLGDAPCIWSRGHPEYSWTQTYQRACQYGHYFRDLGVVAGQHVGVYLYNSPELMFIWMGLLSIGAAPALINYNLGSDALVHCVRLSRSRFLIYDDASDCSSRIHEVGERLRDINVEAIMLSGSGTTGLPKAAPITVARNYPSASLLPKTFGQKPGPNGDRTYYCIPLYHGTGGIAAMNDLMSGISIALAPKFSLSRFWDDCIESGSTIFVYVGELIRYLLSAPASPKDRQHRVRLVWGNGLSPELWTKFQDRFGVSDIGEFYASTEGVLTLLKHYRGGGFGLGAVGHHGWLLRRKFHNDYVPVRIDPETGDIWRSPKTGFAERLPYERGGEILARLPSRSAWAGYWHAEEATQKKLVENVFEKGDLYFRTGDALRRDADGHWYFLDRLGDTYRWKGENVSTTEVGQVLGSHADIAEANVYGVQVPNHDGRAGCAAIALKNAATPDTLDWSRLTSLLRSELPSYAVPVFIRVRETVGGMSTDNHKHNKVPLRDEGVDPRSMGSKVPGSEKDRFFWLPAGASKYVPFTERDWDLLSGQSAARPRL

SEQ ID NO: 33 - protein, 383 aa, Hapsidospora sp.
  source 1..383: note = Hapsidospora chrysogena
MDPSRPHPLSGKLVVELAGLAPGPFCGMLLADYGASVLRIDGPRSPKGDVLARNKSSICIDLKHPPSRKVLLSILSRADVLIDPFRPGVLERLGLSPTEVLLKANARLVVARLTGFRRDGKYQDMAGHDINYLAVSGVLAMLGRAGENPFPPANILGDFAGGGAMCVVGILLALVSRDATGLGQVVEANMVDGSAYLATMPRLATKTPFWGSPRGENVLDGGCPWYATYRTKDPGGKYMAVGALEPHFYEVLVRGLGLDKTDLPPREDRANWPRLRALFEAKFAERTRSEWAEVFDGTDACVTPVLEQGELEKAGFEQRLPVNLGATPGKPILPGQGDWTGRTLAKGHGGEEILRRWIGWERGVDYHVEENSGILVACSREKL

SEQ ID NO: 34 - protein, 332 aa, Hapsidospora sp.
  source 1..332: note = Hapsidospora chrysogena
MTSKVPVFRLDDLKSGKVLTELAEAVTTKGIFYLTESGLVDDDHTSARETCVDFFKNGSEEEKRAVTLADRNARRGFSALEWESTAVVTETGKYSDYSTCYSMGIGGNLFPNRGFEDVWQDYFDRMYGAAKDVARAVLNSVGAPLAGEDIDDFVECDPLLRLRYFPEVPEDRVAEEEPLRMGPHYDLSTITLVHQTACANGFVSLQCEVDGEFVDLPTLPGAMVVFCGAVGTLATGGKVKAPKHRVKSPGRDQRVGSSRTSSVFFLRRKPDFSFNVQQSREWGFNVRIPSERTTFREWLGGNYVNMRRDKPAAAEAAVPAAAPVSTAAPIAT

SEQ ID NO: 35 - protein, 444 aa, Hapsidospora sp.
  source 1..444: note = Hapsidospora chrysogena
MLPSAQVARLKPDPFPPSLSPIPHGAVTFAALAPCHNLPIFSSRQMLRDSLTYSHTSPTMSPQIANRFEASLDAQDIARISLFTLESGVILRDVPVAYKSWGRMNVSRDNCVIVCHTLTSSAHVTSWWPTLFGQGRAFDTSRYFIICLNYLGSPFGSAGPCSPDPDAEGQRPYGAKFPRTTIRDDVRIHRQVLDRLGVRQIAAVVGASMGGMHTLEWAFFGPEYVRKIVPIATSCRQSGWCAAWFETQRQCIYDDPKYLDGEYDVDDQPVRGLETARKIANLTYKSKPAMDERFHMAPGVQAGRNISSQDAKKEINGTDSGNSHRAGQPIEAVSSYLRYQAQKFAASFDANCYIAMTLKFDTHDISRGRAGSIPEALAMITQPALIICARSDGLYSFDEHVEMGRSIPNSRLCVVDTNEGHDFFVMEADKVNDAVRGFLDQSLM

SEQ ID NO: 36 - protein, 561 aa, Hapsidospora sp.
  source 1..561: note = Hapsidospora chrysogena
MANNSGTTTVQLDDVLERSSTLNTLNNIDTVQHHEPRTSFANNRQQAPAGGSSQSDATVGPRHKLVTFAPDDPENPKNWSKAYKWYCTLVVSLTCFVVAFASSVISADIDGVMEEFNVSETAALVSISVFVVGFGVGPMVFAPLSEVYGRRIIYGSKLLFAVIFVIPCAVSKNLGTLLACRAIDGIAFSAPMTLVGGTLADMWKNEERGVPMAAFSAAPFAGPAIGPLVGGYLSDAAGWRWLYWIQLILSGIIWILITFTVPETYAPTILAKRARKLRKSTGDPNHVTEQELDPRPISEQLRVFLTRPFQLLFGELIVLLISLYMSVLYGLLYMFFIAYPIVYKGGKGYSSGKTGLMFIPVAVGVVLSACCSPFVNTHYISLAHFHGGKPPAEVRLIPMMLSCWLIPIGLFIFAWTSYPWLSWAGPAMGGFPVGFGFIFLYNSANNYLVDSYQHQAASALAAKTFIRSFWGAGVVLFTKQMYSRLGDQWASSLIAFLALACCAIPFLFWKYGARIRARSKYAYAGDETPADVEKAKQRPAEAGVLADADDLRRAISYVSNP

SEQ ID NO: 37 - protein, 2531 aa, unidentified
  source 1..2531: note = Glarea lozoyensis
MGDISGSVDPSNMAYEPLAIVGMGMRLPGGIHTAEDFWNLLVEKRSSRCKVPSDRFNIEAFYSPSGRVGTVKMEHGHFLGPTDDLQHFDASFFSMSKKEVEILDPQQRMLLEVVYECMQNAGQSNWRGGNIGCYVGVWGEDWVDIHAKDSQDAGMYRISGGQDFAISNRVSYEYDLKGPSFTIKSGCSSSMIALHEAARAIQAGDCDGAIIAGTNLIISPTMSIAMTEQGVLSPDGACKSFDESADGYARGEAINAIYIKRLSDAIRDGDNIRSVIRATASNCDGKTPGITLPSSESHEAMMRRAYKEACLDPTQTAFVEAHGTGTKIGDPLEATAIARVFSSEKGVYIGSVKPNVGHSEGASGVTSIMKAVLALENRTIPPNINFSTPNPQIPFEASNMKVAVEPIPWPKVQAERASVNSFGIGGANAHVILDSPASMGISSTKRNTTNGLSVNGHSINGNSVNGHSVNGHSTNGHSINGNSVNGHSVNGNSVNGHSTNGHSINGHSANGNSINGHSVNGHSKPRPALLVLSATNTESLRANVVKHQQYIETNPEKLVNIEYNLCNRREHLSNRAFCVTDGLSTLQFSPLTKPKKTPTLVMVFTGQGAQWAEMGKELMADFPSFSQDIDLMNNTLSKLDHPPSWNIKEELLKHESVSCLSKAEFAQPLVTAIQVALVNLLRQFGVKPAAVVGHSSGEIAAAYAANAITANEAIIIAYYRGQVTKGFSRRGGMAAVGLGREDVMSFLNPGVSIACENSSSSVTLSGDEDALNATCEIIKTALPDVFLRPLKVEMAYHSHHMKELGEAYEALLRPHLKSTTPVVPFFSSVSGKVVSKSGTLDAAYWRSNLENPVLFNSAIKLILDTLAQDHLFLEIGPHSALAGPIRQILKANSRKNDTYVTALERGKDCGESILKMIGELYLQHISINFKQVAPNGTVLTDLPLYQWCHDQEYWKESRVSKQWRLRKYPNHEILGSRTVEGNELQPEWRNVLHLDNVPWLRDHQIINDVVFPCAGYLSMACEAIRQISASEDFTFRNIVIQTALVMSDSKSVEMITSLRPVRLTNTLNSVWWDFSISSYNGSLWIKHCGGQIRSGTDNPKFKLPRRIEDHPRSVPSPYPAMKKVGLNYGPTFQGLERLSALPKKMTASATLSKSELSESHYAIHPATIDHCLQLFIVSTCEGTLRNINKLCVPTSIDQLYICGGKSTSGIKAEACGAQTSTGTISGDVVAISGDAIILSLIGGQFSPIEEDSSDEDLDTVAGARLDWKPDLDFVQIDNLIRPRQQSTAATKTLEKFTLLSMIDIGRRISGLVTKSEYLEKFRSWINAQVERASEDRYNLVEDAQALTVLNAGERQMLIAELSKEISNSEVATSGELISRVVKNCEDIMVGKIDGIEVLLPENGLTHFYDSLEHRTDCIDFFTAAGHSKPNLRVLEIGSGTGGTSAVVLKGLTSTAQRMYSTYTYTDISSGFFVEAQERFKDYHGLEYKVLDISKDPTEQGFQEGSYDLIIAANVLHATPSLNTTLGHARKLLAEDGRLFLQELSPQVQFANLIMGVLPGWWLGEADGRANEPYISPERWAVELRKAGFSGCDATVYDAEQPYQFNANIISRPAKVDRIARRITLLYEPNLNIQQIKFSLENKGYSVDLCTIQEEPPAGQDIVSLLELETPMFEKISGTDLALFQRLVKNLGTNHLLWVTRSAQIESYDPRFGMVLGLARTLRSELSLSIATLEIDTVDEVAYNAITNVFDKLKNSSSVSDMNPDYEFVLSKGVVNIGRYHPVSVEQELAVSASQSQAVKLEIGRFGLLQTLRWVPDLQNKVGHDQVIVEPRCAGLNFKDVLVSMGIVSGDGLGLEGSGTVVGVGSEVTDFQVGDRVLYIDQNCFSTRTAIPALRCAKIPSTLSWEEAATMPCVYATVIHSLLNLGRIQKGQSVLIHSACGGIGLAAIQICQNIVGAQIYVTVGNEEKVHYLMDTFGISRDHIFNSRDTSFLPAIKAATNGRGVDVVLNSLSGELLHASWECVAEYGSMVEIGKRDFIGKAQLNMDLFESNRSFFGVDLAKFDAARCQLLLVQMMEFYEKGLIKPIAPMKVFEGAKVEDSFRYMQKGSHIGKIVVTIPEQNTDLPLASIVPKLKLNPDAGYLLVGGLGGLGRAVSTWMVERGARHLIFLSRSAGKSDQDQSFFRELESQDCTVQAFTGSVATFQDVQNAVQRASKPIKGVFQMSMVLNDKPFLEMSCSDWETSVLPKVEGTWHLHHALPKDLDFFVATSSLSGSFGNAGQANYAAANTFLDAFVQYRHSLGLPASVVDIGVMGDIGYVSRNAAIQESLRGAGTYFLQEQDFLDSLNWAVAKSAVKPSLPGQNQLLIGVRSSKSLSDPSNRVSFKRDARMGAYLNTGSSTSANTTNATDQLKSFMSSVETDSSILNVPASLDLVTNEIGVRIYTFMLQPIEDLDVSQTLAALGVDSLVTIEIRNWMKRSFGGLEFSTLEILNAGTIEALGLLTIEGLKRKYEMKDGEAKFSEREDTYLLMKAP

SEQ ID NO: 38 - protein, 281 aa, unidentified
  source 1..281: note = Glarea lozoyensis
MDQNPILIQRHHHRQNFAQKAPPAPLFLIHDGGGTVFSYFLLESLGRTVYGISNPNFETESTWENGIASMAEYYVNLIKATYPSGHILLGGWSLGGLIAIQAAHILSNDPELKVVGIVMIDSTFPVEGQFNKARRLAFMAETSTSPDMKEKTRKCMDEARIQSREWKAPSWRSSATETLDNHSQTINVQIHDHVTPTCPPTVLIRALDDGSNNSRDLNETSPTETSNDSETQALGFDRYQNFNLRYVVNTPGDHFSIFNKENVKELSKKVKEACDKLVMKS

SEQ ID NO: 39 - protein, 321 aa, unidentified
  source 1..321: note = Glarea lozoyensis
MYKISFAGLLRGNDKDVEALAEAATTQGFLELELDCMEAKALQKDVKFLESFAESILDSPEDVKAAYHFHKTGRFRTTGFKPLGSEEGAKAGNPDGFEFFFLPQKEILLSEFREQLNCPPLAMSNIDTLTDCFKHYERTAQMLLQRLTEGLKLGNELLNAHNTSLPCVTNMGLIRYPPQPKESGNFGHIAHTDVGSLTILAATQRGLQTLDNITQKWIWVDPSDECLFVQLGDSLKFLSRGKILPSLHRVLPSDVAPEATKYTIAYFLRPNEEAEITSDDGKVWLYKDYHCRKFDAFARPLGYRPDGEESLISLRDYTGVE

SEQ ID NO: 40 - protein, 328 aa, unidentified
  source 1..328: note = Glarea lozoyensis
MAIQTLDYRDFQYGGQEQHRTFCHNLCETLSTWGFIKIQNTSIPDAVIDELFSYNKKFFALPEHIKQKARHPAAPNPHRGWSAVGQEQLSRIAGFEKDEETDGFVPEYRESFDQGAADDELFPNRWIDEDDLPGFRKFMENYYEMCYNFHTQLLRAISTGLSLPEDLLLSRHQTDTSELRMNHYPAIACENLKFGMRIGEHSDFGTLTLLLQDSTGGLQVEDQKKLGTFIPVESDSRYEVIVNVGDCLQRWTNRRLRSANHRVHLPEGKNFKSDEVLADRYSVAYFGKPDRNVLVDSFPEFCRGGESKYNDHMNALEYNQTKLLRTYA

SEQ ID NO: 41 - protein, 328 aa, unidentified
  source 1..328: note = Glarea lozoyensis
MDYIKQAESTQLSSLSLSRLEGNNAEESKRLLEACAQDGFFYLDLRDHKQLLVDYEALLEIIKQYFNEPLDQKMKDDRKSDTIGYEPVATSAGVLDGLPDYYESFKVSWNQLRDHVQELPTVVETNIEVFDRFAKYVHSILLMILSRLSQTMGRNNDNRFESYHRDSIATRTNLTFLKYPKQDTTEHGVGHNKHTDVGTLTFLLSGQRGLQRLTPEGWCHVEPRSGFAVVNVGDSLRFLSDCVLSSVIHRVLPVGAHQTEDRYTLAYFLRPEDDAVFKDINGNLVSARSWHDRKFDHFRASHNQQKNDTILMGGMEENQKFLQYKFQA

SEQ ID NO: 42 - protein, 332 aa, unidentified
  source 1..332: note = Glarea lozoyensis
MRIETLDFSKFHVGTDAERYEFSTALLTGFANAGFIKLINHGFSQEEISQLFEENRRFFDLTDSIKAQIANEDGPKPQRGWSAVGAEKTGLLNTGGKTNLTKPDIEAPQDAKEHFDIGPSEDTEFPNKWPDNELIPGFQPWLESFYERSRYISFALMEALEIAMHLPKGAFIQKCQGHASELRLNHYPKITVKTLQGGKTSRIWPHTDFGVITLLAQDDVGGLEIQDKDNPTNFLPVSREDVSELVVNIGDTLERWTNGFLKAGLHQVTTPREIVNQSTETVRPRRSIAFFLKAHRQMSVGPLPQFVAEKTPAKYEDITALAYQQRRTAIVY

SEQ ID NO: 43 - protein, 617 aa, unidentified
  source 1..617: note = Glarea lozoyensis
MKNTDERYSMATCCAGGQSLRSKYGERAPNIQLSDRQWPSKKLTKSPIWLSTDLRDGNQALPNPMTLQQKWRMFQLLVSIGFTQIEVSFPCASQTEFDFTRQLIETPGATPDDVTLEVLSPCREDAIATSVRSLKGAKQAILFLYLATSDNYRETVLQLSEAEWLDQAKRCVEYARSITKDDPENCRTKWSFGFGFEDFANARPEAALRLGETIKTAWGPSKDNPVILGLATSVEATTPNVFADQVEYFSRNITEREKVCISIHTHNDRGGAVASAELACLAGGDRVEGCLFGNGERAGNLDLITCSMNMFTQGIETGLDFSDLLEIRRVYEEVTNLPVHPRQPYSGDFYFRAFAGAHQDAIRKGLAKRGRLAAMPSGLTQMSKGMIINGTSNEKIPSSTWRVPYLPLDPADLGFSFDSVIGVNSQSGKGGIAWLIQQGLGFNIPNELAAHYSQIIKKRSVSLERGLAAEEICDFFLEIYDLKSSYNTQLLKRLHSGFSQTIDMQQELEDATAPANNAAMDISKTMGIDVHCTMASSHVISSHDGEQLSNVAFVQCEIEQTTPCVWGVGVALSREEAVNRSLLLAARRKPGTETPDTKTSVNAIIQPLEVYSFSQKA

SEQ ID NO: 44 - protein, 358 aa, unidentified
  source 1..358: note = Glarea lozoyensis
MTENFPLPPLLGVDWDHLGFEPLEVNGHVECTFSTTTSCWTEPVFVTNPYLPVHGLAPGLNYGQQIFEGMKAFRNPSGDVQLFRPDQNALRFARSALRVAIPPVPTDLFLRAVNTAVGMNTDFVPPHGTGASLYIRPMAFASSPTVGLFLASQFKFCVYVLPVSPLHGKATQEGASVLVIEDFDRAAPLGTGNVKVGGNYGPVLGLIDEAKKQGFNLTLHLDSLSHSLIDEFSTSGFIGVLNDGEVPTIVVSDSQQVVSSITVDSICELARAFDWHVQKRPISFLEVARFSEVYAAGTAAVLVPVESILRRSTGEHVVYSVEYSSPTSCFSRLSTALRDIQQGLVPDDRSWIKLVTKP

SEQ ID NO: 45 - protein, 373 aa, unidentified
  source 1..373: note = Glarea lozoyensis
MTKQFDIVVFPGDYGGPEVMAEGIKVLKAIERKHQSDVSFNLKYHLIGGASFDIHDTPITTEALEDAKAASAVLLGAVGGPKWDGTPVPVESGLGRLRKFLDAFGNIRPVNFIAPSLVNCSSFKEHVVSGTDITIVRELTGGIYFGARQEHDGSFNSASDLDHYDRDSIVRAARLAGKLAMSRQPHLPVTSLDKANLLAACGRLWRGVVKEVFETEFPTIKLSHMLIDTAAMNVARRPTSLNGIILTSNMFGDIISDEASAIPGSLGLLPSASLCAIPTVLDASKVRGIYEPIHGSAPDIAGQGIINPTGMILSVAMMLRYSLAMPEAADSIEAAVGKVIEDDCRTSDIGGRASTADFGDAVVIALRNCLDKN

SEQ ID NO: 46 - protein, 880 aa, unidentified
  source 1..880: note = Glarea lozoyensis
MPKPTISYSLNGLPAAAVDSIGSLLEILARIRGIVLTEQSNDETASIIISAPDVHRLVVGSDDSHYDSTTLYIEDFLNRLCEALDAVGKKAEAGAFRHALQLYHTDRELGALKHGESVTNRQLKNDSFEDLEFLVEAWLVALNSEESARKLPAPLPVKSPDTRAMTLAEKILAHHAFSLPSSGGLKSGELGMKHGMVSIGELPSVWRNDRFWLAGDHTVEPRTYDQPRVRELLNGLNDAKQEFKMTENQGSNYTILHTEFVRERAEPGMLALGSDSHTCSAGAVSCLAIGLGAADVMTALATGETWIKVPESIRIDFTGEPAWYIRGKDVILYILKELKRNTFAADRIVEFGGLGARFLSCDARFAITNMCTELGGVTGIFVPDEVTNTFVSGRPQSKYKSNSIYFQPDKDASYAATFQIDLTMVESFIALHPSPDNVVPVSEKLDMSFEGCFIGACTTTEEELVLGALVLEAGLKNGLGLAPGKRMVVPGSIPIVNSLRELGLLEIYAQTGFEQPAVGCSLCLGMGADRAGEGENWISSQNRNFKNRMGKGSTGHICSAATVAASSFSMRLTNPAALLAQVSPDRYKALLESCQSYKNRVRKVKSSKKESVKRSVSVMPSYVEPYLHFRSPLTEKHTQMHAPSGGEGEQCGNLDIIESKIYALGDFVDTDAVSRDLFLHLSDVSGETMTSGVLTMLEIIPAAFILESPTDTLLGSHALEFTNPDFRDKVRQGMRVVVAGKAFGCGSSREEAPRALKGLGVQCVIAKSFSFIYGRNQPSIGLLGINIADDRFYELAKTGAGIKIDVPGRVVRLEGQNFPFVLDDMELSLIKNNGLATAYKKYGKNVFALLCNTKSSLRPSELAELQLKGVSDGMQTSLEW

SEQ ID NO: 47 - protein, 546 aa, unidentified
  source 1..546: note = Glarea lozoyensis
MLSEVIARVELLIGEQTLSGGILTFLFIVVIAHFVLTRFTVHSRFWNSQAWTGVREEWFPKMRAKVRTIGNIRQMLSDGYEGFSKQNKAFALPVIAEKPWLVLPHSCIPELLAKSDSEIDMKIIHEEQLMHEYTTGSLGRHVVDVPIQYDILLRQVNRKLPLLISAFNEELDKSFCHYWGTDTSYSEVNLSETCEKIVTQALNRIFAGKEICRDEGFLEHSRLYSEGVGRNAIMVRMLPPLLRPLLAPFITYSNRKHRDICLRVCLPVIRERVQHTAAKRADAEHKWEPPLDVLQWIIEESFARNDPKELDPRMITQRLLALNFVAIDTTHMSMAHTILDLYRSPNSDDFLVGLREECERVLQANGGQWTKSGLDDLVCVDSTIRESMRYSDLGYISLTRMVVDPKGTQFNANGTNSSSPLSVPPGIRICVPAHAIHRDAALYPSPYEFQAFRFSKAREKYRGTQTELSEPKVSIVTTTDKFLPFGHGRHACPGRFFAAQQMKLMLAYLVQNYDVEKLSTKIQNKIMVGTTKPDASLKIKVKRRKV

SEQ ID NO: 48 - protein, 521 aa, unidentified
  source 1..521: note = Glarea lozoyensis
MIAALFTTNLQLGAVGVFIFALLAFAFNKLTTWEYSIPKEVQWVDRRTQPFSYLRAKARALARSKENTLEAYFRFNKLGKAAALAVPFGRPLLLLPQTFVRWIVDQPESIISLDPIHDDFHVFVGGDLTGDHTVQELLRRELTLNLDKLISVINDEIVCALDDVLGNSPEWKSTSLADDLKTIVARTSNRVFVGKDLCRNKHYISTVKGLALVIMPETVLQDLIPQFLKGPLSRITKAFNNIYGMKKFSSLLLGVVRQRYIDVKDVLEGSGDKTRLPDDLLTWMVQKSIRKGESSANIDKLLVARIAMANLAAIETTTAAMTRSVLDLVTQGSEGGFLKAVQEETLAVVEGCNYEPSKKDVLKLVLTENAIKEALRLQVAFPGLMRQVVAPNGVTLENGLHVPCGTRLGVSAAGIHVDESIYEDPTTYNPGRFLVRDLDPRGDPSPMWKGNENYLAFSLGRRSCPGRWYVTDQLKLTLAHIFSKYEIRFEKAAETASALRKILPGAPQDRVMIRRRSVGKR

SEQ ID NO: 49 - protein, 528 aa, unidentified
  source 1..528: note = Aspergillus terreus
MTVDALTQPHHLLSLAWNDTQQHGSWFAPLVTTSAGLLCLLLYLCSSGRRSDLPVFNPKTWWELTTMRAKRDFDANAPSWIESWFSQNDKPIRFIVDSGYCTILPSSMADEFRKMKELCMYKFLGTDFHSHLPGFDGFKEVTRDAHLITKVVMNQFQTQAPKYVKPLANEASGIITDIFGDSNEWHTVPVYNQCLDLVTRTVTFIMVGSKLAHNEEWLDIAKHHAVTMAIQARQLRLWPVILRPLVHWLEPQGAKLRAQVRRARQLLDPIIQERRAERDACRAKGIEPPRYVDSIQWFEDTAKGKWYDAAGAQLAMDFAGIYGTSDLLIGGLVDIVRHPHLLEPLRDEIRTVIGQGGWTPASLYKLKLLDSCLKESQRVKPVECATMRSYALQDVTFSNGTFIPKGELVAVAADRMSNPEVWPEPAKYDPYRYMRLREDPAKAFSAQLENTNGDHIGFGWHPRACPGRFFASKEIKMMLAYLLIRYDWKVVPDEPLQYYRHSFSVRIHPTTKLMMRRRDEDIRLPGSL

SEQ ID NO: 50 - protein, 3038 aa, unidentified
  source 1..3038: note = Aspergillus terreus
MAQSMYPNEPIVVVGSGCRFPGDANTPSKLWELLQHPRDVQSRIPKERFDVDTFYHPDGKHHGRTNAPYAYVLQDDLGAFDAAFFNIQAGEAESMDPQHRLLLETVYEAVTNAGMRIQDLQGTSTAVYVGVMTHDYETVSTRDLESIPTYSATGVAVSVASNRISYFFDWHGPSMTIDTACSSSLVAVHLAVQQLRTGQSSMAIAAGANLILGPMTFVLESKLSMLSPSGRSRMWDAGADGYARGEAVCSVVLKTLSQALRDGDTIECVIRETGVNQDGRTTGITMPNHSAQEALIKATYAQAGLDITKAEDRCQFFEAHGTGTPAGDPQEAEAIATAFFGHEQVARSDGNERAPLFVGSAKTVVGHTEGTAGLAGLMKASFAVRHGVIPPNLLFDKISPRVAPFYKNLRIPTEATQWPALPPGQPRRASVNSFGFGGTNAHAIIEEYMEPEQNQLRVSNNEDCPPMTGVLSLPLVLSAKSQRSLKIMMEEMLQFLQSHPEIHLHDLTWSLLRKRSVLPFRRAIVGHSHETIRRALEDAIEDGIVSSDFTTEVRGQPSVLGIFTGQGAQWPGMLKNLIEASPYVRNIVRELDDSLQSLPEKYRPSWTLLDQFMLEGEASNVQYATFSQPLCCAVQIVLVRLLEAARIRFTAVVGHSSGEIACAFAAGLISASLAIRIAYLRGVVSAGGARGTPGAMLAAGMSFEEAQEICELDAFEGRICVAASNSPDSVTFSGDANAIDHLKGMLEDESTFARLLKVDTAYHSHHMLPCADPYMQALEECGCAVADAGSPAGSVPWYSSVDAENRQMAARDVTAKYWKDNLVSPVLFSHAVQRAVVTHKALDIGIEVGCHPALKSPCVATIKDVLSGVDLAYTGCLERGKNDLDSFSRALAYLWERFGASSFDADEFMRAVAPDRPCMSVSKLLPAYPWDRSRRYWVESRATRHHLRGPKPHLLLGKLSEYSTPLSFQWLNFVRPRDIEWLDGHALQGQTVFPAAGYIVMAMEAALMIAGTHAKQVKLLEILDMSIDKAVIFDDEDSLVELNLTADVSRNAGEAGSMTISFKIDSCLSKEGNLSLSAKGQLALTIEDVNPRTTSASDQHHLPPPEEEHPHMNRVNINAFYHELGLMGYNYSKDFRRLHNMQRADLRASGTLDFIPLMDEGNGCPLLLHPASLDVAFQTVIGAYSSPGDRRLRCLYVPTHVDRITLVPSLCLATAESGCEKVAFNTINTYDKGDYLSGDIVVFDAEQTTLFQVENITFKPFSPPDASTDHAMFARWSWGPLTPDSLLDNPEYWATAQDKEAIPIIERIVYFYIRSFLSQLTLEERQQAAFHLQKQIEWLEQVLASAKEGRHLWYDPGWENDTEAQIEHLCTANSYHPHVRLVQRVGQHLLPTVRSNGNPFDLLDHDGLLTEFYTNTLSFGPALHYARELVAQIAHRYQSMDILEIGAGTGGATKYVLATPQLGFNSYTYTDISTGFFEQAREQFAPFEDRMVFEPLDIRRSPAEQGFEPHAYDLIIASNVLHATPDLEKTMAHARSLLKPGGQMVILEITHKEHTRLGFIFGLFADWWAGVDDGRCTEPFVSFDRWDAILKRVGFSGVDSRTTDRDANLFPTSVFSTHAIDATVEYLDAPLASSGTVKDSYPPLVVVGGQTPQSQRLLNDIKAIMPPRPLQTYKRLVDLLDAEELPMKSTFVMLTELDEELFAGLTEETFEATKLLLTYASNTVWLTENAWVQHPHQASTIGMLRSIRREHPDLGVHVLDVDAVETFDATFLVEQVLRLEEHTDELASSTTWTQEPEVSWCKGRPWIPRLKRDLARNNRMNSSRRPIYEMIDSSRAPVALQTARDSSSYFLESAETWFVPESVQQMETKTIYVHFSCPHALRVGQLGFFYLVQGHVQEGNREVPVVALAERNASIVHVRPDYIYTEADNNLSEGGGSLMVTVLAAAVLAETVISTAKCLGVTDSILVLNPPSICGQMLLHAGEEIGLQVHLATTSGNRSSVSAGDAKSWLTLHARDTDWHLRRVLPRGVQALVDLSADQSCEGLTQRMMKVLMPGCAHYRAADLFTDTVSTELHSGSRHQASLPAAYWEHVVSLARQGLPSVSEGWEVMPCTQFAAHADKTRPDLSTVISWPRESDEATLPTRVRSIDAETLFAADKTYLLVGLTGDLGRSLGRWMVQHGACHIVLTSRNPQVNPKWLAHVEELGGRVTVLSMDVTSQNSVEAGLAKLKDLHLPPVGGIAFGPLVLQDVMLNNMELPMMEMVLNPKVEGVRILHEKFSDPTSSNPLDFFVMFSSIVAVMGNPGQANYSAANCYLQALAQQRVASGLAASTIDIGAVYGVGFVTRAELEEDFNAIRFMFDSVEEHELHTLFAEAVVAGRRAVHQQEQQRKFATVLDMADLELTTGIPPLDPALKDRITFFDDPRIGNLKIPEYRGAKAGEGAAGSKGSVKEQLLQATNLDQVRQIVIDGLSAKLQVTLQIPDGESVHPTIPLIDQGVDSLGAVTVGTWFSKQLYLDLPLLKVLGGASITDLANEAAARLPPSSIPLVAATDGGAESTDNTSENEVSGREDTDLSAAATITEPSSADEDDTEPGDEDVPRSHHPLSLGQEYSWRIQQGAEDPTVFNNTIGMFMKGSIDLKRLYKALRAVLRRHEIFRTGFANVDENGMAQLVFGQTKNKVQTIQVSDRAGAEEGYRQLVQTRYNPAAGDTLRLVDFFWGQDDHLLVVAYHRLVGDGSTTENIFVEAGQLYDGTSLSPHVPQFADLAARQRAMLEDGRMEEDLAYWKKMHYRPSSIPVLPLMRPLVGNSSRSDTPNFQHCGPWQQHEAVARLDPMVAFRIKERSRKHKATPMQFYLAAYQVLLARLTDSTDLTVGLADTNRATVDEMAAMGFFANLLPLRFRDFRPHITFGEHLIATRDLVREALQHARVPYGVLLDQLGLEVPVPTSNQPAPLFQAVFDYKQGQAESGTIGGAKITEVIATRERTPYDVVLEMSDDPTKDPLLTAKLQSSRYEAHHPQAFLESYMSLLSMFSMNPALKLA

SEQ ID NO: 51 - protein, 363 aa, unidentified
  source 1..363: note = Aspergillus terreus
MGDQPFIPPPQQTALTVNDHDEVTVWNAAPCPMLPRDQVYVRVEAVAINPSDTKMRGQFATPWAFLGTDYAGTVVAVGSDVTHIQVGDRVYGAQNEMCPRTPDQGAFSQYTVTRGRVWAKIPKGLSFEQAAALPAGISTAGLAMKLLGLPLPSPSADQPPTHSKPVYVLVYGGSTATATVTMQMLRLSGYIPIATCSPHNFDLAKSRGAEEVFDYRAPNLAQTIRTYTKNNLRYALDCITNVESTTFCFAAIGRAGGHYVSLNPFPEHAATRKMVTTDWTLGPTIFGEGSTWPAPYGRPGSEEERQFGEDLWRIAGQLVEDGRLVHHPLRVVQGGFDHIKQGMELVRKGELSGEKLVVRLEGP

SEQ ID NO: 52 - protein, 256 aa, unidentified
  source 1..256: note = Aspergillus terreus
MRYQASPALVKAPRALLCIHGAGCSPAIFRVQLSKLRAALRENFEFVYVTAPFPSSAGPGILPVFADLGPYYSWFESSSDNNHNGPSVSERLAAVHDPIRRTIVDWQTQHPHIPIVGAIGFSEGALVTTLLLWQQQMGHLPWLPRMSVALLICPWYQDEASQYMRNEVMKNHDDDNDSKDTEWQEELVIRIPTLHLQGRDDFALAGSKMLVARHFSPREAQVLEFAGQHQFPNRPRDVLEVINRFRKLCVTAQTLE

SEQ ID NO: 53 - protein, 413 aa, unidentified
  source 1..413: note = Aspergillus terreus
MGSIIDAAAAADPVVLMETAFRKAVKSRQIPGAVIMARDCSGNLNYTRCFGARTVRRDECNQLPPLQVDTPCRLASATKLLTTIMALQCMERGLVDLDETVDRLLPDLSAMPVLEGFDDAGNARLRERRGKITLRHLLTHTSGLSYVFLHPLLREYMAQGHLQSAEKFGIQSRLAPPAVNDPGAEWIYGANLDWAGKLVERATGLDLEQYLQENICAPLGITDMTFKLQQRPDMLARRADQTHRNSADGRLRYDDSVYFRADGEECFGGQGVFSGPGSYMKVLHSLLKRDGLLLQPQTVDLMFQPALEPRLEEQMNQHMDASPHINYGGPMPMVLRRSFGLGGIIALEDLDGENWRRKGSLTFGGGPNIVWQIDPKAGLCTLAFFQLEPWNDPVCRDLTRTFEHAIYAQYQQG

SEQ ID NO: 54 - protein, 2532 aa, unidentified
  source 1..2532: note = Aspergillus terreus
MTPLDAPGAPAPIAMVGMGCRFGGGATDPQKLWKLLEEGGSAWSKIPPSRFNVGGVYHPNGQRVGSMHVRGGHFLDEDPALFDASFFNMSTEVASCMDPQYRLILEVVYEALEAAGIPLEQVSGSKTGVFAGTMYHDYQGSFQRQPEALPRYFITGNAGTMLANRVSHFYDLRGPSVSIDTACSTTLTALHLAIQSLRAGESDMAIVAGANLLLNPDVFTTMSNLGFLSSDGISYSFDSRADGYGRGEGVAAIVLKTLPDAVRDGDPIRLIVRETAINQDGRTPAISTPSGEAQECLIQDCYQKAQLDPKQTSYVEAHGTGTRAGDPLELAVISAAFPGQQIQVGSVKANIGHTEAVSGLASLIKVALAVEKGVIPPNARFLQPSKKLLKDTHIQIPLCSQSWIPTDGVRRASINNFGFGGANAHAIVEQYGPFAETSICPPNGYSGNYDGNLGTDQAHIYVLSAKDENSCMRMVSRLCDYATHARPADDLQLLANIAYTLGSRRSNFRWKAVCTAHSLTGLAQNLAGEGMRPSKSADQVRLGWVFTGQGAQWFAMGRELIEMYPVFKEALLECDGYIKEMGSTWSIIEELSRPETESRVDQAEFSLPLSTALQIALVRLLWSWNIQPVAVTSHSSGEAAAAYAIGALTARSAIGISYIRGALTARDRLASVHKGGMLAVGLSRSEVGIYIRQVPLQSEECLVVGCVNSPSSVTVSGDLSAIAKLEELLHADRIFARRLKVTQAFHSSHMNSMTDAFRAGLTELFGADPSDAANASKDVIYASPRTGARLHDMNRLRDPIHWVECMLHPVEFESAFRRMCLDENDHMPKVDRVIEIGPHGALGGPIKQIMQLPELATCDIPYLSCLSRGKSSLSTLRLLASELIRAGFPVDLNAINFPRGCEAARVQVLSDLPPYPWNHETRYWKEPRISQSARQRKGPVHDLIGLQEPLNLPLARSWHNVLRVSDLPWLRDHVVGSHIVFPGAGFVCMAVMGISTLCSSDHESDDISYILRDVNFAQALILPADGEEGIDLRLTICAPDQSLGSQDWQRFLVHSITADKNDWTEHCTGLVRAEMDQPPSSLSNQQRIDPRPWSRKTAPQELWDSLHRVGIRHGPFFRNITCIESDGRGSWCTFAIADTASAMPHAYESQHIVHPTTLDSAVQAAYTTLPFAGSRIKSAMVPARVGCMKISSRLADLEARDMLRAQAKMHSQSPSALVTDVAVFDEADPVGGPVMELEGLVFQSLGASLGTSDRDSTDPGNTCSSWHWAPDISLVNPGWLEKTLGTGIQEHEISLILELRRCSVHFIQEAMESLSVGDVERLSGHLAKFYAWMQKQLACAQNGELGPESSSWTRDSEQARCSLRSRVVAGSTNGEMICRLGSVLPAILRREVDPLEVMMDGHLLSRYYVDALKWSRSNAQASELVRLCCHKNPRARILEIGGGTGGCTQLVVDSLGPNPPVGRYDFTDVSAGFFEAARKRFAGWQNVMDFRKLDIEDDPEAQGFVCGSYDVVLACQVLHATSNMQRTLTNVRKLLKPGGKLILVETTRDELDLFFTFGLLPGWWLSEEPERQSTPSLSPTMWRSMLHTTGFNGVEVEARDCDSHEFYMISTMMSTAVQATPMSCSVKLPEVLLVYVDSSTPMSWISDLQGEIRGRNCSVTSLQALRQVPPTEGQICVFLGEVEHSMLGSVTNDDFTLLTSMLQLAGGTLWVTQGATMKSDDPLKALHLGLLRTMRNESHGKRFVSLDLDPSRNPWTGDSRDAIVSVLDLISMSDEKEFDYAERDGVIHVPRAFSDSINGGEEDGYALEPFQDSQHLLRLDIQTPGLLDSLHFTKRNVDTYEPDKLPDDWVEIEPRAFGLNFRDIMVAMGQLESNVMGFECAGVVTSLSETARTIAPGLAVGDRVCALMNGHWASRVTTSRTNVVRIPETLSFPHAASIPLAFTTAYISLYTVARILPGETVLIHAGAGGVGQAAIILAQLTGAEVFTTAGSETKRNLLIDKFHLDPDHVFSSRDSSFVDGIKTRTRGKGVDVVLNSLAGPLLQKSFDCLARFGRFVEIGKKDLEQNSRLDMSTFVRNVSFSSVDILYWQQAKPAEIFQAMSEVILLWERTAIGLIHPISEYPMSALEKAFRTMQSGQHVGKIVVTVAPDDAVLVRQERMPLFLKPNVSYLVAGGLGGIGRRICEWLVDRGARYLIILSRTARVDPVVTSLQERGCTVSVQACDVADESQLEAALQQCRAEEMPPIRGVIQGAMVLKDALVSQMTADGFHAALRPKVQGSWNLHRIASDVDFFVMLSSLVGVMGGAGQANYAAAGAFQDALAEHRMAHNQPAVTIDLGMVQSIGYVAETDSAVAERLQRIGYQPLHEEEVLDVLEQAISPVCSPAAPTRPAVIVTGINTRPGPHWAHADWMQEARFAGIKYRDPLRDNHGALSLTPAEDDNLHARLNRAISQQESIAVIMEAMSCKLISMFGLTDSEMSATQTLAGIGVDSLVAIELRNWITAKFNVDISVFELMEGRTIAKVAEVVLQRYKA

SEQ ID NO: 55 - protein, 3032 aa, unidentified
  source 1..3032: note = Penicillium citrinum
MDQANYPNEPIVVVGSGCRFPGGVNTPSKLWELLKEPRDVQTKIPKERFDVDTFYSPDGTHPGRTNAPFAYLLQEDLRGFDASFFNIQAGEAETIDPQQRLLLETVYEAVSNAGLRIQGLQGSSTAVYVGMMTHDYETIVTRELDSIPTYSATGVAVSVASNRVSYFFDWHGPSMTIDTACSSSLAAVHLAVQQLRTGESTMAVAAGANLILGPMTFVMESKLNMLSPNGRSRMWDAAADGYARGEGVCSIVLKTLSQALRDGDSIECVIRETGINQDGRTTGITMPNHSAQEALIRATYAKAGLDITNPQERCQFFEAHGTGTPAGDPQEAEAIATAFFGHKDGTIDSDGEKDELFVGSIKTVLGHTEGTAGIAGLMKASFAVRNGVIPPNLLFEKISPRVAPFYTHLKIATEATEWPIVAPGQPRRVSVNSFGFGGTNAHAIIEEYMAPPHKPTAVVTEVTSDADACSLPLVLSSKSQRSMKATLENMLQFLETHDDVDMHDIAYTLLEKRSILPFRRAIAAHNKEVARAALEAAIADGEVVTDFRTDANDNPRVLGVFTGQGAQWPGMLKKLMVGMPFVRGILEELDNSLQTLPEKYRPTWTLYDQLMLEGDASNVRLASFSQPLCCAVQIVLVRLLAAAGIEFSAIVGHSSGEIACAFAAGFISATQAIRIAHLRGVVSAEHASSPSGQTGAMLAAGMSYDDAKELCELEAFEGRVCVAASNSPDSVTFSGDMDAIQHVEGVLEDESTFARILRVDKAYHSHHMHPCAAPYVKALLECDCAVADGQGNDSVAWFSAVHETSKQMTVQDVMPAYWKDNLVSPVLFSQAVQKAVITHRLIDVAIEIGAHPALKGPCLATIKDALAGVELPYTGCLARNVDDVDAFAGGLGYIWERFGVRSIDAEGFVQQVRPDRAVQNLSKSLPTYSWDHTRQYWAESRSTRQHLRGGAPHLLLGKLSSYSTASTFQWTNFIRPRDLEWLDGHALQGQTVFPAAGYIIMAMEAAMKVAGERAAQVQLLEILDMSINKAIVFEDENTSVELNLTAEVTSDNDADGQVTVKFVIDSCLAKESELSTSAKGQIVITLGEASPSSQLLPPPEEEYPQMNNVNIDFFYRELDLLGYDYSKDFRRLQTMRRADSKASGTLAFLPLKDELRNEPLLLHPAPLDIAFQTVIGAYSSPGDRRLRSLYVPTHVDRVTLIPSLCISAGNSGETELAFDTINTHDKGDFLSGDITVYDSTKTTLFQVDNIVFKPFSPPTASTDHRIFAKWVWGPLTPEKLLEDPATLIIARDKEDILTIERIVYFYIKSFLAQITPDDRQNADLHSQKYIEWCDQVQADARAGHHQWYQESWEEDTSVHIEQMCESNSSHPHVRLIQRVGKELISIVRGNGDPLDIMNRDGLFTEYYTNKLAFGSAIHVVQDLVSQIAHRYQSIDILEIGLGTGIATKRVLASPQLGFNSYTCTDISADVIGKAREQLSEFDGLMQFEALDINRSPAEQGFKPHSYDLIIASDVLHASSNFEEKLAHIRSLLKPGGHLVTFGVTHREPARLAFISGLFADRWTGEDETRALSASGSVDQWEHTLKRVGFSGVDSRTLDREDDLIPSVFSTHAVDATVERLYDPLSAPLKDSYPPLVVIGGESTKTERILNDMKAALPHRHIHSVKRLESVLDDPALQPKSTFVILSELDDEVFCNLEEDKFEAVKSLLFYAGRMMWLTENAWIDHPHQASTIGMLRTIKLENPDLGTHVFDVDTVENLDTKFFVEQLLRFEESDDQLLESITWTHEPEVYWCKGRAWVPRLKQDIARNDRMNSSRRPIFGNFNSSKTAIALKEARGASSSMYYLESTETCDSLEDARHAGKATVRVRYALPQAIRVGHLGYFHVVQGSILENTCEVPVVALAEKNGSILHVPRNYMHSLPDNMAEGEDSSFLLSTAAALLAETILSSAQSFGSDASILIMEPPIFCVKAILESAKTYGVQVHLATTLSDVKTIPAPWIRLHAKETDARLKHSLPTNMMAFFDLSTDRTAAGITNRLAKLLPPSCFMYSGDYLIRSTASTYKVSHVEDIPILEHSVAMAKNTVSASTVDDTEKVITATQILLPGQLSVNHNDQRFNLATVIDWKENEVSARICPIDSGNLFSNKKTYLLVGLTGDLGRSLCRWMILHGARHVVLTSRNPRLDPKWIANMEALGGDITVLSMDVANEDSVDAGLGKLVDMKLPPVAGIAFGPLVLQDVMLKNMDHQMMDMVLKPKVQGARILHERFSEQTGSKALDFFIMFSSIVAVIGNPGQSNYGAANAYLQALAQQRCARGLAGSTIDIGAVYGVGFVTRAEMEEDFDAIRFMFDSVEEHELHTLFAEAVVSDQRARQQPQRKTVIDMADLELTTGIPDLDPALQDRIIYFNDPRFGNFKIPGQRGDGGDNGSGSKGSIADQLKQATTLDQVRQIVIDGLSEKLRVTLQVSDGESVDPTIPLIDQGVDSLGAVTVGSWFSKQLYLDLPLLRVLGGASVADLADDAATRLPATSIPLLLQIGDSTGTSDSGASPTPTDSHDEASSATSTDASSAEEDEEQEDDNEQGGRKILRRERLSLGQEYSWRQQQMVKDHTIFNNTIGMFMKGTIDLDRLRRALKASLRRHEIFRTCFVTGDDYSSDLNGPVQVVLKNPENRVHFVQVNNAAEAEEEYRKLEKTNYSISTGDTLRLVDFYWGTDDHLLVIGYHRLVGDGSTTENLFNEIGQIYSGVKMQRPSTQFSDLAVQQRENLENGRMGDDIAFWKSMHSKVSSSAPTVLPIMNLINDPAANSEQQQIQPFTWQQYEAIARLDPMVAFRIKERSRKHKATPMQFYLAAYHVLLARLTGSKDITIGLAETNRSTMEEISAMGFFANVLPLRFDEFVGSKTFGEHLVATKDSVREAMQHARVPYGVILDCLGLNLPTSGEEPKTQTHAPLFQAVFDYKQGQAESGSIGNAKMTSVLASRERTPYDIVLEMWDDPTKDPLIHVKLQSSLYGPEHAQAFVDHFSSILTMFSMNPALKLA

SEQ ID NO: 56 - protein, 2563 aa, unidentified
  source 1..2563: note = Penicillium citrinum
MNNTPAVTATATATATATAMAGSACSNTSTPIAIVGMGCRFAGDATSPQKLWEMVERGGSAWSKVPSSRFNVRGVYHPNGERVGSTHVKGGHFIDEDPALFDAAFFNMTTEVASCMDPQYRLMLEVVYESLESAGITIDGMAGSNTSVFGGVMYHDYQDSLNRDPETVPRYFITGNSGTMLSNRISHFYDLRGPSVTVDTACSTTLTALHLACQSLRTGESDTAIVIGANLLLNPDVFVTMSNLGFLSPDGISYSFDPRANGYGRGEGIAALVIKALPNALRDQDPIRAVIRETALNQDGKTPAITAPSDVAQKSLIQECYDKAGLDMSLTSYVEAHGTGTPTGDPLEISAISAAFKGHPLHLGSVKANIGHTEAASGLASIIKVALALEKGLIPPNARFLQKNSKLMLDQKNIKIPMSAQDWPVKDGTRRASVNNFGFGGSNAHVILESYDRASLALPEDQVHVNGNSEHGRVEDGSKQSRIYVVRAKDEQACRRTIASLRDYIKSVADIDGEPFLASLAYTLGSRRSILPWTSVYVADSLGGLVSALSDESNQPKRANEKVRLGFVFTGQGAQWHAMGRELVNTFPVFKQAILECDGYIKQLGASWNFMEELHRDELTTRVNDAEYSLPLSTAIQIALVRLLWSWGIRPTGITSHSSGEAAAAYAAGALSARSAIGITYIRGVLTTKPKPALAAKGGMMAVGLGRSETNVYISRLNQEDGCVVVGCINSQCSVTVSGDLGAIEKLEKLLHADGIFTRKLKVTEAFHSSHMRPMADAFGASLRDLFNSDNNNDNPNADTSKGVLYSSPKTGSRMTDLKLLLDPTHWMDSMLQPVEFESSLREMCFDPNTKEKAVDVIIEIGPHGALGGPINQVMQDLGLKGTDINYLSCLSRGRSSLETMYRAATELISKGYGLKMDAINFPHGRKEPRVKVLSDLPAYPWNHQTRYWREPRGSRESKQRTHPPHTLIGSRESLSPQFAPKWKHVLRLSDIPWIRDHVVGSSIIFPGAGFISMAIEGFSQVCPPVAGASINYNLRDVELAQALIIPADAEAEVDLRLTIRSCEERSLGTKNWHQFSVHSISGENNTWTEHCTGLIRSESERSHLDCSTVEASRRLNLGSDNRSIDPNDLWESLHANGICHGPIFQNIQRIQNNGQGSFCRFSIADTASAMPHSYENRHIVHPTTLDSVIQAAYTVLPYAGTRMKTAMVPRRLRNVKISSSLADLEAGDALDAQASIKDRNSQSFSTDLAVFDDYDSGSSPSDGIPVIEIEGLVFQSVGSSFSDQKSDSNDTENACSSWVWAPDISLGDSTWLKEKLSTEAETKETELMMDLRRCTINFIQEAVTDLTNSDIQHLDGHLQKYFDWMNVQLDLARQNKLSPASCDWLSDDAEQKKCLQARVAGESVNGEMISRLGPQLIAMLRRETEPLELMMQDQLLSRYYVNAIKWSRSNAQASELIRLCAHKNPRSRILEIGGGTGGCTKLIVNALGNTKPIDRYDFTDVSAGFFESAREQFADWQDVMTFKKLDIESDPEQQGFECATYDVVVACQVLHATRCMKRTLSNVRKLLKPGGNLILVETTRDQLDLFFTFGLLPGWWLSEEPERKSTPSLTTDLWNTMLDTSGFNGVELEVRDCEDDEFYMISTMLSTARKENTTPDTVAESEVLLLHGALRPPSSWLESLQAAICEKTSSSPSINALGEVDTTGRTCIFLGEMESSLLGEVGSETFKSITAMLNNCNALLWVSRGAAMSSEDPWKALHIGLLRTIRNENNGKEYVSLDLDPSRNAYTHESLYAICNIFNGRLGDLSEDKEFEFAERNGVIHVPRLFNDPHWKDQEAVEVTLQPFEQPGRRLRMEVETPGLLDSLQFRDDEGREGKDLPDDWVEIEPKAFGLNFRDVMVAMGQLEANRVMGFECAGVITKLGGAAAASQGLRLGDRVCALLKGHWATRTQTPYTNVVRIPDEMGFPEAASVPLAFTTAYIALYTTAKLRRGERVLIHSGAGGVGQAAIILSQLAGAEVFVTAGTQAKRDFVGDKFGINPDHIFSSRNDLFVDGIKAYTGGLGVHVVLNSLAGQLLQASFDCMAEFGRFVEIGKKDLEQNSRLDMLPFTRDVSFTSIDLLSWQRAKSEEVSEALNHVTKLLETKAIGLIGPIQQHSLSNIEKAFRTMQSGQHVGKVVVNVSGDELVPVGDGGFSLKLKPDSSYLVAGGLGGIGKQICQWLVDHGAKHLIILSRSAKASPFITSLQNQQCAVYLHACDISDQDQVTKVLRLCEEAHAPPIRGIIQGAMVLKDALLSRMTLDEFNAATRPKVQGSWYLHKIAQDVDFFVMLSSLVGVMGGAGQANYAAAGAFQDALAHHRRAHGMPAVTIDLGMVKSVGYVAETGRGVADRLARIGYKPMHEKDVMDVLEKAILCSSPQFPSPPAAVVTGINTSPGAHWTEANWIQEQRFVGLKYRQVLHADQSFVSSHKKGPDGVRAQLSRVTSHDEAISIVLKAMTEKLMRMFGLAEDDMSSSKNLAGVGVDSLVAIELRNWITSEIHVDVSIFELMNGNTIAGLVELVVAKCS

SEQ ID NO: 57 - protein, 518 aa, unidentified
  source 1..518: note = Penicillium citrinum
MLGQVLLTVESYQWVSTPQALVAVAVLLSLIAYRLRGRQSELQVYNPKKWWELTTMRARQDFDTYGPSWIEAWFSKNDKPLRFIVDSGYCTILPSSMADEFRKIKDMCMYKFLADDFHSHLPGFDGFKEICQDAHLVNKVVLNQLQTQAPKYTKPLATLADATIAKLFGKSEEWQTAPVYSNGLDLVTRTVTLIMVGDKICHNEEWLDIAKNHAVSVAVQARQLRVWPMLLRPLAHWFQPQGRKLRDQVRRARKIIDPEIQRRRAEKAACVAKGVQPPQYVDTMQWFEDTADGRWYDVAGAQLAMDFAGIYASTDLFVGALVDIARHPDLIQPLRQEIRTVIGEGGWTPASLFKLKLLDSCMKETQRIKPVECATMRSTALRDITLSNGLFIPKGELAAVAADRMNNPDVWENPENYDPYRFMRMREDPDKAFTAQLENTNGDHIGFGWNPRACPGRFFASKEIKILLAHILIQYDVKPVPGDDDKYYRHAFSVRMHPTTKLMVRRRNEDIPLPHDRC

SEQ ID NO: 58 - protein, 1173 aa, unidentified
  source 1..1173: note = Penicillium citrinum
MVASLLPSRFRGRESMNQQHPLRSGNRALTSTLQFLSKTACLHPIHTVCTIAILASTTYVGLLKDSFFHGPANVDKAEWGSLVEGSRSLITGPQNGWKWQSFDGDADVLGDFNHQALMTLVFPGSYGVASQAASPFLAPLPVNLSVIDLPSTSSPLTAYSKDKVFAFSVEYSSAPELVAAVQEIPNNSADLKLQETQLIEMERQMWIMKAARAHTKRSLAQWVHDTWTESLDLIKSAQTLDVVVMVLGYISMHLTFVSLFLSMKKLGSKVWLATSVLLSSTFAFLLGLDVAIRLGVPMSMRLLSEGLPFLVVIVGFEKSITLTRAVLSYAVQHRKPQKIQSDQGSVTAIAESTINYAVRSAIREKGYNIVCHYVVEILLLVIGAVLGIQGGLQHFCVLAALILFFDCLLLFTFYTAILSIKLEVNRLKRHINMRYALEDEGLSQRTAESVATSNDAQDSARTYLFGNDMKGSSVPKFKFWMVVGFLIVNLVNIGSTLFQASSSGSLSSISSWTESLSGSAIKPPLEPFKVAGSGLDELLFQARGRGQSTMVTVLAPIKYELEYPSIHRGTSQLHEYGVGGKMVGSLLTSLEDPVLSKWVFVALALSVALNSYLFKAARLGIKDPNLPSHPVDPVELDQAESFNAAQNQTPQIQSSLQAPQTRVFTPTTTDSDSDASLVLIKASLKVTKRAEGKTATSELPVSRTQIELDNLLKQNTISELNDEDVVALSLRGKVPGYALEKSLKDCTRAVKVRRSIISRTPATAELTSMLEHSKLPYENYAWERVLGACCENVIGYMPVPVGVAGPIVIDGKSYFIPMATTEGVLVASASRGSKAINLGGGAVTVLTGDGMTRGPCVKFDVLERAGAAKIWLDSDVGQTVMKEAFNSTSRFARLQSMRTTIAGTHLYIRFKTTTGDAMGMNMISKGVEHALNVMATEAGFSDMNIITLSGNYCTDKKPSALNWIDGRGKGIVAEAIIPANVVRDVLKSDVDSMVQLNISKNLIGSAMAGSVGGFNAQAANLAAAIFIATGQDPAQVVESANCITLMNNLRGSLQISVSMPSIEVGTLGGGTILEPQGAMLDMLGVRGSHPTTPGENARQLARIIGSAVLAGELSLCAALAAGHLVKAHMAHNRSAPASSAPSRSVSPSGGTRTVPVPNNALRPSAAATDRARR

SEQ ID NO: 59 - protein, 553 aa, unidentified
  source 1..553: note = Penicillium citrinum
MSEPLPPKEGEPRPQKEESQNDTLEATESKSQHITGLKLGLVVASVTFVAFLMLLDMSIIVTAIPHITSEFHSLNDVGWYGSAYLLANCALQPLAGKLYTLLGLKYTFFAFLCIFELGSVLCGAARSSTMLIVGRAVAGMGGSGLVNGALTILSTAAPKHKQPVLIGVMMGLSQIAIVCGPLLGGAFTQHATWRWCFYINLPIGAVAAFLLLVITIPDRISSTDSELSTDKPMANIKSTLRKLDLVGFVVFAAFATMISLALEWGGSTYTWRSSVIIGLFCGGGFALIAFVLWERHVGDAVAMIPGSVAGKRQVWCSCLFMGFFSGSLLVFSYYLPIYFQAVKDVSPTLSGVYMLPGILGQVIMAMVSGFAIGKTGYYLPWALGSAVLVAIGAGLVSTFQPHTSTVKWVMYQFIAGFGRGCGMQTPIIAIQSTLSPEQGALGISLAVFGQTFGGSLFLDFANLVFGSGLRTGLSKYAPTVDTQAVTAAGATGFRDVVSKNNLPGVVKAYSLAVDHTFYLAVGATACTFVFAFGMGWRKIATKNDTRAVPETDA

SEQ ID NO: 60 - protein, 251 aa, unidentified
  source 1..251: note = Penicillium citrinum
MSPARITDFSPGKPRKALLCIHGAGCSAAIFRVQISKLRVALKNEFEFVYATAPFSSSPGPGVLPVFQGMGPYYTWFQKHHDAVTNTTTPTVGDRVAAVIGPVQKTVQDWSITNPQAPIVGIVAFSEGALVATLLLHQQQMGKLPWFPKMSIAVLICCFYSDEARDYMRAEAQDDDDKLIINVPTLHLHGRQDFALQGSRQMVETHYLPQNADVLEFQGKHNFPNRPSDVQETVKRFQQLYQKVKMSGSFV

SEQ ID NO: 61 - protein, 362 aa, unidentified
  source 1..362: note = Penicillium citrinum
MGVAMTEGSFIPPVKQTILTVNDKDEVVIWDDAPTPKLPADQVYVRIHAVAVNPSDTKMRGDFATPFACLGTDYAGTVVAVGSEITHVKVGDRVFGAQNEMCPRTPEQGAFSQYTITRGRIWAKIPDSMTWEAAASLPAGISTTGLAMKLLGMPLPYSETKPSKKTYVLIYGGSTATATIAMQFMRLSGYTPIATCSHKNFDLAKKNGAEEVFDYRDADCAQKIRDYTRNNLAYALDCIINVESTSTCYKAIGRAGGRYVALNPFPEHAATRKMVTSDWTLGPTIFGEGSTWPAPYGCEASEEVRLFGTELWQVASRLVEEDKLYHHPLRVIDGGLEQVKQGMETVRNGELSGEKIVVRFSV

SEQ ID NO: 62 - protein, 418 aa, unidentified
  source 1..418: note = Penicillium citrinum
MAPSIDVIPTAASTAAGMISDMEAAFKSAVKLKQIPGAVVMARSMNGDIDYTRCFGARTVERDECQRLPPMEIDTPLRLASATKLLTTIMALQCMEQGLVDLDENVNRLLPDLSDMQVLTGFDAAGNAIMRDREGIIKLRHLLTHTSGLSYAFLHPLLQEYMAKGYLKTAEKFGIQSRLAPPAINDPGVEWIYGANLDWAGKLIERATGVDLEEFMQKNICEPLGITDMTFKLQQRPDMLARRSDQTRRNENGSLRYDDSVYFRHDGEECFGGQGVFCGPESYMKVLNSLMKHDGLLLKKDTIELMFQPALDAELEKKMNDHMDTTPHINYGAALPPVMRRNFGLGGIIAMGDLDGHNWRREGSLTFGGGPNIVWQIDPTVGLCTLVVFQLEPWNDPICKDLTRKFEKAMYSQVKCRN

SEQ ID NO: 63 - protein, 459 aa, unidentified
  source 1..459: note = Penicillium citrinum
MSLPHATIPTNLRRRAFRRSCDRCHAQKLKCTGSNANLVRAQCQRCQQAGLRCVYSERLPKRNLHKEAAAGTTRATETSQPMTATSSTVFSSLAETPPPYCSPPTHIGTSALKETLSEPSAATLQFYDTSINFDDPESFPGGWPQPNTFRDDANSNESSGIPDLGYDFEGPLDATAPVSPSLFDLEVEGNSSSGQSNTSNTQRDLFESLSDVSQDLEVILHGVTVEWPKQKILSYPIGDFLNAFGRLLLHLQERVITSSNSSMLDGCLQTKNLFMAVHCYMLSVKIMTSLSQLLLSEVMKAQPCGQKQSTRMDWYWSGSTTRNDNGRAEALPSFHSNLHIGELISHLDPFMHALSSACTTLRVSLRLLSEIETALGIAQEHGAAASIRLVLSDMPSTSWQILGAENKTITPASRLLSVLWSDEAGDEEPKSTKASGKTINVLRRCYKEIFALAKKHNIA

SEQ ID NO: 64 - protein, 1790 aa, unidentified
  source 1..1790: note = Penicillium aethiopicum
MTSAKVLYFSGEIPQGDPEGDQRTLFRKLHLLSKERDHVVLASLLECVTLTLKDECSKLSPQYRDLLPPFESVLDLTDHVVQLRKTPLGGAIERVLVLVFQLGSLVAYHEAHPLEYNFTPASTVIIGRGSGLLSAAAIGLSPSIVMVPSIAKEIARISFRFGLVVDKVCRSLEVSSDEINSDGAWVYCVHGIGEKEARDAVNQFNEIKAYPSTNGASVFNVDDAGNSVSIGGPPKTLEALFSESNIFKKTKNVAMRKIQGMWHTDRVYGPEHVEQIVPKIESARELHVPLISPVSGDPFRETEAGPLLEQIMEEILMERVRWDMIIETVSKQLKQLMPKSVQLVSIQPSHYNQNMLERWKSELPDAAVSGLTMMPAILELALEQSPPKDTRSSKIAVVGMSCRFPGSDTTEEFWERLMLGEDMHRHIPPDRFDVETHVDPTGKRHNTSKTSYGCFVDNPGLFDAMFFGMSPREAEQTDPMQRLALVTAYEALEKAGYVDGRGVIHRKRVGTFYGQASDDYREVNSGQEVGTYFIPGGCRAFGPGRINYFLNFWGPSFSVDTACSSSLAAIQAACSSLWSGDIDMAITGGMNILSNSDVYAGLSQGHFLSPTGGCKTWDEGADGYCRSDGVGSVVLKRLEDAEADNDNILAVVLSAATSHSAEAVSITHPHDAAQALLYNQIVRRAGIDPLEVGYVEMHGTGTQAGDPTEMRSVTSVFAPPHIQGSRPIPLHVGSVKANMGHGEAAAGIMAFVKTMLVFQNGIIPPHIGVKTGLNPALPDLDKAGVVIPFRAANWRPTGTKKRLAMVNNFGAAGGNTAMIIEEAKARPRLCEDIREAHAITISAKTAVSLSLNIKRLVEYIESAQDLSLADVAYTVSARRRHYEYRKSVVVRSLAEAIKHLQPHIETAKSQTPTLVKRPPVAFAFAGQGTFYVGIAAQIYRDSPFFRAQIDQFDNLARRQNFPSFLPAINKTCAHEDLPASSIHLAIVCVEVALARMCMTFGIKPCAVIGHSLGEYAALAVAEVLSDSDTVFLVGTRATILESNCSPYTHGMISVRASVDDISREADGLPFEVSCINGPNETVIGGTVENLEAVADRLSKVGYRKTRLDVPHAYHTAQMDNVVNELIRQSQGIAYNTPKIPIMSPRDSSVIETGANIDSSYLPTSLKKAVDFAGALNAAWEAGVVSKSTVWLELSHHPVCSGFINRTLPNTSLTCSTLHRDSDNWTSLLKTLSSLYEVGLNIDWNEYHRPFEHALRLVSAPTYAWNNKDYWIQYRGDWNLTKGQVLPEAELPAVSGFRTSSIHRLYSENYDSSTAHLLGECNMTDLSLKGVIEGHAMNGYGVASSFLHAEMAFTLARRIQEKASLSTFTGMGINVTNFEYHDPVVKDASSLDPYPIVVDAEANLEMGEVQIKWFNPAIEKWYCHAIAYYEDPSTWLSNWSRTTRLVTSRIDALVAMSNKGMANKLTTSLAYTLFGKLVDYSSMYHTMQWVILNEDEAVAEVVFPADTQGDWAVPPHFIDGVVSLSGFILNGGTHFDNVNNFFITPSWKSMRFAKPLAPGGRYLTYVRMIPEGVDDKGRLGSYVGDVYILQDGEIVGVVEAILFRQWPRIMLNRFFQPVGMAPPAPRVEKKRDAGRGTLPSSSSLQEKTTATAVTAKITARFPGSVITPSRSAPISKSGSSPKIVPQLDYSLLTPRTSPNSDERIEKTDSDSGFEEADGANDVTSRAVEILAEELAVDKGLLTDECEIADIGVDSLMSLVISQKLREDLGIEVRDAFYLEVTTIGDLKKLLS

SEQ ID NO: 65 - protein, 419 aa, unidentified
  source 1..419: note = Penicillium aethiopicum
MASNTSRSAHLAQIITENTANIETYRREQGLPPLSLGPDAPLDVKYPPNVEKCRRAVIDATLELGELATGPVELRLVPGWAIMTMFGVTQFICDFDIARQIPLAGDISYEDLSKAINVAVPVLRQVLRAGMPYHMFYESRPGHVAHTATTKVMASESLISDWTSLYTDVLFPASAGLSKALREEPTASDPSKTGFMVTKGDGESGMYMYFEKHPEEARRFAGVMEAFQKDEAYAVRHLTDSWPSDSQTGKLVDLGGSTGAVAFALAEKFPGLEIVVQDLPGALEAAHVREGKNVSFMPHDFFNEQPVKDADVYMFRWILHNWPDGHVQRILRALVPSLKPGAKVIVFDEIMPPAGTLPLSIERYQRNIDFGMLTLFNSKIRDIVEWKEIITQSDQRFNVTGVRYPENSRLSLIEIVWQP

SEQ ID NO: 66 - protein, 396 aa, unidentified
  source 1..396: note = Penicillium aethiopicum
MTLDQISRIQALVAEIADLSQTFEGPSERFMVDGLPFLRKSEELVTLVQSPAEHATSLIIKTMETAVIRTLLSLNVLQTIPTTGSITLQSLAVATETQESLLERLLRVVTKTGFIIRENGGYSHSHTSLAYAGPLGALFAPCYDEGIRALVRLPEYLSVKDKEEAKNARYSLFTWNEGQEGKATFEILSTMPARTEGIHTLAMNVQHLRPYTGFFDYSKLVSEDRERPVFVDVGGGNGHVIKEILQAFPQIRPEQCVLEDRAETLELARTTGLLPAGVQLLEHDYLTRQPVSNAKAYHLRAVAYNLGDAELVQLLKQIVPVMGADSKVLIAENILFDDNSTVFSTVSDMIMLGIGGKERTEQNFREVLYEAGLTIEGIHRAPGLEYGIMEASLATS

SEQ ID NO: 67 - protein, 378 aa, unidentified
  source 1..378: note = Penicillium aethiopicum
MSTPEQWIQEFEALCSRASILFPSGIEDENIRIRALRIAEKAVHQLHTPMTFAEAQTWAPLELFGAGVACEMGIFDVLSKSSVPLSPVDIATELNTDPALVARIMRLLDAHYMVDQVTLGQYAANAITRDYVQPYRKGNVMTQVALMPSYFALPAWLRDNDYKVRPDANHCAWQVGANTTKTFWEMPRTTQEDDFVTFYPFEAVFSTSNVDDILFVDIGGGLGHQAMRVRSAFPRSRGRIIVQDLPQVTNKITTASLPDVEIMDHDMADPQPVKGARVYYLRGVLHNHADHISIKYLSQFAAAMSPESRLLIHEALATDLNPTKNITRFDLSMLASCGGAQRSEAEQKALLEKVGLEVSGVWSTPRDWSIMEARLKRE

SEQ ID NO: 68 - protein, 377 aa, unidentified
  source 1..377: note = Penicillium aethiopicum
MPKTAFITGANGLSGSAIVEYLCNTTTSDDWGSIIVTSRSPFKSTVMDPRIKFIALDFVNDVSSLVETMKEVCGAVTHAYFCSYLHKDDFAESYTVNKALFENFIAAIDKAAPKLENVTLQTGGKYYNLHVEPVPSPARENDPRRYGPFENFYFTQEDTLAEMQRGKTWSWNVIRPEAIIGANSQPYGLNVALTIAMYFLICRELGSASPMPTNQRYWEGTDDVSYAPLIADLTIFVSTRKSCANEAFNVTNGDYFTWRYMWPRLAASLGAKADSQQCFEKPMPGEGELQLDWSLAEWCKDKRKVWEDLCDRQGLPGAKATFDLAGWAVGDFLYQRTWSATLSVNKARRFGWTGHMDSYQSFVDTFDKFRQLGLIPK

SEQ ID NO: 69 - protein, 504 aa, unidentified
  source 1..504: note = Penicillium aethiopicum
MTVLFILSAGLVAVFGYLVSWFIYCRTLHPLSKVPGPFWPSVTRLWLTYAVSRGELDVVQRDLHRRYGPLVRIAPDEIACADPEAIRKIYSTTSPLNKSDFYHIWDVGAFSKYPNAFAIVDENMHFERRRIVSSVYSMSTVLTLEPYIDNCSRLFVKRMTERTVPHEAIDLGDWFLWYAYDVIGELFFGHSLGFIENRGDEGGFLASLEVMLPVLTIAAASSPLVRGLIMGLFTLSSTARKGLKGMNHIIETARASVDKRASAVAEPGKGERKDLLHNLLNIVSSKGDKLDFGIEDVKNEAFAALTAGADATMIELQAIFYYLVKDRSVYEELRKEVDQAVETGKLSEFPSYSEVVQLPLLKATIKEALRLHPAVGFTMPRVVGQAGIELLGMYIPPGWKVGMNAAVVGRDEGVYGTDANTFRPERWIERTDSDMDRCNNLVFGAGTRTCIGKQIALSEIYKMVPLLIRKFDFALVDPSKSWTTHDYFFNKQSGVQVKVTVRGL

SEQ ID NO: 70 - protein, 533 aa, unidentified
  source 1..533: note = Penicillium aethiopicum
MAIPQSCTVLVAGGGPGGSYTAAALAREGVDVVLLEADCHPRYHIGESLLPSMRYLLRFIDLEDTFEQHGFQKKLGAFFKLNAKSAGYTDFIRANGPNGYSWNVVRSESDEILFRHATKSGAKTFENVSLKSVNFEPYENDKFTSQDKLTNPGRPVSAEWKTKDGCSGTISFDYLVDATGRVGILSTKYLKNRKFNESFRNIAMWGYFKGNIPPSPGTDRENQPISEGMRDGSGWVWMLPLHNGTVSIGAVVRKDIFQAKKKALPEGTTEAQTLASLVALCPTISSYLEPAELASGIRQAADYSYSANAYAGPNFRIVGDAGCFIDPFFSSGHHLALSSALAAATSINACIRGDCNEFDASRWFAKKVDEGYTLFLVVVMAALKQIRMQEQPILSDLDEEGFDRAFTILRPVIQGAADKETAPKAKGESITETIDLCLTALNDLHDTELQRKLTSIVEAKGTPEQEQLLGKLSPDETAALHRMRAMHSILPMGELEDFENSNIDGFKARLEKGSLGLRRERALCRDHAGDLQM

SEQ ID NO: 71 - protein, 251 aa, unidentified
  source 1..251: note = Penicillium aethiopicum
MPATVLITGGNRGLGKGLVATYLSTPDTTVIATVRDPSKCESLSALPKALGSNLLLVKLEVTSKDSITTAIGTLDTHNIGSIDVVIANAGISGPTSSLAEAPVSELQRYIDVNAYGPFELFKAVLPLLRSSNSGNKAKFVCISSAGGSLAAMYNFMPISAYGASKALANFLVKWLALDNKDIIIWAQNPGSVDTDMARDGLDLAKSLGFDLSSLSFTSPEESACAIKKLIDGATTEMSGKFLDHDGSELAW

SEQ ID NO: 72 - protein, 952 aa, unidentified
  source 1..952: note = Gibberella fujikuroi
MPGKIENGTPKDLKTGNDFVSAAKSLLDRAFKSHHSYYGLCSTSCQVYDTAWVAMIPKTRDNVKQWLFPECFHYLLKTQAADGSWGSLPTTQTAGILDTASAVLALLCHAQEPLQILDVSPDEMGLRIEHGVTSLKRQLAVWNDVEDTNHIGVEFIIPALLSMLEKELDVPSFEFPCRSILERMHGEKLGHFDLEQVYGKPSSLLHSLEAFLGKLDFDRLSHHLYHGSMMASPSSTAAYLIGATKWDDEAEDYLRHVMRNGAGHGNGGISGTFPTTHFECSWIIATLLKVGFTLKQIDGDGLRGLSTILLEALRDENGVIGFAPRTADVDDTAKALLALSLVNQPVSPDIMIKVFEGKDHFTTFGSERDPSLTSNLHVLLSLLKQSNLSQYHPQILKTTLFTCRWWWGSDHCVKDKWNLSHLYPTMLLVEAFTEVLHLIDGGELSSLFDESFKCKIGLSIFQAVLRIILTQDNDGSWRGYREQTCYAILALVQARHVCFFTHMVDRLQSCVDRGFSWLKSCSFHSQDLTWTSKTAYEVGFVAEAYKLAALQSASLEVPAATIGHSVTSAVPSSDLEKYMRLVRKTALFSPLDEWGLMASIIESSFFVPLLQAQRVEIYPRDNIKVDEDKYLSIIPFTWVGCNNRSRTFASNRWLYDMMYLSLLGYQTDEYMEAVAGPVFGDVSLLHQTIDKVIDNTMGNLARANGTVHSGNGHQHESPNIGQVEDTLTRFTNSVLNHKDVLNSSSSDQDTLRREFRTFMHAHITQIEDNSRFSKQASSDAFSSPEQSYFQWVNSTGGSHVACAYSFAFSNCLMSANLLQGKDAFPSGTQKYLISSVMRHATNMCRMYNDFGSIARDNAERNVNSIHFPEFTLCNGTSQNLDERKERLLKIATYEQGYLDRALEALERQSRDDAGDRAGSKDMRKLKIVKLFCDVTDLYDQLYVIKDLSSSMK

SEQ ID NO: 73 - protein, 508 aa, unidentified
  source 1..508: note = Pleurotus sapidus
MIPVVLLLLLPIVVFAWRWRKIGHRERFLPPGPPTRFLLGNVQDIPRTLPFVQLTRWAAEYGELVSLKILNQTMIVVNSGPAVKEIMDKQGVFSGNRPQAYLIKRAEGEYPIFSNLDERIFKGSRRAYHTFLTSEAIASYLPMQQVEYARLMHDIASSPEDFIPHIKRMSTSIMVTLLYGKPVSDFGDNKHLLYFDAMKKFTELTDPWAHPPLDIMPILKHVPARWARWKGLCEEAKQLRGAFFDDITDDFEARYRAGERTGSFLEKVLDHPNHFDMTIEEIRGMSRLLMDGGVETSASYIQNFILALACHPACQDKAQAEIDRVIGGERIPTLEDFDDLPYLRALIKEVHRFRPVLPGAVPHMAARDIRYKEHLIPEGAIIVMNTWGIFHDTEVFENPEKFDPERYIKSEYGVKDGASSDGIRDTLSFGAGRRICPGAEMGQRTIAINTMNLLWAFKFTDKTGKCSMDLENYHAGAELSPNAFPCDIKVRDAKRAKLIEETYMYMTA

SEQ ID NO: 74 - protein, 508 aa, unidentified; identical to SEQ ID NO: 73
  source 1..508: note = Pleurotus sapidus

SEQ ID NO: 75 - protein, 252 aa, Desulfitobacterium hafniense
MRLMNKVAIVTGAASGMGKSIALLYAKEGAKVVVSDLNLEGAHKVAEEITSAGGTALAIKTNVAVEEDIQALVDTAVSTYGTVDIFVNNAGIMDNFEPAADIEDKNWERIFAVNTTSVMRATRKVLPIFLEKSSGVIVNVASAGGLNGARAGATYTASKHAVIGFTKNTGFMYAQQGIRCNAIAPGAVETNIGSTITDPHKFGSARAYAGMATNPRMGKPEEIAQVALFLAAEESSFVNGAVIVADGGWSAY

SEQ ID NO: 76 - protein, 521 aa, unidentified
  source 1..521: note = Aspergillus fumigatus
STYKSRPAAFFFTSGDFHSDQFNKAIAGRRDFVRSCRRGHAERNYSGFSLADGTIIWTLKYVPLAASRCLIFLRVIRMQKFSDQVKEMPRRERRVLETLLYALPFLLAQNLVPAILILKTKKNAVLRYLWIFWSVFVFYQWLQIPISHGESGTYVAKVGVQLFIVILQGFNLVLINPLDRDELLQTKVTGPRDDLLRQVYKVARLFTYLRGVRTPWQVKGIPSHPAYLELQPKTPISRSAFLVRQAAIVAWLYLYLNCANSLADRNSSPLSKPIYGLGYLRVSREEWRTRIMTSLMFWFAFLRAAVDIDYRTASILSVGVGLDAPEDWPPLFGRANQAYTLRNFWGTYWHQMFRWPFTATSNYLARELMALPRPSLLERYTNIFFVFLVSGVMHVMSDLLMGISMSQSASILFFCSMAVGVMIEDAVQAAWTRVSDSHRPGGASTVDSDGYAVPCWHKLVGFIWVCVWLSLTTPAWLCPLQMSKEKSLFLVNIPELVGTRKAIAITVGGGLLVKYTFRGEL

SEQ ID NO: 77 - protein, 513 aa, unidentified
  source 1..513: note = Pleurotus sapidus
MASQNMYAIGLSSLAIAALLVVVRHYANKRLSLPFPPGPKPLPVLGNVLDIPSKAPWKTYNDWFKVYGDMIYMNTMGQSILVLGSVERITDLLEKRSVKYSDRPRMTMLNELMGWDWCFGLMPYCTSWRKQRKLFYDQFQTNFLQFRPIQINHTHKFLGNLLESPDNFLQHIRLFFAGTILEVTYGIQVSSYNDPYVTNAEQCLAALASASIPGAFLVDTFPILKHIPLWFPGAGFKRKAKEWAKLSYNLVHGPFNATKEALRQGKALPSAAANMLEAAPEDKPDNYEELVRNCAAISFAAGSDTTVSTLQTFFLAMAMFPEAQKEAQAEIDAIVGHTRFPSFADRPVLPYLEALIMEISRWNTVSPLALPHAFTSDDEYDGYFIPKGTIVMANTWALLNDPSVYPNPGDFNPDRFLMDGKINPEVRDPRDFVFGYGRRACPGRFFSQDNLYSAVTGILASFSITPAQDAQGNDIPLKAEMSNGSLSYPLPFKCKITPRTGYAEQLIRHSNSS

SEQ ID NO: 78 - protein, 343 aa, unidentified
  source 1..343: note = Neocamarosporium betae
MAYTVEPREHSKNTTLPTVAMPPSPPSSFSASFGPFRYDTKEVNFDHWTSTKEKVVTGPYDYIAAKPGKEVRTLLLACFDEWLQVPPESLEVIGQVVRMLHTASLLIDDIQDNSELRRGKPVAQNIFGTALTINSANYVYFLALEKLNSLKNPNITDIFTEELLRLHRGQAMDLYWRDTLTCPTEEEYFEMVANMTGGLFWLMYRMMNAESSMPIDLLPVVELLGVIFQVLDDYKNLCSREYGKLKGFGEDLTEGKFSFPVIHSIRSNPEDLQLLHVLQQKSSNEHVKLYAIEIMESTGSLEYTKHVVENIVSQIQEIIYSTDEGQGRGKGILDLLHKITRLS

SEQ ID NO: 79 - protein, 944 aa, unidentified
  source 1..944: note = Neocamarosporium betae
MVPISTRSEVQDGLIAQARSLITRIVHNSDDVYGFGTLSCTVYDTAWVALVTKHVNGIKHWLFPESFHYILASQCDDGTWCEDKTAQFDGVLNTIAGLLVLKRYRRDPLQLKVDNRDLDSRIKLATSALHSLLEEWDVSTTNNVGFEIIVPTMLDLLVQEDPSLSFELKGREALTEIREAKMNRFQPELLYQGKLMTLTHSLEALLGRIDYDNVARYTVKGSMFASPSSTAAYLMSASTWDDEAETYLRYTITASTGKGSGGVPGVFPTTYFEYTWILSTLFRAGFQSSELDSPELTAMTDTLLKAFKAFSGAIGLDSGIEPDVDDSAKIVTTLNMLGKPAHARYLCDNFEVETHFRVYPRERDPSFSANCNALAAFLHQPDVEVYSSQILKAASYLCERMWNADEKIDDKWNKSHLYSSFLYTQVMTDLMAITEAGRLDGVFTKELLTRVCVTIFQSCLRAMLKQSHDGSWNQSLEETAYAILHLTEARRLCFFEQISEPLENSIYRGITFLTTIDKPPMEYLWSDKVSYGSAYLAETYVLAARRAAESTSVTNLVGFSIWKDNASTKMHKLVGLFHRTPLLKALPKWELQASMIEASIYQGLLQDARLEVLQRPKVDGGEYLSIIPFTWTSCSNSARTNASASHLWELMALSFFTYQVDEFMEAVAGPAFKGRMTHLHAIIDEAVHCSQNRERTPGECENTNHVTSELLQAVRFILDNPTVRKASPYDRNTLLQELRIFLHAHVTQVEDNASFGRERLTGDKALTSYRSQLYRWVHTISSDHIAGPFCFYYATCLLGATLTAHPPNDCFPKSSQKYLAAATCRHLSCMCRMYNDIGSWNRDHREGNLNCLHFPEFSETTSDDAERKASLLTLAQYERKQWCNALQQLEKEMVHGASEPAAARLAKRRACLIDMFCKVTDLYGQIYVLRDVSSVIKDVVRNGE

SEQ ID NO: 80 - protein, 541 aa, unidentified
  source 1..541: note = Neocamarosporium betae
MIKHSLSDCLADPDHLIRIKSYVEANVRFLGLSLVTLLVTIQMFRALGSPLRLNKPLVGRRSILEPRWLVGLRFTKGGRELLRQAYKKYKDEIFKVQCNDTEICVLPHRYVEELRGLPASKVSSPQALYNKGLGSYTGLEVIVESHLHFQAIQGHLTPNLASALGIVLDELQDALKTVLPDCSDEWVPFDVHTVLSELVSRLSSRVFGGLELARNQQWIQLSTAYPRNAFACTMALRMVPRIIRPLLAAVLPTYWRTRSNIRDAKRIVGGIITKRRADEGATDMSAKEHPCDLLQWMMNAAAGTETHADDLAHRLLFISDASVMTTSLLISHCLYDLVAHPEALSCIREEVHNVLREGDNFQKTTLHKMRSLDSALKESQRLNPPFLMTFDRVVREPLLLSDGTQIPVGTHLAMPTDAMLQDSSLLPQGGVAPDQFDPFRYARAREDPENAQRFQLATTEAKSLVFGHGKHACPGRFFASSEAKIILSHLLLLYDFRYPEGKGRPESWLFSENVAIDPNARLLIKKRNDAASNLAMLAKAL

SEQ ID NO: 81 - protein, 486 aa, unidentified
  source 1..486: note = Neocamarosporium betae
MSHFLPTLILTSLTLVAYVLARMIYNVFYHPLSAFPGDAFFCATGLTKAYHMIAGDLQLKVKDMHDKYGSVVRIAPTELSFSYCSAWKDIYGSRGGRELSKFYDFYRVDEAMPQHIISAGKAKHSILRRYLAHGFSENAMKAQEPVILDLVNLLMQRLREHAEEGARVVDVNKWFNFATFEIIGKLTFGADLGNLRNRDWHPWVKGSANNNMVVGFMAAANSVGLGPIIKWCISNEILPRQKYLDELAEMVQKRTGVTVERPDFIQGLLRDDVQLSNGEIVANVEALIGAGSESTATLLTGTVCALLQNPDQLAKVIDEVRSTFRTEDEITLHSVQRLDYMLACLNETFRYYPPVTNGMPRVTPKEGAIIGGRLVPGNTVVAIWQWAICHDPALWKDPYTFRPERFLEAPEFSTDVREALNPFSVGTRNCIGRNLSYAETRLILARLFYYFDLELADPDQDWFGAQKAYLVWDAPALNMYLKPVVR

SEQ ID NO: 82 - protein, 1314 aa, unidentified
  source 1..1314: note = Hormonema carpetanum
MPSYHNTDKTLLGDARQSLQQAVDYSLGCQQPDGHWVAPVMADATFTAQYVFFKHQIPELSLDEDGPEIQRWLLGEQTADGSWTLAPDLPGNLSTTVEAYLALRILGVPKSDQAMLRARDFVVRNGGVEGVRFFTRFFLATFGLVPWTAIPQMPAELILLPTFMFLNIYVLSSWARSTLIPILLVRHHEPVYALPNGQSANNNFLDELWCNPGEKNIPFALPLWDLLRRYQWIEFAFTLLDHILALFGGLRRWPCRHMALKRCTAWLLEHQEESGDWAGFFPPIHGSIWALLLDGFSFQSEVIRLGMEALERLVVIDPKGKWVQSTVSPCWDTALMANALCDAGMSGDTRLAKATQWLRDRQLMVSHGDWRNYANTQQAGGWSFQYFNSFYPDVDDTAVVIMTLIKEDPNCTNSDCVMNGVEWMLGMQSRDGGWGAFDVNNNARWLHKIPFSDMDSLVDPSTSDVTGRILECLGLLLSQRKSPLSPRWRHRLQASSAKAIAFLAKEQESSGAWWGRWGNNYHYGTANVLRGLAWFAQTDPSAQMMCMRTLSWIDETQNADGGWGETLASYVDKSLAGLGRSTAAHTAWALESLLRFRLPSDQAIERGVRWLIDNQQPNVDGYYYGTKWQAGAGQGASWRFDHAYVGTGFPSVLYLGYPYYHHLFPIQALSRYIDKASRQGIETLRIPSSSAVILDRPNVLLMAMGSRGDIQVFLNVTKRLSSCRVRIATHPAHQAKVEGHGFEFYDVGGSPEVFSAALANGHGILRSIIDGRFRELQHLLRSIYRSFWVAALDDVQSHSPLKPESSSRPFIADVVVSGPSTSVHVHAAERAQAPLVIISTQPAIITGDFQSPLTMSRAQFNPSRLWNRISFHMLAFFDWLSFGPSFNRMRANSYQLRSLDLAWALFEFVKVSVPHVCLWSTSLAPKPEDWDNNVIIAGYSSMSDDADDVASKSLQAFLETRQPVVAISFGSATIEDPMKLIKLMSAALVKVGASSVVCRSWDSSFEAEADLPSNVFLVDSIPHGWLLQHVEGFVHHGGAGHTAAGAKAGVSQLVMPQFLDQFFWATKVSEMGLGPTPLPLRELFLDELAPRMEDLLSSKYTKACTNMALQLRGDVDGADVAGDEILRQVEAITTCCIFPELSAHWYCTESDLSLSGAAAASLVSSKEIQWQDLELRPAKDWEQQWRTVRSSSNLVRIWRAIVQLLYGFTTTILALFGWLKGTHGYLDGDRHLIKMEDPLRQARLEQAQFDLHLIHQACDSGSSTSLDAKIIENWKARKAVVIHEVFDDDSGASESSRSSLDGGHADSVLDIEEK

SEQ ID NO: 83 - protein, 505 aa, unidentified
  source 1..505: note = Hormonema carpetanum
MVHLASIVSDLGFWPTVAGTVATYLFYQIVATVYDVYFGPMSNIPGPKLWAASHIPYILMLWNGEDASTKLALHEKYGPVVRVSPTELSYREAQAWKDIYGHQTRGKKSFPKDSRFYAPAVNGAPSILNADDPTHTRHRRILAHAFSDKALKEQEPLLKKWSDMLVSKLKENVEKSPSTPLDLVSWFNFTTFDIMADLTFAEPLYMLEGSTYSPWVRTIFGSLKLGSRLRAVRILPGMTTFINMIMPNSMRQKQVEHFKYSADRVDKRLANKNLDRPDLWAQVLKRVDVDGMSMSVAEMHSNSAIFMLAGTETTATLLSGLTYHLLQNPATMSKLVNEVRSTFPTRDDINVETLQRLPYLNACLEEGMRMYPPVATGLPRLTPKGGAAICGTWIPEDVAVSVSQWSTYQCEENFHKAKLFIPERWMGEDEQFANDNHAAFQPFSTGPRNCIGRNLAYHEARLLLCEVLWNFDLTLDEKSEDWLNQPVFTVWEKDPLWVRLQVASR

SEQ ID NO: 84 - protein, 551 aa, unidentified
  source 1..551: note = Hormonema carpetanum
MSAPRTALSSARQLSNGLIRRTNPRKRFLPLPSTVRSFTNNKPLRSQATAAQEPIVESERPSPVTSVNQPLSFPCLDALDTKTQSLTKQKKSLSGPEPSYAAGETEHFHSASPILLDWGGVLPEFDIAYETWGTLNADKSNAILLHTGLSASSHAKSTAKNPKPGWWEKFIGPGAPLDTDKYHIICTNVLGGCYGSTGPSSIDPLAENNTSSAQKDRYATRFPILTMQDMVRAQFRLLDWMGIDALYASVGSSMGGMQSLAACVLEPSRVGKVVSVSGCARSHPYSIAMRHTQRQVLMNDPAWSSTRGNYYPSIPPHAGMKLAREIATVTYRSGPEWEQRFGRRRADPSKPPALCPDFLIETYLDHAGEKWCLEYDANSLLYVSKAMDLFDLGDEHMRTLDGVRKSNAWKLDGFAAGTAKEEEKAGLCNLTLPDTPYEEQDETSSELLASSPEEAKRARETSSAALIAEEPPEDLVKGLQALSGTPALVMGVASDILFPAWQQREIAAALRKAGNRRVTHVELGEDRSLFGHDTFLLDLEGVGGEIRRFLG

SEQ ID NO: 85 - protein, 400 aa, unidentified
  source 1..400: note = Hormonema carpetanum
MVESSRWYSVPETLSAKAGLPIVSLPFNNQIQTSPFSVRSSYYLPLQTVNMSPSLVAPRQKALSLRHPTMAGKEYLEEFSKKYIIDVLDVPNREEWLKQLPKKVAKDGPYSALIIGMGTGPYEPFDAEFCQALLPDLKLVVSASAGYNEFDVSWMTENNIWFCNTVDAVSEATADMAIFLTLAALRDTTNAERSARAGNWKAGFVPCRDPTGLRLGIIGMGAIGKHIARKAAVFNLEIVYHNRKRLQEADETTYKATYCPTLASLLETSDIVSISVPLNDETTGMISESEIALMKDSSILINTARGAVVDEAALIEALESGKIWRAGLDVFCNEPDINPYFKTSDKVVVQPHLGGLTAVAFRKAYRECFENVASFFDTGKPLAPVNDVTRAKGTPTASLP

SEQ ID NO: 86 - protein, 493 aa, unidentified
  source 1..493: note = Hormonema carpetanum
MATSVEADTKSQVNLKVANDGGESASETGVVDVYDDPLIHNSIRKKLDLKLLPLLSAMYLFNAIDRSNLGNAKTDGLEKDLHMKGNEYSITLVLFYVTFCLLDVPANMLLKKFSGKIMLPTLMMGWGSMTLIQCAVHNWGGLIACRLLMGAFEAGFMAGVVYYLTTFYRRNELALRISIFYGAATIAGAFSGLLAYGVFQINHPSIPGWKFLMIIEGSATILLASFAYWHLPSSVLSCKWFTEEEKHVAEQRMLHDGSIQTDEKFALKTALANLLDWKIALYAVIGISYGVASASVGNFLPQMVQRLGFGTVKTNLYTVAPYCVGCVILLAQCTSSDHFRERSTHLAGAMLLTFVGFILLITLDTEAQPGPTYFACFLLAAGAFTPSCIFHSWHNNNTPSENGRAAVTGFMVGASNSGGIISSLAFASKTAPKYIPALIVTATFQGVGIVLVLGFGAWFRWDNRRRDRVQGVRIRTGDVATVSVDDASWRWTA

SEQ ID NO: 87 - protein, 563 aa, unidentified
  source 1..563: note = Hormonema carpetanum
MEFLEYLTPITSHQWGIGSVFLLISIPLIVTRLLTTFWSWRELSAANQKAPGQKRGPTRPTTIPLIGHIVSFLLDGPGFLYNTARYYGRGIPVRVHLATFPSYVISGPELVSAFLKDSTRSLTPMKRSLNYMEHAFGCPHDLVKSFATSDDKDMEQQIHSALQNMLSGPRLGILSERYQQSVKSQVLSVDAGIGDEWVELPDLCTFVETHVFQAATRTIFGPSIVDLNPTLAKDFWNFNKRVKTMFLGIPAWANRTAIRARDDMTEDIKRWQRHAEKNCNIDEIPEDVEWEEYYGSKSTRIRQQLLTKRGITNESARAAENLSMMWATNANSIPAACWFLLQTLHDPLLQARVRKELDAVRIHDTEETNKAAAFKFDITGLTESPLCQSVYAEVLRLGVAAMIVREPTHDNLALGDWKFKKDEFISVPTNNELMDPDFWNSGTPKDPHPLDKFWADRFLVHPDDPQSGPRKDAKKQKAKSDGKPYFSMDGCTWNWIPYGGGAHLCPGRNFAKREIMLTSAIMLTAFDIELLTDTLPKHDKSLFGFGTLPPIGKAPCRIRRRQL

SEQ ID NO: 88 - protein, 565 aa, unidentified
  source 1..565: note = Hormonema carpetanum
MEVSNLNFALVLLSIAYAFGTAVMRTQKHSPSFLHSVPVVGLRKQAFSITRASLRQLFGGITTLLEGYSQYTQHDQPFAMYDLSSRQETLVPVKDVKWFAEQPDSQLSSHGVRQERHAVEHLHMGVDVPATMHFIERITGDRLTRNLDLLAQPMHEEIQRCIETEFGNDPEEWKEINVYDSVQDIVMPTMSRVFLGLPLCRDTKVVNALKRYVMALGLATIFIGSLPRLVKSVLAGLVKIPLAYYRRQTLAVLVPLIRRQLEHPAEGDSDSEQETGFLRSCAKISEKNAVGGIGNVVEPEIIAQWIMWLAFAGSSSTIIQATNLLLDLANCPKDMEVIQQLRQEAVSSLKSAEDWKDPQSFNKQVISDSAIRESLRFHPILIKGLTKEVVPSSGITLPNGTQMSKGSWVGIPVLGIHRDKRYYPDPETYDPFRFVPFRLNGELQAGTAWGSKLDAAKPTTTYLGFGYGRHACPGRWFATLMLKMILAEIVLHYEIEATAPPPQMRVIGDAALPPMSATIRPIALVKTFHLPSSAPKKIARDTKKPHSKFFSRQCPVSPQNWNLVE

SEQ ID NO: 89 - protein, 295 aa, unidentified
  source 1..295: note = Hormonema carpetanum
MTAAVQSPTVNTTLNYFREVADGGLSEIRHGTVGSTRMKYNTQPVVVNDIRSNEGEFSLDKQGFQLVTSATKVKAFDEKTVKEQYYEELIDLIKETTGASFVLPMNHLVRQQLWESIHEIPADADDAAIVDTTAAPAMSVHIDQTPEGAELILQMLMQADAGRLGKTRWGIINAWRPLKLIRREPLAVCDVRSVPDSDLRVLGVIYPLPDGMVYNGSSDLKSDTWNVAANPEHKWYYASNMTPDEVLLLKMYDTKLDGRARKCPHSAFKRSYDEGPARESIETRCLVFWEDQERE

SEQ ID NO: 90 - protein, 614 aa, unidentified
  source 1..614: note = Hormonema carpetanum
MAARLTAVIALGVAFVSASPAYSNTTSSAYLCNPAHSYPSGVSCDATARTLITPAPSSPSSAYFCNPAHSYPGGVSCDATARTLITPAPSASSSAYLCNPAHSYPDNVTCDATARTLITPAPSASSSVYLCNPAHSYPGNVTCDATARTLVTPTLSSSSAYLCNPAHSYPGGVSCDATARTLFTPAPSPVTLTEVVTSFTTFCPSPTTLVAGNKTYTVTKPTTLTITDCPGGCTLTRTAVPSPVTLTEIVTSFTTFCPSPTTLTAGGKTYTVTKATTLTITDCPGGCTLTKAIVPSPVTVTEVVKSFTTFCPAPTTLTAGGKTYTITKPTTLTFTECPGGCTLTKTAIPVAYTTTEAVSTFTTYCPQPTTITVDKQTITVTAATTLSVPVTQTLTKPVLPVTSAPSASASSRAYLCNPAHSYPGGVSCDATARTLIYPSTKDAVTGAALITTIVPAVPAQTGVASGNCAPVYVTITIKEIQTVTVGLGSPVYFTSSSQAPSVSVDTPVYITGSSYFHNTSAPAPTTNANATSSAYLCNPAHSYPGGVSCDATARTLIYPSTSTASFSSISGIPGGCNPAHPGSCPSSYFDTTIPAQATASATKATRRGSKMVRA

SEQ ID NO: 91 - protein, 696 aa, unidentified
  source 1..696: note = Hormonema carpetanum
MTIHSPWETSHSKKGLIDIGTHRLFARVSGPRRKPGDPVILIECGGGSISLLWTGVMRALSPTYRIFTYSRAGLEGSDLPPNRGPRTAVEMADDFEKLLDVAGIKSPYIIVGHSYAGVQIRTWMHVHDRMEDIVGAVFVDTATEYTYPRMPIREPQFWFLMEQIPNPAHVLGYDRVHKFFNKEQWEEVLSTDLGHEEKDVDASAAQLKTWKQFERQIMEDKPVSVIKGYFAEDLRRLCVEAERIGAGTPAQRQYVMENLDSLDRVFEETETEQLRLSSCARMVHAAPGSGHTIVFSDPEIVMNEIKWVVEQYEAKRSIATIKGDLSNITAPPFLLASQSTCEFPSYWAEHPSIFVAPALEPDPKKRALLVLRWFLAALKRQQYAGRDEKEGVKKPLNAFLGELFFAQWQDQSGTTKLVSEQVSHHPPITACYLWNDEHGVRADGFACQSITFSGTVNIKQMGHAILHIDKYDEDYLIPLPNVKVQGLLTGTPYPELVGSYQIACSSGFVADIDFSGKKLFGMSGTKNMLHAALYSADDKQRENPIYTIEGAWNDKFTIRDESTGENVETYDTAANPATILEVAPLDLQDPWESRKAWGETISSLNGGKMQGASDAKSKVEQGQRAMRKQDEQNGSKWEPVFFSNEPRDERYIRLTAVAGTSTEEHSQSGFWKFDKDRAERARKPYHGSLRPDNVES

SEQ ID NO: 92 - protein, 745 aa, unidentified
  source 1..745: note = Waltergamsia fusidioides
MPSADVLMVRGSSYHVDTKAASAYRTDPSKWRLKVHEGRHMWEYHSGEEETEKGDHDKSSALPPHSFAEAYWLGHGDYELPSMNKPTCAKEALDNGWEFFKRLQLEDGHWGCNDDGPLFVTSGIVIASHIVGVDFPQPMKDEMIRYLFNFANDQGGWGLWINSPSTVFGTTINYVMLRILGVSPSHPVMVSARAALHRMGGATQLPTWGKFWMCVLGVYEWDGMIPLIPEPLLAPQCLPLNPGQWWVHTRNVFVSMSYLYGHRFTAPVTQLTRELREELYCDVAYDDIDWYAQRTSISDNDRLFPPTWLQRSLSGALGLYQSVRIPFLRRKALDEALFQVELEVHNTNYLCIAPVSFASNMLVMLHAHGAGSHWVRGMVDRIIDPMWMCREGMAASGTNGTSLWDTVFTVQAALDGGLAGRSENHDTMLRALRFIDVSQIRENPLGVSQGYRQPTRGAWPFSTRDQAYAVSDTTAETVRAVIQLQSLDTMPKLVSDDRLKEAVDLILGMENKHGGGGFSAFETLRAPKALERLNITELYTDVMTENLYPECTSSVLLCLETFSRAFPDHRPAEVQSCVGRCVRYLMDAQLPCGGWIASWGVCFTYATMFALEGLATAGLRQSNSPVCRDACLFLLRHQNPDGGWGEDLESLREKRYVQDPAGSQVTCTAYALAGLIAAHCADREAVARGVGWLVGQQQETGEWLPGSLEGIFAAPGGMRYPNYKFHFTLATLGKYVERYGEKKGW

SEQ ID NO: 93 - protein, 490 aa, unidentified
  source 1..490: note = Waltergamsia fusidioides
MLSPFTVASIGILFLAVAFSGVRYFIFPRGPKLPFPIVAVEDGGMDYRTAVEHGYATYPDQPFLLQSKTKSLVLLPPKYFHEIRSIPESQWSARLSARNLFQSRHTRIGDNSHRVVQTVKTDLTRHIGKTLDALQDEMEFAAGQNIGTCDDWTPILPYPALLRIVSLISGRVLVGLPFSRTEQWTQISINFTMLCFGAAQKYMSYPAYMRPFVVPFLDVTRSIHKQRAVATQLLAPVIEEYKRKASKPGYEEPSNMIEWALESAKDDDDRVGSAEEQADVQLTLSMAAIHTTSTTVLYCLYDLCSHPEYVEPLRQELESVLAETDGRLVKRNMAKLRKLDSFIKESQRLSPPGLVAMQRITVSDITLSDGSVLPKGAAVAVPSWSVTRDEKLWTNPTTFDGFRFEKLREEPGAEHKYQFATTGPDSLSFGYGPQACPGRFFASNEIKVLLAHILLNYDCKLEGGKRPDNITRDLTILPNMGVKILFKKRG

SEQ ID NO: 94 - protein, 518 aa, unidentified
  source 1..518: note = Waltergamsia fusidioides
MKGLTDQAGTIILVLIGIYILWQCIQGFVLSPLRDIPGPSLARVTTLRAIRNRLPRRVVQAALADFASYGDMYVSRPDTITISHPRDVRAVLGAKDSWKIDVYHGLDDPVLKNLVTLSHPQAAAQKRRQISPFLNHNAYLAEMEAVILEHGAQSLVAKWDAALAESPRGRIEINYRNDTQLATFNIMSALAFGRKLSGQADSSVIVAWIAATAVYIGVSINFRALLRFPLSLAIAPWLSRYRDFVEFGKESVRLRREAISSGALSKDQAPKDMLQAFINAQDPDSKVSMSSQEVQAESVGMQLAGSETTSASLTWALHLFTLHPSALQRATSEVRGQFDGKTDLLRYADCRALPFLESFVYETFRYAPITSGFMPRSLPKAIELQGHYIPAGTKIAFNLLALNNRKDIWDDPEQFIPDRFMGNEERKRNVFAFSYGPRSCIGRNLAWMEIMTILANMLLRYDLELPEDSVYGPKNVDDKGIPRLMESQCHIVFAPVHPERDCRLRLSKRNEMNIGVAR

SEQ ID NO: 95 - protein, 538 aa, unidentified
  source 1..538: note = Waltergamsia fusidioides
MLASTLHHFQDLLGTVSRYEYVSAACVCIVLMLSYRLIYQLFFSPLRNIPGSAWSRLFASHSVLKRVLADGAKSVQKDYEAYGDVYVLKPNGVCISDPKDIKTVLTSYEFVKTDVYEMLDIKGRPSIFTTRVAAEASQRRRQIGPYMNHGYLSRMESLILHHSIVSIKKKWDALLDKADKDGDGDSIAVNFRDDTQYATFDTIGMVAFGRDFRALETNDPTIIRWIEATGFYLGMTKNFPFLKWWPFSGLIRAKKAAYESFVSYADKSVELRRESMEKDSDAHANSNSKSDATKPVDLLQAFLDAEDPESEQRVRMTPHQVSTESIAMQLAGSESTSFVVSWVLHLLTLYPKFLNRAVDEVRGQFGPDHLLGFDDCNKKLPFLTACIYETLRYSPITSGFMPRVNRSKGITVQGHYIPPGTEIAVNMIGAHANKKVWDKPHLYDPTRFLDNEDAKRNVLAFSYGHRNCIGRNLAWVEMMVILANILKDYDISLPAGSVCGPHNTDELGRPRIMPTKSTLFTTPKYPERDCLIIVRRRA

SEQ ID NO: 96 - protein, 510 aa, unidentified
  source 1..510: note = Waltergamsia fusidioides
MSNGVKSSTYVSILAAGLATVLVYRLLHATLFSPLRRVPGPLIARLTSKRGHVNNFSGQVDSRVQKDRQKYGDVYVFKPNAVCVNDPADVRAILGSQDFRKADFFDVFDDGSIKNIVSQKDPTLAGRRRRQIGPYLNYAYLNKMEPVIQRHGFQALQAKWDKLIDEGKGKPAEVNYRLDTQLVTFDIMSALAFGRDPDSISHGSSSITKWTAIIMNLLDYSIVLALISLVPFSTIMRPWKSMYRDLALYSKDAVSSRKALLSEVSPESAPADMLQAFIEAEDPDSKIKMTAPEVQAECIMMMLAGSETTSSAIMWVFHLLLLHPDKLRKAVDEVRGEFASDHLILHREVLSRLPFIEACVYEALRVSPTTAGVTPRISYTRGIELQGHYIPPGTEIFVNLRSPNMSPETWETPDKFHPERFVGDEAKKNLFTFSYGPRNCIGRNLAWVEMLTIVANVLNNYDIALAPGTPFGPGKLGANGEPEMLPARCFIASFPANAERDCRMRITRRK

SEQ ID NO: 97 - protein, 335 aa, unidentified
  source 1..335: note = Waltergamsia fusidioides
MVQVLLTGGTGFIASHIIEKLLAKGHSVVTTVRSQEKGQKILDAHQDAGNRLQVVIVPDMASVGAFDEAVRVPGTEVVMHVASPCTLTYTDAQKDIIEPALTGTNSLLRSIHRDAPQVRRVILTSSISAMLDVRNPATTFSEQTWNPADPADIHLSKGMAYMIAKTLTERAAWDYVRVEKPPFDLVTVNPPLVLGPLMRHLATADSISLSNAALADAIRGQWKEEIPPAGPVNLWVDVRDVAAAHLAAMERGDASGKRLFTVGGRFSNAEIARILRDGAYSFHERLPGQEVKGDEGVKGISYSSEETDKLLAIDWIPLEKTIADFVKSIQDVDLK

SEQ ID NO: 98 - protein, 269 aa, unidentified
  source 1..269: note = Waltergamsia fusidioides
MSLKEVAIVTGGAAGVGLAAAKILAKRGYIVVVSDVAVEQGHEAVAAINQEFGQGTAIFIACDLGKTEEIDQLLASVVAQFSRFSVLVNNAGYLRAPFLAISAEQVEQTIAVNLTAPIYATQQAIKIWNDDNQGGNGDGEGAKGNVVNITSSSSFKTHASIAAYGAAKAGAAQFTFACRSFGPSIRVNAVAPTAIATAFDKNASFRVPTDKTGPGYTPEEDMRNMGLARLQPEQVAEAAFECIDDETRFGTVAWLDATEGQKIHTSFAG

SEQ ID NO: 99 - protein, 496 aa, unidentified
  source 1..496: note = Waltergamsia fusidioides
MDGDAKDGFVSTLPRVTYPLSPLDIVVSHLYISNCFFFEGPELGFDSGCQYDNDHDDGTSFAVEQLLQSGLYRALEKFPILVGELRPSGLNTMEIAVEPERPNMPVWETTYDSVVSFDHCQTRGFHPDSWPEGLTLDGEPLLVTDDTCQSAKLLRIRVCRFAGNTGVAVLVRVAHAVFDAKGVVAFINYWATCCREEKQKKQKQQPSLGSPSGKDESVHLPESILSRDAMYRKLPIDIRPTPMSKLLSPISWVLVAILGWLAFTMRDYLTQGDSESHLFAISRKDLDAIRSEAKEASSIVISDNDVIVALFTMAYAQSREDGSKKPQSKSKVKALVPCDFRHRLDIPETFSGNCAVGLFVTASSDQLLQPIEHATLIQVASVCRKTVDSADRGVIEQLIRRAMESIKWLGPKARVLYSLMVCQAFSNQSRLGFYSSDFGFGPPVLAVPLAYPQTLAVVVPSPPESDDVYVWLSLKREQMVQLMGNRCFHSLVRVVY

SEQ ID NO: 100 - protein, 405 aa, unidentified
  source 1..405: note = Psilocybe cubensis
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSF

SEQ ID NO: 101 - protein, 473 aa, unidentified
  source 1..473: note = Psilocybe cyanescens
MQVLPACQSSALKTLCPSPEAFRKLGWLPTSDEVYNEFIDDLTGRTCNEKYSSQVTLLKPIQDFKTFIENDPIVYQEFISMFEGIEQSPTNYHELCNMFNDIFRKAPLYGDLGPPVYMIMARIMNTQAGFSAFTKESLNFHFKKLFDTWGLFLSSKNSRNVLVADQFDDKHYGWFSERAKTAMMINYPGRTFEKVFICDEHVPYHGFTSYDDFFNRRFRDKDTDRPVVGGVTDTTLIGAACESLSYNVSHNVQSLDTLVIKGEAYSLKHLLHNDPFTPQFEHGSIIQGFLNVTAYHRWHSPVNGTIVKIVNVPGTYFAQAPYTIGSPIPDNDRDPPPYLKSLVYFSNIAARQIMFIEADNKDIGLIFLVFIGMTEISTCEATVCEGQHVNRGDDLGMFHFGGSSFALGLRKDSKAKILEKFAKPGTVIRINELVASVRKALGLRKDCRAEIVEKFTEPGTVIRINEVVAALKA

SEQ ID NO: 102 - protein, 362 aa, unidentified
  source 1..362: note = Psilocybe cubensis
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 103 - protein, 361 aa, unidentified
  source 1..361: note = Psilocybe cyanescens
MTFDLKTEEGLLSYLTKHLSLDVAPNGVKRLSGGFVNVTWRVGLNAPYHGHTSIILKHAQPHLSSDIDFKIGVERSAYEYQALKIVSANSSLLGSSDIRVSVPEGLHYDVVNNALIMQDVGTMKTLLDYVTAKPPISAEIASLVGSQIGAFIARLHNLGRENKDKDDFKFFSGNIVGRTTADQLYQTIIPNAAKYGIDDPILPIVVKELVEEVMNSEETLIMADLWSGNILLQFDENSTELTRIWLVDWELCKYGPPSLDMGYFLGDCFLVARFQDQLVGTSMRQAYLKSYARNVKEPINYAKATAGIGAHLVMWTDFMKWGNDEEREEFVKKGVEAFHEANEDNRNGEITSILVKEASRT

SEQ ID NO: 104 - protein, 309 aa, unidentified
  source 1..309: note = Psilocybe cubensis
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 105 - protein, 309 aa, unidentified
  source 1..309: note = Psilocybe cyanescens
MHIRNPYRDGVDYQALAEAFPALKPHVTVNSDNTTSIDFAVPEAQRLYTAALLHRDFGLTITLPEDRLCPTVPNRLNYVLWVEDILKVTSDALGLPDNRQVKGIDIGTGASAIYPMLACSRFKTWSMVATEVDQKCIDTARLNVIANNLQERLAIIATSVDGPILVPLLQANSDFEYDFTMCNPPFYDGASDMQTSDAAKGFGFGVNAPHTGTVLEMATEGGESAFVAQMVRESLNLQTRCRWFTSNLGKLKSLYEIVGLLREHQISNYAINEYVQGATRRYAIAWSFIDVRLPDHLSRPSNPDLSSLF

SEQ ID NO: 106 - protein, 508 aa, unidentified
  source 1..508: note = Psilocybe cubensis
MIAVLFSFVIAGCIYYIVSRRVRRSRLPPGPPGIPIPFIGNMFDMPEESPWLTFLQWGRDYNTDILYVDAGGTEMVILNTLETITDLLEKRGSIYSGRLESTMVNELMGWEFDLGFITYGDRWREERRMFAKEFSEKGIKQFRHAQVKAAHQLVQQLTKTPDRWAQHIRHQIAAMSLDIGYGIDLAEDDPWLEATHLANEGLAIASVPGKFWVDSFPSLKYLPAWFPGAVFKRKAKVWREAADHMVDMPYETMRKLAPQGLTRPSYASARLQAMDLNGDLEHQEHVIKNTAAEVNVGGGDTTVSAMSAFILAMVKYPEVQRKVQAELDALTNNGQIPDYDEEDDSLPYLTACIKELFRWNQIAPLAIPHKLMKDDVYRGYLIPKNTLVFANTWAVLNDPEVYPDPSVFRPERYLGPDGKPDNTVRDPRKAAFGYGRRNCPGIHLAQSTVWIAGATLLSAFNIERPVDQNGKPIDIPADFTTGFFRHPVPFQCRFVPRTEQVSQSVSGP

SEQ ID NO: 107 - protein, 507 aa, unidentified
  source 1..507: note = Psilocybe cyanescens
MIVLLVSLVLAGCIYYANARRVRRSRLPPGPPGIPLPFIGNMFDMPSESPWLRFLQWGRDYHTDILYLNAGGTEIIILNTLDAITDLLEKRGSMYSGRLESTMVNELMGWEFDLGFITYGERWREERRMFAKEFSEKNIRQFRHAQIKAANQLVRQLIKTPDRWSQHIRHQIAAMSLDIGYGIDLAEDDPWIAATQLANEGLAEASVPGSFWVDSFPALKYLPSWLPGAGFKRKAKVWKEGADHMVNMPYETMKKLTVQGLARPSYASARLQAMDPDGDLEHQEHVIRNTATEVNVGGGDTTVSAVSAFILAMVKYPEVQRQVQAELDALTSKGVVPNYDEEDDSLPYLTACVKEIFRWNQIAPLAIPHRLIKDDVYRGYLIPKNALVYANSWAVLNDPEEYPNPSEFRPERYLSSDGKPDPTVRDPRKAAFGYGRRNCPGIHLAQSTVWIAGATLLSVFNIERPVDGNGKPIDIPATFTTGFFRHPEPFQCRFVPRTQEILKSVSG

SEQ ID NO: 108 - DNA, 11328 bp, synthetic construct
  source 1..11328: mol_type = other DNA
atgactcaacttaagcctcctaatggtactactcctattggtttttctgctactacttctcttaatgcttctggttcttcttctgttaagaatggtactattaagccttctaatggtatttttaagccttctactagagatactatggatccttgttctggtaatgctgctgatggttctattagagttagatttagaggtggtattgaaagatggaaggaatgtgttaatcaagttcctgaaagatgtgatctttctggtcttactactgattctactagatatcaacttgcttctactggttttggtgatgcttctgctgcttatcaagaaagacttatgactgttcctgttgatgttcatgctgctcttcaagaactttgtcttgaaagaagagtttctgttggttctgttattaatttttctgttcatcaaatgcttaagggttttggtaatggtactcatactattactgcttctcttcatagagaacaaaatcttcaaaattcttctccttcttgggttgtttctcctactattgttactcatgaaaatagagatggttggtctgttgctcaagctgttgaatctattgaagctggtagaggttctgaaaaggaatctgttactgctattgattctggttcttctcttgttaagatgggtctttttgatcttcttgtttcttttgttgatgctgatgatgctagaattccttgttttgattttcctcttgctgttattgttagagaatgtgatgctaatctttctcttactcttagattttctgattgtctttttaatgaagaaactatttgtaattttactgatgctcttaatattcttcttgctgaagctgttattggtagagttactcctgttgctgatattgaacttctttctgctgaacaaaagcaacaacttgaagaatggaataatactgatggtgaatatccttcttctaagagacttcatcatcttattgaagaagttgttgaaagacatgaagataagattgctgttgtttgtgatgaaagagaacttacttatggtgaacttaatgctcaaggtaattctcttgctagatatcttagatctattggtattcttcctgaacaacttgttgctctttttcttgataagtctgaaaagcttattgttactattcttggtgtttggaagtctggtgctgcttatgttcctattgatcctacttatcctgatgaaagagttagatttgttcttgatgatactaaggctagagctattattgcttctaatcaacatgttgaaagacttcaaagagaagttattggtgatagaaatctttgtattattagacttgaacctcttcttgcttctcttgctcaagattcttctaagtttcctgctcataatcttgatgatcttcctcttacttctcaacaacttgcttatgttacttatacttctggtactactggttttcctaagggtatttttaagcaacatactaatgttgttaattctattactgatctttctgctagatatggtgttgctggtcaacatcatgaagctattcttcttttttctgcttgtgtttttgaaccttttgttagacaaactcttatggctcttgttaatggtcatcttcttgctgttattaatgatgttgaaaagtatgatgctgatactcttcttccttttattagaagacattctattacttatcttaatggtactgcttctgttcttcaagaatatgatttttctgattgtccttctcttaatagaattattcttgttggtgaaaatcttactgaagctagatatcttgctcttagacaaagatttaagaatagaattcttaatgaatatggttttactgaatctgcttttgttactgctcttaagatttttgatcctgaatctactagaaaggatacttctcttggtagacctgttagaaatgttaagtgttatattcttaatccttctcttaagagagttcctattggtgctactggtgaacttcatattggtggtcttggtatttctaagggttatcttaatagacctgaacttactcctcatagatttattcctaatccttttcaaactgattgtgaaaagcaacttggtattaattctcttatgtataagactggtgatcttgctagatggcttcctaatggtgaagttgaatatcttggtagagctgattttcaaattaagcttagaggtattagaattgaacctggtgaaattgaaactatgcttgctatgtatcctagagttagaacttctcttgttgtttctaagaagcttagaaatggtcctgaagaaactactaatgaacatcttgttggttattatgtttgtgattctgcttctgtttctgaagctgatcttctttcttttcttgaaaagaagcttcctagatatatgattcctactagacttgttcaactttctcaaattcctgttaatgttaatggtaaggctgatcttagagctcttcctgctgttgatatttctaattctactgaagttagatctgatcttagaggtgatactgaaattgctcttggtgaaatttgggctgatgttcttggtgctagacaaagatctgtttctagaaatgataatttttttagacttggtggtcattctattacttgtattcaacttattgctagaattagacaaagactttctgtttctatttctgttgaagatgtttttgctactagaactcttgaaagaatggctgatcttcttcaaaataagcaacaagaaaagtgtgataagcctcatgaagctcctactgaacttcttgaagaaaatgctgctactgataatatttatcttgctaattctcttcaacaaggttttgtttatcattatcttaagtctatggaacaatctgatgcttatgttatgcaatctgttcttagatataatactactctttctcctgatctttttcaaagagcttggaagcatgctcaacaatcttttcctgctcttagacttagattttcttgggaaaaggaagtttttcaacttcttgatcaagatcctcctcttgattggagatttctttattttactgatgttgctgctggtgctgttgaagatagaaagcttgaagatcttagaagacaagatcttactgaaagatttaagcttgatgttggtagactttttagagtttatcttattaagcattctgaaaatagatttacttgtcttttttcttgtcatcatgctattcttgatggttggtctcttcctcttctttttgaaaaggttcatgaaacttatcttcaacttcttcatggtgataatcttacttcttctatggatgatccttatactagaactcaaagatatcttcatgctcatagagaagatcatcttgatttttgggcaggagtggtacagaaaatcaacgagcggtgcgacatgaacgccctactcaacgagaggagtaggtacaaagtacagttggcagactacgaccaggtacaggagcagcgtcagttaacaatcgcattgagcggcgacgcatggctcgccgacttgaggcagacatgctcagcacaggggataaccttacacagtatcctgcagttcgtgtggcacgcggtcttgcacgcctacggaggaggcacgcacacaataaccggcacaacaataagtggccgaaacttgccaatcttgggcatcgagcgtgcggtaggaccatacatcaacacactaccgctcgtcttggaccacagtacattcaaagacaaaacaatcatggaagcaatagaggacgtgcaggccaaagtgaacgtcatgaacagccgtgggaacgtggagctaggaaggttacacaaaacggacctaaaacacgggctgttcgactcattgttcgtgctcgagaactacccaaacctggacaaaagtcgcaccttggagcaccagacagagttgggatactccatcgagggcggaacagagaaactgaactaccccctcgcagtaatcgcaagagaggtggagacaaccggtggattcacagtgtcaatatgctacgcctcagagttgttcgaagaggtgatgatatcagagttgctacacatggtacaggacaccttaatgcaggtcgcaaggggattaaacgagccagtcggaagcttagagtacctcagcagcatacagctcgagcagctggccgcgtggaacgccactgaggccgagttcccagacacaacattgcacgagatgttcgagaacgaggccagtcagaaaccagacaaaatcgcagtcgtctacgaggagacatccttgacgtaccgtgagttgaacgagcgggcgaaccgtatggcgcaccagttacgctccgacgtgtcaccaaacccaaacgaggtcatagcactagtgatggacaaatccgagcacatgatcgtcaacatattagcggtgtggaaatcaggcggagcatacgtaccaatcgacccaggatacccaaacgaccgtatccagtacatcctggaagacacacaggcgttggccgtgatcgcagacagctgctacctaccgcgaataaaagggatggcagcctcaggaaccttgctatacccaagcgtgttaccagccaacccagactccaaatggagtgtatcaaacccctcaccattaagtcgatccacagacttagcctacatcatctacaccagtgggacgacaggacgtccaaaaggagtcacagtcgagcaccacggagtagtaaacttacaggtgagtctcagcaaagtgttcggcctacgtgacacagacgacgaggtcatactcagcttctcaaactacgtattcgaccacttcgtagagcagatgaccgacgcaatattgaacggccagacattactcgtcttgaacgacggaatgcgtggagacaaagagcgattataccggtacatcgagaagaaccgtgtcacctacttgtcaggaaccccttccgtagtgtcaatgtacgagttctcccgattcaaagaccacttacgaagggtagactgcgtaggagaggcattcagtgagccagtcttcgacaaaatacgagagacattccacggattggtgatcaacggatacgggccaacggaagtatcaatcacaacccacaaacggctatacccattccccgagcgtcgtatggacaaatccatcggccagcaggtacacaacagcacatcatacgtcctgaacgaggacatgaaacgtacccccataggagcagtcggggagttatacctgggtggcgagggcgtagtacggggataccacaaccgtgccgacgtgacagcagagcgtttcataccaaacccattccagagtgaagaggacaaacgtgagggacgcaactccaggctctacaaaacaggggacctagtgaggtggataccagggagttcaggcgaggtcgagtacctcggcaggaacgacttccaggtaaagatccgcgggctgcgaatagagttaggagagatagaggccatcttaagtagttaccacgggataaaacagagtgtggtgatcgcaaaagactgccgtgagggagcccagaaattcctggtgggctactacgtcgccgacgcggccctaccaagcgccgcaatcaggcgtttcatgcagagtaggctaccagggtacatggtcccgtccaggctcatactcgtgagcaaattccccgtcacccccagcggaaaattagacaccaaagcattaccgccagcagaagaggagtccgagatagacgtggtcccaccacgtagcgagatcgagcggtccttatgcgacatctgggcagagttgttggagatgcacccagaggagatcggaatatacagtgacttcttcagcttgggcggggacagtttaaaatccaccaaattgagtttcatgatacacgagtcattcaacagggcagtgagcgtcagtgccctgttctgccaccgtacagtggaagcacagacccacttgatcttaaacgacgcagcagacgtccacgagatcacaccaatcgactgcaacgacacacagatgatcccagtgagccgagcacaggagaggttgttattcatacacgagttcgagaacggatcaaacgcatacaacatagacgcagcattcgagttgccaggaagcgtagacgcatccctattggagcaggcattgcggggcaacttagcgaggcacgaggcactacggacactcctggtgaaagaccacgccaccggcatatacctacagaaagtcttgagcccagacgaggcacagggaatgttctccgtcaacgtggacacagccaaacaggtcgagcgattagaccaggagatcgcgtccttaagccagcacgtgttccgcttagacgacgagctcccatgggaagcgcgtatactaaaattagagtcaggcggcttgtacctcatattagcattccaccacacatgcttcgacgcgtggtcattgaaagtcttcgagcaggagttacgagcattgtacgcagcattgcagaaaaccaaaagcgccgcaaacttaccagccctgaaagcacagtacaaagagtacgccttgtaccaccgacgacagctcagcggcgaccgcatgcgaaacttgtcagacttctggttaaggaaattgatcggactggagccactgcagctcatcacagaccgtccacgcccagtacagttcaaatacgacggggacgacttgagtatagagctctcaaagaaagagaccgagaacctgcgtggagtcgcgaaacgctgcaaaagctccctgtacgtggtcttggtgagtgtgtactgcgtaatgctagcttcttatgctaatcaatctgatgtttctgttggtattcctgtttctcatagaactcatcctcaatttcaatctgttattggtttttttgttaatcttgttgttcttagagttgatatttctcaatctgctatttgtggtcttattagaagagttatgaaggaacttgttgatgctcaacttcatcaagatatgccttttcaagaagttactaagcttcttcaagttgataatgatccttctagacatcctcttgttcaaaatgtttttaattttgaatctagagctaatggtgaacatgatgctagatctgaagatgaaggttctcttgcttttaatcaatatagacctgttcaacctgttgattctgttgctaagtttgatcttaatgctactgttactgaacttgaatctggtcttagagttaattttaattatgctacttctctttttaataagtctactattcaaggttttcttcatacttatgaatatcttcttagacaactttctgaactttctgctgaaggtattaatgaagatactcaactttctcttgttagacctactgaaaatggtgatcttcatcttcctcttgctcaatctcctcttgctactactgctgaagaacaaaaggttgcttctcttaatcaagcttttgaaagagaagcttttcttgctgctgaaaagattgctgttgttcaaggtgatagagctctttcttatgctgatcttaatggtcaagctaatcaacttgctagatatattcaatctgtttcttgtattggtgctgatgatggtattgctcttatgcttgaaaagtctattgatactattatttgtattcttgctatttggaaggctggtgctgcttatgttcctcttgatcctacttatcctcctggtagagttcaacttattcttgaagaaattaaggctaaggctgttcttgttcattcttctcatgcttctaagtgtgaaagacatggtgctaaggttattgctgttgattctcctgctattgaaactgctgtttctcaacaatctgctgctgatcttcctactattgcttctcttggtaatcttgcttatattatttttacttctggtacttctggtaagcctaagggtgttcttgttgaacaaaaggctgttcttcttcttagagatgctcttagagaaagatattttggtagagattgtactaagcatcatggtgttctttttctttctaattatgtttttgatttttctgttgaacaacttgttctttctgttctttctggtcataagcttattgttcctcctgctgaatttgttgctgatgatgaattttatagaatggcttctactcatggtctttcttatctttctggtactccttctcttcttcaaaagattgatcttgctagacttgatcatcttcaagttgttactgctgctggtgaagaacttcatgctactcaatatgaaaagatgagaagaagatttaatggtcctatttataatgcttatggtgttactgaaactactgtttataatattattgctgaatttactactaattctatttttgaaaatgctcttagagaagttcttcctggtactagagcttatgttcttaatgctgctcttcaacctgttccttttgatgctgttggtgaactttatcttgctggtgattctgttactagaggttatcttaatcaacctcttcttactgatcaaagatttattcctaatcctttttgtaaggaagaagatattgctatgggtagatttgctagactttataagactggtgatcttgttagatctagatttaatagacaacaacaacctcaacttgaatatcttggtagaggtgatcttcaaattaagatgagaggttatagaattgaaatttctgaagttcaaaatgttcttacttcttctcctggtgttagagaaggtgctgttgttgctaagtatgaaaataatgatacttattctagaactgctcattctcttgttggttattatactactgataatgaaactgtttctgaagctgatattcttacttttatgaaggctagacttcctacttatatggttccttctcatctttgttgtcttgaaggtgctcttcctgttactattaatggtaagcttgatgttagaagacttcctgaaattattaatgattctgctcaatcttcttattctcctcctagaaatattattgaagctaagatgtgtagactttgggaatctgctcttggtatggaaagatgtggtattgatgatgatctttttaagcttggtggtgattctattacttctcttcatcttgttgctcaaattcataatcaagttggttgtaagattactgttagagatatttttgaacatagaactgctagagctcttcatgatcatgtttttatgaaggattctgatagatctaatgttactcaatttagaactgaacaaggtcctgttattggtgaagctcctcttcttcctattcaagattggtttctttctaaggctcttcaacatcctatgtattggaatcatactttttatgttagaactcctgaacttgatgttgattctctttctgctgctgttagagatcttcaacaatatcatgatgtttttagaatgagacttaagagagaagaagttggttttgttcaatcttttgctgaagatttttctcctgctcaacttagagttcttaatgttaaggatgttgatggttctgctgctgttaatgaaattcttgatggttggcaatctggttttaatcttgaaaatggtcctattggttctattggttatcttcatggttatgaagatagatctgctagagtttggttttctgttcatcatatggctattgatactgtttcttggcaaattcttgttagagatcttcaaactctttatagaaatggttctcttggttctaagggttcttcttttagacaatgggctgaagctattcaaaattataaggcttctgattctgaaagaaatcattggaataagcttgttatggaaactgcttcttctatttctgctcttcctacttctactggttctagagttagactttctagatctctttctcctgaaaagactgcttctcttattcaaggtggtattgatagacaagatgtttctgtttatgattctcttcttacttctgttggtcttgctcttcaacatattgctcctactggtccttctatggttactattgaaggtcatggtagagaagaagttgatcaaactcttgatgtttctagaactatgggttggtttactactatgtatccttttgaaattcctagactttctactgaaaatattgttcaaggtgttgttgctgtttctgaaagatttagacaagttcctgctagaggtgttggttatggtactctttatggctacacacagcacccattgccacaggtaacagtgaactacctcggacagctcgcaaggaaacagtcaaaaccaaaagagtgggtgttggccgtgggagacaacgagttcgagtacgggctaatgacatcaccagaggacaaagaccggagtagttccgcagtggacgtcacggcagtgtgcatcgacggaacaatgatcatagacgtcgacagtgcctggtcattagaggagtccgagcagttcatcagcagcatcgaggaaggcctaaacaaaatattagacggacgagcaagtcagcagacaagccggttcccagacgtaccacagcccgcagagacatacacaccctacttcgagtacctagagccaccgaggcagggaccaacattgttcctcttgccaccaggggaaggtggcgcagagagttacttcaacaacatagtgaaaaggctccgtcagaccaacatggtagtcttcaacaactactacttgcacagcaaacgtttgcgtaccttcgaggagttggcagagatgtacttggaccaggtccgcggaatacagccacacggaccgtaccacttcatagggtggagcttcggcgggatcttagccatggagatgtccaggcgcctggtggcatcagacgagaaaatagggttcctgggaatcatagacacctacttcaacgtgcgaggggcgacacgtacgataggattaggagacaccgagatcctcgacccaatccaccacatctacaacccagacccagccaacttccagcggctcccatcagccacagaccggatagtgttattcaaagcaatgcgaccaaacaacaaatacgagagcgagaaccagcgtcgcctctacgagtactacgacggcacacgcttgaacgggttagactccttgctaccaagcgactcagacgtgcagttggtacccctaacggacgacacacacttctcatgggtcgggaacccgcagcaggtagagcagatgtgcgccacaatcaaagagcacctggcaaggtac

SEQ ID NO: 109 - DNA, 993 bp, synthetic construct
  source 1..993: mol_type = other DNA
atggcttcaactccaaaagccaatgtaccaaaaatagatgtgtctccccttttcggggacaatatggaggaaaagatgaaggttgcccgagctattgatgctgccagccgtgacaccgggttcttctacgcagtcaatcatggtgtggatgtgaaacgactatccaataagacaagggaattccacttttctatcacagatgaagaaaagtgggatctcgctattagagcctacaataaagaacaccaagatcagataagggcaggatactacctgtcaattccggagaaaaaggcagttgaatcattctgctaccttaaccccaactttaaacctgatcatcctcttatccagtctaaaactcccactcatgaagtaaatgtttggccggacgagaagaaacatcctggtttccgcgagtttgcagagcaatattactgggatgtgttcgggctctcgtctgctttgctgagaggttatgctcttgcgttaggaaaggaggaagatttttttagcaggcactttaagaaggaagacgctttatccagtgttgttcttattcgttatccttaccttaatccatatccacctgcagcaattaagacggctgaggatggcaccaaattgagttttgaatggcatgaagatgtttctttgattactgtcctgtatcagtcagatgttgctaacttgcaggttgagatgccacagggttacctcgatattgaggcggacgacaacgcttaccttgtaaattgtggtagctatatggcacatatcacaaacaattattatccagctccgatacaccgggtcaagtgggtgaatgaggagagacaatccttgcctttttttgtcaatttgggatttaatgatactgtacaaccttgggatcctagtaaggaagacggtaagaccgatcaaagaccaatctcttatggcgattatctacagaacggattagttagtctaattaacaaaaatggacaaaca

SEQ ID NO: 110 - DNA, 1071 bp, synthetic construct
  source 1..1071: mol_type = other DNA
atgttacacatactctgtcaaggtactccctttgaaattggctacgaacatggatctgctgccaaagccgttatagctagaagtattgacttcgccgtcgatttgattcgagggaagacaaagaagactgatgaagagcttaaacaggtattgtcgcaactggggagagttattgaggaaagatggccaaaatactatgaagagattcgtggtattgcaaagggagctgaaagagatgtctccgagattgttatgcttaatacacgcacggaatttgcatacggtctaaaggcagcacgtgatggatgtaccactgcttattgtcaacttccaaatggagccctgcaaggtcaaaactgggatttcttttctgctacaaaagaaaatctgataagattaacgatccgtcaggcaggattaccgacaatcaaattcataaccgaggctggaatcattgggaaggttggatttaacagtgctggggtagcagttaattataacgcccttcatcttcagggtcttcgacccaccggagttccttcacatattgctttgaggatagcgctcgaaagtacttctccttcccaggcttatgaccggattgtggagcaaggtggaatggcagctagcgcttttattatggtgggaaatggtcacgaggcatttggtttggaattctcacctacatctattcgaaagcaggtgttggatgctaatggtaggatggttcatacaaaccattgcttgcttcaacacggcaaaaatgagaaggaactcgatccattaccggattcatggaatcggcaccagagaatggagttcctattggatgggttcgatggtactaaacaagcatttgcccagctatgggctgacgaagataattatcctttttcaatctgccgcgcttacgaggaaggcaagagcagaggcgcgactctgttcaatatcatctacgaccatgcacgtagagaagcaactgtgaggttgggccggccaaccaaccctgatgagatgtttgtcatgaggtttgacgaggaagatgagaggtctgcgctcaacgctaggctt

SEQ ID NO: 111 - DNA, 1644 bp, synthetic construct
  source 1..1644: mol_type = other DNA
atggaggctccacggtcagatcaggctcatacagatgctaccacgccaatggaggctatacggacaacttctctcgggactaacaactatggaccagtacctgatgattacctcgacctaccagtacgcgaagtaaatgatggcgccgatttacgagaatacataaccgagacgaggacaggtgaaatcatcaagccgatcaagtccaatgttactgggaaaacagaagattggaaaatggtgactttcacaatagacgatcctgagaatccaaagaactggtcgaaggcttttaagtggtattgtaccatggtggttgcattcacctgctttgttgttgccttttgcagtagtgtcattacagctgatgttgagggacctattgaggaatttggtatcggtagggaagcatcattggttgttataacggttttcgttattggatttggtctaggacccatggtctttgctccgatgtcagagatcgtagggagacgacctgtctacgctctgacacttgcattggctgttattttcgtaattccttgtgcggtttcaaagaacattggaactttaattgtatgcaggttgatagacggaatcgcttttagtgctccaatgactttggtcggtggtactctagctgatttgtggaaaagtgaagagcgtggggttccgatggcagcattcagcgctgctccattcattggtcctgctattggcccacttgttggtggctacctggctgacaattgtggttggaggtggctatattggatacaacttattttggcctttgtggcctgggtgatgatcacattcactgtgcccgaaacttttgcgccaattttgttgaaaaagcgagcacagaaacttagaaaggctgaggatgaccctaagtatacaactgaaaccgaactcgatgctagacccatgggagagaagctgagaatctttttatttcgccctttccaacttctgttcctcgagcccattgtattattcatttctctatacatgtcagtgatctatgggttgctttacatgtttttcgttgcctatccgattgtttatatgggcggcaaaggatggagcgcgtctaatacaggtctcatgttcattcctctggcgattggtgtgatattcagcgcatgttgtgctccttttgtcaacaatcactacctcaaggtctctgtggcatacggtggcaagcctccagctgaaaaaagattgattccaatgatgtgggcttgttggtgcataccttctgggctttttgtgttcgcttggacatcctatcccgatcttcactggatgggaccagcgatgggagggtttcttattggagtcggcgttattttattgtataattctgcaaataattaccttgttgatacctatcagcatcaagcagcatctgctttagcagccaaaacctttatcaggtctatatggggtgcttgcactgttctgttcactgaacaaatgtacgaacgtcttggtgaccagtgggccagtacgcttcttgcatttatcggattggcatgttgcgccattccttatgtgttttatttcaagggagagtccattagacgtttttcgaaatttgccttttcagacgatgaggaaaaagcaataaaggca

SEQ ID NO: 112 - DNA, 2496 bp, synthetic construct
  source 1..2496: mol_type = other DNA
atgtcattgacagcttctgctgcttcagctgctcttactcttttggctgatggacctgatgatggatctaagaggccttggaggcctaatgatccagttaaagagcaaagagatctttatacacaattcgttatctctactgctttgggtctttcagcttttcttgctttttgtattcttagacctaagtggacagaactttatgctgctagaagaagacaaagaaatgctgcttcaagacttccagaacttccagatacactttttggatggattccagttcttcatcaaattactgaagaggaagttcttcaatcagctggacttgatgcttatgtttttctttctttctttaagttcgccattagatttcttttggctgtttttattttcgctgttgctattattctcccaatgcattataaatacaccggtcaatatggagttcctggatgggataatccacctggaaataagacaacttcaccaattgatggatctgaaaaagagaagccagttactgatcctgcttatttgtggatatatgttttgtttgcttacgttttctccggtcttgctatatatatgcttttggatgagacaaaggttattattagaactagacaaacctacttgggaaatcaaacatcaacaactgatagaactatcagattgtctggtattccacatgatttgggtactgaggataagattaaggaatttgttgagggtcttagagttggtaaagttgaatcaattactgtttgtagaaagtggagagaacttgatgaattgattgatgagagaatgaaagttatcagagagttggagagagcttggacaaaacatattggttataagaggcctaagaatgatggtaatgctttgccattgacagagcaacaaccaagagatgctgatgatgaaagatctggattgttgtcaggacatgataatgaacatgtttctggttattctaacgaaagaccaaaagttagaatctggtatggtttgtttaagcttagatttcgtatgattgacgctattgattattacgaagaaaagcttagaaagatcgatgagtatattcaaaacgctagagagaaagagtatagaacaacagaaattgctttcgttactatggagtctattgctgcttctcaaatgttggttcaagctattcttgatcctcatccaatgcaaatgtttgctagattggctccagctcctgctgatgttatttggaaaaatacttatctctccagaacaagaagaatggttcaatcttggtcaattacatttgttatcggttttcttaccgttttctggtcagttttgcttgttcctattgcttcacttttggagcttaagacattggagacaattgttccaagattggctgaatttttgcaagagcatccaattattaagtcacttgttcaaactggtttgccaacacttgctttttcacttttgacagttgctgttccatatttgtatgaatggttgtctaataaccagggaatggtttctagaggtgacgttgaattgtctgttatttctaaaaacttcttcttctccttcttcaatctttttcttcttttcactgtgttcggaactgcttctggtttttatggtttttgggagtctcttagagatgcttttaaagattctactactatcgctcttgctttggctaattctcttgaaggtcttgctccattttatattaatttgcttatcctccagggattgggattgtttccatttcgtcttcttgaatttggatctgttgctttgtatccatttcaatttttgtctgctagaactcctagagaatatgctgagctttctacaccacctaagttttcttatggtttttcaattcctcagacaattttgattctcgttatttgtgttgtgtactcagtttttccttcttcatggcttatttgtttgtttggtttgatatatttcaccgttggaaaattcatatataagtaccaactcttgtacgctatggatcatcaacaacattctactggaagagcttggccaatgatttgtaatagagtttttgttggtcttttagttcaccaacttgctatgattggtgttcttgctttgagaagagctattactagatctcttcttttggttccattgttgggttttactgtttggttttcttattggtttggtagaacttatgagcctcttatgaagtttattgctttgaagtcaatcaacagagatcaacctggtggtggagatatttcaccttctccttcatcaactctttctccaccatctggattggatagagattcacttccaattagaattggaggtcaagatttggagcttagacttaaaaaatacgttaacccatcattgatcgttcctcttcatgctgcttggcttccaggtagaaatccagctagaggtaatggtaatggtgcttctgcttttgaagttcatcaaacacaaaatgtt

SEQ ID NO: 113 - DNA, 1524 bp, synthetic construct
  source 1..1524: mol_type = other DNA
atgaaggatggtgaggagacaccttcagttgatggttcaacatcagcttctaatagagagaaacttggaacagatttggaaattggaccagttgatctttctgatggaggaaaagaagaaaaagttaaggatccaaatctcgttgattgggatggaccagatgatcctgagaatcctcttaattggacttcaaagagaaaaattaccgctacatgttcaattgctttgattacttttctcactcctttgggttcatcaatgtttgctccaggagttggacaattggttaaagattttaatgtgacttccactgagctttcatcatttgttgtttctgtttatctccttggttattgttttggtccacttattattgctccactttcagaactttatggaagacaatatgtttaccatgtttgtaatatcctctacgttatttggactattgcttgtgcttttgctcctgagattggatctcttgttgtttttagatttttcgctggacttgctggttcatgtcctttgacaattggagctggttctattgctgatatgtttgttcaagagcaaagaggtggtgctatggctgcttgggctcttggaccattgattggtccagttgttggtcctgttgctggtgcttatcttgctcaagctaagggttggagatggtctttttatgttcttgctatggctgcaggtgctattactatttcttctctttttagtatcagggaatcatatgctccaactcttttggctagaaagactaagaaattgcaaaaggaaacaggtaatatgaacttgagatcagctcttgatactggtagaacacctaaagagttgtttttgtattcaatcgttaggccaactaagatgctttttagatcaccaattgttttccttttgtcactttatgttggagttatatatggttacttgtaccttttgttcacaactattacttcagttttccaacaacaatacaacttttcacaaggagctgttggacttacatatcttggacttggagttggttctttgattggattgtttttgattggagctacttcagatagacttttgaattatcttgctgctaagaatggagaaaagaaacctgagtatagacttccaccaatggttccaggagctatttttgttcctatttcattgtttatgtacggatggactgcttattatcaaacacattggattgttccaatcattggaacatcttttcttggtactggtatgatgattacttttatgtgtgtttcaacctaccttgttgatgcttttactaattatgctgcttcagttatggctgctaatacagtttttagatcattggctggagctcttcttcctcttgctggaccaaagatgtatgctgttttgggtcttggttggggaaattctttgcttggttttattgctcttgctttttgtgctttgccagttattttctggatatatggagaaagaatcagaacttcacctaaatttcaagttactttc

SEQ ID NO: 114 - DNA, 1032 bp, synthetic construct
  source 1..1032: mol_type = other DNA
atggttcaagatacttcttctgcttctacttctcctattcttactagatggtatattgatactagacctcttactgcttctactgctgctcttcctcttcttgaaactcttcaacctgctgatcaaatttctgttcaaaagtattatcatcttaaggataagcatatgtctcttgcttctaatcttcttaagtatctttttgttcatagaaattgtagaattccttggtcttctattgttatttctagaactcctgatcctcatagaagaccttgttatattcctccttctggttctcaagaagattcttttaaggatggttatactggtattaatgttgaatttaatgtttctcatcaagcttctatggttgctattgctggtactgcttttactcctaattctggtggtgattctaagcttaagcctgaagttggtattgatattacttgtgttaatgaaagacaaggtagaaatggtgaagaaagatctcttgaatctcttagacaatatattgatattttttctgaagttttttctactgctgaaatggctaatattagaagacttgatggtgtttcttcttcttctctttctgctgatagacttgttgattatggttatagacttttttatacttattgggctcttaaggaagcttatattaagatgactggtgaagctcttcttgctccttggcttagagaacttgaattttctaatgttgttgctcctgctgctgttgctgaatctggtgattctgctggtgattttggtgaaccttatactggtgttagaactactctttataagaatcttgttgaagatgttagaattgaagttgctgctcttggtggtgattatctttttgctactgctgctagaggtggtggtattggtgcttcttctagacctggtggtggtcctgatggttctggtattagatctcaagatccttggagaccttttaagaagcttgatattgaaagagatattcaaccttgtgctactggtgtttgtaattgtctttct

SEQ ID NO: 115 - DNA, 6570 bp, synthetic construct
  source 1..6570: mol_type = other DNA
atgaattttcataagggtcaacctaaggaagatcttagagttctttttggtcctcaatgtcctgatattactgattctattactcatattagagatgctatttctaaggatcctactggtcttggttttcttactaatattcttgatgaacttccttctctttggcctactattgctggtgcttggcctgctcttaagaatgttgaaggtgaaaatcaacttcttgctcttggtagactttttgaacatgaatctgaagttagagttgaagcttctaatcttatgatgactcctattactgttatgagacatgttgttgatttttggaatcttcaagatgttgctactcatcctgcttttccttcttcttctctttctgaaactgaaatgcctagaattgttgatactcaaggtttttgtgttggtcttcttgctgctattgctgttgcttgttctagaaatactcaagaatttcaatatgttgcttctaatgctattagactttctctttgtattggtgctcttgttgatcttgatgaaattctttgtggttctactacttctcttgctgttagatgggaatctgttgaagattttaatcatcttgaaaagattcttaataataatactgaaattcctaagggttatacttcttgttatactgatgttaagtctgttactattactattcctaatgattctgctgaaagagttaagcaagaaattcatgatcatggtcttagaactaagcaactttctcttagaggtagatttcatcatgaagctcatagagaaggtattcaacatattatgaagctttgtatgaatgattctagatttaagcttcctagatctgatgctcttcttactcctcttagatcttctcaaggtggtgaaatttttcaacaagaagctcttcttcatactgttgctcttgattctattctttgtgctaaggctaattggtatgatgttgtttctgctcttattaattctactgaaatgactgttgatcaatctagacttctttctattggtcctgaagaatttgttcctagatctgctagatctagatctgttgctagaagagaacttgaatcttatggtatgcaaggttttgctaatgaatctcctcaaccttctactgcttctctttctaattctgttcaaacttttgattctagacctcaagctgctgaagcttctcctattgctattactggtatggcttgtagatatcctaatgctgatactcttgctcaactttgggaacttcttgaacttggtagatgtactgttaagtctcctcctgaatctagatttcatatgtctgatcttcaaagagaacctaagggtcctttttggggtcattttcttgaaagacctgatgtttttgatcatagattttttaatatttctgctagagaagctgaatctatggatcctcaacaaagagttgctcttcaagttgcttatgaagctatggaatctgctggttatcttggttggcaacctaatagactttctcaagatattggttgttatgttggtgttggttctgaagattatactgaaaatgttgcttctagaaatgctaatgctttttctattactggtactcttcaatcttttattgctggtagaatttctcatcattttggttggtctggtccttctatttctcttgatactgcttgttcttctgctgctgttgctattcatcttgcttgtaaggctcttcaaactaatgattgtaagattgctcttgctggtggtgttaatgttcttactaatcctagagtttatcaaaatctttctgctgcttcttttctttctccttctggtgcttgtaagccttttgatgcttctgctgatggttattgtagaggtgaaggtgctggtctttttgttcttagacctcttcaagatgctattgataatggtgatcctattcttggtgttattgctggttctgctgttaatcaaggttctaataattctcctattactgttcctgatgctgaagctcaaagatttctttataataaggctatgtctcttgctggtgtttctcctgatgaagttacttatgttgaagctcatggtactggtactcaagttggtgatcctattgaacttgattctcttagaagaacttttggtggtcctcatagaagaaataatcttcatattggttctattaagggtaatattggtcatactgaaacttcttctggtgctgctggtcttcttaagactattcttatgcttcaacaacaaagaattcctagacaagctaattttaatcaacttaatcctaaggttaagtctcttactcctgatagacttgttattgcttctgaatctactgaatgggtttctactaagagagttgctatggtttctaattatggtgcttctggttctaatgctgctcttattgttaaggaacatgctcctattggttctgaacaaaatggtactgctcctgaatatattcaaaataagcttgcttataatcttgctatgaagcaaaatagagatcttcctcttaatcttactttttctacttcttctgatactacttctcttggtgctagacttgaagctatttctactggtgcttctgctgatcttattcaaaagagaccttctaatgaacctcctgttgttctttgttttggtggtcaaaatggtcttactgctactatttctaaggaagtttttgatgcttctgctcttcttagaactcatcttgaagattgtgaagaagttggtagaactcttggtcttctttctctttttcctactattttttcttctgctcctattactaatattattcatcttcattttattcttttttctattcaatatgcttctgctaaggcttggcttgattctggtcttcgtgtcagccgtatcgtagggcattctttcggccaactcaccgcattaagtgtcgccggatccttatccgtccgagacggcatccacttagtcacagagagggcgcgcttgatagagagtagttggggaccagagagcggcataatgttggcagtggaaggaacagagatagaggtgcagcagttactggaccagaccgtagagtcaatagaggcaatcgagaacgcagcagcaaggacaccatccgccagcaacctcaggttgacacggttacagaactcccacgcattccactcacgattggtcgacagtatagtaccaggaatcatggaagtggccggcagcttggtgtaccagacgccaataataccaatcgaggcatgctccgcatcaggagactggtcaacaataaccgcagcggagatagtggagcactcccgtatgccagtgtacttcaggcgcgcagtggagagggtggcagagaaattgcaggcaccagcagtgtggctcgaggcagggagcgcaagtcccataatacccatggtccgcagggtgttggagtcatcctctgtcgcgaacacataccacaaaatcgacttgggtggaagttcaggagcacagaacctagcaaacgtgacgagtgcactgtgggcccagggagtacacgtacagttctggccattcgaccgtgcccagcacgcgagtttcaaatggatgaacctacccccataccagttcgcacagaactcccactgggtcgacttcgaccccgcagcattcagtagtgccggaccatcatccggaaaacagagtgccggccaagaggcaggattgctgtgccagctgtccgagagtccggacgagcgcctctaccacgtcaacatacaggacgcattgtaccgtgcctgcacccaggggcacgcagtattgaaccagacattatgcccggcctcaatgtacatggagatggtcctgcgcgccgccgcgtccatattcccaaccggaaacgcaagtgagccagccatgtcacacatagaggacctgacaatcagcagtccactcgtcctcgacccacaggggaaagtcttcctgcggctaacacgtgacggcgcgggcccaacacgtccctggctgttcagcatattcagctcagagagtaacgaccacacattagtgcacgccgaggggacagtctgcctgcaccaggagaggtcccgtgcgttagcgaggttccagagtatggacaggttgctcgactcagcacgtagtaaaaccatagaggccgacccagcatcaaacggcttaaaaggctccacggtctacgcagcactcgagtcagtaacaaactacggcgactacttccgaggcgtgaaacaggtcttcgcaaacggccgtgaggcctcaggattagtaagcatgatgccgagcacaacagagacaaactgcaacccaatattgttggacaacttcctacaggtcgcaggaatacacgtaaactgcttaagtgatcgtcagtcatcagaggtcttcgtctgcaacgcgataggagagacattcgtgataaactcactattaaaacaggagaacggagcctcacccagcacatggaaagtatacacgtcatacgtcaggccatccaaaacagagatagcatgcgacatatacgtcatggactgccagacggacacattgagcgcagccatgatgggagtgcggttcacaagcgtgagtatacgcagtctgacgagggcattggcaaaattgaacaacaacgtgttggagacagcagaagcacagagcgtggtggaagccgcaatccccgccgagcaaagcgtggtaacagcaacaccgagcgccccagccgcagacggacatgcagcaaacgacctcgcaacagtacaggagatgttatgcgagttgttcggcgtcagtgtagctgaggtaagtccctcagtctcattagtcgacatcggagtagacagtttaatgagcacagaggtcttaagtgagatcaagaaacgtttccaggtggacatgtcatacacgacattggtcgacataccaaacatacagggcttagtagagcacatattcccagggcactcacacgcggcaccatcaaggccggtagtcgagaaagcaccagtacagtccgtggcccctcaggcagtctcccatgtcccgacaccagcgagtaacggcccaccattggtgtcagtcgctcgacagtgtttcgacaccacccacgccgcagtgagtcacaccagcgacgcccactgggccggattcttccacacgacataccccaaacagatgtcattgttgacagcctacatcttagaggcattcagggccttaggatcatccctggaagcgagcgagccaaacgaggtgttaacgccaatcgcggttctcccaaggcacgagcagttgcgtaaacacctgtacaaaatcctggactcagtagggttggtacggcagatgagtaccggcgagctgacactccgtcagagcgggttcgagtgggtcgactggacaaacaacgagacagtggagtcaaacgccctacgggtgatcgtcgcatccccaacagggaacagctcagccgcgacaatgagcccaagcaaaccgatcaaaatggagacagtcgtgtggggtgagcgagacaacttacagttgcgtgccgacatctactacccagagacggtggacacaacaaggaaacagcggccaatcgcattgatgatccacggcggcggacacgtcatgttgagcaggaaagacatcaggcccgcacagacccagaccctgctggacgcaggcttcttgccggtaagtatcgactaccgactatgcccagaggtaagtcttgccgaggggcccatggcggacgcaagggacgcactgagctgggtcaggcgtgtactgcccaacatcccactgttaagggcagacatacggccagacggcaatcaagttgttgctattggttggtctactggtggtcatcttgctatgactcttccttttactgctcctgctgctggtattcctgctcctgatgctgttcttgctttttattgtcctactaattatgaagatcctttttggtctaatcctaattttccttttggtcaaactgttgcttctaatgaaatggaatatgatgtttgggaaggtcttcaatctatgcctattgctggttataatcctgctcttaaggaaagacctcttggtggttggatgtctactagagatcctagatctagaattgctcttcatatgaattggactggtcaaactcttcctgttcttcttaaggcttgtactattaagggtaatactgaaaagtgttctcctgatgatctttctagacctactgaagaagatattcaagctgtttctcctaattatcaaattagagttggtagatataatactcctacttttcttattcatggtacttctgatgatcttgttccttgtgctcaaactgaatctactcatggtgctcttactgcttctggtgttgaagctgaacttagagttgttcaagaagctgctcatctttttgatctttatcctgcttctcatgctggtcaagaagctaaggctgctgttgctgaaggttatgaatttcttagaactcatgttcaacag

SEQ ID NO: 116 - DNA, 2559 bp, synthetic construct
  source 1..2559: mol_type = other DNA
atggaatctctttctcttacttggattactgctattgctgttgttctttatcttgttcaaagatatgttagatcttattggagacttaaggatattcctggtcctgttcttgctaagcttactgatcttcaaagagtttggtgggttaagactggtagagctcatgaatttcatagggacatgcatgcaatgtatggtcctattgttagatttggtcctaatatggtttctgtttctgatcctagagttattcctactatttacccctctagacccggtttccctaaaggagatttttatagaactcaaaagccttatactagaaataagggtgctatgcctgctgtttttaatactcaagatgaagatcttcataagcaacttagatctcctattgcttctctttattctatgactaatgttgttagacttgaacctcttgttgatgaaactcttactgttctttctaagcaacttgatgaaagatttgttggtactaatgataagccttttgatcttggtgattggcttcaatattttgcttttgattctatgggtactcttactttttctagaagatatggttttcttgaacaaggtcgggatatgcatggcattcttcaagaaatatggaattttatgactagagttgctgttatgggtcaaattccttggtttgatgagatttggaataagaattcttttattactctttttaagagacctactggttttggtgttcttaaggttgttgataattttatttctcaaagagtttcttctagagaaaatgatgaaaaggctgatgaaaaggatatgctttctcaatttcttaatattcaagcttctaatcctcattctattatgccttgggctcctagagcttggactttttctaatgttatggctggttctgattctactgctaacgttatgagaacgatgatgtataatcttcttgttgatagagatactcttaagtctcttagagctgaacttcttgaagctgaatcttctaatggtctttctagatcacttccttcttgggatggtgttagatctcttccttatcttgatgcttgcgttctagaagctcttagacttcatcctcctttttgtcttccttttgaaagagttgttcctgaaggtggtattactgtttgtgaaacttatcttcctgctggtactgttgttggtatttctccttatcttgctaatagagataagcaaacttttggtgatgatgctgataagtggagaccttctagatggcttgatctttctagagaagatagagttaagcttgaaaattctattcttacttttggtgctggtcggagaacttgtcttggtaagaatatagctatccttgaaatcaagaagttatttcctatgttgcttttaaactatgagattgaaatcgttaatcccgaaaattaccaaactactaatgcttggttttttagacaatggggtcttcatgctgttattagaaagcttcctgctcctgaaagagatgatactattgaacaaaaggcttctattcctcctgctcttaatattcctccttcttcttctactgttgatgttagaattattgattctggtactcttcttgatcttagacctgatcttttttggactcctgatcttcctggtcttcttaaggttactgctcctacttattgttttcttatttctaatggttctagacatgttctttttgatcttgctgttagacaagattgggaaaatttacccccgtcaattgtggctatgattaagtctcaaactgttattcaagaacctagaaatatttctgatgttcttgattctgatgaatcttctcttggtattagatctaaggatatagaagctataatttggtctcatgctcattttgatcatattggtgatccttctactttccccccatctacggaacttgttgttggtcctggtattagagatactcattggcctggttttcctacaaaccctgacgcgattaatcttaacactgatattcaaggtcggaacgttagagaaatttcttttgaaaagactcaaaagggtgctactaagattggttcttttgatgctatggattattttggtgatggttctttttatcttcttgatgctgctggtcattctgttggtcatattggtgctcttgctagagttactacttctcctgattcttttgtttttatgggtggtgattcttgtcatcatgctggtgttcttagacctactaagtatcttccttgtcctcttgattctggtgatacttctcttccttgtaagtctgattctgtttttactctttctcctgctcttcctactgattatactgctgctcttagaactgttgaaaatattaaggaattggatgcttgtgaagatgtttttgttgttcttgctcatgatgctactcttaagggtaaggttgatttctacccttctaagattaatgattggaaggctaaggaatatggtaagaagactaagtggcttttttataaagatatcgaaaacgcaattgaaggtcaaaag

SEQ ID NO: 117 - DNA, 993 bp, synthetic construct
  source 1..993: mol_type = other DNA
atgacaaatgcagtggaggactctggtcctagggacctgctatttctactgatcagtacctctaggtttaataggtatatgccttattatactatgatggctgctgtatgggcgacttttattgccggcgctctcaaattacaacaagatccagaaagcttatcaatagagtttattttgtacaaggcgggtctctgttttgtacactgtctgctactttgcggtgctggaaacacatggaacgatcttgtggatagagatattgacgccagagtggcacgcacaaagatgcggcctttggcatctggaaaggttactctcactgaggcattgctttggatgacaggtcaatatttcttgtctgtcaaaatgttagatctcattttggatggacgaaatatctggactctcatgcttccattgaccgcctcaatcatgctttacccctacctaaaaagacctatattcagtaaggtattcgtttacccgcaatatattctgggtttagctattggttatcccgcaattactgggtgggcttccatcaccggctcagaagagcctcttggagacatcattaagcattgcataccaatatgtcttcttgttttcttctggtgtgtgtacttcaacaccgcatacagccaccaagatagcgtagatgaccgaaaaatgaatataaattctgcttacgtcattgcaggccagagaattcgtctgttccttgcctttttgagtgttctcccacttttaacaattccatatataatctctacgataaattcaccgtggttgtgggtctcctggatggctacatggactgtttccattgttatgcagattgctcagtttgattcgcagaagcttgaaagtggtgggcgtatccattgggataactttttattgggattgtggacgattgttgcttgcatggttgaagtcggattgcagaaagtggaattttggaagaatgttgaggggtatattaaactt

SEQ ID NO: 118 - DNA, 1269 bp, synthetic construct
  source 1..1269: mol_type = other DNA
atgtctctttctcttcctcctgctctttctgaacttgctagagctcttccttattctagaactcaatggcttcctattcttgttggttttcttattggttatcctcttcttattaaggctcttagatataagagacttggtgaaatgaagaagaagttttattttcctactagagaatctatggctgaaatgactgatgaagaagcttttcttattcaaaaggaaatggctcaacttgaatttccttttatgtttcttacttctggtcaatttgctctttttagaacttatggtattcctactatttctcatcttcttactaagactggtcaattttctaagcctgaaacttcttttaagagatatactgatactgctgctcttattggtgaaatggttgaaaattctcctacttctcaaagagcttttatttctgttgctagaactagatttcttcattctggttatcaagcgtctggtaagattcttgatgctgatcttctttatactcttgctctttttgctgttcaacctgttagatttattgaaaattttgaatggagaactctttctgatcttgaactttgtgctattggtactttctggaagtctcttggtgatgctcttggtatttcttctgaaattcttccatctggtaagactggttttaaggatggtattcaatggcttgaagaagttgatgtttggtctcaagattatgaagctaagtatatggttcctgatcctaagaatagagaatctgctgatcaagctactgctgttcttctttataatcttcctaagattcttcatcctattggtcttcaatttacttcttatatgatggatgatagacttagaaaggctatgctttatgaagctccttctcctggttggtctgctgttttttcttctcttcttgctactagaaagtttgttcttagatatctttctcctcctagacctgctgctcttgctgtttctaatattgctcaaaagcctgataaggatgatagatatcatagaatgtcttgggatgctcttcctttttatattagacctactttttggaatagatggggtcctatggcttggatttcttggcttatggctcatcctgttcctggtgatcatggtcaaaagtattatcctcaaggttatcatattcaagatattggtcctaagtattttgaaggtaagggtcataaggaaattcaagaaatgatgaaggaacttaagatttctagaactggtaagtgtccttttcat

SEQ ID NO: 119 - DNA, 1194 bp, synthetic construct
  source 1..1194: mol_type = other DNA
atgtctgctgcttcaccagcttctattattcaagagttggcttctgctgctaagcaatatgaaaataatgaatctggtgctagagaggctcttattgctcaatctagagctcttattgcatctcttgaagttccatctgaattcattcaacatacattttggtcacaacctgctctttctgctattgttagacttgctacagatgttaatctttttcaatacttgaaggacgctcaagaagaaggtcttaatgctgaggctttggcttcaaagactggtatggatgtttctttgtttgctagacttgctagacatttggttgctatgaatgttattacttctagaaacggtgttttctatggtacagctttgtcaaatggattggctgctgagaattatcaacaatcaattagattctgccacgatgtttcaagaccatcatttggtgcttttccatcatttttcaaaggaaatggatacaagacacctgctcttggtactactgatggaccttttcaatcagctcataaggttgatatttcttttcctcaatggttggttggtaatccaccatatcttcaatattttaactcctatatgtccgcttatagagctggtaaacctaattggtgtgataatggtttttacccagttgctgatagattgcttaatggatttgatgcttcagtttcagatgttcttttggttgatgttggtggaggtagaggtcatgatattgctacttttggttctcaattttctcctcttcctggtagacttgttttgcaagatagagagcaagttattaattctatcccagctgatgaatctagacaatttgaagctactacacatgatattttcactactcaacctgttaaacatgctagagcttattatatgcattcagttcctcatggttttggagatgaagatgctgttaaaattatggctaatcttgttcctgctttggctaagggttattcaagagtgcttcttaatgaaattgtggttgatgaagaaagaccagttatgtctgctactaatatggatcttattatgcttgctcatatgggagctaaagaaagaactgaggctgattggagatcaattttgactagagctggtttgaaggttgttaatatatattcatacccaggagttgctgaatcattgattgaagctgaattggct

SEQ ID NO: 120 - DNA, 2121 bp, synthetic construct
  source 1..2121: mol_type = other DNA
atgttttttacacaaccaccgcatctcgttaaggcggaagagctcaaacaagagcctcctaagggcactgcctacagtgttgctatacccggaactgagcagcccggaagaagtcgggtatatagagcatggaatgcacaaaaggaacttttgacaacattggatccccaggtgactacggcacatgacatgtttgagtctactgctaatcggcaacccaagaatcattgcttaggatggcgtccgtataattcaacgacaaaaacctgggacccataccagtggcttacatatgaaacagtgcaaaagaggagagcagcctttggtgcaggtctagtagaactccaccaaaaacacaactgccaccgtccaggccaatatggtgttgggttgtgggcgcaaaaccgcccagaatggcagatcactgatctggcttgtataagccagagtctttacagtgtctccatatacgatgtccttgccccggatgccaccgaatatattattaatcacgctgaactgaactgtgtggtcacatctctacctcatattcctactcttctgaagttaaaatcttccttacctaacttaaaaatgattgttagtcttgacccgctcgatggacctgagcagaacggccactcaaagcgggcactgcttgagagtatggcagctggtctggatcttgcaatttatacgattgatcaggttgaggaactgggcttggcttcaaagagggggtacaatgctccttctgcgtcagatattgtgactattaattatacttccggaacaaccggacctccaaagggggttgttcttacacatggcaatgccgtggctgctacttcatgcggtcttaccactatcggccaagctcgaggagatacaatgtgttcttatctaccattggcacatatctatgctaggctcgcagaacacactgctttctggggtggtgcacgaattggatatttccatggcaatattgttgaacttgttgacgatttgaaattgttgaaaccaactggtttcatgtcggtaccaagactatatagccgatttggtaccgcgataagagctgccacggttgaacaacctggcttcaagggtgctttgtcccgacatatcgttgcagctaagatggctaacatgaaaaatcccgatccatctaaggcaacaataaagcatgctctttacgataggatctgggctaagaaggttgctgcagctctaggtttagagagggcaaaatatatgatttccggttctgcgcctctggaccctacacttcataactttcttagggtggcaaccggtaccgacgttgtgcaggggtacggtcttactgagtcatacgcatcggctacagctcaatctacgcaagatctatcatctggaaactgtggaagattggcaccctgtaccgaagcttgtttggtttctttacctgatatggaatactctgttgaagataaaccatttccacgtggggagttgctgctgaggggaaacaatatgttcagggaatattacaaaaatgacgacgagacctcaaaggctgtcaccgaagatggatggttccgcactggtgatgtatgcactgtggatgcccagggtcgctttatcattattgacagacggaaaaatgttttaaaactcgcccagggagagtacataagtccagagcgtttggagggtgtcatcctctcagaacttgggtatgtcgcccaggcatacgtgcatggagacagcagcgagactttcttggttggaatatttggtgtagctcctgacttgtttgctccatatgccagcaaagtccttgggaaaaccattgcaccgactgatgtggaaggactcaaggagcacttgaacgatgataagttaagaagggcggtgctaagagatttggaaagagtagccaaaaagcataaatttgcaggatatgagagggttcgtaatgtatcgctcaaggttgatcctttcacagttgaaaataatttacttacaccaactcttaagctcaaaagaccacctgtcgtgaagatgtacagaacacttttggatcagttatatgggcaggctaatgaggaacaatctgctcctcgtgctaagttg

SEQ ID NO: 121 - DNA, 1194 bp, synthetic construct
  source 1..1194: mol_type = other DNA
atgtctactgagaagtttacaatcacagagcatcttgttcctggttcacatattagagaatatccaggttctactgttaatcaagaggatgttcttaaaatccatgttaaacaatacaccccaaaaagagaaggacctgttccagatgatgctattacttttattgctacacatggagttggacttccaaaggaactttatgaaccactttgggatgagttgcttgatcaagcttctggttttcatattagagctatttggatggctgatgttgcttcaatgaatcaatcaggtattcataatgaggataagctttctatggattgtattacaaaccttgcttatttgcatccaagactttttactacacttcttttgcttgatcctcttattcaattgtcacctccttcattgggatttggtactgatgctccttctgctattaattatacattgtggagagatgatgtgtggccatctagagaggttgctattagagctaatagagctattatgcaaggaatggatcctagatgtcttgatagaatgactaagcatttctttagagatttgcctacacctctttatccagatgttgaagctattaaggctctttttggaactacagctgattcaacaactactccagttacacttactacaccaaagtatcatgagttggttgctcaaattagacaaaattttaacgctagagatcctaagactggtagaattgaggttccaagagatacacatgctgatatggatcctcttgttgcttatattccattgtatagacctgagccaagatcaacttttagaagacttgaaacattgaggccttcatgtctttgggttattgctggagctacatttcttaatattgatgaaatcagggagggagttaaaatttgtggatcaggaattggtggttctggaggagttccagatggtagagttagagaggttgttttgcctggatttggacatttgatgccttttcaagaagttaaaacagttgctgagacttgtattgtttggcttcaacaagagatggatagatttcgtcaaactgaaagacaatggaaagaagatagagatggaaaatctcatttggctgttgaggagaattggtataaggttttgaaaccaattccatctggtagaaaaaagagaaatgataaaggaaagctc

SEQ ID NO: 122 - DNA, 1644 bp, synthetic construct
  source 1..1644: mol_type = other DNA
atggttgaaattcttgattatactaaggctcttgaagttcttaaggaatatccttctggtgatggtcttcatgttgatactcttcttgattctgataatcatggtgctcttacttataatgattttcttattcttcctgggtctataactttcagtgctgctgatgtttctcttgatactaaggttactagaagatttactattaaggctcctcttctttcttctcctatggatactgttactgaacataatatggctattcatatggctcttcttggtggtcttggtgttattcataataattgtcctcctgatgatcaagctgaaatggttagaaaggttaagagatatgaaaatggttttattcttgatcctgttgttctttctccttctactactgtagctgaagctaaggaacttaagactaagtggaattttggtggttttcctgttactggtaagactcattacctttctagttttggtaagcttgcttcttctgattctttttctcttcttgaaaagggtactcttcattctaagcttcttggtattgttacttctagagatattcaatttcataagactcctgaagatcctgttactgctgttatgtctactgaacttgttactgctcctgctggcactactctcgctgaagctaatgaagttcttagatcttctaagaagggtaagcttcctattgttgataaggatggtcttcttgtttctcttctttctagatctgatcttatgaagaatattcattatcctcttgcttctaagcttccttctaagcaacttctttgtgctgctgctatttctactcatgatgctgataaggttagacttcaaaagcttgttgatgctggtctggatatagtcgttgttgattcgtcgcaaggtaattctatgtaccaaattgctatgattaagtggattaagtctacttttcctgatattgatattattgctggtaatattgttactagagaacaagctgctgctcttattgctgctggtgctgatggtcttagaattggtatgggtagcggttcggcttgtattactcaagaagttatggctgttggtagacctcaagctgcttctgttagatctgtttctgcttttgctgctagatttggtgttcctactattgctgatggtggtgttcaaaatcttggtcatattgttaagggtcttgctcttggtgcttctgctgttatgatgggttctcttcttgctggtactactgaatctcctggtgaatattatgtttctaatgaaggtcaacttgttaaggcttttagagggatgggttctattgctgttatggaagataagggtaagtctggtggtggtaagaatgctggtgcttctagatatttttctgaaaatgataaggttaaggttgctcaaggtgttgctggttctgttgttgatagaggttcgattacacagtatgttccttatcttgttgctggtattcaacattctcttcaagatattggtgttcaagatcttgaagctcttcataatggtgttaataatggtcaagttagatttgaaatgagatctgcttctgctcaaactgaaggtaatgttcatggtcttcattctcatgaaaagaagctttattcttct

(19) United States
(12) Patent Application Publication
(10) Pub. No.: US 2025/0320515 A1
(43) Pub. Date: Oct. 16, 2025

(54) GREENHOUSE-BASED PRODUCTION OF FUNGAL-DERIVED MEDICINAL COMPOUNDS

(71) Applicant: University of North Texas, Denton, TX (US)

(72) Inventors: Elizabeth Skellam, Providence Village, TX (US); Kent Chapman, Denton, TX (US)

(21) Appl. No.: 19/169,341

(22) Filed: Apr. 3, 2025

Related U.S. Application Data
(60) Provisional application No. 63/633,417, filed on Apr. 12, 2024.

(51) Int. Cl. C12N 15/82; C07K 7/64

(52) U.S. Cl. CPC C12N 15/8257; C07K 7/645; C12N 15/8205

(57) ABSTRACT

The present disclosure provides compositions and methods for producing fungal-derived medicinal compounds in plants. The present disclosure further provides modified plants, seeds, cells, and plant parts comprising intact biosynthetic pathways required for the production of fungal-derived medicinal compounds. Aspects of the disclosure further relate to methods for producing, growing, and breeding modified plants.

### CROSS-REFERENCE TO RELATED APPLICATIONS

This application claims priority to U.S. Provisional Application Ser. No. 63/633,417, filed Apr. 12, 2024, which is incorporated herein by reference in its entirety.

### INCORPORATION OF SEQUENCE LISTING

A sequence listing containing the file named “UTNT003US_ST26.xml” which is 243 kilobytes (measured in MS-Windows®) and created on Mar. 31, 2025, and comprises 122 sequences, is incorporated herein by reference in its entirety.

### FIELD OF THE INVENTION

This present disclosure relates to the field of agriculture and plant genetics. In particular, the present disclosure relates to methods and compositions for producing fungal-derived medicinal compounds in plants.

### BACKGROUND OF THE INVENTION

Industrial scale fermentation technology was implemented less than 100 years ago with the discovery of penicillin and has been applied to produce numerous other commodities and high-value chemicals. Furthermore, recombinant DNA technology and metabolic engineering capabilities are accelerating the use of microbial fermentation to produce industrially important plant pharmaceuticals such as opioids, artemisinic acid, noscapine, and cannabinoids. There are numerous reports and reviews of microbial production of plant based small molecules, where Saccharomyces cerevisiae and Escherichia coli are preferred biomanufacturing hosts due to their fast growth rates and simple secondary metabolism. However, many technical challenges remain to be overcome to optimize their use as universal hosts. These challenges include low titers of production i.e., milligrams per liter as opposed to grams per liter and high input of human capital e.g., large teams of people. Furthermore, specialized infrastructures that include deep fermentation tanks and factory facilities mean that industrial, technological, economic, and social barriers to production exist.

Currently plants are used in molecular farming to produce biopharmaceuticals such as Taliglucerase alfa, recombinant proteins, and vaccines. Over 50% of pharmaceuticals are derived from natural sources, and plants are the major producers of these biologically active molecules. The methods and compositions of the present disclosure demonstrate that plants can be engineered to produce a wide range of pharmaceuticals in larger quantities, rather than continuing to produce medicines via fermentation. As natural resources are depleted, the population continues to live longer, and CO₂ levels continue to rise, alternative long-term strategies are required to provide essentials such as food, clean water, and medicine. Considering that plants will continue to play a significant role in our future, including reducing the rate of depletion of natural resources, they are the ideal platform for the production of medicinal compounds.

### SUMMARY OF THE INVENTION

In one aspect the present disclosure provides a modified plant, seed, cell, or plant part comprising at least one recombinant polynucleotide molecule comprising at least one nucleotide sequence encoding at least one polypeptide having an amino acid sequence with at least about 85% sequence identity to an amino acid sequence selected from the group consisting of SEQ ID NOs:1-107, wherein the modified plant, seed, cell, or plant part is capable of producing a medicinal compound selected from the group consisting of penicillin, mycophenolic acid, cyclosporin, cephalosporin, pneumocandin B0, lovastatin, compactin, griseofulvin, pleuromutilin, aphidicolin, enfumafungin, fusidic acid, psilocybin B.

In another aspect, the present disclosure provides a method for producing a modified plant cell, the method comprising introducing at least one heterologous polynucleotide molecule into a plant cell, wherein the heterologous polynucleotide molecule comprises at least one nucleotide sequence encoding at least one polypeptide having an amino acid sequence with at least about 85% sequence identity to an amino acid sequence selected from the group consisting of SEQ ID NOs:1-107. In one embodiment, method may further comprise selecting at least one plant cell comprising the at least one heterologous polynucleotide molecule. In another embodiment, the method may further comprise regenerating at least one modified plant or plant part from the at least one selected plant cell or a descendant thereof comprising the at least one heterologous polynucleotide molecule. The at least one heterologous polynucleotide molecule, in yet another embodiment, comprises at least one nucleotide sequence encoding at least one polypeptide having an amino acid sequence with at least about 90%, about 91%, about 92%, about 93%, about 94%, about 95%, about 96%, about 97%, about 98%, about 99%, or 100% sequence identity to an amino acid sequence selected from the group consisting of SEQ ID NOs:1-107. The methods of the present disclosure may comprise, in yet another embodiment, introducing at least two, at least three, at least four, at least five, at least six, at least seven, at least eight, at least nine, at least ten, at least eleven, or at least twelve heterologous polynucleotide molecules into the plant cell. In one embodiment, the at least one heterologous polynucleotide molecule comprises a promoter functional in a plant cell. The at least one heterologous polynucleotide molecule, in another embodiment, comprises a targeting nucleotide sequence or encodes a targeting polypeptide sequence. The plant cell, in yet another embodiment, is a Nicotiana benthamiana, a corn, or a soybean plant cell.

In one embodiment, the modified plant, seed, cell, or plant part comprises a first nucleotide sequence encoding a polypeptide having an amino acid sequence with at least about 85% sequence identity to SEQ ID NO:1, a second nucleotide sequence encoding a polypeptide having an amino acid sequence with at least about 85% sequence identity to SEQ ID NO:2, a third nucleotide sequence encoding a polypeptide having an amino acid sequence with at least about 85% sequence identity to SEQ ID NO:3, a fourth nucleotide sequence encoding a polypeptide having an amino acid sequence with at least about 85% sequence identity to SEQ ID NO:4, a fifth nucleotide sequence encoding a polypeptide having an amino acid sequence with at least about 85% sequence identity to SEQ ID NO:5, and a sixth nucleotide sequence encoding a polypeptide having an amino acid sequence with at least about 85% sequence identity to SEQ ID NO:6, wherein the modified plant, seed, cell, or plant part is capable of producing penicillin. In another embodiment, the modified plant, seed, cell, or plant part comprises a first nucleotide sequence encoding a polypeptide having an amino acid sequence with at least about 85% sequence identity to SEQ ID NO:7, a second nucleotide sequence encoding a polypeptide having an amino acid sequence with at least about 85% sequence identity to SEQ ID NO:8, a third nucleotide sequence encoding a polypeptide having an amino acid sequence with at least about 85% sequence identity to SEQ ID NO:9, a fourth nucleotide sequence encoding a polypeptide having an amino acid sequence with at least about 85% sequence identity to SEQ ID NO:10, a fifth nucleotide sequence encoding a polypeptide having an amino acid sequence with at least about 85% sequence identity to SEQ ID NO:11, a sixth nucleotide sequence encoding a polypeptide having an amino acid sequence with at least about 85% sequence identity to SEQ ID NO:12, a seventh nucleotide sequence encoding a polypeptide having an amino acid sequence with at least about 85% sequence identity to SEQ ID NO:13, an eighth nucleotide sequence encoding a polypeptide having an amino acid sequence with at least about 85% sequence identity to SEQ ID NO:14 (MpaH), and a ninth nucleotide sequence encoding a polypeptide having an amino acid sequence with at least about 85% sequence identity to SEQ ID NO:15, wherein the modified plant, seed, cell, or plant part is capable of producing mycophenolic acid. In yet another embodiment, the modified plant, seed, cell, or plant part comprises a first nucleotide sequence encoding a polypeptide having an amino acid sequence with at least about 85% sequence identity to SEQ ID NO:16, a second nucleotide sequence encoding a polypeptide having an amino acid sequence with at least about 85% sequence identity to SEQ ID NO:17, a third nucleotide sequence encoding a polypeptide having an amino acid sequence with at least about 85% sequence identity to SEQ ID NO:18, a fourth nucleotide sequence encoding a polypeptide having an amino acid sequence with at least about 85% sequence identity to SEQ ID NO:19, a fifth nucleotide sequence encoding a polypeptide having an amino acid sequence with at least about 85% sequence identity to SEQ ID NO:20, a sixth nucleotide sequence encoding a polypeptide having an amino acid sequence with at least about 85% sequence identity to SEQ ID NO:21, a seventh nucleotide sequence encoding a polypeptide having an amino acid sequence with at least about 85% sequence identity to SEQ ID NO:22, an eighth nucleotide sequence encoding a polypeptide having an amino acid sequence with at least about 85% sequence identity to SEQ ID NO:23, a ninth nucleotide sequence encoding a polypeptide having an amino acid sequence with at least about 85% sequence identity to SEQ ID NO:24, a tenth nucleotide sequence encoding a polypeptide having an amino acid sequence with at least about 85% sequence identity to SEQ ID NO:25, an eleventh nucleotide sequence encoding a polypeptide having an amino acid sequence with at least about 85% sequence identity to SEQ ID NO:26, and a twelfth nucleotide sequence encoding a polypeptide having an amino acid sequence with at least about 85% sequence identity to SEQ ID NO:27, wherein the modified plant, seed, cell, or plant part is capable of producing cyclosporin. In still yet another embodiment the modified plant, seed, cell, or plant part comprises a first nucleotide sequence encoding a polypeptide having an amino acid sequence with at least about 85% sequence identity to SEQ ID NO:28, a second nucleotide sequence encoding a polypeptide having an amino acid sequence with at least about 85% sequence identity to SEQ ID NO:29, a third nucleotide sequence encoding a polypeptide having an amino acid sequence with at least about 85% sequence identity to SEQ ID NO:30, a fourth nucleotide sequence encoding a polypeptide having an amino acid sequence with at least about 85% sequence identity to SEQ ID NO:31, a fifth nucleotide sequence encoding a polypeptide having an amino acid sequence with at least about 85% sequence identity to SEQ ID NO:32, a sixth nucleotide sequence encoding a polypeptide having an amino acid sequence with at least about 85% sequence identity to SEQ ID NO:33, a seventh nucleotide sequence encoding a polypeptide having an amino acid sequence with at least about 85% sequence identity to SEQ ID NO:34, an eighth nucleotide sequence encoding a polypeptide having an amino acid sequence with at least about 85% sequence identity to SEQ ID NO:35, and a ninth nucleotide sequence encoding a polypeptide having an amino acid sequence with at least about 85% sequence identity to SEQ ID NO:36, wherein the modified plant, seed, cell, or plant part is capable of producing cephalosporin.

In one embodiment, the modified plant, seed, cell, or plant part comprises a first nucleotide sequence nucleotide sequence encoding a polypeptide having an amino acid sequence with at least about 85% sequence identity to SEQ ID NO:37, a second nucleotide sequence encoding a polypeptide having an amino acid sequence with at least about 85% sequence identity to SEQ ID NO:38, a third nucleotide sequence encoding a polypeptide having an amino acid sequence with at least about 85% sequence identity to SEQ ID NO:39, a fourth nucleotide sequence encoding a polypeptide having an amino acid sequence with at least about 85% sequence identity to SEQ ID NO:40, a fifth nucleotide sequence encoding a polypeptide having an amino acid sequence with at least about 85% sequence identity to SEQ ID NO:41, a sixth nucleotide sequence encoding a polypeptide having an amino acid sequence with at least about 85% sequence identity to SEQ ID NO:42, a seventh nucleotide sequence encoding a polypeptide having an amino acid sequence with at least about 85% sequence identity to SEQ ID NO:43, an eighth nucleotide sequence encoding a polypeptide having an amino acid sequence with at least about 85% sequence identity to SEQ ID NO:44, a ninth nucleotide sequence encoding a polypeptide having an amino acid sequence with at least about 85% sequence identity to SEQ ID NO:45, a tenth nucleotide sequence encoding a polypeptide having an amino acid sequence with at least about 85% sequence identity to SEQ ID NO:46, an eleventh nucleotide sequence encoding a polypeptide having an amino acid sequence with at least about 85% sequence identity to SEQ ID NO:47, and a twelfth nucleotide sequence encoding a polypeptide having an amino acid sequence with at least about 85% sequence identity to SEQ ID NO:48, wherein the modified plant, seed, cell, or plant part is capable of producing pneumocandin B0. In another embodiment, the modified plant, seed, cell, or plant part comprises a first nucleotide sequence encoding a polypeptide having an amino acid sequence with at least about 85% sequence identity to SEQ ID NO:49, a second nucleotide sequence encoding a polypeptide having an amino acid sequence with at least about 85% sequence identity to SEQ ID NO:50, a third nucleotide sequence encoding a polypeptide having an amino acid sequence with at least about 85% sequence identity to SEQ ID NO:51, a fourth nucleotide sequence encoding a polypeptide having an amino acid sequence with at least about 85% sequence identity to SEQ ID NO:52, a fifth nucleotide sequence encoding a polypeptide having an amino acid sequence with at least about 85% sequence identity to SEQ ID NO:53, and a sixth nucleotide sequence encoding a polypeptide having an amino acid sequence with at least about 85% sequence identity to SEQ ID NO:54, wherein the modified plant, seed, cell, or plant part is capable of producing lovastatin. In yet another embodiment, the modified plant, seed, cell, or plant part comprises a first nucleotide sequence encoding a polypeptide having an amino acid sequence with at least about 85% sequence identity to SEQ ID NO:55, a second nucleotide sequence encoding a polypeptide having an amino acid sequence with at least about 85% sequence identity to SEQ ID NO:56, a third nucleotide sequence encoding a polypeptide having an amino acid sequence with at least about 85% sequence identity to SEQ ID NO:57, a fourth nucleotide sequence encoding a polypeptide having an amino acid sequence with at least about 85% sequence identity to SEQ ID NO:58, a fifth nucleotide sequence encoding a polypeptide having an amino acid sequence with at least about 85% sequence identity to SEQ ID NO:59, a sixth nucleotide sequence encoding a polypeptide having an amino acid sequence with at least about 85% sequence identity to SEQ ID NO:60, a seventh nucleotide sequence encoding a polypeptide having an amino acid sequence with at least about 85% sequence identity to SEQ ID NO:61, an eighth nucleotide sequence encoding a polypeptide having an amino acid sequence with at least about 85% sequence identity to SEQ ID NO:62, and a ninth nucleotide sequence encoding a polypeptide having an amino acid sequence with at least about 85% sequence identity to SEQ ID NO:63, wherein the modified plant, seed, cell, or plant part is capable of producing compactin. In still yet another embodiment, the modified plant, seed, cell, or plant part comprises a first nucleotide sequence encoding a polypeptide having an amino acid sequence with at least about 85% sequence identity to SEQ ID NO:64, a second nucleotide sequence encoding a polypeptide having an amino acid sequence with at least about 85% sequence identity to SEQ ID NO:65, a third nucleotide sequence encoding a polypeptide having an amino acid sequence with at least about 85% sequence identity to SEQ ID NO:66, a fourth nucleotide sequence encoding a polypeptide having an amino acid sequence with at least about 85% sequence identity to SEQ ID NO:67, a fifth nucleotide sequence encoding a polypeptide having an amino acid sequence with at least about 85% sequence identity to SEQ ID NO:68, a sixth nucleotide sequence encoding a polypeptide having an amino acid sequence with at least about 85% sequence identity to SEQ ID NO:69, a seventh nucleotide sequence encoding a polypeptide having an amino acid sequence with at least about 85% sequence identity to SEQ ID NO:70), and an eighth nucleotide sequence encoding a polypeptide having an amino acid sequence with at least about 85% sequence identity to SEQ ID NO:71, wherein the modified plant, seed, cell, or plant part is capable of producing griseofulvin.

In one embodiment, the modified plant, seed, cell, or plant part comprises a first nucleotide sequence encoding a polypeptide having an amino acid sequence with at least about 85% sequence identity to SEQ ID NO:72, a second nucleotide sequence encoding a polypeptide having an amino acid sequence with at least about 85% sequence identity to SEQ ID NO:73, a third nucleotide sequence encoding a polypeptide having an amino acid sequence with at least about 85% sequence identity to SEQ ID NO:74, a fourth nucleotide sequence encoding a polypeptide having an amino acid sequence with at least about 85% sequence identity to SEQ ID NO:75, a fifth nucleotide sequence encoding a polypeptide having an amino acid sequence with at least about 85% sequence identity to SEQ ID NO:76, and a sixth nucleotide sequence encoding a polypeptide having an amino acid sequence with at least about 85% sequence identity to SEQ ID NO:77, wherein the modified plant, seed, cell, or plant part is capable of producing pleuromutilin. In another embodiment, the modified plant, seed, cell, or plant part comprises a first nucleotide sequence encoding a polypeptide having an amino acid sequence with at least about 85% sequence identity to SEQ ID NO:78, a second nucleotide sequence encoding a polypeptide having an amino acid sequence with at least about 85% sequence identity to SEQ ID NO:79, a third nucleotide sequence encoding a polypeptide having an amino acid sequence with at least about 85% sequence identity to SEQ ID NO:80, and a fourth nucleotide sequence encoding a polypeptide having an amino acid sequence with at least about 85% sequence identity to SEQ ID NO:81, wherein the modified plant, seed, cell, or plant part is capable of producing pleuromutilin. In yet another embodiment, the modified plant, seed, cell, or plant part comprises a first nucleotide sequence encoding a polypeptide having an amino acid sequence with at least about 85% sequence identity to SEQ ID NO:82, a second nucleotide sequence encoding a polypeptide having an amino acid sequence with at least about 85% sequence identity to SEQ ID NO:83, a third nucleotide sequence encoding a polypeptide having an amino acid sequence with at least about 85% sequence identity to SEQ ID NO:84, a fourth nucleotide sequence encoding a polypeptide having an amino acid sequence with at least about 85% sequence identity to SEQ ID NO:85, a fifth nucleotide sequence encoding a polypeptide having an amino acid sequence with at least about 85% sequence identity to SEQ ID NO:86, a sixth nucleotide sequence encoding a polypeptide having an amino acid sequence with at least about 85% sequence identity to SEQ ID NO:87, a seventh nucleotide sequence encoding a polypeptide having an amino acid sequence with at least about 85% sequence identity to SEQ ID NO:88, an eighth nucleotide sequence encoding a polypeptide having an amino acid sequence with at least about 85% sequence identity to SEQ ID NO:89, a ninth nucleotide sequence encoding a polypeptide having an amino acid sequence with at least about 85% sequence identity to SEQ ID NO:90, and a tenth nucleotide sequence encoding a polypeptide having an amino acid sequence with at least about 85% sequence identity to SEQ ID NO:91, wherein the modified plant, seed, cell, or plant part is capable of producing enfumafungin. In still yet another embodiment, the modified plant, seed, cell, or plant part comprises a first nucleotide sequence encoding a polypeptide having an amino acid sequence with at least about 85% sequence identity to SEQ ID NO:92, a second nucleotide sequence encoding a polypeptide having an amino acid sequence with at least about 85% sequence identity to SEQ ID NO:93, a third nucleotide sequence encoding a polypeptide having an amino acid sequence with at least about 85% sequence identity to SEQ ID NO:94, a fourth nucleotide sequence encoding a polypeptide having an amino acid sequence with at least about 85% sequence identity to SEQ ID NO:95, a fifth nucleotide sequence encoding a polypeptide having an amino acid sequence with at least about 85% sequence identity to SEQ ID NO:96, a sixth nucleotide sequence encoding a polypeptide having an amino acid sequence with at least about 85% sequence identity to SEQ ID NO:97, a seventh nucleotide sequence encoding a polypeptide having an amino acid sequence with at least about 85% sequence identity to SEQ ID NO:98, and an eighth nucleotide sequence encoding a polypeptide having an amino acid sequence with at least about 85% sequence identity to SEQ ID NO:99, wherein the modified plant, seed, cell, or plant part is capable of producing fusidic acid. In one embodiment, the modified plant, seed, cell, or plant part comprises a first nucleotide sequence encoding a polypeptide having an amino acid sequence with at least about 85% sequence identity to SEQ ID NO:100 or SEQ ID NO:101, a second nucleotide sequence encoding a polypeptide having an amino acid sequence with at least about 85% sequence identity to SEQ ID NO:102 or SEQ ID NO:103, a third nucleotide sequence encoding a polypeptide having an amino acid sequence with at least about 85% sequence identity to SEQ ID NO:104 or SEQ ID NO:105, and a fourth nucleotide sequence encoding a polypeptide having an amino acid sequence with at least about 85% sequence identity to SEQ ID NO:106 or SEQ ID NO:107, wherein the modified plant, seed, cell, or plant part is capable of producing psilocybin.

In one embodiment, the at least one recombinant polynucleotide molecule comprises at least one nucleotide sequence encoding at least one polypeptide having an amino acid sequence with at least about 90%, about 91%, about 92%, about 93%, about 94%, about 95%, about 96%, about 97%, about 98%, about 99%, or 100% sequence identity to an amino acid sequence selected from the group consisting of SEQ ID NOs:1-107. In another embodiment, the modified plant, seed, cell, or plant part comprises at least one polypeptide having an amino acid sequence with at least about 90%, about 91%, about 92%, about 93%, about 94%, about 95%, about 96%, about 97%, about 98%, about 99%, or 100% sequence identity to an amino acid sequence selected from the group consisting of SEQ ID NOs:1-107. In yet another embodiment, the modified plant, seed, cell, or plant part comprises at least two, at least three, at least four, at least five, at least six, at least seven, at least eight, at least nine, at least ten, at least eleven, or at least twelve polypeptides having an amino acid sequence with at least about 90%, about 91%, about 92%, about 93%, about 94%, about 95%, about 96%, about 97%, about 98%, about 99%, or 100% sequence identity to an amino acid sequence selected from the group consisting of SEQ ID NOs:1-107. The modified plant, seed, cell, or plant part, in still yet another embodiment, comprises at least two, at least three, at least four, at least five, at least six, at least seven, at least eight, at least nine, at least ten, at least eleven, or at least twelve recombinant polynucleotide molecules.

In one embodiment, the modified plant, seed, cell, or plant part comprises at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:1-6, wherein the modified plant, seed, cell, or plant part is capable of producing penicillin. In another embodiment, the modified plant, seed, cell, or plant part comprises at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:7-15, wherein the modified plant, seed, cell, or plant part is capable of producing mycophenolic acid. In yet another embodiment, the modified plant, seed, cell, or plant part comprises at least two, at least three, at least four, at least five, at least six, at least seven, at least eight, at least nine, at least ten, at least eleven, or at least twelve nucleotide sequences encoding at least two, at least three, at least four, at least five, at least six, at least seven, at least eight, at least nine, at least ten, at least eleven, or at least twelve polypeptides having an amino acid sequence with at least about 85% sequence identity to an amino acid sequence selected from the group consisting of SEQ ID NOs:16-27, wherein the modified plant, seed, cell, or plant part is capable of producing cyclosporin. In still yet another embodiment, the modified plant, seed, cell, or plant part comprises at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:28-36, wherein the modified plant, seed, cell, or plant part is capable of producing cephalosporin.

In one embodiment, the modified plant, seed, cell, or plant part comprises at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:37-48, wherein the modified plant, seed, cell, or plant part is capable of producing pneumocandin B0. In another embodiment, the modified plant, seed, cell, or plant part comprises at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:49-54, wherein the modified plant, seed, cell, or plant part is capable of producing lovastatin. In yet another embodiment, the modified plant, seed, cell, or plant part comprises at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:55-63, wherein the modified plant, seed, cell, or plant part is capable of producing compactin. In still yet another embodiment, the modified plant, seed, cell, or plant part comprises at least two, at least three, at least four, at least five, at least six, at least seven, or at least eight nucleotide sequences encoding at least two, at least three, at least four, at least five, at least six, at least seven, or at least eight polypeptides having an amino acid sequence with at least about 85% sequence identity to an amino acid sequence selected from the group consisting of SEQ ID NOs:64-71, wherein the modified plant, seed, cell, or plant part is capable of producing griseofulvin.

In one embodiment, the modified plant, seed, cell, or plant part comprises at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:72-77, wherein the modified plant, seed, cell, or plant part is capable of producing pleuromutilin. In another embodiment, the modified plant, seed, cell, or plant part comprises at least two, at least three, or at least four nucleotide sequences encoding at least two, at least three, or at least four polypeptides having an amino acid sequence with at least about 85% sequence identity to an amino acid sequence selected from the group consisting of SEQ ID NOs:78-81, wherein the modified plant, seed, cell, or plant part is capable of producing pleuromutilin. In yet another embodiment, the modified plant, seed, cell, or plant part comprises at least two, at least three, at least four, at least five, at least six, at least seven, at least eight, at least nine, or at least ten nucleotide sequences encoding at least two, at least three, at least four, at least five, at least six, at least seven, at least eight, at least nine, or at least ten polypeptides having an amino acid sequence with at least about 85% sequence identity to an amino acid sequence selected from the group consisting of SEQ ID NOs:82-91, wherein the modified plant, seed, cell, or plant part is capable of producing enfumafungin. In still yet another embodiment, the modified plant, seed, cell, or plant part comprises at least two, at least three, at least four, at least five, at least six, at least seven, or at least eight nucleotide sequences encoding at least two, at least three, at least four, at least five, at least six, at least seven, or at least eight polypeptides having an amino acid sequence with at least about 85% sequence identity to an amino acid sequence selected from the group consisting of SEQ ID NOs:92-99, wherein the modified plant, seed, cell, or plant part is capable of producing fusidic acid. In one embodiment, the modified plant, seed, cell, or plant part comprises at least two, at least three, or at least four nucleotide sequences encoding at least two, at least three, or at least four polypeptides having an amino acid sequence with at least about 85% sequence identity to an amino acid sequence selected from the group consisting of SEQ ID NOs:100-107, wherein the modified plant, seed, cell, or plant part is capable of producing psilocybin.

The present disclosure further provides, in one embodiment, a plant commodity product produced from a modified plant, seed, cell, or plant part of the present disclosure, wherein the plant commodity product comprises the recombinant polynucleotide molecule. In another embodiment, the present disclosure provides a medicinal compound produced from a modified plant, seed, cell, or plant part of the present disclosure. The at least one recombinant polynucleotide molecule, in yet another embodiment, comprises a heterologous promoter functional in a plant cell. The at least one recombinant polynucleotide molecule, in still yet another embodiment, comprises a targeting nucleotide sequence or encodes a targeting polypeptide sequence. Any plant capable of genetic modification as described herein may be used according to the embodiments of the present disclosure, non-limiting examples of which include, a Nicotiana benthamiana, a corn, or a soybean plant.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a schematic of the penicillin G biosynthesis pathway in Penicillium chrysogenum. Domains within ACVS include adenylation (A), thiolation (T), condensation (C), epimerization (E), and thioesterase (TE).

FIG. 2 shows a schematic of mycophenolic acid biosynthesis in P. brevicompactin. SAT=starter unit: ACP transacylase, KS=ketosynthase, AT=acyltransferase, PT=product template, ACP=acyl carrier protein, and MT=methyltransferase.

FIG. 3 shows a schematic for the construction and transient expression of fungal genes in plant cells. As an example, the construction of ACVS with an N-terminal eGFP fusion is shown. Multiple plasmids can be co-expressed.

### BRIEF DESCRIPTION OF THE SEQUENCES

SEQ ID NO:1—representative amino acid sequence of an N-(5-Amino-5-carboxypentanoyl)-L-cysteinyl-D-valine synthase (ACV synthetase).

SEQ ID NO:2—representative amino acid sequence of an isopenicillin N synthase (IPNS).

SEQ ID NO:3—representative amino acid sequence of an isopenicillin N acyltransferase (IAT).

SEQ ID NO:4—representative amino acid sequence of a transporter paaT.

SEQ ID NO:5—representative amino acid sequence of a vacuolar transmembrane transporter PenV.

SEQ ID NO:6—representative amino acid sequence of a transporter PenM.

SEQ ID NO:7 representative amino acid sequence of a phosphopantetheinyl transferase (NpgA).

SEQ ID NO:8—representative amino acid sequence of a cytosolic polyketide synthase MpaC.

SEQ ID NO:9—representative amino acid sequence of a P450-hydrolase fusion enzyme MpaDE.

SEQ ID NO:10—representative amino acid sequence of a prenyltransferase MpaA.

SEQ ID NO:11—representative amino acid sequence of an oxygenase MpaB.

SEQ ID NO:12—representative amino acid of an O-methyltransferase MpaG.

SEQ ID NO:13—representative amino acid sequence of an acyl-CoA ligase PbACL891.

SEQ ID NO:14—representative amino acid sequence of an acyl-CoA hydrolase MpaH.

SEQ ID NO:15—representative amino acid sequence of a mycophenolic acid resistance protein MpaF.

SEQ ID NO:16—representative amino acid of a cyclosporin synthetase SimA.

SEQ ID NO:17—representative amino acid sequence of an alanine racemase SimB.

SEQ ID NO:18—representative amino acid sequence of a cyclophilin SimC.

SEQ ID NO:19—representative amino acid sequence of a transporter SimD.

SEQ ID NO:20—representative amino acid sequence of a thioesterase SimE.

SEQ ID NO:21—representative amino acid sequence of a cytochrome B2-like protein SimF.

SEQ ID NO:22—representative amino acid sequence of a polyketide synthase SimG.

SEQ ID NO:23—representative amino acid sequence of protein SimH.

SEQ ID NO:24—representative amino acid sequence of cytochrome P450 protein SimI.

SEQ ID NO:25—representative amino acid sequence of aminotransferase SimJ.

SEQ ID NO:26—representative amino acid sequence of protein SimK.

SEQ ID NO:27—representative amino acid sequence of transcription factor SimL.

SEQ ID NO:28—representative amino acid sequence of a N-(5-amino-5-carboxypentanoyl)-L-cysteinyl-D-valine synthase (ACVS) (NRPS).

SEQ ID NO:29—representative amino acid sequence of an isopenicillin N synthase (IPNS).

SEQ ID NO:30—representative amino acid sequence of a transporter CefP.

SEQ ID NO:31—representative amino acid sequence of a transporter CefM.

SEQ ID NO:32—representative amino acid sequence of an isopenicillin CoA synthetase CefD1.

SEQ ID NO:33—representative amino acid sequence of an isopenicillin CoA epimerase CefD2.

SEQ ID NO:34—representative amino acid sequence of a bifunctional deacetoxycephalosporin C synthase/hydroxylase CefEF.

SEQ ID NO:35—representative amino acid sequence of an acetyl coenzyme A: deacetylcephalosporin C acetyltransferase CefG.

SEQ ID NO:36—representative amino acid sequence of a transporter CefT.

SEQ ID NO:37—representative amino acid sequence of polyketide synthase GLPKS4 (HRPKS).

SEQ ID NO:38—representative amino acid sequence of a thioesterase GLHYD.

SEQ ID NO:39—representative amino acid sequence of a clavaminate synthase-like protein GLOXY1.

SEQ ID NO:40—representative amino acid sequence of a clavaminate synthase-like protein GLOXY2.

SEQ ID NO:41—representative amino acid sequence of a clavaminate synthase-like protein GLOXY3.

SEQ ID NO:42—representative amino acid sequence of a clavaminate synthase-like protein GLOXY4.

SEQ ID NO:43—representative amino acid sequence of an aldolase GLHtyA.

SEQ ID NO:44—representative amino acid sequence of a D-amino acid aminotransferase-like PLP-dependent enzyme GLHtyB.

SEQ ID NO:45—representative amino acid sequence of an isocitrate/isopropylmalate dehydrogenase-like protein GLHtyC.

SEQ ID NO:46—representative amino acid sequence of an aconitase GLHtyD.

SEQ ID NO:47—representative amino acid sequence of a cytochrome P450 GLP450-1.

SEQ ID NO:48—representative amino acid sequence of a cytochrome P450 GLP450-2.

SEQ ID NO:49—representative amino acid sequence of a cytochrome P450 monooxygenase LovA.

SEQ ID NO:50—representative amino acid sequence of a lovastatin nonaketide synthase LovB.

SEQ ID NO:51—representative amino acid sequence of an enoyl reductase LovC.

SEQ ID NO:52—representative amino acid sequence of a thioesterase LovG.

SEQ ID NO:53—representative amino acid sequence of an acyltransferase LovD.

SEQ ID NO:54—representative amino acid sequence of a polyketide synthase LovF.

SEQ ID NO:55—representative amino acid sequence of a compactin nonaketide synthase MlcA.

SEQ ID NO:56—representative amino acid sequence of a compactin diketide synthase MlcB.

SEQ ID NO:57—representative amino acid sequence of a cytochrome P450 MlcC.

SEQ ID NO:58—representative amino acid sequence of a 3-hydroxy-3-methylglutaryl coenzyme A reductase MlcD.

SEQ ID NO:59—representative amino acid sequence of a transporter MlcE.

SEQ ID NO:60—representative amino acid sequence of a thioesterase MlcF.

SEQ ID NO:61—representative amino acid sequence of an enoyl reductase MlcG.

SEQ ID NO:62—representative amino acid sequence of an acyltransferase MlcH.

SEQ ID NO:63—representative amino acid sequence of a transcription factor MlcR.

SEQ ID NO:64—representative amino acid sequence of a polyketide synthase GsfA.

SEQ ID NO:65—representative amino acid sequence of an O-methyltransferase GsfB.

SEQ ID NO:66—representative amino acid sequence of an O-methyltransferase GsfC.

SEQ ID NO:67—representative amino acid sequence of an O-methyltransferase GsfD.

SEQ ID NO:68—representative amino acid sequence of a NAD-dependent epimerase/dehydratase GsfE.

SEQ ID NO:69—representative amino acid sequence of a cytochrome P450 GsfF.

SEQ ID NO:70—representative amino acid sequence of a halogenase GsfI.

SEQ ID NO:71—representative amino acid sequence of a ketoreductase GsfK.

SEQ ID NO:72—representative amino acid sequence of a terpene cyclase Pl-cyc.

SEQ ID NO:73—representative amino acid sequence of a cytochrome P450 P1-P450-1.

SEQ ID NO:74—representative amino acid sequence of a cytochrome P450 P1-P450-2.

SEQ ID NO:75—representative amino acid sequence of a SDR family oxidoreductase Pl-sdr.

SEQ ID NO:76—representative amino acid sequence of an acetyl transferase Pl-atf.

SEQ ID NO:77—representative amino acid sequence of a cytochrome P450 P1-P450-3.

SEQ ID NO:78—representative amino acid sequence of a geranylgeranyl diphosphate synthase PbGGS.

SEQ ID NO:79—representative amino acid sequence of a terpene cyclase PbACS.

SEQ ID NO:80—representative amino acid sequence of a cytochrome P450 PbP450-2.

SEQ ID NO:81—representative amino acid sequence of a cytochrome P450 PbP450-1.

SEQ ID NO:82—representative amino acid sequence of a terpene cyclase/glycosyltransferase EfuA.

SEQ ID NO:83—representative amino acid sequence of a cytochrome P450 EfuB.

SEQ ID NO:84—representative amino acid sequence of an acetyl transferase AfuC.

SEQ ID NO:85—representative amino acid sequence of a dehydrogenase EfuE.

SEQ ID NO:86—representative amino acid sequence of a transporter EfuF.

SEQ ID NO:87—representative amino acid sequence of a cytochrome P450 EfuG.

SEQ ID NO:88—representative amino acid sequence of a cytochrome P450 EfuH.

SEQ ID NO:89—representative amino acid sequence of a desaturase EfuI.

SEQ ID NO:90—representative amino acid sequence of a resistance protein EfuJ.

SEQ ID NO:91—representative amino acid sequence of an oxysterol binding protein EfuK.

SEQ ID NO:92—representative amino acid sequence of a terpene cyclase FusA.

SEQ ID NO:93—representative amino acid sequence of a cytochrome P450 FusB1.

SEQ ID NO:94—representative amino acid sequence of a cytochrome P450 FusB2.

SEQ ID NO:95—representative amino acid sequence of a cytochrome P450 FusB3.

SEQ ID NO:96—representative amino acid sequence of a cytochrome P450 FusB4.

SEQ ID NO:97—representative amino acid sequence of a short-chain dehydrogenase/reductase FusC1.

SEQ ID NO:98—representative amino acid sequence of a short-chain dehydrogenase/reductase FusC2.

SEQ ID NO:99—representative amino acid sequence of an acyltransferase FusD.

SEQ ID NO:100—representative amino acid sequence of a tryptophan decarboxylase PsiD.

SEQ ID NO:101—representative amino acid sequence of a tryptophan decarboxylase PsiD.

SEQ ID NO:102—representative amino acid sequence of a kinase PsiK.

SEQ ID NO:103—representative amino acid sequence of a kinase PsiK.

SEQ ID NO:104—representative amino acid sequence of a methyltransferase PsiM.

SEQ ID NO:105—representative amino acid sequence of a methyltransferase PsiM.

SEQ ID NO:106—representative amino acid sequence of a cytochrome P450 PsiH.

SEQ ID NO:107—representative amino acid sequence of a cytochrome P450 PsiH.

SEQ ID NO:108—representative codon optimized sequence encoding the amino acid sequence of SEQ ID NO:1.

SEQ ID NO:109—representative codon optimized sequence encoding the amino acid sequence of SEQ ID NO:2.

SEQ ID NO:110—representative codon optimized sequence encoding the amino acid sequence of SEQ ID NO:3.

SEQ ID NO:111—representative codon optimized sequence encoding the amino acid sequence of SEQ ID NO:4.

SEQ ID NO:112—representative codon optimized sequence encoding the amino acid sequence of SEQ ID NO:5.

SEQ ID NO:113—representative codon optimized sequence encoding the amino acid sequence of SEQ ID NO:6.

SEQ ID NO:114—representative codon optimized sequence encoding the amino acid sequence of SEQ ID NO:7.

SEQ ID NO:115—representative codon optimized sequence encoding the amino acid sequence of SEQ ID NO:8.

SEQ ID NO:116—representative codon optimized sequence encoding the amino acid sequence of SEQ ID NO:9.

SEQ ID NO:117—representative codon optimized sequence encoding the amino acid sequence of SEQ ID NO:10.

SEQ ID NO:118—representative codon optimized sequence encoding the amino acid sequence of SEQ ID NO:11.

SEQ ID NO:119—representative codon optimized sequence encoding the amino acid sequence of SEQ ID NO:12.

SEQ ID NO:120—representative codon optimized sequence encoding the amino acid sequence of SEQ ID NO:13.

SEQ ID NO:121—representative codon optimized sequence encoding the amino acid sequence of SEQ ID NO:14.

SEQ ID NO:122—representative codon optimized sequence encoding the amino acid sequence of SEQ ID NO:15.

### DETAILED DESCRIPTION OF THE INVENTION

The present disclosure provides methods and compositions for producing fungal derived medicinal compounds in plants. These medicinal compounds are currently produced in fungal hosts by large scale fermentation, which requires expensive infrastructure, costly maintenance, and has negative environmental impacts. The present disclosure provides a significant advance in the art by demonstrating that crop plants are a sustainable medicine production platform for traditionally fungal derived medicines. Crop plants are currently used to produce endogenous pharmacoactive compounds such as morphine and cannabinoids and foreign protein components for vaccines. The methods and compositions of the present disclosure allow for the reconstitution of complete fungal metabolic pathways for the production of medicinal compounds within a plant host. Non-limiting examples such medicinal compounds include penicillin, mycophenolic acid, cyclosporin, cephalosporin, pneumocandin B0, lovastatin, compactin, griseofulvin, pleuromutilin, aphidicolin, enfumafungin, fusidic acid, and psilocybin (Table 1).

TABLE 1
Exemplary Medicinal Compounds Produced by Fungi.

| | Molecule | | |
|---|---|---|---|
| Name | Type | Purpose | Pathway Enzymes |
| Cyclosporin 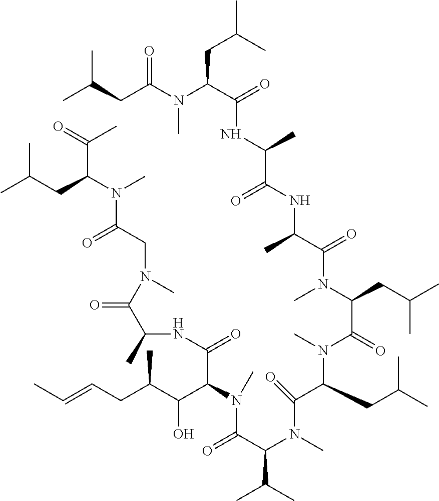 | Peptide | Immunosuppressant | SimA SimB SimC SimD SimE SimF SimG SimH SimI SimJ SimK SimL See, Yang et al. mBio 9(5): https://doi.org/10.1128/mbio. 01211-18, 2018. |
| Cephalosporin 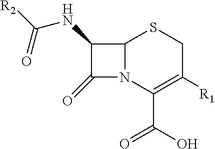 | Peptide | Antibiotic | ACVS IPNS CefP CefM IPN-CoA synthetase IPN-CoA epimerase CefD3 DAOCS DACS DACA CefT See, Martin et al. Adv Biochem Eng Biotechnol 88:91-109, 2004. |
| Pneumocandin B0 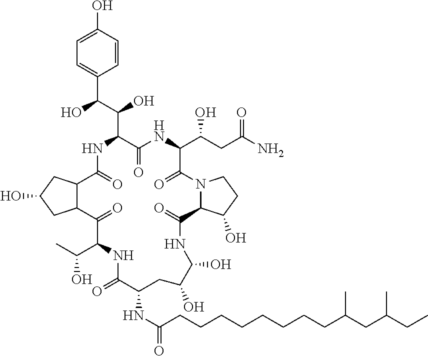 [SMILES: CCC(C)CC(C)CCCCCCCCC(=O)N[C@H]1C[C@@H](O)[C@@H](O)NC(=O)[C@@H]2[C@@H](O)CCN2C(=O)[C@H]([C@H](O)CC(N)=O)NC(=O)[C@H]([C@H](O)[C@@H](O)c2ccc(O)cc2)NC(=O)C2C[C@@H](O)CC2C(=O)[C@H]([C@@H](C)O)NC1=O] | Peptide | Antifungal | GLPKS4 GLHYD GLOXY1 GLOXY2 GLOXY3 GLOXY4 GLHty A GLHty B GLHty C GLHty D GLP450-1 GLP450-2 See, Chen et al. ACS Chem. Biol. 11, 10, 2724-2733, 2016. |
| Lovastatin 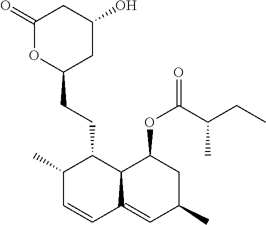 [SMILES: CC[C@H](C)C(=O)O[C@H]1C[C@@H](C)C=C2C=C[C@H](C)[C@H](CC[C@@H]3C[C@@H](O)CC(=O)O3)[C@H]21] | Polyketide | Cholesterol-lowering | LovA LovB LovC LovG LovD LovF See, Itoh et al. ACS Synth. Biol. 7, 12, 2783-2789, 2018. |
| Compactin 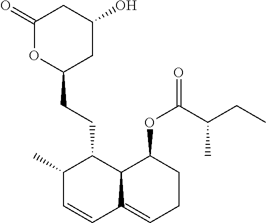 [SMILES: CC[C@H](C)C(=O)O[C@H]1CCC=C2C=C[C@H](C)[C@H](CC[C@@H]3C[C@@H](O)CC(=O)O3)[C@H]21] | Polyketide | Cholesterol-lowering | MlcA MlcB MlcC MlcD MlcE MLcF MlcG MlcH MlcR See, Itoh et al. ACS Synth. Biol. 7, 12, 2783-2789, 2018. |
| Griseofulvin 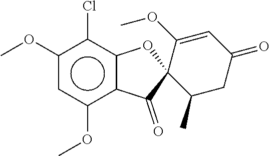 | Polyketide | Antifungal | GsfA GsfB GsfC GsfD GsfE GsfF GsfI GsfK See, Cacho et al. ACS Chem. Biol. 8, 10, 2322-2330, 2013. |
| Pleuromutilin 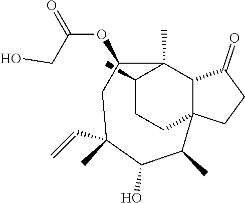 | Terpene | Antibiotic | Pl-cyc Pl-P450-1 Pl-P450-2 Pl-sdr Pl-atf Pl-P450-3 See, Alberti et al. Nature Communications. Volume 8, Article number: 1831, 2017. |
| Aphidicolin 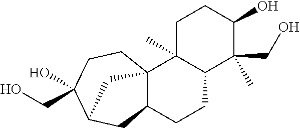 [SMILES: C[C@@]1(CO)[C@H](O)CC[C@@]2(C)[C@H]1CC[C@H]1C[C@@H]3C[C@@]12CC[C@]3(O)CO] | Terpene | Antiviral | PbGGS PbACS PbP450-2 PbP450-1 See, Fujii et al. Biosci Biotechnol Biochem 75(9): 1813-7, 2011. |
| Enfumafungin 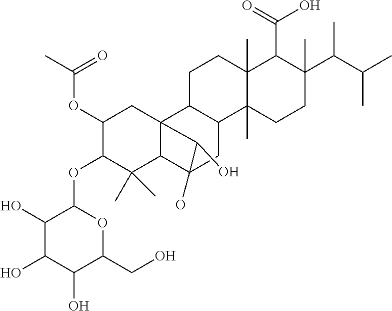 [SMILES: CC(=O)OC1CC23C4CCC5(C)C(C(=O)O)C(C)(C(C)C(C)C)CCC5(C)C4CC(O)(C2O)C3C(C)(C)C1OC1OC(CO)C(O)C(O)C1O] | Terpene | Antifungal | EfuA EfuB AfuC EfuE EfuF EfuG EfuH EfuI EfuJ EfuK See, Kuhnert et al. Environ Microbiol. 20(9): 3325-3342, 2018. |
| Fusidic acid 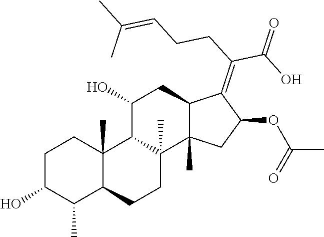 | Terpene | Antibiotic | FusA FusB1 FusB2 FusB3 FusB4 FusC1 FusC2 FusD See, Cao et al. Acta Pharmaceutica Sinica B Volume 9, Issue 2, 433-442, 2019. |
| Psilocybin 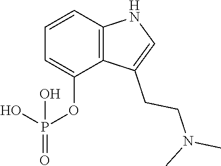 [SMILES: CN(C)CCc1c[nH]c2cccc(OP(=O)(O)O)c12] | Alkaloid | Psychedelic | PsiD PsiK PsiM PsiH |

### A. Overview of Penicillin and Mycophenolic Acid Biosynthesis in Plants

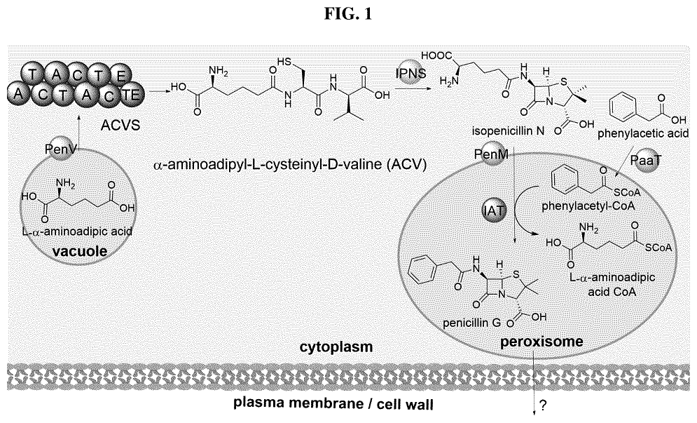

In penicillin biosynthesis the non-ribosomal peptide synthetase (NRPS; ACVS) selects, activates, and condenses L-α-aminoadipic acid, L-cysteine, and L-valine to synthesize α-aminoadipyl-L-cysteinyl-D-valine (ACV), the first enzyme-free intermediate in the pathway (FIG. 1). Isopenicillin N synthase (IPNS) then converts ACV to isopenicillin N. Both ACVS and IPNS are located in the cytoplasm. Isopenicillin N is then transported into peroxisomes and converted to penicillin G through the action of isopenicillin N acyltransferase (IAT). Three transporters are required for shuttling intermediates between compartments: PenV transports L-α-aminoadipic acid from the vacuole to the cytoplasm; PenM transports isopenicillin N from the cytoplasm to peroxisomes; and PaaT transports phenylacetic acid from the cytoplasm to peroxisomes (FIG. 1). The final transport of penicillin G from peroxisomes outside cells and

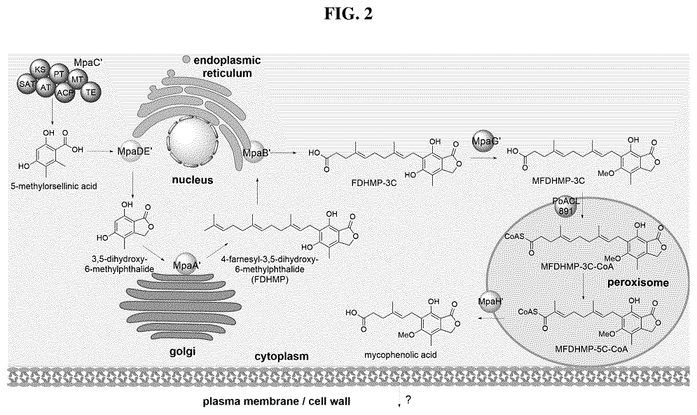

The biosynthesis of mycophenolic acid was elucidated in Penicillium brevicompactum where the cytoplasmic non-reducing polyketide synthase (PKS) MpaC′ synthesizes 5-methylorsellinic acid (FIG. 2). The dual function P450/hydrolase MpaDE′, localized in the endoplasmic reticulum, converts 5-methylorsellinic acid to 3,5-dihydroxy-7-(hydroxymethyl)-6-methylbenzoic acid and then to 3,5-dihydroxy-6-methylphthalide. The prenyltransferase MpaA′ farnesylates 3,5-dihydroxy-6-methylphthalide to 4-farnesyl-3,5-dihydroxyphthalide (FDHMP) at the Golgi complex, and the endoplasmic-reticulum associated oxidase MpaB′ catalyzes the oxidative cleavage of FDHMP resulting in FDHMP-3C. The cytoplasmic O-methyltransferase MpaG′ synthesizes MFDHMP-3C. The acyl-CoA ligase PbACL891 converts MFDHMP-3C to its acyl-CoA ester MFDHMP-3C-CoA, which is subsequently shortened via β-oxidation, controlled by the acyl-CoA hydrolase MpaH′ located within peroxisomes, and finally released as mycophenolic acid (Zhang et al, Proc. Nat. Acad. Sci., 116(27): 13305-13310, 2019). No transporters have been identified and it is unknown how mycophenolic acid is secreted into media.

The pathways for penicillin G and mycophenolic acid biosynthesis have been well-studied in fungi. These pathways require the action of several different classes of coordinating enzymes located in different subcellular compartments with analogous subcellular compartments in plant cells (Martin, Fungal Biol. Biotechnol. 7:6, 2020; Zhang et al, Proc. Nat. Acad. Sci., 116(27): 13305-13310, 2019). Importantly, penicillin and mycophenolic acid are not toxic towards plants and therefore can be accumulated at high levels. Furthermore, all required metabolic precursors for the production of penicillin and mycophenolic acid are present in plant cells. Chemical standards and analytical methods required to assess production yields of penicillin and mycophenolic acid in plants are also readily available (Aldeek et al. J. Agric. Food Chem. 63 (26): 5993-6000, 2015). Plants are known to use single domain Type III polyketide synthases (PKS) for the biosynthesis of polyketide compounds, such as medicinally important cannabinoids, flavonoids, and alkaloids. Although in silico analyses have predicted NRPS-like tri-domain enzymes in Oryza sativa and Arabidopsis thaliana, but their functions are unknown. A larger NRPS-like enzyme, DWA1, has been characterized in japonica rice, and has been shown to regulate drought-induced cuticular wax deposition in rice. Therefore, plants do not appear to possess the multifunctional megasynthase machinery for the de novo biosynthesis of polyketides or small peptides similar to those found in fungi. As such, the introduction of functional NRPS and Type I PKS enzymes into plants represents a significant scientific and technical advance. The methods and compositions of the present disclosure allow for further expansion of the types of microbial bioactive small molecules that can be produced by harnessing the energy efficiency of photosynthesis and redirecting plant metabolism toward valuable secondary metabolites. The methods and compositions of the present disclosure further enable the study of numerous non-plant biosynthetic pathways that may be challenging to investigate in the natural host and enable the production of complex natural products that cannot be chemically synthesized on an industrial scale.

### B. Nucleic Acids and Proteins

The present disclosure provides plants, seeds, cells, and plant parts comprising the nucleic acid or proteins of the present disclosure. As used herein, the term “wild type” refers to the endogenous version of a molecule that naturally occurs in an organism. In some aspects of the present disclosure, a wild-type version of a protein or polypeptide may be employed. In many aspects of the present disclosure, however, a recombinant protein or polypeptide is employed. The terms recombinant protein and recombinant polypeptide may be used interchangeably with the terms modified protein, modified polypeptide, and variant. In certain embodiments, a recombinant protein refers to a protein having an altered chemical structure or amino acid sequence compared to the wild-type protein. In some aspects, a recombinant protein may have at least one modified activity or function compared to the wild-type protein. As is known in the art, proteins may have multiple activities or functions. In some embodiments, the function of a recombinant protein may be altered with respect to one activity or function but retain the activity or function of the wild-type protein in other respects. In certain embodiments, the proteins of the present disclosure may include those which comprise a mutation compared to the wild-type protein. In one embodiment, the mutation may comprise an insertion, a deletion, a truncation, or at least one amino acid substitution.

The nucleotide and protein sequences for various genes have been previously disclosed, and may be found in computerized databases known in the art. Two such databases are the National Center for Biotechnology Information's GenBank and GenPept databases (on the World Wide Web at ncbi.nlm.nih.gov/) and The Universal Protein Resource (UniProt; on the World Wide Web at uniprot.org). The coding regions for these genes may be amplified or expressed using techniques known in the art or those disclosed herein.

As is known in the art, amino acid residues may be changed in a polypeptide sequence to create an equivalent, or even improved, second-generation variant polypeptide. For example, certain amino acids may be substituted for other amino acids in a polypeptide sequence without appreciable loss of binding capacity or specificity to structures such as, for example, binding sites on substrate molecules. Since the binding capacity, the binding specificity, and the nature of a protein define the functional activity of a protein, certain amino acid substitutions can be made in a protein sequence, and/or in its corresponding DNA coding sequence, such that the resultant variant protein comprises similar or desirable properties of the original protein. Thus, in some embodiments, the polynucleotide and polypeptide sequences of the present disclosure may comprise various amino acid or nucleic acid substitutions, deletions, and/or insertions without appreciable loss of biological utility or activity. As used herein the term “functionally equivalent codon” refers to codons that encode the same amino acid, such as the six different codons known in the art which code for arginine. As used herein, the terms “neutral substitutions” and “neutral mutations” refer to a change in a polypeptide sequence, or the encoding nucleotide sequence, such that the sequence comprises or encodes a biologically equivalent amino acid compared to that found in the original sequence. In certain embodiments, any amino acid of any polypeptide described herein may be substituted with any biologically equivalent amino acid. Biologically equivalent amino acids are known in the art.

Nucleic acid or amino acid sequence variants of the disclosure may comprise, in some embodiments, a substitution, an insertion, or a deletion. A polypeptide variant of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the polypeptide, as compared to the referenced polypeptide or to the wild-type polypeptide, including any range derivable therebetween. A variant polypeptide may comprise, for example, an amino acid sequence having at least 50%, 60%, 70%, 80%, or 90% sequence identity to a sequence comprising or encoded by any one of SEQ ID NOs:1-122 or fragments thereof, including all ranges derivable therebetween. A polypeptide may include, for example, at least 1, 2, 3, 4, 5, 6, 7, 8, 9, 10, 11, 12, 13, 14, 15, 16, 17, 18, 19, or 20 amino acid substitutions.

It also will be understood that amino acid and nucleic acid sequences may include additional residues, such as additional N- or C-terminal amino acids, or 5′ or 3′ sequences, respectively, and yet still be essentially identical to the sequences provided by the present disclosure. Such essentially identical sequences, in some embodiments, may maintain the biological activity described herein for the sequences of the present disclosure. The addition of terminal sequences particularly applies to nucleic acid sequences that may, for example, include various non-coding sequences flanking either of the 5′ or 3′ portions of the coding region.

Deletion variants typically lack one or more amino acid residues compared to the protein from which the variant was derived, the native protein, or the wild-type protein. In certain embodiments, individual amino acid residues may be deleted, or a number of contiguous amino acids may be deleted. In one embodiment, a stop codon may be introduced, for example by substitution or insertion, into an encoding nucleic acid sequence to generate a truncated protein variant.

Insertional variants typically involve the addition of one or more amino acid residues at a non-terminal point of a polypeptide. Terminal additions may also be generated and can include fusion proteins. Non-limiting examples of such fusion proteins include multimers or concatemers of one or more polypeptides provided by the present disclosure.

Substitutional variants typically comprise the exchange of one amino acid for another at one or more sites within the polypeptide. Substitutional variants may be designed to modulate one or more properties of the polypeptide, with or without the loss of other functions or properties of the polypeptide. Amino acid substitutions may be conservative amino acid substitutions. As used herein, the term “conservative amino acid substitution” refers to an amino acid substitution wherein one amino acid is replaced with another amino acid having similar chemical properties. Conservative amino acid substitutions may involve, for example, the exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may, in some embodiments, encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be non-conservative. As used herein, the term “non-conservative amino acid substitution” refers to an amino acid substitution that affects a function of the polypeptide. Non-conservative amino acid substitutions typically involve substituting an amino acid residue with one that is chemically dissimilar. Non-conservative amino acid substitutions may include, for example, the substitution of a polar or charged amino acid for a nonpolar or uncharged amino acid, and vice versa. Non-conservative substitutions may also include, for example, the substitution of a member of one of the amino acid classes for a member from another class.

One skilled in the art can determine suitable polypeptide variants, as set forth herein, using well-known techniques. For example, one skilled in the art may identify suitable areas of the polypeptide molecule that may be changed without affecting activity by targeting regions not believed to be critical for activity. The skilled artisan will also be able to identify amino acid residues and portions of the polypeptide molecules that are conserved among similar proteins or polypeptides. In some embodiments, regions of a polypeptide molecule that may be important for biological activity or for structure may be subject to conservative amino acid substitutions without significantly altering the biological activity or adversely affecting the protein structure.

When making conservative or non-conservative amino acid substitutions, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. These values are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the disclosure, those that are within ±1 are included, and in other aspects of the disclosure, those within ±0.5 are included.

As is known in the art, the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In some embodiments, amino acid substitutions based upon similar hydrophilicity values may include the substitution of amino acids whose hydrophilicity values are within ±2 of each other. In one embodiment, the substitution of amino acids whose hydrophilicity values are within ±1 or within ±0.5 are included.

In certain aspects, the present disclosure provides polynucleotide molecules that encode the polypeptide molecules describes herein. Non-limiting example of such polynucleotide molecules include isolated polynucleotide segments, recombinant vectors, and recombinant polynucleotide molecules. A nucleic acid molecule is the “complement” of another nucleic acid molecule if they exhibit complete complementarity. As used herein, two molecules exhibit “complete complementarity” if when aligned every nucleotide of the first molecule is complementary to every nucleotide of the second molecule. Two molecules are “minimally complementary” if they can hybridize to one another with sufficient stability to permit them to remain annealed to one another under at least conventional “low stringency” conditions. Similarly, the molecules are “complementary” if they can hybridize to one another with sufficient stability to permit them to remain annealed to one another under conventional “high stringency” conditions. Departures from complete complementarity are therefore permissible, as long as such departures do not completely preclude the capacity of the molecules to form a double-stranded structure. As used herein, with respective to a given sequence, a “complement,” a “complementary sequence” and a “reverse complement” are used interchangeably. All three terms refer to the inversely complementary sequence of a nucleotide sequence, i.e., to a sequence complementary to a given sequence in reverse order of the nucleotides.

Appropriate stringency conditions that promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

By convention, the DNA sequences of the present disclosure and fragments thereof are disclosed with reference to only one strand of the two complementary DNA sequence strands. By implication and intent, the complementary sequences of the sequences provided here (the sequences of the complementary strand), also referred to in the art as the reverse complementary sequences, are within the scope of the disclosure and are expressly intended to be within the scope of the subject matter claimed. Thus, as used herein reference to any one of SEQ ID NOs:108-122 and fragments thereof include and refer to the sequence of the complementary strand and fragments thereof.

A polynucleotide molecule of the present disclosure may, in some embodiments, comprise a contiguous nucleic acid sequence that encodes all or part of a polypeptide described herein. In certain embodiments, a polypeptide described herein may be encoded by variant nucleic acid sequences that encode the same or a substantially similar protein.

The polynucleotide molecules and fragments thereof provided by the present disclosure may, in some embodiments be combined with other polynucleotide molecules which comprise elements, such as promoters, polyadenylation signals, additional restriction enzyme sites, multiple cloning sites, other coding segments, and the like, such that the overall length of the polynucleotide molecule may vary considerably. The polynucleotide molecule provided by the present disclosure can be any length. In some cases, a nucleic acid sequence may encode a polypeptide sequence that comprises additional heterologous coding sequences. In one embodiment, the additional heterologous coding sequence may serve as a peroxisome, endoplasmic reticulum, or Golgi localization signal.

In some aspects of the present disclosure, a polypeptide or polynucleotide of the present disclosure may comprise an amino acid sequence or be encoded by a nucleotide sequence comprising a sequence having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs:1-122, including any range derivable therebetween. In certain embodiments, a polypeptide or polynucleotide of the present disclosure comprise or be encoded by a fragment of a sequence having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs:1-122, including any range derivable therebetween. A polypeptide or polynucleotide of the present disclosure may comprise, for example, at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275,300, 325, 350, 375, 400, 425, 450, 475, 500, 525, 550, 575, 600, 625, 650, 675, 700, 725, 750, 775, 800, 825, 850, 875, 900, 925, 950, 1000, 1100, 1200, 1300, 1400, 1500, 1600, 1700, 1800, 1900, 2000, 2500, 3000, 3500, 4000, 4500, 5000, 5500, or more nucleotides or amino acid residues of a sequence having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs:1-122, including any range derivable therebetween.

### C. Genome Editing

The present disclosure provides, in certain embodiments, plants, plant parts, plant cells, and seeds produced through genome modification using site-specific integration or genome editing. Genome editing can be used to make one or more edit(s) or mutation(s) at a desired target site in the genome of a plant, such as to change expression and/or activity of one or more genes, or to integrate an insertion sequence or transgene at a desired location in a plant genome. Any site or locus within the genome of a plant may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within a plant genome that is bound and cleaved by a site-specific nuclease to introduce a double-stranded break (DSB) or single-stranded nick into the nucleic acid backbone of the polynucleotide sequence and/or its complementary DNA strand within the plant genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion, substitution, inversion, or duplication. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system or the CRISPR/Cpf1 system), a TALE (transcription activator-like effector)-endonuclease (TALEN), a recombinase, or a transposase. As used herein, “editing” or “genome editing” refers to generating a targeted mutation, deletion, insertion, substitution, inversion, or duplication of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, insertion, substitution, inversion, or duplication, whereas “edits” or “genomic edits” refers to two or more targeted mutation(s), deletion(s), insertion(s), substitution(s), inversion(s), and/or duplication(s), with each “edit” being introduced via a targeted genome editing technique.

According to some embodiments, a site-specific nuclease may be co-delivered with a donor template molecule to serve as a template for making a desired edit, mutation or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease provided herein may be selected from the group consisting of a zinc-finger nuclease (ZFN), a TALE-endonuclease (TALEN), a meganuclease, an RNA-guided endonuclease (e.g., Cas9 and Cpf1), a recombinase, a transposase, or any combination thereof. See, e.g., Khandagale et al. (Plant Biotechnol Rep 10:327-343, 2016); and Gaj et al. (Trends Biotechnol. 31(7):397-405, 2013). Zinc finger nucleases (ZFN) are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site but may typically be composed of 3-4 (or more) zinc-fingers. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., U.S. Patent App. Pub. Nos. 2005/0064474, 2009/0117617, and 2012/0142062. The Foklnuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, Sbfl, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al., PLoS One 8:e82539, 2013). MutH is capable of introducing strand-specific nicks in DNA (see Gabsalilow et al., Nucleic Acids Research. 41:e83, 2013). TevI introduces double-stranded breaks in DNA at targeted sites (see Beurdeley et al., Nature Communications 4:1762, 2013).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al. (Nucleic Acids Research 40: W117-122, 2012); Cermak et al. (Nucleic Acids Research 39:e82, 2011); and tale-nt.cac.cornell.edu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

A site-specific nuclease may be an RNA-guided nuclease. In an aspect, the targeted genome editing described herein may comprise the use of an RNA-guided endonuclease. As used herein, an “RNA-guided nuclease” refers to an RNA-guided DNA endonuclease associated with the CRISPR system. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a, see e.g., Safari, F. et al., Cell Biosci 9:36, 2019), CasX, CasY, and homologs or modified versions of any thereof, as well as Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 enzyme. According to some embodiments, an RNA-guided endonuclease is a Cpf1 enzyme.

The CRISPR system, in its native context, provide bacteria and archaea with immunity to invading foreign nucleic acids and relies on an RNA-guided endonuclease to cleave the invading DNA or RNA into short sequence fragments and incorporating them into the bacterial CRISPR genomic locus. The incorporated short sequences, referred to as “protospacers,” and flanking direct repeats are transcribed and processed into CRISPR RNAs (crRNAs). These crRNAs hybridize with trans-activating crRNAs (tracrRNAs) to activate the RNA-guided Cas endonuclease to form a ribonucleoprotein (RNP) complex that is guided to a target site. A prerequisite for cleavage of the target site, however, is the presence of a conserved genomic protospacer-adjacent motif sequence recognized by the Cas endonuclease. A “protospacer adjacent motif” (PAM) herein refers to a short nucleotide sequence adjacent to a target sequence (protospacer) that is recognized (targeted) by a guide polynucleotide/Cas endonuclease system described herein. A PAM may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2(2):59-70, 2014). The Cas endonuclease may not successfully recognize a target DNA sequence if the target DNA sequence is not followed by a PAM sequence. The sequence and length of a PAM sequence herein can differ depending on the Cas endonuclease used. The PAM sequence can be of any length but is typically 1, 2, 3, 4, 5, 6, 7, 8, 9, 10, 11, 12, 13, 14, 15, 16, 17, 18, 19 or 20 nucleotides long.

CRISPR/Cas9, which is the CRISPR system from Streptococcus pyogenes, was adapted for use in eukaryotes and has been widely used for gene editing in plants. The CRISPR/Cas9 system requires both crRNA and tracrRNA to guide the Cas9 protein to recognize and cleave the target DNA double helix. Cas9 recognizes the genomic PAM sequence 5′-NGG-3′ (where N is any nucleotide) and, when located on the sense (+) strand adjacent to the target site, will create a blunt-end DSB at the target site, specifically the 5′-end of the PAM site. Cas9 has been observed to recognize other PAM sequences, such as 5′-NAG-3′ and 5′-NGA-3,′ which may result in cleavage of non-specific DNA sequences. However, the corresponding sequence of the guide RNA (i.e., immediately downstream (3′) to the targeting sequence of the guide RNA) may generally not be complementary to the genomic PAM sequence.

Recently, the CRISPR/Cpf1 system was discovered as an alternative to the CRISPR/Cas9 system for genome editing. While CRISPR/Cpf1 functions in a manner similar to CRISPR/Cas9, it is an even simpler system than CRISPR/Cas9. CRISPR/Cpf1 requires only one crRNA molecule and no tracrRNA to cleave DNA. Cpf1 recognizes the genomic PAM sequence 5′-TTTV-3′ (where V is A, G, or C) or 5′-TTN-3′, depending on the Cpf1 ortholog. See e.g., Alok et al. (Front. Plant Sci. 11:264, 2020). When Cpf1 recognizes the genomic PAM located on the sense (+) strand adjacent to the target site, it will generate a staggered DSB with a 4 or 5-nt 5′ overhang at the target site, specifically the 3′-end of the PAM site.

The RNA-guided nuclease may be delivered as a protein with or without a guide RNA, or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the plant via base-pairing or hybridization to cause a DSB or nick at or near the target site. The guide RNA may be transformed or introduced into a plant cell or tissue as a gRNA molecule, or as a recombinant DNA molecule, construct or vector comprising a transcribable DNA sequence encoding the guide RNA operably linked to a promoter. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). A “single-chain guide RNA” (or “sgRNA”) is an RNA molecule comprising a crRNA covalently linked to a tracrRNA by a linker sequence, which may be expressed as a single RNA transcript or molecule. The guide RNA comprises a guide or targeting sequence (also referred to herein as a “spacer sequence”) that is identical or complementary to a target site within the plant genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

In addition to the guide sequence, a guide RNA may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2(2):59-70, 2014). However, the corresponding sequence of the guide RNA (i.e., immediately downstream (3′) to the targeting sequence of the guide RNA) may generally not be complementary to the genomic PAM sequence.

In some embodiments, a site-specific nuclease is a recombinase. Non-limiting examples of recombinases that may be used include a serine recombinase attached to a DNA recognition motif, a tyrosine recombinase attached to a DNA recognition motif, or any recombinase enzyme known in the art attached to a DNA recognition motif. In certain embodiments, the site-specific nuclease is a recombinase or transposase, which may be a DNA transposase or recombinase attached or fused to a DNA binding domain. Non-limiting examples of recombinases include a tyrosine recombinase selected from the group consisting of a Cre recombinase, a Gin recombinase, a Flp recombinase, and a Tnp1 recombinase attached to a DNA recognition motif provided herein. In one aspect of the present disclosure, a Cre recombinase or a Gin recombinase provided herein is tethered to a zinc-finger DNA-binding domain, a TALE DNA-binding domain, or a Cas9 nuclease. In another aspect, a serine recombinase selected from the group consisting of a PhiC31 integrase, an R4 integrase, and a TP-901 integrase may be attached to a DNA recognition motif provided herein. In yet another aspect, a DNA transposase selected from the group consisting of a TALE-piggyBac and TALE-Mutator may be attached to a DNA binding domain provided herein.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such a genomic locus. The DSB or nick created by the non-RNA-guided site-specific nuclease may lead to knockdown of gene expression, or a change in the activity of the protein encoded by the endogenous gene, via repair of the DSB or nick, which may result in a mutation or insertion of a sequence at the site of the DSB or nick through cellular repair mechanisms. Such cellular repair mechanism may be guided by a donor template molecule.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of a plant cell via repair of a nick or DSB in the genome of a plant cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes or transcribable DNA sequences.

Without being limiting, a gene/transgene or transcribable DNA sequence of a donor template may include, for example, a sequence that encodes a polypeptide comprising a sequence having at least about 70% sequence identity to any one of SEQ ID NOs:1-107 or a fragment thereof, a sequence encoding a cellular localization signal, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter operably linked to a coding sequence, gene, or transcribable DNA sequence, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region or sequence, 3′-UTR, and/or polyadenylation signal, which may each be operably linked to a coding sequence, gene (or transgene) or transcribable DNA sequence encoding a non-coding RNA, a guide RNA, an mRNA and/or protein. A donor template may be a single-stranded or double-stranded DNA or RNA molecule or plasmid.

An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant.

Any method known in the art for site-directed integration may be used with the present disclosure. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

As used herein, the term “insertion” as it relates to a mutation, refers to the addition of one or more extra nucleotides into the DNA. Insertions in the coding region of a gene may alter splicing of the mRNA (splice site mutation) or cause a shift in the reading frame (frameshift), both of which can significantly alter the gene product.

As used herein, the term “deletion” as it relates to a mutation refers to the removal of one or more nucleotides from the DNA. Like insertion mutations, these mutations can alter the reading frame of the gene.

As used herein, the term “substitution” as it relates to a mutation refers to an exchange of a single nucleotide for another.

As used herein, the term “inversion” refers to reversing the orientation of a chromosomal segment. An inversion can be accompanied by a loss of nucleotides flanking either one or both sites of the inversion due to DNA repair mechanisms occurring at the cut and ligation sites during the formation of an inversion.

As used herein, the term “duplication” refers to the creation of multiple copies of chromosomal regions, increasing the dosage of the genes located within them.

As used herein, a “missense mutation” refers to a single nucleotide change that results in a codon that codes for a different amino acid. For example, the codon “CGU” encodes an arginine amino acid. If a missense mutation changes the G to a U, producing a “CUU” codon, the codon now encodes a leucine amino acid. Missense mutations can be caused by an insertion, deletion, substitution, duplication, or inversion. The frameshift, missense, or nonsense mutations described herein lead to loss of function or expression of a targeted gene. A “loss-of-function mutation” is a mutation in the coding sequence of a gene, which causes the function of the gene product, usually a protein, to be either reduced or completely absent, e.g., reduction of peptidase activity. A loss-of-function mutation can, for instance, be caused by the truncation of the gene product. A phenotype associated with an allele with a loss of function mutation can be either recessive or dominant.

Similarly, such targeted mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, substitutions, inversions, and/or duplications, relative to the targeted genomic sequence at or near the site of the DSB or nick. For example, targeted mutations of a gene may be achieved by deleting, inserting, substituting, inverting, or duplicating at least a portion of the gene, such as by introducing a frame shift or premature stop codon into the coding sequence of the gene or introducing a modification into a transcribable DNA sequence. A deletion of a portion of a gene may also be introduced by generating DSBs or nicks at two target sites and causing a deletion of the intervening target region flanked by the target sites. A modification of a targeted gene may result in expression of a non-functional protein, interfering protein, or a protein having reduced, disrupted, or altered activity as compared to a protein expressed from the gene lacking the modification.

### D. Constructs for Genome Editing

Recombinant DNA constructs and vectors are provided comprising a polynucleotide sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a plant expressible promoter. For RNA-guided endonucleases, recombinant DNA constructs and vectors are further provided comprising a polynucleotide sequence encoding a guide RNA, wherein the guide RNA comprises a guide sequence of sufficient length having a percent identity or complementarity to a target site within the genome of a plant. A polynucleotide sequence of a recombinant DNA construct and vector that encodes a site-specific nuclease or a guide RNA may be operably linked to a plant expressible promoter, such as an inducible promoter, a constitutive promoter, a tissue-specific promoter, etc.

In an aspect, vectors comprising polynucleotides encoding a site-specific nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In an aspect, vectors comprising polynucleotides encoding a Cpf1 nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, “locus” is a chromosomal locus or region where a polymorphic nucleic acid, trait determinant, gene, or marker is located. A “locus” can be shared by two homologous chromosomes to refer to their corresponding locus or region. As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele.

As used herein, a “wild-type gene” or “wild-type allele” refers to a gene or allele having a sequence or genotype that is most common in a particular plant species, or another sequence or genotype having only natural variations, polymorphisms, or other silent mutations relative to the most common sequence or genotype that do not significantly impact the expression and activity of the gene or allele. Indeed, a “wild type” gene or allele contains no variation, polymorphism, or any other type of mutation that substantially affects the normal function, activity, expression, or phenotypic consequence of the gene or allele relative to the most common sequence or genotype.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions, inversions, duplications, or any desired combinations of such changes in a native polynucleotide or amino acid sequence.

As used herein, the term “expression” refers to the biosynthesis of a gene product, and typically the transcription and/or translation of a nucleotide sequence, such as an endogenous gene, a heterologous gene, a transgene or an RNA and/or protein coding sequence, in a cell, tissue, organ, or organism, such as a plant, plant part or plant cell, tissue or organ.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other.

Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc. The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

Reference in this application to an “isolated DNA molecule” or an “isolated polynucleotide,” or an equivalent term or phrase, is intended to mean that the DNA molecule or polynucleotide is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding a protein or any naturally occurring variant of that protein would be an isolated nucleotide sequence so long as the nucleotide sequence was not within the DNA of the organism in which the sequence encoding the protein is naturally found. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As commonly understood in the art, the term “promoter” can generally refer to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present disclosure can thus include variants or fragments of promoter sequences that are similar in composition, but not identical to, other promoter sequence(s) known or provided herein. A promoter provided herein, or variant or fragment thereof, may comprise a “minimal promoter” which provides a basal level of transcription and is comprised of a TATA box or equivalent DNA sequence for recognition and binding of the RNA polymerase II complex for initiation of transcription. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc.

As used herein, a “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature without human introduction—e.g., the promoter or regulatory sequence has a different origin relative to the associated polynucleotide sequence and/or the promoter or regulatory sequence is not naturally occurring in a plant species to be transformed with the promoter or regulatory sequence.

As used herein, an “endogenous gene” or an “endogenous locus” refers to a gene or locus at its natural and original chromosomal location.

As used herein, in the context of a protein-coding gene, an “exon” refers to a segment of a DNA or RNA molecule containing information coding for a protein or polypeptide sequence.

As used herein, an “intron” of a gene refers to a segment of a DNA or RNA molecule, which does not contain information coding for a protein or polypeptide, and which is first transcribed into an RNA sequence but then spliced out from a mature RNA molecule.

As used herein, an “untranslated region (UTR)” of a gene refers to a segment of an RNA molecule or sequence (e.g., a mRNA molecule) expressed from a gene (or transgene) but excluding the exon and intron sequences of the RNA molecule. An “untranslated region (UTR)” also refers to a DNA segment or sequence encoding such a UTR segment of an RNA molecule.

An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, a “transcribable region” or “transcribable DNA sequence” refers to a nucleic acid sequence expressed from a gene (or transgene).

As used herein, a “transcription termination sequence” refers to a nucleic acid sequence containing a signal that triggers the release of a newly synthesized transcript RNA molecule from an RNA polymerase complex and marks the end of transcription of a gene or locus.

The terms “percent identity,” “% identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

Degeneracy of the genetic code provides the possibility to substitute at least one base of the protein encoding sequence of a gene with a different base without causing the amino acid sequence of the polypeptide produced from the gene to be changed. When optimally aligned, homolog proteins, or their corresponding nucleotide sequences, have typically at least about 70%, at least about 75%, at least about 80%, at least about 85%, at least about 90%, at least about 92%, at least about 93%, at least about 94%, at least about 95%, at least about 96%, at least about 97%, at least about 98%, at least about 99%, or even at least about 99.5% identity over the full length of a protein or its corresponding nucleotide sequence identified as being associated with imparting an altered phenotype when expressed in plant cells.

Homologs are inferred from sequence similarity, by comparison of protein sequences, for example, manually or by use of a computer-based tool. For optimal alignment of sequences to calculate their percent identity, various pairwise or multiple sequence alignment algorithms and programs are known in the art, such as ClustalW or Basic Local Alignment Search Tool® (BLAST), etc., that can be used to compare the sequence identity or similarity between two or more nucleotide or protein sequences. BLAST can also be used, for example, to search query protein sequences of a base organism against a database of protein sequences of various organisms, to find similar sequences. The generated summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and an RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, a “fragment” of a polynucleotide refers to a sequence comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule or protein as disclosed herein. Methods for producing such fragments from a starting promoter molecule are well known in the art. Fragments of a DNA molecule or protein may exhibit the activity of the DNA molecule or protein from which they are derived.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (proA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. Plant transformation may also be carried out in the absence of selection during one or more steps or stages of culturing, developing or regenerating transformed explants, tissues, plants and/or plant parts.

### E. Transformation Methods

Methods and compositions are provided for transforming a plant cell, tissue or explant with a recombinant DNA molecule or construct encoding one or more molecules required for targeted genome editing (e.g., guide RNA(s) and/or site-directed nuclease(s)). Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are bacterially mediated transformation, such as Agrobacterium-mediated or Rhizobium-mediated transformation, and microprojectile or particle bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616. Other methods for plant transformation, such as microinjection, electroporation, vacuum infiltration, pressure, sonication, silicon carbide fiber agitation, PEG-mediated transformation, etc., are also known in the art.

Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants, also referred to as R₀ plants. As used herein, “R₀ plant” refers to an initial regenerated transformant. As used herein, “R₁ seed” refers to seed produced from selfing R₀ plants. As used herein, “R₁ plant” refers to a plant grown from R₁ seed. As used herein, “R₂ seed” refers to seed produced from selfing R₁ plants. As used herein, “R₂ plant” refers to a plant grown from R₂ seed. Following one to two generations of self-crossing of R₀ plants, plants homozygous for edited alleles of the region may be produced. Furthermore, such modified plants may be crossed with a different WT male plant line to produce hybrid plants.

Any suitable method or technique for transformation of a plant cell known in the art may be used according to present methods. In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes.

As used herein, the terms “regeneration” and “regenerating” refer to a process of growing or developing a plant from one or more plant cells through one or more culturing steps. Transformed or edited cells, tissues or explants containing a DNA sequence insertion or edit may be grown, developed or regenerated into transgenic plants in culture, plugs, or soil according to methods known in the art. Certain embodiments of the disclosure therefore relate to methods and constructs for regenerating a plant from a cell with modified genomic DNA resulting from genome editing. The regenerated plant can then be used to propagate additional plants.

According to an aspect of the present disclosure, regenerated plants or a progeny plant, plant part or seed thereof can be screened or selected based on a marker, trait, or phenotype produced by the edit or mutation, or by the site-directed integration of an insertion sequence, transgene, etc., in the developed or regenerated plant, or a progeny plant, plant part or seed thereof. If a given mutation, edit, trait or phenotype is recessive, one or more generations or crosses (e.g., selfing) from the initial R₀ plant may be necessary to produce a plant homozygous for the edit or mutation so the trait or phenotype can be observed. Progeny plants, such as plants grown from R₁ seed or in subsequent generations, can be tested for zygosity using any known zygosity assay, such as by using a single nucleotide polymorphism (SNP) assay, DNA sequencing, thermal amplification, or polymerase chain reaction (PCR), and/or Southern blotting that allows for the distinction between heterozygote, homozygote and wild-type plants.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot; PCR (including amplicon sequencing which consists of the generation of one or more unique PCR products across the genomic region of interest for further sequencing analysis, e.g., using Next-Gen Sequencing techniques known in the art. Sequence data from each sample is then mapped to a reference sequence to identify consensus differences); enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot. In a further aspect, a method of detection provided herein comprises PCR. In a further aspect, a method of detection provided herein comprises amplicon sequencing. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acids can be isolated using techniques routine in the art. For example, nucleic acids can be isolated using any method including, without limitation, recombinant nucleic acid technology, and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide (including amplicon sequencing), Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme orribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis. Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

### F. Genetically Modified Plants

As used herein, “modified” in the context of a plant, seed, plant part, plant cell, and/or plant genome, refers to a plant, seed, plant part, plant cell, and/or plant genome comprising an engineered change in the expression level and/or endogenous sequence of one or more genes of interest relative to a wild-type or control plant, plant seed, plant part, plant cell, and/or plant genome. Indeed, the term “modified” may further refer to a plant, plant seed, plant part, plant cell, and/or plant genome having one or more deletions and/or one or more nucleotide substitutions or nucleotide insertions introduced through chemical mutagenesis, transposon insertion or excision, or any other known mutagenesis technique, or introduced through genome editing. In an aspect, a modified plant, plant seed, plant part, plant cell, and/or plant genome can comprise one or more transgenes. In particular embodiments, a modified plant, seed, plant part, plant cell, and/or plant genome may comprise a transgene encoding one or more polypeptides having a sequence with at least about 70% sequence identity to any one of SEQ ID NOs:1-107. For clarity, therefore, a modified plant, plant seed, plant part, plant cell, and/or plant genome includes but is not limited to a mutated, edited, and/or transgenic plant, plant seed, plant part, plant cell.

Modified plants, plant parts, seeds, etc., may have been subjected to mutagenesis, genome editing or site-directed integration, genetic transformation, or a combination thereof. Such “modified” plants, plant seeds, plant parts, and plant cells include plants, plant seeds, plant parts, and plant cells that are offspring or derived from “modified” plants, plant seeds, plant parts, and plant cells that retain a polynucleotide molecule encoding a polypeptide having at least about 70% sequence identity to any one of SEQ ID NOs:1-107. A modified seed provided herein may give rise to a modified plant provided herein. A modified plant, plant seed, plant part, plant cell, or plant genome provided herein may comprise a recombinant DNA construct or vector or genome edit as provided herein.

A “modified plant product” may be any product made from a modified plant, plant part, plant cell, or plant chromosome provided herein, or any portion or component thereof. For example, in some embodiments a modified plant product may be a commodity product produced from a modified plant or part thereof containing the recombinant DNA molecule as described herein, such as those encoding any one of SEQ ID NOs:1-107. In some embodiments, commodity products contain a detectable amount of DNA comprising a DNA sequence selected from the group consisting of SEQ ID NOs: 108-122 or fragments or variants thereof. As used herein, a “commodity product” refers to any composition or product which is comprised of material derived from a modified plant, seed, plant cell, or plant part containing the DNA molecule as described herein. Commodity products include but are not limited to processed seeds, grains, plant parts, and meal, protein concentrate, protein isolate, grain, starch, flour, biomass, or seed oil. A commodity product containing a detectable amount of DNA corresponding to the recombinant DNA molecule as described herein is contemplated. Detection of one or more of this DNA in a sample may be used for determining the content or the source of the commodity product. Any standard method of detection for DNA molecules may be used, including methods of detection disclosed herein.

Modified plants may be further crossed to themselves or other plants to produce modified plant seeds and progeny. A modified plant may also be prepared by crossing a first plant comprising a DNA sequence or construct or an edit (e.g., a genomic deletion) with a second plant lacking the DNA sequence or construct or edit. For example, a DNA sequence or inversion may be introduced into a first plant line that is amenable to transformation or editing, which may then be crossed with a second plant line to introgress the DNA sequence or edit (e.g., deletion) into the second plant line. Progeny of these crosses can be further backcrossed into the desirable line multiple times, such as through 6 to 8 generations or back crosses, to produce a progeny plant with substantially the same genotype as the original parental line, but for the introduction of the DNA sequence or edit. A modified plant, plant cell, or seed provided herein may be a hybrid plant, plant cell, or seed. As used herein, a “hybrid” is created by crossing two plants from different varieties, lines, inbreds, or species, such that the progeny comprises genetic material from each parent. Skilled artisans recognize that higher order hybrids can be generated as well.

A modified plant, plant part, plant cell, or seed provided herein may be of an elite variety or an elite line. An “elite variety” or an “elite line” refers to a variety that has resulted from breeding and selection for superior agronomic performance.

As used herein, the term “control plant” (or likewise a “control” plant seed, plant part, plant cell, and/or plant genome) refers to a plant (or plant seed, plant part, plant cell, and/or plant genome) that is used for comparison to a modified plant (or modified plant seed, plant part, plant cell, and/or plant genome) and has the same or similar genetic background (e.g., same parental lines, hybrid cross, inbred line, testers, etc.) as the modified plant (or plant seed, plant part, plant cell, and/or plant genome), except for genome edit(s) and/or transgenes comprising a nucleotide sequence encoding a sequence having at least about 70% sequence identity to any one of SEQ ID NOs:1-107 or a fragment thereof. For example, a control plant may be an inbred line that is the same as the inbred line used to make the modified plant, or a control plant may be the product of the same hybrid cross of inbred parental lines as the modified plant, except for the absence in the control plant of any transgenic events or genome edit(s), and/or any transgenes comprising a nucleotide sequence encoding a sequence having at least about 70% sequence identity to any one of SEQ ID NOs:1-107 or a fragment thereof. Similarly, an “unmodified control plant” refers to a plant that shares a substantially similar or essentially identical genetic background as a modified plant, but without the one or more engineered changes to the genome (e.g., mutation or edit) of the modified plant. For purposes of comparison to a modified plant, plant seed, plant part, plant cell, and/or plant genome, a “wild-type plant” (or likewise a “wild-type” plant seed, plant part, plant cell, and/or plant genome) refers to a non-transgenic and non-genome edited control plant, plant seed, plant part, plant cell, and/or plant genome. As used herein, a “control” plant, plant seed, plant part, plant cell, and/or plant genome may also be a plant, plant seed, plant part, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

As used herein, the term “activity” refers to the biological function of a gene or protein. A gene or a protein may provide one or more distinct functions. A reduction, disruption, or alteration in “activity” thus refers to a lowering, reduction, or elimination of one or more functions of a gene or a protein in a plant, plant cell, or plant tissue at one or more stage(s) of plant development, as compared to the activity of the gene or protein in a wild-type or control plant, cell, or tissue at the same stage(s) of plant development. Additionally, an increase in “activity” thus refers to an elevation of one or more functions of a gene or a protein in a plant, plant cell, or plant tissue at one or more stage(s) of plant development, as compared to the activity of the gene or protein in a wild-type or control plant, cell, or tissue at the same stage(s) of plant development.

According to some embodiments, a modified plant is provided having a polypeptide level of a polypeptide comprising any one of SEQ ID NOs:1-107 that is increased in at least one plant tissue by at least 1%, at least 5%, at least 10%, at least 20%, at least 25%, at least 30%, at least 40%, at least 50%, at least 60%, at least 70%, at least 75%, at least 80%, at least 90%, or 100%, as compared to a control plant, including all ranges and values derivable therebetween. According to some embodiments, a modified plant is provided having a polypeptide level of a polypeptide comprising any one of SEQ ID NOs:1-107 that is increased in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant, including all ranges and values derivable therebetween.

The present disclosure relates to a plant with improved economically important characteristics, including but not limited to, production of fungal-derived medicinal compounds.

As used herein, a “plant” includes a whole plant, explant, plant part, seedling, or plantlet at any stage of regeneration or development.

As used herein, a “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

An “embryo” is a part of a plant seed, consisting of precursor tissues (e.g., meristematic tissue) that can develop into all or part of an adult plant. An “embryo” may further include a portion of a plant embryo.

A “meristem” or “meristematic tissue” comprises undifferentiated cells or meristematic cells, which are able to differentiate to produce one or more types of plant parts, tissues or structures, such as all or part of a shoot, stem, root, leaf, seed, etc.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description. Any embodiment of the present disclosure may be used in combination with any other embodiment described herein.

All references herein are incorporated herein by reference in their entirety.

### Examples

The following examples are included to illustrate embodiments of the present disclosure. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Expressing Megasynthases from Microorganisms in Plants

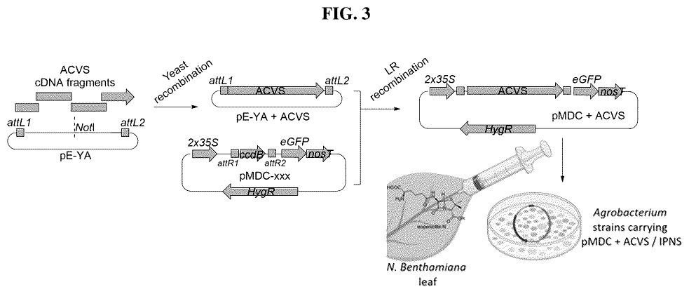

cDNAs encoding ACVS, MpaC′ and accessory enzymes (FIG. 1 and FIG. 2) were codon optimized for expression in tobacco (Nicotiana benthamiana) and synthesized. Due to the size of the megasynthases the cDNAs are synthesized in 3 to 4 overlapping fragments and then assembled into pE-YA using yeast recombination. These cDNAs are introduced into the gateway compatible vector series pMDC for plant expression by LR recombination (FIG. 3). These vectors support high level expression from the CaMV35S promoter and can be cloned in-frame with a GFP (or other fluorescent protein) tag at the N- or C-terminus. The fluorescent tag allows for confirmation of expression and cytosolic localization by confocal laser scanning fluorescence microscopy. Vectors are transformed into Agrobacterium tumefaciencs for delivery of transgenes into N. benthamiana leaf cells. Co-infiltration of leaves with mixtures of vectors (each in a separate Agro culture) facilitates combination of multiple pathway components to test for synthesis of fungal metabolites (i.e., co-expression of ACVS, IPNS, and a fungal phosphopantetheinyl transferase (PPTase) for isopenicillin N production; and co-expression of MpaC′ and a PPTase for 5-methylorsellinic acid synthesis). RT-PCR is used to confirm expression of the full-length cDNA and Western blotting of leaf protein extracts using antibodies to the fluorescent tag offers independent confirmation of formation of a fusion protein of the predicted size. Untagged versions of the transgene may also be generated. Comparison of the distribution for the chimeric GFP fusion proteins and co-expressed free mCherry confirms a cytoplasmic location for each of the proteins separately. Absence of GFP from the nucleus for the fusion proteins indicates an intact chimeric protein, since free GFP (or the free mCherry), but not the fusion protein, is small enough to diffuse into the nucleus.

Alternatively, megasynthases such as NRPS and Type I PKS enzymes may be synthesized using an enzyme deconstruction approach that individually expresses subsets of domains. Such enzyme deconstruction approaches are routinely used to study these large enzymes in heterologous hosts such as E. coli and yeast (Crawford et al. Science. 320(5873): 243-246, 2008; Newman et al. J. Am. Chem. Soc. 136(20):7348-7362, 2014)

### Example 2: Chemical Analysis to Confirm Production and Export of Fungal Megasynthase Products in N. benthamiana

Co-expression of ACVS, IPNS, and a fungal phosphopantethienyltransferase (PPTase; e.g., NpgA) leads to isopenicillin N synthesis in N. benthamiana, whereas co-expression of MpaC′ and NpgA leads to the synthesis of 5-methylorsellinic acid. The production of these metabolites is confirmed by liquid chromatography-mass spectrometry (LCMS) analysis, using established protocols. Briefly, N. benthamiana leaves are collected after Agrobacterium infiltration and lyophilized. The sample is homogenized, suspended in PBS buffer, and extracted using hexane, according to literature methods (Aldeek et al. J. Agric. Food Chem. 63 (26): 5993-6000, 2015). The samples are compared to a commercially available standard of isopenicillin N or 5-methylorsellinic acid, respectively, for quantification via regression analysis. Since beta-lactam stability is dependent on the presence of acids, bases, nucleophiles and oxidizing agents, and beta-lactamase activity has been reported in plants, monitoring for hydrolyzed products, such as 6-aminopenicillanic acid, is also performed. Isopenicillin N can also be chemically hydrolysed to establish its LCMS profile to generate known standards, if necessary. In some embodiments, enriched growth media may be utilized to provide further substrates, nutrients, and/or precursors for synthesis of fungal metabolites.

### Example 3: Analysis of Molecular Consequences Using Metabolomics and Transcriptomics

Studies are performed to determine the effects on metabolic flux, perturbations of other metabolic pathways, and whether the presence of foreign enzymes leads to toxic effects in fundamental plant processes or induces internal defenses. The N. benthamiana metabolomic and transcriptomic data are compared in parallel before, during, and after transient expression of the fungal megasynthases compared to mock-inoculated N. benthamiana controls in triplicate experiments (three leaves combined from each of three separate plants represents an individual replicate). For untargeted metabolomics, intracellular compounds are extracted from leaves using cold methanol: chloroform: water (1:2.5:1, v/v/v). Metabolites from the polar phase are separated through hydrophilic interaction chromatography (HILIC) whereas semi-polar and apolar compounds are resolved through reverse phase chromatography. An AB Sciex 6600+TripleTOF high resolution mass spectrometer (HRMS) is used to detect the metabolites in positive and negative ionization modes and acquire their exact mass as well as their spectrum. Data collected for each peak/metabolite is processed using the publicly available MSDIAL platform in order to identify the metabolites. For targeted metabolomics, water-soluble compounds such as sugars, sugar alcohols, organic acids, amino acids, and phosphorylated metabolites are extracted using boiling water and quantified using an Agilent 1290 Infinity II HPLC coupled to an AB Sciex QTRAP 6500+MS. In parallel, known quantities of commercially available external standards are injected to achieve an absolute quantification of the metabolites present in the samples. Multiple internal standards are added at the time of extraction and are used to normalize the data before statistical analyses. Statistical analyses are performed through the online platform MetaboAnalyst 5.0 to pinpoint metabolic perturbations. For transcriptomic analysis, RNA is isolated from leaves using the Trizol reagent and further purified by LiCl precipitation. RNA-Seq libraries are generated using the TruSeq library protocol. The RNA-Seq libraries are quantified by capillary electrophoresis and sequencing on an Illumina NextSeq 500. After initial quality control processes, the high-quality sequencing reads from the leaf samples are aligned to the reference N. benthamiana genome using a splice-aware aligner such as Bowtie2 version 2.1.0 and TopHat2 version 2.0.10. Differentially expressed genes between the treatments are identified using DESeq within R version 3.1.2 in Bioconductor version 3.0.

### Example 4: Cloning and Heterologous Expression of ACVS, IPNS, PenDE, PenM, and PaaT with Subcellular Targeting Sequences

Using the methodology described in Example 1, the final biosynthetic gene penDE, encoding isopenicillin N acyltransferase (IAT), and the two transporters PenM and PaaT are cloned using synthetic cDNA sequences. These genes also include either a native peroxisome targeting signal (PTS) or an engineered PTS (e.g., PEX26 mPTS or PEX22 mPTS) to ensure that IAT, PenM, and PaaT are optimally localized in the peroxisome. Cloning of PenV into plants is not necessary because plants catabolize lysine to α-aminoadipic acid and excess supply can be generated through existing lysine biosynthesis pathways. If the native peroxisomal ABC transporter of the plant of interest is capable of transporting phenylacetic acid, PaaT does not need to be cloned into the plant. The peroxisomal ABC plant transporters utilize a range of acyl-CoAs, including the hormone precursors indole butyric acyl CoA and 12-oxo-phytodienoyl CoA. Following plasmid construction using the pMDC series without GFP, and confirmation by sequencing, plasmids are co-transformed via mixed Agrobacterium cultures, each containing a separate coding sequence. Expression is monitored via RT-PCR. Using the extraction and detection conditions described in Example 2 LCMS is used to monitor for penicillin G and its potential hydrolysis products.

### Example 5: Cloning and Heterologous Expression of MpaC′, MpaDE′, MpaA′, MpaB′, MpaG′, PbACL891, and MpaH′ with Subcellular Targeting Sequences

The biosynthesis of mycophenolic acid has different considerations compared to the biosynthesis of penicillin. The components of the mycophenolic acid biosynthesis pathway are located in several additional subcellular compartments, including the peroxisomes, endoplasmic reticulum (ER), and Golgi apparatus. Therefore, in addition to using PTS signals to locate PbACL891 and MpaH′ to the peroxisomes, endoplasmic reticulum signal peptides for MpaDE‘and MpaB’, and a Golgi signal peptide sequence for MpaA′ may also be used. The inherent signal/targeting sequences within the fungal proteins, however, are likely to direct the proteins to the desired compartment, as there is cross-kingdom recognition of such localization sequences. This may be confirmed by colocalization with organelle markers: CFP-HDEL (ER), sialyltransferase (ST-GFP/Golgi), and mCherry-SKL (peroxisomes). If localization of fungal proteins is not achieved based on co-localization with markers, then known sequences appended to the appropriate position on the proteins (N-terminus for co-translation into the ER; C-terminus for ER-retention; an internal transmembrane segment for Golgi; or C-terminus for peroxisomes) may be utilized. The plasmids, constructed using codon optimized cDNAs, are co-transformed and analyzed as described in Example 4. For chemical analysis LC-HRMS is utilized to monitor for mycophenolic acid, 5-methylorsellinic acid, and intermediates identified in FIG. 2.

### Example 6: Confocal Microscopy to Determine Expression and Localization

Expression and subcellular location of the tailoring enzymes (i.e., IAT, PenM, and PaaT for the penicillin pathway; MpaC′, MpaDE′, MpaA′, MpaB′, MpaG′, PbACL891, and MpaH′ for the mycophenolic acid pathway) is analyzed by including eGFP in the expression plasmids at either the N- or C-terminus. Utilizing mRuby3-PTS1, a reporter that targets the peroxisome and marks this organelle, it can be individually investigated whether IAT, PenM, PaaT, and MpaH′ localize to peroxisomes, using confocal fluorescence microscopy. Similarly, correct localization of the Mpa enzymes can be determined using mCherry-HDEL/ST reporters and fluorescence microscopy.

### Example 7: Plant-based Manufacture of Fungal Medicines Utilizing a Full Greenhouse

Following generation of desired metabolites, production is scaled-up to evaluate larger scale production requirements and costs. To investigate the efficiency of fungal medicine production in plants multiple plants are utilized in two different environments: 1) a high-end climate-controlled greenhouse setting; and 2) a standard indoor room with lighted shelving. Batch-wise infiltration of multiple plants, rather than single leaves, using mixtures of Agrobacterium containing different combinations of biosynthetic genes may be performed as described in (Reed et al. Metabolic Engineering. 42:185-193, 2017). After five days incubation, leaves are harvested, flash-frozen, lyophilized, weighed, and prepared for chemical analysis. Specifically, the time to maximum penicillin/mycophenolic acid production per plant, costs per plant (i.e., power inputs, cultivation materials), yield, and scalability (i.e., production per plant; % of penicillin/mycophenolic acid per dry weight of leaf) are assessed. Data obtained allow for initial estimates of greenhouse-based fungal-derived medicine production.

### Example 8: Analysis of Efficiency of Plant and Fungal Transporters for Metabolite Translocation

Fungal transporters may function properly in planta since plant and yeast transporters often are interchangeable. The use of the endogenous peroxisomal ABC transporters alone, like PXA1, that are known be promiscuous in their ability to transport a variety of structurally diverse substrates into peroxisomes may be utilized to ensure that substrates and intermediates can permeate the plant peroxisomes membranes. The amount of synthesized metabolites using these endogenous transporters is compared to the amount accumulated with expression of fungal transporters to determine whether there is enhanced accumulation. The influence of upregulating factors that support transport by PXA1 is also analyzed. The comparative gene identifier 58 (CGI58) protein, for example, has been shown to interact with and support the efficient transport of substrates by PXA1 (James et al. Proc. Nat. Acad. Sci., 107(41):17833-17838, 2010), including auxin and jasmonic acid precursors. Upregulation of this factor in N. benthamiana, along with added expression of various transporters, may increase the capacity for metabolite production.

### Example 9: Factors Important for the Economic Feasibility of Replacing Fermentation Processes with Plant-Based Medicines

A detailed cost comparison of the production processes of the current deep vat fermentation process and the production processes for plant-grown medicines is conducted. A cost-benefit analysis for plant-grown penicillin using a sensitivity analysis to determine production feasibility and market opportunities is conducted. A pro forma analysis is made to estimate the cost-saving projections of the new industry for 10/20/30 years out. Special attention is given to the potential savings generated as production yields and scales change. It is also determined if there are potential savings related to the costs of waste disposal, factory maintenance, and energy requirements in greenhouse production vs. future open field, crop-produced medicines. Potential value creation through soil stabilization, water filtration, carbon offset markets, and cap and trade permit schemes is also considered.

### Example 10: Agrobacterium-Mediated Heterologous Transient Expression and Subcellular Location in Leaves of Nicotiana benthamiana

Fungal DNA coding sequences fused with green fluorescence protein were expressed and produced in leaves of Nicotiana benthamiana (wild tobacco relative) and their correct subcellular locations were targeted. Fusion proteins were for the mycophenolic acid (Table 1) and penicillin biosynthesis pathways (Table 2). DNA coding sequences were coned into plant expression vectors and introduced by infiltration and Agrobacterium tumefaciens-mediated transformation for transient expression, protein production, and subcellular localization using standard molecular cell biology techniques. Subcellular localization was confirmed using confocal laser scanning microscopy.

TABLE 1
Transient Expression and Subcellular Location of Mycophenolic
Acid Pathway Enzymes in Leaves of Nicotiana benthamiana.

| | | CDS | | Transient |
|---|---|---|---|---|
| | | length | | expression in |
| Enzyme | Origin species | (kb) | Cloning | N. benthamiana |
| MpaC | Penicillium | 7.347 | Cloned in | Low expression |
| | brevicompactum | | pMDC84 | Localized in |
| | | | | cytoplasm |
| MpaB | P. brevicompactum | 1.269 | Cloned in | Good expression |
| | | | pMDC84 | ER localization |
| MpaA | P. brevicompactum | 0.996 | Cloned in | Good expression |
| | | | pDGB3 | Golgi localization |
| MpaDE | P. brevicompactum | 2.559 | Cloned in | Good expression |
| | | | pMDC84 | ER localization |
| MpaG | P. brevicompactum | 1.197 | Cloned in | Low expression |
| | | | pDGB3 | Localized in |
| | | | | cytoplasm |
| PbACL | P. brevicompactum | 2.124 | Cloned in | Good Expression |
| | | | pDGB3 | Localized in |
| | | | | peroxisomes |

TABLE 2
Transient Expression and Subcellular Location of Penicillin
Pathway Enzymes in Leaves of Nicotiana benthamiana.

| | | CDS | | Transient |
|---|---|---|---|---|
| | | length | Cloning | expression in |
| Enzyme | Origin species | (kb) | vector | N. benthamiana |
| ACVS | Penicillium | 11.331 | Cloned in | Low expression |
| | chrysogenum | | pMDC84 | Localized in |
| | | | | cytoplasm |
| NpgA | Aspergillus | 1.035 | Cloned in | Good Expression |
| | nidulans | | pMDC83 | Localized in |
| | | | | cytoplasm |
| IPNS | P. chrysogenum | 0.996 | Cloned in | Good Expression |
| | | | pMDC84 | Localized in |
| | | | | cytoplasm |
| IAT | P. chrysogenum | 1.077 | Cloned in | Good Expression |
| | | | pMDC84 | Localized in |
| | | | | peroxisome |
| PaaT | P. chrysogenum | 1.647 | Cloned in | Good Expression |
| | | | pMDC84 | Localized in |
| | | | | peroxisome |

All of the methods disclosed and claimed herein can be made and executed without undue experimentation in light of the present disclosure. While the compositions and methods of this invention have been described in terms of preferred embodiments or aspects, it will be apparent to those of skill in the art that variations may be applied to the methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A modified plant, seed, cell, or plant part comprising at least one recombinant polynucleotide molecule comprising at least one nucleotide sequence encoding at least one polypeptide having an amino acid sequence with at least about 85% sequence identity to an amino acid sequence selected from the group consisting of SEQ ID NOs:1-107, wherein the modified plant, seed, cell, or plant part is capable of producing a medicinal compound selected from the group consisting of penicillin, mycophenolic acid, cyclosporin, cephalosporin, pneumocandin B0, lovastatin, compactin, griseofulvin, pleuromutilin, aphidicolin, enfumafungin, fusidic acid, psilocybin B.

2. The modified plant, seed, cell, or plant part of claim 1, wherein the plant, seed, cell, or plant part comprises:
a) a first nucleotide sequence encoding a polypeptide having an amino acid sequence with at least about 85% sequence identity to SEQ ID NO:1, a second nucleotide sequence encoding a polypeptide having an amino acid sequence with at least about 85% sequence identity to SEQ ID NO:2, a third nucleotide sequence encoding a polypeptide having an amino acid sequence with at least about 85% sequence identity to SEQ ID NO:3, a fourth nucleotide sequence encoding a polypeptide having an amino acid sequence with at least about 85% sequence identity to SEQ ID NO:4, a fifth nucleotide sequence encoding a polypeptide having an amino acid sequence with at least about 85% sequence identity to SEQ ID NO:5, and a sixth nucleotide sequence encoding a polypeptide having an amino acid sequence with at least about 85% sequence identity to SEQ ID NO:6, wherein the modified plant, seed, cell, or plant part is capable of producing penicillin;
b) a first nucleotide sequence encoding a polypeptide having an amino acid sequence with at least about 85% sequence identity to SEQ ID NO:7, a second nucleotide sequence encoding a polypeptide having an amino acid sequence with at least about 85% sequence identity to SEQ ID NO:8, a third nucleotide sequence encoding a polypeptide having an amino acid sequence with at least about 85% sequence identity to SEQ ID NO:9, a fourth nucleotide sequence encoding a polypeptide having an amino acid sequence with at least about 85% sequence identity to SEQ ID NO:10, a fifth nucleotide sequence encoding a polypeptide having an amino acid sequence with at least about 85% sequence identity to SEQ ID NO:11, a sixth nucleotide sequence encoding a polypeptide having an amino acid sequence with at least about 85% sequence identity to SEQ ID NO:12, a seventh nucleotide sequence encoding a polypeptide having an amino acid sequence with at least about 85% sequence identity to SEQ ID NO:13, an eighth nucleotide sequence encoding a polypeptide having an amino acid sequence with at least about 85% sequence identity to SEQ ID NO:14 (MpaH), and a ninth nucleotide sequence encoding a polypeptide having an amino acid sequence with at least about 85% sequence identity to SEQ ID NO:15, wherein the modified plant, seed, cell, or plant part is capable of producing mycophenolic acid;
c) a first nucleotide sequence encoding a polypeptide having an amino acid sequence with at least about 85% sequence identity to SEQ ID NO:16, a second nucleotide sequence encoding a polypeptide having an amino acid sequence with at least about 85% sequence identity to SEQ ID NO:17, a third nucleotide sequence encoding a polypeptide having an amino acid sequence with at least about 85% sequence identity to SEQ ID NO:18, a fourth nucleotide sequence encoding a polypeptide having an amino acid sequence with at least about 85% sequence identity to SEQ ID NO:19, a fifth nucleotide sequence encoding a polypeptide having an amino acid sequence with at least about 85% sequence identity to SEQ ID NO:20, a sixth nucleotide sequence encoding a polypeptide having an amino acid sequence with at least about 85% sequence identity to SEQ ID NO:21, a seventh nucleotide sequence encoding a polypeptide having an amino acid sequence with at least about 85% sequence identity to SEQ ID NO:22, an eighth nucleotide sequence encoding a polypeptide having an amino acid sequence with at least about 85% sequence identity to SEQ ID NO:23, a ninth nucleotide sequence encoding a polypeptide having an amino acid sequence with at least about 85% sequence identity to SEQ ID NO:24, a tenth nucleotide sequence encoding a polypeptide having an amino acid sequence with at least about 85% sequence identity to SEQ ID NO:25, an eleventh nucleotide sequence encoding a polypeptide having an amino acid sequence with at least about 85% sequence identity to SEQ ID NO:26, and a twelfth nucleotide sequence encoding a polypeptide having an amino acid sequence with at least about 85% sequence identity to SEQ ID NO:27, wherein the modified plant, seed, cell, or plant part is capable of producing cyclosporin;
d) a first nucleotide sequence encoding a polypeptide having an amino acid sequence with at least about 85% sequence identity to SEQ ID NO:28, a second nucleotide sequence encoding a polypeptide having an amino acid sequence with at least about 85% sequence identity to SEQ ID NO:29, a third nucleotide sequence encoding a polypeptide having an amino acid sequence with at least about 85% sequence identity to SEQ ID NO:30, a fourth nucleotide sequence encoding a polypeptide having an amino acid sequence with at least about 85% sequence identity to SEQ ID NO:31, a fifth nucleotide sequence encoding a polypeptide having an amino acid sequence with at least about 85% sequence identity to SEQ ID NO:32, a sixth nucleotide sequence encoding a polypeptide having an amino acid sequence with at least about 85% sequence identity to SEQ ID NO:33, a seventh nucleotide sequence encoding a polypeptide having an amino acid sequence with at least about 85% sequence identity to SEQ ID NO:34, an eighth nucleotide sequence encoding a polypeptide having an amino acid sequence with at least about 85% sequence identity to SEQ ID NO:35, and a ninth nucleotide sequence encoding a polypeptide having an amino acid sequence with at least about 85% sequence identity to SEQ ID NO:36, wherein the modified plant, seed, cell, or plant part is capable of producing cephalosporin;
e) a first nucleotide sequence nucleotide sequence encoding a polypeptide having an amino acid sequence with at least about 85% sequence identity to SEQ ID NO:37, a second nucleotide sequence encoding a polypeptide having an amino acid sequence with at least about 85% sequence identity to SEQ ID NO:38, a third nucleotide sequence encoding a polypeptide having an amino acid sequence with at least about 85% sequence identity to SEQ ID NO:39, a fourth nucleotide sequence encoding a polypeptide having an amino acid sequence with at least about 85% sequence identity to SEQ ID NO:40, a fifth nucleotide sequence encoding a polypeptide having an amino acid sequence with at least about 85% sequence identity to SEQ ID NO:41, a sixth nucleotide sequence encoding a polypeptide having an amino acid sequence with at least about 85% sequence identity to SEQ ID NO:42, a seventh nucleotide sequence encoding a polypeptide having an amino acid sequence with at least about 85% sequence identity to SEQ ID NO:43, an eighth nucleotide sequence encoding a polypeptide having an amino acid sequence with at least about 85% sequence identity to SEQ ID NO:44, a ninth nucleotide sequence encoding a polypeptide having an amino acid sequence with at least about 85% sequence identity to SEQ ID NO:45, a tenth nucleotide sequence encoding a polypeptide having an amino acid sequence with at least about 85% sequence identity to SEQ ID NO:46, an eleventh nucleotide sequence encoding a polypeptide having an amino acid sequence with at least about 85% sequence identity to SEQ ID NO:47, and a twelfth nucleotide sequence encoding a polypeptide having an amino acid sequence with at least about 85% sequence identity to SEQ ID NO:48, wherein the modified plant, seed, cell, or plant part is capable of producing pneumocandin B0;
f) a first nucleotide sequence encoding a polypeptide having an amino acid sequence with at least about 85% sequence identity to SEQ ID NO:49, a second nucleotide sequence encoding a polypeptide having an amino acid sequence with at least about 85% sequence identity to SEQ ID NO:50, a third nucleotide sequence encoding a polypeptide having an amino acid sequence with at least about 85% sequence identity to SEQ ID NO:51, a fourth nucleotide sequence encoding a polypeptide having an amino acid sequence with at least about 85% sequence identity to SEQ ID NO:52, a fifth nucleotide sequence encoding a polypeptide having an amino acid sequence with at least about 85% sequence identity to SEQ ID NO:53, and a sixth nucleotide sequence encoding a polypeptide having an amino acid sequence with at least about 85% sequence identity to SEQ ID NO:54, wherein the modified plant, seed, cell, or plant part is capable of producing lovastatin;
g) a first nucleotide sequence encoding a polypeptide having an amino acid sequence with at least about 85% sequence identity to SEQ ID NO:55, a second nucleotide sequence encoding a polypeptide having an amino acid sequence with at least about 85% sequence identity to SEQ ID NO:56, a third nucleotide sequence encoding a polypeptide having an amino acid sequence with at least about 85% sequence identity to SEQ ID NO:57, a fourth nucleotide sequence encoding a polypeptide having an amino acid sequence with at least about 85% sequence identity to SEQ ID NO:58, a fifth nucleotide sequence encoding a polypeptide having an amino acid sequence with at least about 85% sequence identity to SEQ ID NO:59, a sixth nucleotide sequence encoding a polypeptide having an amino acid sequence with at least about 85% sequence identity to SEQ ID NO:60, a seventh nucleotide sequence encoding a polypeptide having an amino acid sequence with at least about 85% sequence identity to SEQ ID NO:61, an eighth nucleotide sequence encoding a polypeptide having an amino acid sequence with at least about 85% sequence identity to SEQ ID NO:62, and a ninth nucleotide sequence encoding a polypeptide having an amino acid sequence with at least about 85% sequence identity to SEQ ID NO:63, wherein the modified plant, seed, cell, or plant part is capable of producing compactin;
h) a first nucleotide sequence encoding a polypeptide having an amino acid sequence with at least about 85% sequence identity to SEQ ID NO:64, a second nucleotide sequence encoding a polypeptide having an amino acid sequence with at least about 85% sequence identity to SEQ ID NO:65, a third nucleotide sequence encoding a polypeptide having an amino acid sequence with at least about 85% sequence identity to SEQ ID NO:66, a fourth nucleotide sequence encoding a polypeptide having an amino acid sequence with at least about 85% sequence identity to SEQ ID NO:67, a fifth nucleotide sequence encoding a polypeptide having an amino acid sequence with at least about 85% sequence identity to SEQ ID NO:68, a sixth nucleotide sequence encoding a polypeptide having an amino acid sequence with at least about 85% sequence identity to SEQ ID NO:69, a seventh nucleotide sequence encoding a polypeptide having an amino acid sequence with at least about 85% sequence identity to SEQ ID NO:70), and an eighth nucleotide sequence encoding a polypeptide having an amino acid sequence with at least about 85% sequence identity to SEQ ID NO:71, wherein the modified plant, seed, cell, or plant part is capable of producing griseofulvin;
i) a first nucleotide sequence encoding a polypeptide having an amino acid sequence with at least about 85% sequence identity to SEQ ID NO:72, a second nucleotide sequence encoding a polypeptide having an amino acid sequence with at least about 85% sequence identity to SEQ ID NO:73, a third nucleotide sequence encoding a polypeptide having an amino acid sequence with at least about 85% sequence identity to SEQ ID NO:74, a fourth nucleotide sequence encoding a polypeptide having an amino acid sequence with at least about 85% sequence identity to SEQ ID NO:75, a fifth nucleotide sequence encoding a polypeptide having an amino acid sequence with at least about 85% sequence identity to SEQ ID NO:76, and a sixth nucleotide sequence encoding a polypeptide having an amino acid sequence with at least about 85% sequence identity to SEQ ID NO:77, wherein the modified plant, seed, cell, or plant part is capable of producing pleuromutilin;
j) a first nucleotide sequence encoding a polypeptide having an amino acid sequence with at least about 85% sequence identity to SEQ ID NO:78, a second nucleotide sequence encoding a polypeptide having an amino acid sequence with at least about 85% sequence identity to SEQ ID NO:79, a third nucleotide sequence encoding a polypeptide having an amino acid sequence with at least about 85% sequence identity to SEQ ID NO:80, and a fourth nucleotide sequence encoding a polypeptide having an amino acid sequence with at least about 85% sequence identity to SEQ ID NO:81, wherein the modified plant, seed, cell, or plant part is capable of producing pleuromutilin;
k) a first nucleotide sequence encoding a polypeptide having an amino acid sequence with at least about 85% sequence identity to SEQ ID NO:82, a second nucleotide sequence encoding a polypeptide having an amino acid sequence with at least about 85% sequence identity to SEQ ID NO:83, a third nucleotide sequence encoding a polypeptide having an amino acid sequence with at least about 85% sequence identity to SEQ ID NO:84, a fourth nucleotide sequence encoding a polypeptide having an amino acid sequence with at least about 85% sequence identity to SEQ ID NO:85, a fifth nucleotide sequence encoding a polypeptide having an amino acid sequence with at least about 85% sequence identity to SEQ ID NO:86, a sixth nucleotide sequence encoding a polypeptide having an amino acid sequence with at least about 85% sequence identity to SEQ ID NO:87, a seventh nucleotide sequence encoding a polypeptide having an amino acid sequence with at least about 85% sequence identity to SEQ ID NO:88, an eighth nucleotide sequence encoding a polypeptide having an amino acid sequence with at least about 85% sequence identity to SEQ ID NO:89, a ninth nucleotide sequence encoding a polypeptide having an amino acid sequence with at least about 85% sequence identity to SEQ ID NO:90, and a tenth nucleotide sequence encoding a polypeptide having an amino acid sequence with at least about 85% sequence identity to SEQ ID NO:91, wherein the modified plant, seed, cell, or plant part is capable of producing enfumafungin;
l) a first nucleotide sequence encoding a polypeptide having an amino acid sequence with at least about 85% sequence identity to SEQ ID NO:92, a second nucleotide sequence encoding a polypeptide having an amino acid sequence with at least about 85% sequence identity to SEQ ID NO:93, a third nucleotide sequence encoding a polypeptide having an amino acid sequence with at least about 85% sequence identity to SEQ ID NO:94, a fourth nucleotide sequence encoding a polypeptide having an amino acid sequence with at least about 85% sequence identity to SEQ ID NO:95, a fifth nucleotide sequence encoding a polypeptide having an amino acid sequence with at least about 85% sequence identity to SEQ ID NO:96, a sixth nucleotide sequence encoding a polypeptide having an amino acid sequence with at least about 85% sequence identity to SEQ ID NO:97, a seventh nucleotide sequence encoding a polypeptide having an amino acid sequence with at least about 85% sequence identity to SEQ ID NO:98, and an eighth nucleotide sequence encoding a polypeptide having an amino acid sequence with at least about 85% sequence identity to SEQ ID NO:99, wherein the modified plant, seed, cell, or plant part is capable of producing fusidic acid; or
m) a first nucleotide sequence encoding a polypeptide having an amino acid sequence with at least about 85% sequence identity to SEQ ID NO:100 or SEQ ID NO:101, a second nucleotide sequence encoding a polypeptide having an amino acid sequence with at least about 85% sequence identity to SEQ ID NO:102 or SEQ ID NO:103, a third nucleotide sequence encoding a polypeptide having an amino acid sequence with at least about 85% sequence identity to SEQ ID NO:104 or SEQ ID NO:105, and a fourth nucleotide sequence encoding a polypeptide having an amino acid sequence with at least about 85% sequence identity to SEQ ID NO:106 or SEQ ID NO:107, wherein the modified plant, seed, cell, or plant part is capable of producing psilocybin.

3. The modified plant, seed, cell, or plant part of claim 1, wherein the at least one recombinant polynucleotide molecule comprises at least one nucleotide sequence encoding at least one polypeptide having an amino acid sequence with at least about 90%, about 91%, about 92%, about 93%, about 94%, about 95%, about 96%, about 97%, about 98%, about 99%, or 100% sequence identity to an amino acid sequence selected from the group consisting of SEQ ID NOs:1-107.

4. The modified plant, seed, cell, or plant part of claim 1, wherein the modified plant, seed, cell, or plant part comprises at least one polypeptide having an amino acid sequence with at least about 90%, about 91%, about 92%, about 93%, about 94%, about 95%, about 96%, about 97%, about 98%, about 99%, or 100% sequence identity to an amino acid sequence selected from the group consisting of SEQ ID NOs:1-107.

5. The modified plant, seed, cell, or plant part of claim 4, wherein the modified plant, seed, cell, or plant part comprises at least two, at least three, at least four, at least five, at least six, at least seven, at least eight, at least nine, at least ten, at least eleven, or at least twelve polypeptides having an amino acid sequence with at least about 90%, about 91%, about 92%, about 93%, about 94%, about 95%, about 96%, about 97%, about 98%, about 99%, or 100% sequence identity to an amino acid sequence selected from the group consisting of SEQ ID NOs:1-107.

6. The modified plant, seed, cell, or plant part of claim 1, wherein the modified plant, seed, cell, or plant part comprises at least two, at least three, at least four, at least five, at least six, at least seven, at least eight, at least nine, at least ten, at least eleven, or at least twelve recombinant polynucleotide molecules.

7. The modified plant, seed, cell, or plant part of claim 1, wherein the modified plant, seed, cell, or plant part comprises:
a) at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:1-6, wherein the modified plant, seed, cell, or plant part is capable of producing penicillin;
b) at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:7-15, wherein the modified plant, seed, cell, or plant part is capable of producing mycophenolic acid;
c) at least two, at least three, at least four, at least five, at least six, at least seven, at least eight, at least nine, at least ten, at least eleven, or at least twelve nucleotide sequences encoding at least two, at least three, at least four, at least five, at least six, at least seven, at least eight, at least nine, at least ten, at least eleven, or at least twelve polypeptides having an amino acid sequence with at least about 85% sequence identity to an amino acid sequence selected from the group consisting of SEQ ID NOs:16-27, wherein the modified plant, seed, cell, or plant part is capable of producing cyclosporin;
d) at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:28-36, wherein the modified plant, seed, cell, or plant part is capable of producing cephalosporin;
e) at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:37-48, wherein the modified plant, seed, cell, or plant part is capable of producing pneumocandin B0;
f) at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:49-54, wherein the modified plant, seed, cell, or plant part is capable of producing lovastatin;
g) at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:55-63, wherein the modified plant, seed, cell, or plant part is capable of producing compactin;
h) at least two, at least three, at least four, at least five, at least six, at least seven, or at least eight nucleotide sequences encoding at least two, at least three, at least four, at least five, at least six, at least seven, or at least eight polypeptides having an amino acid sequence with at least about 85% sequence identity to an amino acid sequence selected from the group consisting of SEQ ID NOs:64-71, wherein the modified plant, seed, cell, or plant part is capable of producing griseofulvin;
i) at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:72-77, wherein the modified plant, seed, cell, or plant part is capable of producing pleuromutilin;
j) at least two, at least three, or at least four nucleotide sequences encoding at least two, at least three, or at least four polypeptides having an amino acid sequence with at least about 85% sequence identity to an amino acid sequence selected from the group consisting of SEQ ID NOs:78-81, wherein the modified plant, seed, cell, or plant part is capable of producing pleuromutilin;
k) at least two, at least three, at least four, at least five, at least six, at least seven, at least eight, at least nine, or at least ten nucleotide sequences encoding at least two, at least three, at least four, at least five, at least six, at least seven, at least eight, at least nine, or at least ten polypeptides having an amino acid sequence with at least about 85% sequence identity to an amino acid sequence selected from the group consisting of SEQ ID NOs:82-91, wherein the modified plant, seed, cell, or plant part is capable of producing enfumafungin;
l) at least two, at least three, at least four, at least five, at least six, at least seven, or at least eight nucleotide sequences encoding at least two, at least three, at least four, at least five, at least six, at least seven, or at least eight polypeptides having an amino acid sequence with at least about 85% sequence identity to an amino acid sequence selected from the group consisting of SEQ ID NOs:92-99, wherein the modified plant, seed, cell, or plant part is capable of producing fusidic acid; or
m) at least two, at least three, or at least four nucleotide sequences encoding at least two, at least three, or at least four polypeptides having an amino acid sequence with at least about 85% sequence identity to an amino acid sequence selected from the group consisting of SEQ ID NOs:100-107, wherein the modified plant, seed, cell, or plant part is capable of producing psilocybin.

8. A plant commodity product produced from the modified plant, seed, cell, or plant part of claim 1, wherein said plant commodity product comprises said at least one recombinant polynucleotide molecule.

9. A medicinal compound produced from the modified plant, seed, cell, or plant part of claim 1.

10. The modified plant, seed, cell, or plant part of claim 1, wherein the at least one recombinant polynucleotide molecule comprises a heterologous promoter functional in a plant cell.

11. The modified plant, seed, cell, or plant part of claim 1, wherein the at least one recombinant polynucleotide molecule comprises a targeting nucleotide sequence or encodes a targeting polypeptide sequence.

12. The modified plant, seed, cell or plant part of claim 1, wherein the modified plant, seed, cell, or plant part is a Nicotiana benthamiana, a corn, or a soybean plant, seed, cell, or plant part.

13. A method for producing a modified plant cell, the method comprising introducing at least one heterologous polynucleotide molecule into a plant cell, wherein said heterologous polynucleotide molecule comprises at least one nucleotide sequence encoding at least one polypeptide having an amino acid sequence with at least about 85% sequence identity to an amino acid sequence selected from the group consisting of SEQ ID NOs:1-107.

14. The method of claim 13, the method further comprising selecting at least one plant cell comprising said at least one heterologous polynucleotide molecule.

15. The method of claim 13, the method further comprising regenerating at least one modified plant or plant part from the at least one selected plant cell or a descendant thereof comprising said at least one heterologous polynucleotide molecule.

16. The method of claim 15, wherein the modified plant or plant part comprises:
a) a first nucleotide sequence encoding a polypeptide having an amino acid sequence with at least about 85% sequence identity to SEQ ID NO:1, a second nucleotide sequence encoding a polypeptide having an amino acid sequence with at least about 85% sequence identity to SEQ ID NO:2, a third nucleotide sequence encoding a polypeptide having an amino acid sequence with at least about 85% sequence identity to SEQ ID NO:3, a fourth nucleotide sequence encoding a polypeptide having an amino acid sequence with at least about 85% sequence identity to SEQ ID NO:4, a fifth nucleotide sequence encoding a polypeptide having an amino acid sequence with at least about 85% sequence identity to SEQ ID NO:5, and a sixth nucleotide sequence encoding a polypeptide having an amino acid sequence with at least about 85% sequence identity to SEQ ID NO:6, wherein the modified plant, seed, cell, or plant part is capable of producing penicillin;
b) a first nucleotide sequence encoding a polypeptide having an amino acid sequence with at least about 85% sequence identity to SEQ ID NO:7, a second nucleotide sequence encoding a polypeptide having an amino acid sequence with at least about 85% sequence identity to SEQ ID NO:8, a third nucleotide sequence encoding a polypeptide having an amino acid sequence with at least about 85% sequence identity to SEQ ID NO:9, a fourth nucleotide sequence encoding a polypeptide having an amino acid sequence with at least about 85% sequence identity to SEQ ID NO:10, a fifth nucleotide sequence encoding a polypeptide having an amino acid sequence with at least about 85% sequence identity to SEQ ID NO:11, a sixth nucleotide sequence encoding a polypeptide having an amino acid sequence with at least about 85% sequence identity to SEQ ID NO:12, a seventh nucleotide sequence encoding a polypeptide having an amino acid sequence with at least about 85% sequence identity to SEQ ID NO:13, an eighth nucleotide sequence encoding a polypeptide having an amino acid sequence with at least about 85% sequence identity to SEQ ID NO:14 (MpaH), and a ninth nucleotide sequence encoding a polypeptide having an amino acid sequence with at least about 85% sequence identity to SEQ ID NO:15, wherein the modified plant, seed, cell, or plant part is capable of producing mycophenolic acid;
c) a first nucleotide sequence encoding a polypeptide having an amino acid sequence with at least about 85% sequence identity to SEQ ID NO:16, a second nucleotide sequence encoding a polypeptide having an amino acid sequence with at least about 85% sequence identity to SEQ ID NO:17, a third nucleotide sequence encoding a polypeptide having an amino acid sequence with at least about 85% sequence identity to SEQ ID NO:18, a fourth nucleotide sequence encoding a polypeptide having an amino acid sequence with at least about 85% sequence identity to SEQ ID NO:19, a fifth nucleotide sequence encoding a polypeptide having an amino acid sequence with at least about 85% sequence identity to SEQ ID NO:20, a sixth nucleotide sequence encoding a polypeptide having an amino acid sequence with at least about 85% sequence identity to SEQ ID NO:21, a seventh nucleotide sequence encoding a polypeptide having an amino acid sequence with at least about 85% sequence identity to SEQ ID NO:22, an eighth nucleotide sequence encoding a polypeptide having an amino acid sequence with at least about 85% sequence identity to SEQ ID NO:23, a ninth nucleotide sequence encoding a polypeptide having an amino acid sequence with at least about 85% sequence identity to SEQ ID NO:24, a tenth nucleotide sequence encoding a polypeptide having an amino acid sequence with at least about 85% sequence identity to SEQ ID NO:25, an eleventh nucleotide sequence encoding a polypeptide having an amino acid sequence with at least about 85% sequence identity to SEQ ID NO:26, and a twelfth nucleotide sequence encoding a polypeptide having an amino acid sequence with at least about 85% sequence identity to SEQ ID NO:27, wherein the modified plant, seed, cell, or plant part is capable of producing cyclosporin;
d) a first nucleotide sequence encoding a polypeptide having an amino acid sequence with at least about 85% sequence identity to SEQ ID NO:28, a second nucleotide sequence encoding a polypeptide having an amino acid sequence with at least about 85% sequence identity to SEQ ID NO:29, a third nucleotide sequence encoding a polypeptide having an amino acid sequence with at least about 85% sequence identity to SEQ ID NO:30, a fourth nucleotide sequence encoding a polypeptide having an amino acid sequence with at least about 85% sequence identity to SEQ ID NO:31, a fifth nucleotide sequence encoding a polypeptide having an amino acid sequence with at least about 85% sequence identity to SEQ ID NO:32, a sixth nucleotide sequence encoding a polypeptide having an amino acid sequence with at least about 85% sequence identity to SEQ ID NO:33, a seventh nucleotide sequence encoding a polypeptide having an amino acid sequence with at least about 85% sequence identity to SEQ ID NO:34, an eighth nucleotide sequence encoding a polypeptide having an amino acid sequence with at least about 85% sequence identity to SEQ ID NO:35, and a ninth nucleotide sequence encoding a polypeptide having an amino acid sequence with at least about 85% sequence identity to SEQ ID NO:36, wherein the modified plant, seed, cell, or plant part is capable of producing cephalosporin;
e) a first nucleotide sequence nucleotide sequence encoding a polypeptide having an amino acid sequence with at least about 85% sequence identity to SEQ ID NO:37, a second nucleotide sequence encoding a polypeptide having an amino acid sequence with at least about 85% sequence identity to SEQ ID NO:38, a third nucleotide sequence encoding a polypeptide having an amino acid sequence with at least about 85% sequence identity to SEQ ID NO:39, a fourth nucleotide sequence encoding a polypeptide having an amino acid sequence with at least about 85% sequence identity to SEQ ID NO:40, a fifth nucleotide sequence encoding a polypeptide having an amino acid sequence with at least about 85% sequence identity to SEQ ID NO:41, a sixth nucleotide sequence encoding a polypeptide having an amino acid sequence with at least about 85% sequence identity to SEQ ID NO:42, a seventh nucleotide sequence encoding a polypeptide having an amino acid sequence with at least about 85% sequence identity to SEQ ID NO:43, an eighth nucleotide sequence encoding a polypeptide having an amino acid sequence with at least about 85% sequence identity to SEQ ID NO:44, a ninth nucleotide sequence encoding a polypeptide having an amino acid sequence with at least about 85% sequence identity to SEQ ID NO:45, a tenth nucleotide sequence encoding a polypeptide having an amino acid sequence with at least about 85% sequence identity to SEQ ID NO:46, an eleventh nucleotide sequence encoding a polypeptide having an amino acid sequence with at least about 85% sequence identity to SEQ ID NO:47, and a twelfth nucleotide sequence encoding a polypeptide having an amino acid sequence with at least about 85% sequence identity to SEQ ID NO:48, wherein the modified plant, seed, cell, or plant part is capable of producing pneumocandin B0;
f) a first nucleotide sequence encoding a polypeptide having an amino acid sequence with at least about 85% sequence identity to SEQ ID NO:49, a second nucleotide sequence encoding a polypeptide having an amino acid sequence with at least about 85% sequence identity to SEQ ID NO:50, a third nucleotide sequence encoding a polypeptide having an amino acid sequence with at least about 85% sequence identity to SEQ ID NO:51, a fourth nucleotide sequence encoding a polypeptide having an amino acid sequence with at least about 85% sequence identity to SEQ ID NO:52, a fifth nucleotide sequence encoding a polypeptide having an amino acid sequence with at least about 85% sequence identity to SEQ ID NO:53, and a sixth nucleotide sequence encoding a polypeptide having an amino acid sequence with at least about 85% sequence identity to SEQ ID NO:54, wherein the modified plant, seed, cell, or plant part is capable of producing lovastatin;
g) a first nucleotide sequence encoding a polypeptide having an amino acid sequence with at least about 85% sequence identity to SEQ ID NO:55, a second nucleotide sequence encoding a polypeptide having an amino acid sequence with at least about 85% sequence identity to SEQ ID NO:56, a third nucleotide sequence encoding a polypeptide having an amino acid sequence with at least about 85% sequence identity to SEQ ID NO:57, a fourth nucleotide sequence encoding a polypeptide having an amino acid sequence with at least about 85% sequence identity to SEQ ID NO:58, a fifth nucleotide sequence encoding a polypeptide having an amino acid sequence with at least about 85% sequence identity to SEQ ID NO:59, a sixth nucleotide sequence encoding a polypeptide having an amino acid sequence with at least about 85% sequence identity to SEQ ID NO:60, a seventh nucleotide sequence encoding a polypeptide having an amino acid sequence with at least about 85% sequence identity to SEQ ID NO:61, an eighth nucleotide sequence encoding a polypeptide having an amino acid sequence with at least about 85% sequence identity to SEQ ID NO:62, and a ninth nucleotide sequence encoding a polypeptide having an amino acid sequence with at least about 85% sequence identity to SEQ ID NO:63, wherein the modified plant, seed, cell, or plant part is capable of producing compactin;
h) a first nucleotide sequence encoding a polypeptide having an amino acid sequence with at least about 85% sequence identity to SEQ ID NO:64, a second nucleotide sequence encoding a polypeptide having an amino acid sequence with at least about 85% sequence identity to SEQ ID NO:65, a third nucleotide sequence encoding a polypeptide having an amino acid sequence with at least about 85% sequence identity to SEQ ID NO:66, a fourth nucleotide sequence encoding a polypeptide having an amino acid sequence with at least about 85% sequence identity to SEQ ID NO:67, a fifth nucleotide sequence encoding a polypeptide having an amino acid sequence with at least about 85% sequence identity to SEQ ID NO:68, a sixth nucleotide sequence encoding a polypeptide having an amino acid sequence with at least about 85% sequence identity to SEQ ID NO:69, a seventh nucleotide sequence encoding a polypeptide having an amino acid sequence with at least about 85% sequence identity to SEQ ID NO:70), and an eighth nucleotide sequence encoding a polypeptide having an amino acid sequence with at least about 85% sequence identity to SEQ ID NO:71, wherein the modified plant, seed, cell, or plant part is capable of producing griseofulvin;
i) a first nucleotide sequence encoding a polypeptide having an amino acid sequence with at least about 85% sequence identity to SEQ ID NO:72, a second nucleotide sequence encoding a polypeptide having an amino acid sequence with at least about 85% sequence identity to SEQ ID NO:73, a third nucleotide sequence encoding a polypeptide having an amino acid sequence with at least about 85% sequence identity to SEQ ID NO:74, a fourth nucleotide sequence encoding a polypeptide having an amino acid sequence with at least about 85% sequence identity to SEQ ID NO:75, a fifth nucleotide sequence encoding a polypeptide having an amino acid sequence with at least about 85% sequence identity to SEQ ID NO:76, and a sixth nucleotide sequence encoding a polypeptide having an amino acid sequence with at least about 85% sequence identity to SEQ ID NO:77, wherein the modified plant, seed, cell, or plant part is capable of producing pleuromutilin;
j) a first nucleotide sequence encoding a polypeptide having an amino acid sequence with at least about 85% sequence identity to SEQ ID NO:78, a second nucleotide sequence encoding a polypeptide having an amino acid sequence with at least about 85% sequence identity to SEQ ID NO:79, a third nucleotide sequence encoding a polypeptide having an amino acid sequence with at least about 85% sequence identity to SEQ ID NO:80, and a fourth nucleotide sequence encoding a polypeptide having an amino acid sequence with at least about 85% sequence identity to SEQ ID NO:81, wherein the modified plant, seed, cell, or plant part is capable of producing pleuromutilin;
k) a first nucleotide sequence encoding a polypeptide having an amino acid sequence with at least about 85% sequence identity to SEQ ID NO:82, a second nucleotide sequence encoding a polypeptide having an amino acid sequence with at least about 85% sequence identity to SEQ ID NO:83, a third nucleotide sequence encoding a polypeptide having an amino acid sequence with at least about 85% sequence identity to SEQ ID NO:84, a fourth nucleotide sequence encoding a polypeptide having an amino acid sequence with at least about 85% sequence identity to SEQ ID NO:85, a fifth nucleotide sequence encoding a polypeptide having an amino acid sequence with at least about 85% sequence identity to SEQ ID NO:86, a sixth nucleotide sequence encoding a polypeptide having an amino acid sequence with at least about 85% sequence identity to SEQ ID NO:87, a seventh nucleotide sequence encoding a polypeptide having an amino acid sequence with at least about 85% sequence identity to SEQ ID NO:88, an eighth nucleotide sequence encoding a polypeptide having an amino acid sequence with at least about 85% sequence identity to SEQ ID NO:89, a ninth nucleotide sequence encoding a polypeptide having an amino acid sequence with at least about 85% sequence identity to SEQ ID NO:90, and a tenth nucleotide sequence encoding a polypeptide having an amino acid sequence with at least about 85% sequence identity to SEQ ID NO:91, wherein the modified plant, seed, cell, or plant part is capable of producing enfumafungin;
l) a first nucleotide sequence encoding a polypeptide having an amino acid sequence with at least about 85% sequence identity to SEQ ID NO:92, a second nucleotide sequence encoding a polypeptide having an amino acid sequence with at least about 85% sequence identity to SEQ ID NO:93, a third nucleotide sequence encoding a polypeptide having an amino acid sequence with at least about 85% sequence identity to SEQ ID NO:94, a fourth nucleotide sequence encoding a polypeptide having an amino acid sequence with at least about 85% sequence identity to SEQ ID NO:95, a fifth nucleotide sequence encoding a polypeptide having an amino acid sequence with at least about 85% sequence identity to SEQ ID NO:96, a sixth nucleotide sequence encoding a polypeptide having an amino acid sequence with at least about 85% sequence identity to SEQ ID NO:97, a seventh nucleotide sequence encoding a polypeptide having an amino acid sequence with at least about 85% sequence identity to SEQ ID NO:98, and an eighth nucleotide sequence encoding a polypeptide having an amino acid sequence with at least about 85% sequence identity to SEQ ID NO:99, wherein the modified plant, seed, cell, or plant part is capable of producing fusidic acid; or
m) a first nucleotide sequence encoding a polypeptide having an amino acid sequence with at least about 85% sequence identity to SEQ ID NO:100 or SEQ ID NO:101, a second nucleotide sequence encoding a polypeptide having an amino acid sequence with at least about 85% sequence identity to SEQ ID NO:102 or SEQ ID NO:103, a third nucleotide sequence encoding a polypeptide having an amino acid sequence with at least about 85% sequence identity to SEQ ID NO:104 or SEQ ID NO:105, and a fourth nucleotide sequence encoding a polypeptide having an amino acid sequence with at least about 85% sequence identity to SEQ ID NO:106 or SEQ ID NO:107, wherein the modified plant, seed, cell, or plant part is capable of producing psilocybin.

17. The method of claim 13, wherein the at least one heterologous polynucleotide molecule comprises at least one nucleotide sequence encoding at least one polypeptide having an amino acid sequence with at least about 90%, about 91%, about 92%, about 93%, about 94%, about 95%, about 96%, about 97%, about 98%, about 99%, or 100% sequence identity to an amino acid sequence selected from the group consisting of SEQ ID NOs:1-107.

18. The method of claim 13, the method comprising introducing at least two, at least three, at least four, at least five, at least six, at least seven, at least eight, at least nine, at least ten, at least eleven, or at least twelve heterologous polynucleotide molecules into said plant cell.

19. The method of claim 15, wherein the modified plant or plant part comprises:
a) at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:1-6, wherein the modified plant, seed, cell, or plant part is capable of producing penicillin;
b) at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:7-15, wherein the modified plant, seed, cell, or plant part is capable of producing mycophenolic acid;
c) at least two, at least three, at least four, at least five, at least six, at least seven, at least eight, at least nine, at least ten, at least eleven, or at least twelve nucleotide sequences encoding at least two, at least three, at least four, at least five, at least six, at least seven, at least eight, at least nine, at least ten, at least eleven, or at least twelve polypeptides having an amino acid sequence with at least about 85% sequence identity to an amino acid sequence selected from the group consisting of SEQ ID NOs:16-27, wherein the modified plant, seed, cell, or plant part is capable of producing cyclosporin;
d) at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:28-36, wherein the modified plant, seed, cell, or plant part is capable of producing cephalosporin;
e) at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:37-48, wherein the modified plant, seed, cell, or plant part is capable of producing pneumocandin B0;
f) at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:49-54, wherein the modified plant, seed, cell, or plant part is capable of producing lovastatin;
g) at least two, at least three, at least four, at least five, at least six, at least seven, at least eight, or at least nine nucleotide sequences encoding at least two, at least three, at least four, at least five, at least six, at least seven, at least eight, or at least nine polypeptides having an amino acid sequence with at least about 85% sequence identity to an amino acid sequence selected from the group consisting of SEQ ID NOs:55-63, wherein the modified plant, seed, cell, or plant part is capable of producing compactin;
h) at least two, at least three, at least four, at least five, at least six, at least seven, or at least eight nucleotide sequences encoding at least two, at least three, at least four, at least five, at least six, at least seven, or at least eight polypeptides having an amino acid sequence with at least about 85% sequence identity to an amino acid sequence selected from the group consisting of SEQ ID NOs:64-71, wherein the modified plant, seed, cell, or plant part is capable of producing griseofulvin;
i) at least two, at least three, at least four, at least five, or at least six nucleotide sequences encoding at least two, at least three, at least four, at least five, or at least six polypeptides having an amino acid sequence with at least about 85% sequence identity to an amino acid sequence selected from the group consisting of SEQ ID NOs:72-77, wherein the modified plant, seed, cell, or plant part is capable of producing pleuromutilin;
j) at least two, at least three, or at least four nucleotide sequences encoding at least two, at least three, or at least four polypeptides having an amino acid sequence with at least about 85% sequence identity to an amino acid sequence selected from the group consisting of SEQ ID NOs:78-81, wherein the modified plant, seed, cell, or plant part is capable of producing pleuromutilin;
k) at least two, at least three, at least four, at least five, at least six, at least seven, at least eight, at least nine, or at least ten nucleotide sequences encoding at least two, at least three, at least four, at least five, at least six, at least seven, at least eight, at least nine, or at least ten polypeptides having an amino acid sequence with at least about 85% sequence identity to an amino acid sequence selected from the group consisting of SEQ ID NOs:82-91, wherein the modified plant, seed, cell, or plant part is capable of producing enfumafungin;
l) at least two, at least three, at least four, at least five, at least six, at least seven, or at least eight nucleotide sequences encoding at least two, at least three, at least four, at least five, at least six, at least seven, or at least eight polypeptides having an amino acid sequence with at least about 85% sequence identity to an amino acid sequence selected from the group consisting of SEQ ID NOs:92-99, wherein the modified plant, seed, cell, or plant part is capable of producing fusidic acid; or
m) at least two, at least three, or at least four nucleotide sequences encoding at least two, at least three, or at least four polypeptides having an amino acid sequence with at least about 85% sequence identity to an amino acid sequence selected from the group consisting of SEQ ID NOs:100-107, wherein the modified plant, seed, cell, or plant part is capable of producing psilocybin.

20. The method of claim 13, wherein the at least one heterologous polynucleotide molecule comprises a promoter functional in a plant cell.

21. The method of claim 13, wherein the at least one heterologous polynucleotide molecule comprises a targeting nucleotide sequence or encodes a targeting polypeptide sequence.

22. The method of claim 13, wherein the plant cell is a Nicotiana benthamiana, a corn, or a soybean plant cell.
